(19) United States
(12) Patent Application Publication
(10) Pub. No.: US 2011/0158985 A1
(43) Pub. Date: Jun. 30, 2011

(54) METHODS OF PREVENTING AND TREATING RSV INFECTIONS AND RELATED CONDITIONS

(75) Inventors: GENEVIEVE LOSONSKY, PHOENIX, MD (US); EDWARD M. CONNOR, GAITHERSBURG, MD (US); JAMES F. YOUNG, POTOMAC, MD (US); HERREN WU, BOYDS, MD (US); WILLIAM DALL'ACQUA, GAITHERSBURG, MD (US)

(73) Assignee: MEDIMMUNE, LLC, GAITHERSBURG, MD (US)

(21) Appl. No.: 12/969,514

(22) Filed: Dec. 15, 2010

Related U.S. Application Data
(63) Continuation of application No. 12/559,375, filed on Sep. 14, 2009, now abandoned.
Application No. 12/559,375 is a continuation of application No. 11/263,230, filed on Oct. 31, 2005, now abandoned.
(60) Provisional application No. 60/623,821, filed on Oct. 29, 2004.
(60) Provisional application No. 60/675,724, filed on Apr. 27, 2005.
(60) Provisional application No. 60/681,233, filed on May 13, 2005.
(60) Provisional application No. 60/718,719, filed on Sep. 21, 2005.
(60) Provisional application No. 60/727,043, filed on Oct. 14, 2005.
(60) Provisional application No. 60/727,042, filed on Oct. 14, 2005.

(51) Int. Cl. A61K 39/42; A61P 31/14

(52) U.S. Cl. 424/133.1

(57) ABSTRACT

The present invention provides methods for preventing, managing, treating and/or ameliorating a Respiratory Syncytial Virus (RSV) infection (e.g., acute RSV disease, or a RSV upper respiratory tract infection (URI) and/or lower respiratory tract infection (LRI)), otitis media (preferably, stemming from, caused by or associated with a RSV infection, such as a RSV URI and/or LRI), and/or a symptom or respiratory condition relating thereto (e.g., asthma, wheezing, and/or reactive airway disease (RAD)) in a subject, comprising administering to said human an effective amount of one or more antibodies that immunospecifically bind to one or more RSV antigens with a high affinity and/or high avidity. In some embodiments, one or more antibodies comprise a modified IgG constant domain, or FcRn-binding fragment thereof resulting in longer in vivo serum half-life. In particular embodiments the methods of the invention comprising administering to subject an effective amount of one or more modified antibodies that immunospecifically bind to one or more RSV antigens with an association rate (kₒₙ) of at least 2×10⁵ M⁻¹ s⁻¹ and a dissociation rate (k_{off}) of less than 5×10⁻⁴ s⁻¹.

### CROSS-REFERENCE TO RELATED APPLICATIONS

This application claims priority to each of U.S. Provisional No. 60/623,821 (Attorney Docket No. 10271-149-888) filed Oct. 29, 2004 by Genevieve Losonsky entitled “Methods of Administering/Dosing Anti-RSV Antibodies for the Prophylaxis and Treatment of Upper Respiratory Tract and Middle Ear Infections;” U.S. Provisional No. 60/675,724 (Attorney Docket No. 10271-156-888) filed Apr. 27, 2005 by Genevieve Losonsky entitled “Methods of Administering/Dosing Anti-RSV Antibodies for Prophylaxis and Treatment of Upper Respiratory Tract and Middle Ear Infections;” U.S. Provisional No. 60/681,233 (Attorney Docket No. 10271-162-888) filed May 13, 2005 by Genevieve Losonsky entitled “Methods of Administering/Dosing Anti-RSV Antibodies for Prophylaxis and Treatment of RSV Infections and Respiratory Conditions;” U.S. Provisional No. 60/718,719 (Attorney Docket No. RS108P4) filed Sep. 21, 2005 by Genevieve Losonsky entitled “Methods of Administering/Dosing Anti-RSV Antibodies for Prophylaxis and Treatment of RSV Infections and Respiratory Conditions;” U.S. Provisional No. 60/727,043 (Attorney Docket No. 10271-165-888) filed Oct. 14, 2005 entitled “Methods of Preventing and Treating RSV Infections and Related Conditions;” and U.S. Provisional No. 60/727,042 (Attorney Docket No. 10271-174-888) filed Oct. 14, 2005 by Genevieve Losonsky entitled “Methods of Administering/Dosing Anti-RSV Antibodies for Prophylaxis and Treatment of RSV Infections and Respiratory Conditions;” each of which is incorporated herein by reference in its entirety.

### 1. INTRODUCTION

The present invention provides antibodies that immuno specifically bind to a respiratory syncytial virus (RSV) antigen with high affinity and/or high avidity. In some embodiments, the antibodies are modified antibodies that have increased in vivo half lives due to the presence of an IgG constant domain or a portion thereof that binds FcRn, having one or more amino acid modifications that increase the affinity of the constant domain, or fragment thereof, for the FcRn. The invention also provides methods of preventing, managing, treating and/or ameliorating a RSV infection (e.g., acute RSV disease, or a RSV upper respiratory tract infection (URI) and/or lower respiratory tract infection (LRI)), said methods comprising administering to a human subject an effective amount of one or more of the antibodies (e.g., one or more modified or unmodified antibodies) provided herein. The present invention also provides methods for preventing, treating, managing, and/or ameliorating an ear infection (such as otitis media), or a symptom thereof, which is associated with or caused by a RSV infection. The present invention further provides methods for preventing, treating, managing, and/or ameliorating respiratory conditions, including, but not limited to, asthma, wheezing, reactive airway disease (RAD), or a combination thereof, which are associated with or caused by a RSV infection.

### 2. BACKGROUND OF THE INVENTION

### 2.1 Respiratory Syncytial Virus

Respiratory infections are common infections of the upper respiratory tract (e.g., nose, ears, sinuses, and throat) and lower respiratory tract (e.g., trachea, bronchial tubes, and lungs). Symptoms of upper respiratory infection include runny or stuffy nose, irritability, restlessness, poor appetite, decreased activity level, coughing, and fever. Viral upper respiratory infections cause and/or are associated with sore throats, colds, croup, and the flu. Clinical manifestations of a lower respiratory infection include shallow coughing that produces sputum in the lungs, fever, and difficulty breathing.

Respiratory syncytial virus (RSV) is one of the leading causes of respiratory disease worldwide. In the United States, it is responsible for tens of thousands of hospitalizations and thousands of deaths per year (see Black, C. P., Resp. Care 2003 48(3):209-31 for a recent review of the biology and management of RSV). Infants and children are most at risk for serious RSV infections which migrate to the lower respiratory system, resulting in pneumonia or bronchiolitis. In fact, 80% of childhood bronchiolitis cases and 50% of infant pneumonias are attributable to RSV. The virus is so ubiquitous and highly contagious that almost all children have been infected by two years of age. Although infection does not produce lasting immunity, reinfections tend to be less severe so that in older children and healthy adults RSV manifests itself as a cold or flu-like illness affecting the upper and/or lower respiratory system, without progressing to serious lower respiratory tract involvement. However, RSV infections can become serious in elderly or immunocompromised adults. (Evans, A. S., eds., 1989, Viral Infections of Humans. Epidemiology and Control, 3^{rd} ed., Plenum Medical Book, New York at pages 525-544; Falsey, A. R., 1991, Infect. Control Hosp. Epidemiol. 12:602-608; and Garvie et al., 1980, Br. Med. J. 281:1253-1254; Hertz et al., 1989, Medicine 68:269-281).

At present, there is no vaccine against RSV, nor is there any commercially available effective treatment. Recent clinical data has failed to support the early promise of the antiviral agent ribavirin, which is the only drug approved for treatment of RSV infection (Black, C. P., Resp. Care 2003 48(3):209-31). Consequently, the American Academy of Pediatrics issued new guidelines suggesting that use of ribavirin be restricted to only the most severe cases (Committee on Infectious Disease, American Academy of Pediatrics. 1996. Pediatrics 97:137-140; Randolph, A. G., and E. E. Wang., 1996, Arch. Pediatr. Adolesc. Med. 150:942-947).

While a vaccine or commercially available effective treatment are not yet available, some success has been achieved in the area of prevention for infants at high risk of serious lower respiratory tract disease caused by RSV, as well as a reduction of LRI. In particular, there are two immunoglobulin-based therapies approved to protect high-risk infants from serious LRI: RSV-IGIV (RSV-immunoglobulin intravenous, also known as RespiGam™) and palivizumab (SYNAGIS®). However, neither RSV-IGIV nor palivizumab has been approved for use other than as a prophylactic agent for serious lower respiratory tract acute RSV disease.

RSV is easily spread by physical contact with contaminated secretions. The virus can survive for at least half an hour on hands and for hours on countertops and used tissues. The highly contagious nature of RSV is evident from the risk factors associated with contracting serious infections. One of the greatest risk factors is hospitalization, where in some cases in excess of 50% of the staff on pediatric wards were found to be infected (Black, C. P., Resp. Care 2003 48(3):209-31). Up to 20% of these adult infections are asymptomatic but still produce substantial shedding of the virus. Other risk factors include attendance at day care centers, crowded living conditions, and the presence of school-age siblings in the home. Importantly, an agent that is effective at clearing the virus from the upper and/or lower respiratory tract is likely to be effective in preventing its transmission. Thus, one promising approach to preventing serious RSV infections and subsequent disease is the development of therapies to either clear the virus or reduce viral load from the upper respiratory tract, thereby preventing the progression of the virus to the lower respiratory tract.

Although RSV-IVIG and palivizumab represent significant advances in the prevention of lower respiratory tract acute RSV disease and mitigation of lower respiratory tract infection, neither has demonstrated efficacy at permissible doses against the virus in the upper respiratory tract and therefore the possible prevention of progression of RSV infection to the lower respiratory tract. In fact, RSV-IVIG failed to clear nasal RSV when administered as a nasal spray in amounts that were effective to clear pulmonary RSV in every animal of the treatment group (Prince et al., U.S. Pat. No. 4,800,078, issued Jan. 24, 1989). The interperitoneal route of administration also failed to clear RSV from the upper respiratory tract with the same efficacy as the lower respiratory tract. It has recently been noted that the immune response elicited by upper respiratory tract infections differs from that induced by lower respiratory infections (van Benten I. J. et al., J. Med. Virol. 2003 October; 71(2):290-7). Thus, a need exists for the prevention of acute RSV disease in the lungs via treatment of RSV URI and/or prevention and/or reduction of the progression of the virus to the lower respiratory tract.

### 2.2 Otitis Media

Otitis media is an infection or inflammation of the middle ear. This inflammation often begins when infections that cause sore throats, colds, or other respiratory or breathing problems spread to the middle ear. These can be viral or bacterial infections. RSV is the principal virus that has been correlated with otitis media. Seventy-five percent of children experience at least one episode of otitis media by their third birthday. Almost half of these children will have three or more ear infections during their first 3 years. It is estimated that medical costs and lost wages because of otitis media amount to $5 billion a year in the United States (Gates G A, 1996, Cost-effectiveness considerations in otitis media treatment. Otolaryngol Head Neck Sur. 114 (4): 525-530). Although otitis media is primarily a disease of infants and young children, it can also affect adults.

Otitis media not only causes severe pain but may result in serious complications if it is not treated. An untreated infection can travel from the middle ear to the nearby parts of the head, including the brain. Although the hearing loss caused by otitis media is usually temporary, untreated otitis media may lead to permanent hearing impairment. Persistent fluid in the middle ear and chronic otitis media can reduce a child's hearing at a time that is critical for speech and language development. Children who have early hearing impairment from frequent ear infections are likely to have speech and language disabilities.

Although many physicians recommend the use of antibiotics for the treatment of ear infections, antibiotic resistance has become an important problem in effective treatment of the disease and do not treat otitis media of viral etiology. Further, new therapies are needed to prevent or treat viral infections that are associated with otitis media, particularly RSV.

### 2.3 Asthma and Reactive Airway Disease (RAD)

About 12 million people in the U.S. have asthma and it is the leading cause of hospitalization for children. The Merck Manual of Diagnosis and Therapy (17th ed., 1999).

Asthma is an inflammatory disease of the lung that is characterized by airway hyperresponsiveness (“AHR”), bronchoconstriction (i.e., wheezing), eosinophilic inflammation, mucus hypersecretion, subepithelial fibrosis, and elevated IgE levels. Asthmatic attacks can be triggered by environmental triggers (e.g., acarids, insects, animals (e.g., cats, dogs, rabbits, mice, rats, hamsters, guinea pigs, mice, rats, and birds), fungi, air pollutants (e.g., tobacco smoke), irritant gases, fumes, vapors, aerosols, chemicals, or pollen), exercise, or cold air. The cause(s) of asthma is unknown. However, it has been speculated that family history of asthma (London et al., 2001, Epidemiology 12(5):577-83), early exposure to allergens, such as dust mites, tobacco smoke, and cockroaches (Melen et al., 2001, 56(7):646-52), and respiratory infections (Wenzel et al., 2002, Am J Med, 112(8):672-33 and Lin et al., 2001, J Microbiol Immuno Infect, 34(4):259-64), such as RSV, may increase the risk of developing asthma. A review of asthma, including risk factors, animal models, and inflammatory markers can be found in O'Byrne and Postma (1999), Am. J. Crit. Care. Med. 159:S41-S66, which is incorporated herein by reference in its entirety.

Current therapies are mainly aimed at managing asthma and include the administration of β-adrenergic drugs (e.g., epinephrine and isoproterenol), theophylline, anticholinergic drugs (e.g., atropine and ipratorpium bromide), corticosteroids, and leukotriene inhibitors. These therapies are associated with side effects such as drug interactions, dry mouth, blurred vision, growth suppression in children, and osteoporosis in menopausal women. Cromolyn and nedocromil are administered prophylatically to inhibit mediator release from inflammatory cells, reduce airway hyperresponsiveness, and block responses to allergens. However, there are no current therapies available that prevent the development of asthma in subjects at increased risk of developing asthma. Thus, new therapies with fewer side effects and better prophylactic and/or therapeutic efficacy are needed for asthma.

Reactive airway disease is a broader (and often times synonymous) characterization for asthma-like symptoms, and is generally characterized by chronic cough, sputum production, wheezing or dyspenea.

### 2.4 Wheezing

Wheezing (also known as sibilant rhonchi) is generally characterized by a noise made by air flowing through narrowed breathing tubes, especially the smaller, tight airways located deep within the lung. It is a common symptom of RSV infection, and secondary RSV conditions such as asthma and brochiolitis. The clinical importance of wheezing is that it is an indicator of airway narrowing, and it may indicate difficulty breathing.

Wheezing is most obvious when exhaling (breathing out), but may be present during either inspiration (breathing in) or exhalation. Wheezing most often comes from the small bronchial tubes (breathing tubes deep in the chest), but it may originate if larger airways are obstructed.

Citation or discussion of a reference herein shall not be construed as an admission that such is prior art to the present invention.

### 3. SUMMARY OF THE INVENTION

The present invention provides antibodies with a high affinity and/or high avidity for a RSV antigen, such as RSV F protein, that are effective in reducing upper as well as lower respiratory tract RSV infections at dosages less than or about equal to the dosage of palivizumab used to prevent only lower respiratory tract infection.

Additionally, the present invention provides an antibody with high affinity and/or high avidity for a RSV antigen (e.g., RSV F antigen) for the prevention, treatment and/or amelioration an upper respiratory tract RSV infection (URI) and/or lower respiratory tract RSV infection (LRI), wherein the antibody comprises one or more amino acid modifications in the IgG constant domain, or FcRn-binding fragment thereof (preferably a modified Fc domain or hinge-Fc domain) that increases the in vivo half-life of the IgG constant domain, or FcRn-binding fragment thereof (e.g., Fc or hinge-Fc domain), and any molecule attached thereto, and increases the affinity of the IgG, or FcRn-binding fragment thereof containing the modified region, for FcRn (i.e., a “modified antibody”). The amino acid modifications may be any modification of a residue (and, in some embodiments, the residue at a particular position is not modified but already has the desired residue), preferably at one or more of residues 251-256, 285-290, 308-314, 385-389, and 428-436. In other embodiments, the antibody comprises a tyrosine at position 252 (252Y), a threonine at position 254 (254T), and/or a glutamic acid at position 256 (256E) (numbering of the constant domain according to the EU index in Kabat et al. (1991). Sequences of proteins of immunological interest. (U.S. Department of Health and Human Services, Washington, D.C.) 5^{th} ed. (“Kabat et al.”)) in the constant domain, or FcRn-binding fragment thereof. In other embodiments, the antibodies comprise 252Y, 254T, and 256E (see EU index in Kabat et al., supra) in the constant domain, or FcRn-binding fragment thereof (hereafter “YTE” see, e.g., FIG. 35).

The present invention provides methods of preventing, managing, treating, neutralizing, and/or ameliorating a RSV infection (e.g., acute RSV disease, or a RSV URI and/or LRI) in a subject comprising administering to said subject an effective amount of an antibody provided herein (a modified or unmodified antibody) which immunospecifically binds to a RSV antigen with high affinity and/or high avidity. Because a lower and/or longer-lasting serum titer of the antibodies of the invention will be more effective in the prevention, management, treatment and/or amelioration of a RSV infection (e.g., acute RSV disease, or a RSV URI and/or LRI) than the effective serum titer of known antibodies (e.g., palivizumab), lower and/or fewer doses of the antibody can be used to achieve a serum titer effective for the prevention, management, treatment and/or amelioration of a RSV infection (e.g., acute RSV disease, or a RSV URI and/or LRI), for example one or more doses per RSV season. The use of lower and/or fewer doses of an antibody of the invention that immunospecifically binds to a RSV antigen reduces the likelihood of adverse effects and are safer for administration to, e.g., infants, over the course of treatment (for example, due to lower serum titer, longer serum half-life and/or better localization to the upper respiratory tract and/or lower respiratory tract as compared to known antibodies (e.g., palivizumab).

Accordingly, the invention provides antibodies, and methods of using the antibodies, having an increased potency and/or having increased affinity and/or increased avidity for a RSV antigen (preferably RSV F antigen) as compared to a known RSV antibody (e.g., palivizumab). In some embodiments, the antibodies comprise a modified IgG constant domain, or FcRn-binding fragment thereof (preferably, Fc domain or hinge-Fc domain), which results in increased in vivo serum half-life (i.e., a modified antibody of the invention), as compared to antibodies that do not comprise a modified IgG constant domain, or FcRn-binding fragment thereof, e.g., as compared to an the antibody that does not comprise the modification (i.e., an unmodified antibody), or as compared to another RSV antibody, such as palivizumab. In certain embodiments, the antibody is administered once per RSV season.

In one aspect, the invention provides a method of preventing, managing, treating and/or ameliorating a RSV infection (e.g., acute RSV disease, or a RSV URI and/or LRI) and/or a symptom or respiratory condition relating thereto (e.g., asthma, wheezing, and/or RAD), the method comprising administering to a subject (e.g., in need thereof) an effective amount of an antibody described herein (i.e., an antibody of the invention), such as an antibody that does not comprise a modified IgG constant domain (e.g., MEDI-524) or such as a modified antibody that does comprise a modified IgG constant domain (e.g., MEDI-524-YTE). In some embodiments, both upper and lower respiratory tract RSV infections and/or acute RSV disease, can be managed, treated, or ameliorated. In other embodiments, the symptom or respiratory condition relating to the RSV infection is asthma, wheezing, RAD, or a combination thereof. The methods of the invention also encompass the prevention of secondary conditions associated with or caused by a RSV URI and/or LRI.

In a second aspect, the invention provides methods of preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD), the method comprising administering to a subject an effective amount of one or more antibodies of the invention and an effective amount of one or more therapies other than an antibody of the invention. In some embodiments, the antibody is a modified antibody (e.g., MEDI-524-YTE).

In a third aspect, the invention provides methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) in a subject, said methods comprising administering to said subject at least a first dose of an antibody of the invention so that said subject has a serum antibody titer of from about 0.1 μg/ml to about 800 μg/ml. In some embodiments, the serum antibody titer is present in the subject for several hours, several days, several weeks, and/or several months. In one embodiment, the first dose of an antibody of the invention is administered in a sustained release formulation, and/or by pulmonary or intranasal delivery. In certain embodiments, the antibody is a modified antibody.

In a fourth aspect, the invention provides methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) in a subject, said methods comprising administering to said subject a first dose of an antibody of the invention so that said subject has a nasal turbinate and/or nasal secretion antibody concentration of from about 0.01 μg/ml about 2.5 μg/ml. In some embodiments, the nasal turbinate and/or nasal secretion antibody concentration is present in the subject for several hours, several days, several weeks, and/or several months. The first dose of an antibody of the invention can be a prophylactically or therapeutically effective dose. In one embodiment, the first dose of an antibody of the invention is administered in a sustained release formulation, and/or by pulmonary or intranasal delivery. In certain embodiments, the antibody is a modified antibody.

In a fifth aspect, the invention provides methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) in a subject, said methods comprising administering an effective amount of an antibody of the invention (e.g., a modified antibody), wherein the effective amount results in a reduction in RSV titer in the nasal turbinate and/or nasal secretion. The reduction of RSV titer in the nasal turbinate and/or nasal secretion may be as compared to a negative control (such as placebo), as compared to another therapy (including, but not limited to treatment with palivizumab), or as compared to the titer in the patient prior to antibody administration.

In a sixth aspect, the invention provides methods of neutralizing RSV in the upper and/or lower respiratory tract or in the middle ear using an antibody of the invention to achieve a prophylactically or therapeutically effective serum titer. In some embodiments, the antibody is a modified antibody.

In a seventh aspect, the invention provides high potency antibodies, including modified antibodies, that can be used in accordance with the methods of the invention that have a high affinity and/or high avidity for a RSV antigen, such as the RSV F antigen. In one embodiment, the antibodies have a several-fold higher affinity for a RSV antigen than a known anti-RSV antibody (e.g., palivizumab) as assessed by techniques described herein or known to one of skill in the art (e.g., a BIAcore assay).

In an eighth aspect, the antibodies (including, e.g., modified antibodies) used in accordance with the methods of the invention immunospecifically bind to one or more RSV antigens (e.g., RSV F antigen) and have an association rate constant or kₒₙ, rate (antibody (Ab)+antigen (Ag)—kₒₙ—>Ab−Ag) of from about 10⁵ M⁻¹ s⁻¹ to about 10¹⁰ M⁻¹ s⁻¹ In some embodiments, the antibody is a high potency antibody having a kₒₙ of from about 10⁵ M⁻¹ s⁻¹ to about 10⁸ M⁻¹ s⁻¹, preferably about 2.5×10⁵ or 5×10⁵ M⁻¹ s⁻¹, and more preferably about 7.5×10⁵ M⁻¹s⁻¹. Such antibodies may also have a high affinity (e.g., about 10⁹ M⁻¹) or may have a lower affinity. In one embodiment, the antibodies that can be used in accordance with the methods of the invention immunospecifically bind to a RSV antigen (e.g., RSV F antigen) and have a kₒₙ rate that is at least 1.5-fold higher than a known anti-RSV antibody (e.g., palivizumab).

In a ninth aspect, the antibodies (including, e.g., modified antibodies) used in accordance with the methods of the invention immunospecifically bind to one or more RSV antigens (e.g., RSV F antigen) and have a k_{off} rate (Ab−Ag—K_{off}—>Ab+Ag) of from less than 5×10⁻¹ s⁻¹ to less than 10×10⁻¹⁰ s⁻¹. In one embodiment, the antibodies used in accordance with the methods of the invention immunospecifically bind to a RSV antigen (e.g., RSV F antigen) and have a k_{off} rate that is at least 1.5-fold lower than a known anti-RSV antibody (e.g., palivizumab).

In a tenth aspect, the antibodies (including, e.g., modified antibodies) that can be used in accordance with the methods of the invention immunospecifically bind to one or more RSV antigens (e.g., RSV F antigen) and have an affinity constant or Kₐ (kₒₙ/k_{off}) of from about 10² M⁻¹ to about 5×10¹⁵M⁻¹, preferably at least 10⁴ M⁻¹. In some embodiments, the antibody is a high potency antibody having a Kₐ of about 10⁹ M⁻¹, preferably about 10¹⁰ M⁻¹, and more preferably about 10¹¹ M⁻¹.

In an eleventh aspect, the antibodies, including, e.g., modified antibodies of the invention, used in accordance with the methods of the invention immunospecifically bind to one or more RSV antigens (e.g., RSV F antigen) and have a dissociation constant or K_{d} (k_{off}/kₒₙ) of from about 5×10⁻²M to about 5×10⁻¹⁶M.

In a twelfth aspect, the antibodies that can be used in accordance with the methods of the invention immunospecifically bind to one or more RSV antigens (e.g., RSV F antigen) have a dissociation constant (K_{d}) of between about 25 pM and about 3000 pM as assessed using an assay described herein or known to one of skill in the art (e.g., a BIAcore assay).

In a thirteenth aspect, the antibodies, including, e.g., modified antibodies of the invention, used in accordance with the methods of the invention immunospecifically bind to one or more RSV antigens (e.g., RSV F antigen) and have a median inhibitory concentration (IC₅₀) of about 6 nM to about 0.01 nM in an in vitro microneutralization assay. In certain embodiments, the microneutralization assay is a microneutralization assay described herein (for example, as described in Examples 6.4, 6.8, and 6.18 herein) or as in Johnson et al., 1999, J. Infectious Diseases 180:35-40. In some embodiments, the antibody has an IC₅₀ of less than 3 nM, preferably less than 1 nM in an in vitro microneutralization assay.

In a fourteenth aspect, the antibodies of the invention (e.g., modified antibodies) can be used to prevent, manage, treat and/or ameliorate a RSV infection (e.g., acute RSV disease or a RSV URI and/or LRI), otitis media (preferably stemming from, caused by or associated with a RSV infection, such as a RSV URI and/or LRI), and/or a symptom or respiratory condition relating thereto (e.g., asthma, wheezing and/or RAD), said method comprising intranasally administering an effective amount of the antibodies of the invention, wherein the prevention, management, treatment and/or amelioration is post-infection.

In a fifteenth aspect, antibodies, including, e.g., modified antibodies, of the invention have reduced or no cross-reactivity with human tissue. In certain embodiments, an antibody of the invention (e.g., a modified MEDI-524 antibody, such as MEDI-524-YTE) has reduced cross-reactivity with human tissue (e.g., skin and/or lung tissue) as compared to another anti-RSV antibody (such as A4B4).

In a sixteenth aspect, the invention provides methods of prophylactically administering one or more antibodies (e.g., a modified or unmodified antibody) of the invention to a subject (e.g., an infant, an infant born prematurely, an immunocompromised subject, a medical worker). In some embodiments, an antibody of the invention is administered to a subject so as to prevent a RSV infection from being transmitted from one individual to another, or to lessen the infection that is transmitted. In some embodiments, the subject has been exposed to (and may or may not be asymptomatic), or is likely to be exposed to another individual having RSV infection. Preferably the antibody is administered to the subject intranasally once or more times per day (e.g., one time, two times, four times, etc.) for a period of about one to two weeks after potential or actual exposure to the RSV-infected individual. In certain embodiments, the antibody is administered at a dose of between about 60 mg/kg to about 0.025 mg/kg, and more preferably from about 0.025 mg/kg to 15 mg/kg.

In preferred embodiments, the methods of the invention encompass the use of antibodies comprising the VH domain and/or VL domain of A4B4L1FR-S28R (MEDI-524) (FIG. 13). In preferred embodiments, the methods of the invention encompass the use of antibodies comprising the VH chain and/or VL chain of A4B4L1FR-S28R (MEDI-524) (FIG. 13). In certain embodiments, the antibody comprises a modified Fc domain, or FcRn-binding fragment thereof, wherein the antibody has increased affinity for the FcRn receptor relative to the Fc domain of A4B4L1FR-S28R (MEDI-524) that does not comprise a modified Fc domain (i.e., unmodified A4B4LIFR-S28R).

In preferred embodiments, the methods of the invention encompass the use of modified antibodies, for example any antibody described herein, that comprises a modified IgG, such as a modified IgG1, constant domain, wherein the modified IgG constant domain comprises a modification of a residue (and, in some embodiments, an unmodified residue), preferably at one or more of residues 251-256, 285-290, 308-314, 385-389, and 428-436, that increases the in vivo half-life of the IgG constant domain, or FcRn-binding fragment thereof (e.g., Fc or hinge-Fc domain), and any molecule attached thereto, and increases the affinity of the IgG, or fragment thereof, for FcRn. In certain embodiments, the IgG constant domain comprises the YTE modification. In some embodiments, a modified antibody of the invention (and methods of using the antibody thereof) comprises a VH and/or VL domain(s) of A4B4L1FR-S28R (MEDI-524) (FIG. 13) and a modified IgG, such as a modified IgG1, constant domain, wherein the Fc domain comprises the YTE modification. In some embodiments, a modified antibody of the invention (and methods of using the antibody thereof) comprises a VH and/or VL chain(s) of A4B4L1FR-S28R (MEDI-524) (FIG. 13) and a modified IgG, such as a modified IgG1, constant domain, wherein the Fc domain comprises the YTE modification. In other embodiments, a modified antibody of the invention comprises any VH and/or VL domain(s) of an antibody listed in Table 2 and a modified IgG, such as a modified IgG1, constant domain, wherein the Fc domain comprises the YTE modification. In other embodiments, a modified antibody of the invention comprises any VH and/or VL chain(s) of an antibody listed in Table 2 and a modified IgG, such as a modified IgG1, constant domain, wherein the Fc domain comprises the YTE modification.

### 3.1 TERMINOLOGY

The term “about” or “approximately” means within 20%, preferably within 10%, and more preferably within 5% (or 1% or less) of a given value or range.

As used herein, “administer” or “administration” refers to the act of injecting or otherwise physically delivering a substance as it exists outside the body (e.g., an antibody of the invention) into a patient, such as by, but not limited to, pulmonary (e.g., inhalation), mucosal (e.g., intranasal), intradermal, intravenous, intramuscular delivery and/or any other method of physical delivery described herein or known in the art. When a disease, or symptoms thereof, are being treated, administration of the substance typically occurs after the onset of the disease or symptoms thereof. When a disease, or symptoms thereof, are being prevented, administration of the substance typically occurs before the onset of the disease or symptoms thereof.

In the context of a polypeptide, the term “analog” as used herein refers to a polypeptide that possesses a similar or identical function as a RSV polypeptide, a fragment of a RSV polypeptide, or an anti-RSV antibody but does not necessarily comprise a similar or identical amino acid sequence of a RSV polypeptide, a fragment of a RSV polypeptide, or an anti-RSV antibody, or possess a similar or identical structure of a RSV polypeptide, a fragment of a RSV polypeptide, or an antibody. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a RSV polypeptide, a fragment of a RSV polypeptide, or an antibody described herein; (b) a polypeptide encoded by a nucleotide sequence that hybridizes under stringent conditions to a nucleotide sequence encoding a RSV polypeptide, a fragment of a RSV polypeptide, or an antibody described herein of at least 5 amino acid residues, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues (see, e.g., Maniatis et al. (1982) Molecular Cloning: A Laboratory Manual, Cold Spring Harbor Press, Cold Spring Harbor, N.Y.); and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a RSV polypeptide, a fragment of a RSV polypeptide, or an antibody described herein. A polypeptide with similar structure to a RSV polypeptide, a fragment of a RSV polypeptide, or an antibody described herein refers to a polypeptide that has a similar secondary, tertiary or quaternary structure of a RSV polypeptide, a fragment of a RSV, or an antibody described herein. The structure of a polypeptide can determined by methods known to those skilled in the art, including but not limited to, X-ray crystallography, nuclear magnetic resonance, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X 100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score 50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The terms “antibodies that immunospecifically bind to a RSV antigen,” “anti-RSV antibodies” and analogous terms as used herein refer to antibodies, including both modified antibodies (i.e., antibodies that comprise a modified IgG (e.g., IgG1) constant domain, or FcRn-binding fragment thereof (e.g., the Fc-domain or hinge-Fc domain)) and unmodified antibodies (i.e., antibodies that do not comprise a modified IgG (e.g., IgG1) constant domain, or FcRn-binding fragment thereof (e.g., the Fc-domain or hinge-Fc domain)), that specifically bind to a RSV polypeptide. An antibody or a fragment thereof that immunospecifically binds to a RSV antigen may be cross-reactive with related antigens. Preferably, an antibody or a fragment thereof that immunospecifically binds to a RSV antigen does not cross-react with other antigens. An antibody or a fragment thereof that immunospecifically binds to a RSV antigen can be identified, for example, by immunoassays, BIAcore, or other techniques known to those of skill in the art. An antibody or a fragment thereof binds specifically to a RSV antigen when it binds to a RSV antigen with higher affinity than to any cross-reactive antigen as determined using experimental techniques, such as radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

Antibodies of the invention include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific, bispecific, etc.), Fab fragments, F(ab′) fragments, disulfide-linked Fvs (sdFv), anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen-binding site that immunospecifically binds to a RSV antigen (preferably, a RSV F antigen) (e.g., one or more complementarity determining regions (CDRs) of an anti-RSV antibody). The antibodies of the invention can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule. In preferred embodiments, modified antibodies of the invention are IgG antibodies, or a class (e.g., human IgG1) or subclass thereof.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

In the context of a polypeptide, the term “derivative” as used herein refers to a polypeptide that comprises an amino acid sequence of a RSV polypeptide, a fragment of a RSV polypeptide, or an antibody that immunospecifically binds to a RSV polypeptide which has been altered by the introduction of amino acid residue substitutions, deletions or additions. The term “derivative” as used herein also refers to a RSV polypeptide, a fragment of a RSV polypeptide, or an antibody that immunospecifically binds to a RSV polypeptide which has been chemically modified, e.g., by the covalent attachment of any type of molecule to the polypeptide. For example, but not by way of limitation, a RSV polypeptide, a fragment of a RSV polypeptide, or an antibody may be chemically modified, e.g., by glycosylation, acetylation, pegylation, phosphorylation, amidation, derivatization by known protecting/blocking groups, proteolytic cleavage, linkage to a cellular ligand or other protein, etc. A derivative of a RSV polypeptide, a fragment of a RSV polypeptide, or an antibody may be chemically modified by chemical modifications using techniques known to those of skill in the art, including, but not limited to specific chemical cleavage, acetylation, formylation, metabolic synthesis of tunicamycin, etc. Further, a derivative of a RSV polypeptide, a fragment of a RSV polypeptide, or an antibody may contain one or more non-classical amino acids. A polypeptide derivative possesses a similar or identical function as a RSV polypeptide, a fragment of a RSV polypeptide, or an antibody described herein.

The term “effective amount” as used herein refers to the amount of a therapy (e.g., a modified or other antibody of the invention) which is sufficient to reduce and/or ameliorate the severity and/or duration of a RSV infection (e.g., acute RSV disease or RSV URI and/or LRI), otitis media, and/or a symptom or respiratory condition relating thereto (including, but not limited to, asthma, wheezing, RAD, or a combination thereof); prevent the advancement or progression of a RSV URI to a LRI, a clinically significant acute RSV disease in the lungs, otitis media and/or a symptom or respiratory condition relating thereto (e.g., prevent the progression of an upper respiratory tract RSV infection to a lower respiratory tract RSV infection); prevent the recurrence, development, or onset of a RSV infection (e.g., acute RSV disease, or RSV URI and/or LRI), otitis media, and/or a symptom or respiratory condition relating thereto (including, but not limited to, asthma, wheezing, RAD, or a combination thereof); and/or enhance and/or improve the prophylactic or therapeutic effect(s) of another therapy (e.g., a therapy other than an antibody of the invention). Non-limiting examples of effective amounts of an antibody of the invention are provided in Section 5.3, infra. In some embodiments, the effective amount of an antibody of the invention is about 0.025 mg/kg, about 0.05 mg/kg, about 0.10 mg/kg, about 0.20 mg/kg, about 0.40 mg/kg, about 0.80 mg/kg, about 1.0 mg/kg, about 1.5 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg or about 60 mg/kg. In one embodiment, an effective amount of an antibody of the invention is about 15 mg of the antibody per kg of body weight of the subject.

The term “effective neutralizing titer” as used herein refers to the amount of antibody which corresponds to the amount present in the serum of animals (human or cotton rat) that has been shown to be either clinically efficacious (in humans) or to reduce virus by 99% in, for example, cotton rats. The 99% reduction is defined by a specific challenge of, e.g., 10³ pfu, 10⁴ pfu, 10⁵ pfu, 10⁶ pfu, 10⁷ pfu, 10⁸ pfu, or 10⁹ pfu of RSV.

The term “elderly” as used herein refers to a human subject who is age 65 or older.

The term “epitopes” as used herein refers to fragments of a RSV polypeptide having antigenic or immunogenic activity in an animal, preferably a mammal, and most preferably in a human. An epitope having immunogenic activity is a fragment of a RSV polypeptide (e.g., RSV F protein) that elicits an antibody response in an animal. An epitope having antigenic activity is a fragment of a RSV polypeptide to which an antibody immunospecifically binds as determined by any method well known in the art, for example, by the immunoassays described herein. Antigenic epitopes need not necessarily be immunogenic.

The term “excipients” as used herein refers to inert substances which are commonly used as a diluent, vehicle, preservatives, binders, or stabilizing agent for drugs and includes, but not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). Also see Remington's Pharmaceutical Sciences (by Joseph P. Remington, 18th ed., Mack Publishing Co., Easton, Pa.), which is hereby incorporated in its entirety.

The term “FcRn receptor” or “FcRn” as used herein refers to an Fc receptor (“n” indicates neonatal) which is known to be involved in transfer of maternal IgGs to a fetus through the human or primate placenta, or yolk sac (rabbits) and to a neonate from the colostrum through the small intestine. It is also known that FcRn is involved in the maintenance of constant serum IgG levels by binding the IgG molecules and recycling them into the serum. The binding of FcRn to IgG molecules is pH-dependent with optimum binding at pH 6.0. FcRn comprises a heterodimer of two polypeptides, whose molecular weights are approximately 50 kD and 15 kD, respectively. The extracellular domains of the 50 kD polypeptide are related to major histocompatibility complex (MHC) class I α-chains and the 15 kD polypeptide was shown to be the non-polymorphic β₂-microglobulin (β₂-m). In addition to placenta and neonatal intestine, FcRn is also expressed in various tissues across species as well as various types of endothelial cell lines. It is also expressed in human adult vascular endothelium, muscle vasculature and hepatic sinusoids and it is suggested that the endothelial cells may be most responsible for the maintenance of serum IgG levels in humans and mice. The amino acid sequences of human FcRn and murine FcRn are indicated by SEQ ID NO:337 (FIG. 21A) and SEQ ID NO:338 (FIG. 21B), respectively. Homologs of these sequences having FcRn activity are also included.

In the context of a peptide or polypeptide, the term “fragment” as used herein refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a RSV polypeptide or an antibody that immunospecifically binds to a RSV polypeptide. In a specific embodiment, a fragment of a RSV polypeptide or an antibody of that immunospecifically binds to a RSV antigen retains at least 1, at least 2, or at least 3 functions of the polypeptide or antibody.

The term “fusion protein” as used herein refers to a polypeptide that comprises an amino acid sequence of an antibody and an amino acid sequence of a heterologous polypeptide or protein (i.e., a polypeptide or protein not normally a part of the antibody (e.g., a non-anti-RSV antigen antibody)).

The term “high potency” as used herein refers to antibodies that exhibit high potency as determined in various assays for biological activity (e.g., neutralization of RSV) such as those described herein. For example, high potency antibodies of the invention have an IC₅₀ value less than 5 nM, less than 4 nM, less than 3 nM, less than 2 nM, less than 1.75 nM, less than 1.5 nM, less than 1.25 nM, less than 1 nM, less than 0.75 nM, less than 0.5 nM, less than 0.25 nM, less than 0.1 nM, less than 0.05 nM, less than 0.025 nM, or less than 0.01 nM, as measured by a microneutralization assay. In certain embodiments, the microneutralization assay is a microneutralization assay described herein (for example, as described in Examples 6.4, 6.8, and 6.18 herein) or as in Johnson et al., 1999, J. Infectious Diseases 180:35-40. Further, high potency antibodies of the invention result in at least a 75%, preferably at least a 95% and more preferably a 99% lower RSV titer in a cotton rat 5 days after challenge with 10⁵ pfu relative to a cotton rat not administered said antibodies. In certain embodiments of the invention, high potency antibodies of the present invention exhibit a high affinity and/or high avidity for one or more RSV antigens (e.g., antibodies having an affinity of at least 2×10⁸ M⁻¹, preferably between 2×10⁸M⁻¹ and 5×10¹²M⁻¹, such as at least 2.5×10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, or at least 5×10¹²M⁻¹ for one or more RSV antigens).

The term “host” as used herein refers to an animal, preferably a mammal, and most preferably a human.

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “human infant” as used herein refers to a human less than 24 months, preferably less than 16 months, less than 12 months, less than 6 months, less than 3 months, less than 2 months, or less than 1 month of age.

The term “human infant born prematurely” as used herein refers to a human born at less than 40 weeks gestational age, preferably less than 35 weeks gestational age, wherein the infant is less than 6 months old, preferably less than 3 months old, more preferably less than 2 months old, and most preferably less than 1 month old.

The terms “IgG Fc region,” “Fc region,” “Fc domain,” “Fc fragment” and other analogous terms as used herein refers the portion of an IgG molecule that correlates to a crystallizable fragment obtained by papain digestion of an IgG molecule. The Fc region consists of the C-terminal half of the two heavy chains of an IgG molecule that are linked by disulfide bonds. It has no antigen binding activity but contains the carbohydrate moiety and the binding sites for complement and Fc receptors, including the FcRn receptor (see below). For example, an Fc fragment contains the entire second constant domain CH2 (residues 231-340 of human IgG1, see, e.g., FIG. 20B) (e.g., SEQ ID NO:339) and the third constant domain CH3 (residues 341-447 of human IgG1, see, e.g., FIG. 20B) (e.g., SEQ ID NO:340). All numbering used herein is according to the EU Index (Kabat et al. (1991) Sequences of proteins of immunological interest. (U.S. Department of Health and Human Services, Washington, D.C.) 5^{th} ed.), unless otherwise indicated.

The term “IgG hinge-Fc region” or “hinge-Fc fragment” as used herein refers to a region of an IgG molecule consisting of the Fc region (residues 231-447, see, e.g., FIG. 20B) and a hinge region (residues 216-230; e.g., SEQ ID NO:341, see, e.g., FIG. 20B) extending from the N-terminus of the Fc region, according to the EU Index (Kabat et al. (1991) Sequences of proteins of immunological interest. (U.S. Department of Health and Human Services, Washington, D.C.) 5^{th} ed.). An example of the amino acid sequence of the human IgG1 hinge-Fc region is SEQ ID NO:342 (see also FIGS. 20A and 20B).

The term “immunomodulatory agent” and variations thereof including, but not limited to, immunomodulatory agents, as used herein refer to an agent that modulates a host's immune system. In certain embodiments, an immunomodulatory agent is an immunosuppressant agent. In certain other embodiments, an immunomodulatory agent is an immunostimulatory agent. In accordance with the invention, an immunomodulatory agent used in the combination therapies of the invention does not include an anti-RSV antibody or fragment thereof. Immunomodulatory agents include, but are not limited to, small molecules, peptides, polypeptides, proteins, fusion proteins, antibodies, inorganic molecules, mimetic agents, and organic molecules.

As used herein, the term “in combination” in the context of the administration of other therapies refers to the use of more than one therapy. The use of the term “in combination” does not restrict the order in which therapies are administered to a subject with an infection. A first therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject which had, has, or is susceptible to a RSV infection, otitis media or a respiratory condition related thereto. Any additional therapy can be administered in any order with the other additional therapies. In certain embodiments, the antibodies of the invention can be administered in combination with one or more therapies (e.g., therapies that are not the antibodies of the invention that are currently administered to prevent, treat, manage, and/or ameliorate a RSV infection (e.g., acute RSV disease or a RSV URI and/or LRI, otitis media, and/or a symptom or respiratory condition or other symptom related thereto). Non-limiting examples of therapies that can be administered in combination with an antibody of the invention include analgesic agents, anesthetic agents, antibiotics, or immunomodulatory agents or any other agent listed in the U.S. Pharmacopoeia and/or Physician's Desk Reference.

As used herein, the terms “infection” and “RSV infection” refer to all stages of RSV's life cycle in a host (including, but not limited to the invasion by and replication of RSV in a cell or body tissue), as well as the pathological state resulting from the invasion by and replication of a RSV. The invasion by and multiplication of a RSV includes, but is not limited to, the following steps: the docking of the RSV particle to a cell, fusion of a virus with a cell membrane, the introduction of viral genetic information into a cell, the expression of RSV proteins, the production of new RSV particles and the release of RSV particles from a cell. An RSV infection may be an upper respiratory tract RSV infection (URI), a lower respiratory tract RSV infection (LRI), or a combination thereof. In specific embodiments, the pathological state resulting from the invasion by and replication of a RSV is an acute RSV disease. The term “acute RSV disease” as used herein refers to clinically significant disease in the lungs or lower respiratory tract as a result of an RSV infection, which can manifest as pneumonia and/or bronchiolitis, where such symptoms may include hypoxia, apnea, respiratory distress, rapid breathing, wheezing, cyanosis, etc. Acute RSV disease requires an affected individual to obtain medical intervention, such as hospitalization, administration of oxygen, intubation and/or ventilation.

The term “inorganic salt” as used herein refers to any compounds containing no carbon that result from replacement of part or all of the acid hydrogen or an acid by a metal or a group acting like a metal and are often used as a tonicity adjusting compound in pharmaceutical compositions and preparations of biological materials. The most common inorganic salts are NaCl, KCl, NaH₂PO₄, etc.

The term “in vivo half-life” as used herein refers to a biological half-life of a particular type of IgG molecule or its fragments containing FcRn-binding sites in the circulation of a given animal and is represented by a time required for half the quantity administered in the animal to be cleared from the circulation and/or other tissues in the animal. When a clearance curve of a given IgG is constructed as a function of time, the curve is usually biphasic with a rapid α-phase which represents an equilibration of the injected IgG molecules between the intra- and extra-vascular space and which is, in part, determined by the size of molecules, and a longer β-phase which represents the catabolism of the IgG molecules in the intravascular space. The term “in vivo half-life” practically corresponds to the half-life of the IgG molecules in the β-phase. As used herein, “increased in vivo serum half-life” or “extended in vivo serum half-life” of an antibody that comprises a modified IgG constant domain, or FcRn-binding fragment thereof (preferably the Fc domain or the hinge-Fc domain), refers to an increase in in vivo serum half-life of the antibody as compared to an antibody that does not comprise a modified IgG constant domain, or FcRn-binding fragment thereof (e.g., as compared to an the antibody that does not comprise the one or more modifications in the constant domain, or FcRn-binding fragment thereof (i.e., an unmodified antibody), or as compared to another RSV antibody, such as palivizumab).

An “isolated” or “purified” antibody is substantially free of cellular material or other contaminating proteins from the cell or tissue source from which the protein is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). When the antibody is recombinantly produced, it is also preferably substantially free of culture medium, i.e., culture medium represents less than about 20%, 10%, or 5% of the volume of the protein preparation. When the antibody is produced by chemical synthesis, it is preferably substantially free of chemical precursors or other chemicals, i.e., it is separated from chemical precursors or other chemicals which are involved in the synthesis of the protein. Accordingly such preparations of the antibody have less than about 30%, 20%, 10%, 5% (by dry weight) of chemical precursors or compounds other than the antibody of interest. In a preferred embodiment, antibodies of the invention are isolated or purified.

An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, a nucleic acid molecule(s) encoding an antibody of the invention is isolated or purified.

The term “lower respiratory” tract refers to the major passages and structures of the lower respiratory tract including the windpipe (trachea) and the lungs, including the bronchi, bronchioles, and alveoli of the lungs.

As used herein, the term “low tolerance” refers to a state in which the patient suffers from side effects from a therapy so that the patient does not benefit from and/or will not continue therapy because of the adverse effects and/or the harm from side effects outweighs the benefit of the therapy.

The phrase “low to undetectable levels of aggregation” as used herein refers to samples containing no more than 5%, no more than 4%, no more than 3%, no more than 2%, no more than 1% and most preferably no more than 0.5% aggregation by weight of protein as measured by high performance size exclusion chromatography (HPSEC).

The term “low to undetectable levels of fragmentation” as used herein refers to samples containing equal to or more than 80%, 85%, 90%, 95%, 98% or 99% of the total protein, for example, in a single peak as determined by HPSEC, or in two peaks (heavy- and light-chains) by reduced Capillary Gel Electrophoresis (rCGE), representing the non-degraded antibody or a non-degraded fragment thereof, and containing no other single peaks having more than 5%, more than 4%, more than 3%, more than 2%, more than 1%, or more than 0.5% of the total protein in each. The term “reduced Capillary Gel Electrophoresis” as used herein refers to capillary gel electrophoresis under reducing conditions sufficient to reduce disulfide bonds in an antibody or fragment thereof.

As used herein, the terms “manage,” “managing,” and “management” refer to the beneficial effects that a subject derives from a therapy (e.g., a prophylactic or therapeutic agent), which does not result in a cure of the infection. In certain embodiments, a subject is administered one or more therapies (e.g., prophylactic or therapeutic agents, such as an antibody of the invention) to “manage” a RSV infection (e.g., acute RSV disease or RSV URI and/or LRI), one or more symptoms thereof, or a respiratory condition associated with, potentiated by, or potentiating a RSV infection, so as to prevent the progression or worsening of the infection.

As used herein, the term “modified antibody” encompasses any antibody described herein that comprises one or more “modifications” to the amino acid residues at given positions of the antibody constant domain (preferably an IgG and more preferably an IgG1 constant domain), or FcRn-binding fragment thereof wherein the antibody has an increased in vivo half-life as compared to known anti-RSV antibodies (e.g., palivizumab) and/or as compared to the same antibody that does not comprise one or more modifications in the IgG constant domain, or FcRn-binding fragment thereof, as a result of, e.g., one or more modifications in amino acid residues identified to be involved in the interaction between the constant domain, or FcRn-binding fragment thereof (preferably, an Fc domain or hinge-Fc domain), of said antibodies and the Fc Receptor neonate (FcRn). Due to natural variations in IgG constant domain sequences (see, e.g., Kabat et al., supra), in certain instances, a first amino acid residue may be substituted with a second amino acid residue at a given position (for example, in the sequence shown in FIG. 20B, the Met at position 252 may be substituted with a Tyr) or, alternatively, the second residue may be already present in antibody at the given position, in which case substitution is not necessary (for example, the Met at position 252 remains a Met). Thus, the term “modified antibody” also encompasses antibodies that naturally comprise one or more of the recited residues at the indicated positions (e.g., the residues are already present in the recited position in the molecule without modification). Numbering of constant domain positions is according to the EU Index (Kabat et al. (1991) Sequences of proteins of immunological interest. (U.S. Department of Health and Human Services, Washington, D.C.) 5^{th} ed.). Exemplary human IgG1 constant domain hinge, CH2 and CH3 regions are shown in FIG. 20B, with numbering according to the EU Index as in Kabat et al., supra. In preferred embodiments, the modified antibody comprises modifications to the amino acid residues of the Fc domain or hinge-Fc domain, most preferably of an IgG1 constant domain. In some embodiments, a “modified antibody” of the invention (e.g., one that comprises a modified IgG constant domain, Fc domain, or FcRn-binding fragment thereof and has increased in vivo half-life) has increased affinity for the FcRn relative to the same antibody without a modified IgG constant domain, Fc domain, or FcRn-binding fragment thereof. In other embodiments, a modified antibody of the invention (e.g., one that comprises a modified IgG constant domain, Fc domain, or FcRn-binding fragment thereof and has increased in vivo half-life) has increased affinity for the FcRn relative to the Fc domain of palivizumab. As used herein, a “modified antibody” may or may not be a high potency, high affinity and/or high avidity modified antibody. In certain embodiments, the modified antibody is a high potency antibody, and most preferably a high potency, high affinity modified antibody. In preferred embodiments, the modified antibodies comprise a modified IgG (e.g., IgG1) constant domain, or FcRn binding fragment thereof, comprising a Tyr at position 252, a Thr at position 254, and a Glu at position 256 (“YTE”) (see FIG. 35), with numbering according to the EU Index as in Kabat et al., supra, (see also FIG. 20B).

As used herein, one or more “modifications to the amino acid residues” in the context of a constant domain, or FcRn-binding fragment thereof, of an antibody of the invention refers to any mutation, substitution, insertion or deletion of one or more amino acid residues of the sequence of the constant domain, or FcRn-binding fragment thereof (preferably, Fc domain or hinge-Fc domain) of the antibody. Preferably, the one or more modifications are substitutions. In preferred embodiments, the one or more modifications are at positions 251-256, 285-290, 308-314, 385-389, and 428-436, with numbering according to the EU Index as in Kabat et al., supra (see also FIG. 20B). In certain preferred embodiments, an IgG constant domain comprises a Y at position 252 (252Y), a T at position 254 (254T), and/or an E at position 256 (256E). Due to natural variations in IgG constant domain sequences (see, e.g., Kabat et al., supra), in certain instances, a first amino acid residue may be substituted with a second amino acid residue at a given position (for example, in the sequence shown in FIG. 20B, the Met at position 252 may be substituted with a Tyr) or, alternatively, the second residue may be already present in antibody at the given position, in which case substitution is not necessary (for example, the Met at position 252 remains a Met). Thus, discussions herein of exemplary “modifications” in an IgG constant domain, for example, 252Y, 254T, and/or 256E, are meant to encompass both molecules that naturally comprise the recited residues at the indicated positions (e.g., the residues are already present in the recited position in the molecule) and/or molecules that are modified (e.g., by amino acid substitution) to comprise the recited residues at the indicated positions. Numbering of amino acid positions used herein is according to the EU Index, as in Kabat et al. (1991) Sequences of proteins of immunological interest. (U.S. Department of Health and Human Services, Washington, D.C.) 5^{th} ed. (“Kabat et al.”).

As used herein, the term “palivizumab standard reference” and analogous terms refer to commercially available lyophilized palivizumab, as described in the Physicians' Desk Reference, 56^{th} edition, 2002. Reconstituted palivizumab may contain, e.g., the following excipients: 47 mM histidine, 3.0 mM glycine and 5.6% manitol and the active ingredient, the antibody, at a concentration of 100 milligrams per ml solution.

As used herein, the terms “peptide,” “polypeptide,” and “protein” are used to refer to amino acid sequences of various approximate lengths. For example, a peptide refers to a chain of two or more amino acids joined by peptide bonds, generally of less than about 50 amino acid residues, while a polypeptide refers to a longer chain of amino acids. In the context of a polypeptide that is a portion of a protein, the polypeptide is a chain of amino acids that is less in length than the length of the protein. It is appreciated that the terms “peptide” and “polypeptide” are not meant to refer to a precise length of a chain of amino acid residues and that in certain contexts, the two terms may be used interchangeably.

The term “pharmaceutically acceptable” as used herein means being approved by a regulatory agency of the Federal or a state government, or listed in the U.S. Pharmacopia, European Pharmacopia or other generally recognized pharmacopia for use in animals, and more particularly in humans.

The term “polyol” as used herein refers to a sugar that contains many —OH groups compared to a normal saccharide.

As used herein, the terms “prevent,” “preventing,” and “prevention” refer to the total or partial inhibition of RSV infection (e.g., acute RSV disease or RSV URI and/or LRI); the total or partial inhibition of the development or onset of disease progression of RSV from the upper respiratory tract to the lower respiratory tract and/or LRI, acute RSV disease, otitis media, and/or a symptom or respiratory condition related thereto in a subject; the total or partial inhibition of the progression of an upper respiratory tract RSV infection to a lower respiratory tract RSV infection, otitis media or a respiratory condition related thereto resulting from the administration of a therapy (e.g., a prophylactic or therapeutic agent); the total or partial inhibition of an upper and/or lower tract RSV infection, otitis media or a symptom or respiratory condition related thereto resulting from the administration of a combination of therapies (e.g., a combination of prophylactic or therapeutic agents); the total or partial inhibition of RSV infection; the total or partial inhibition of acute RSV disease.

As used herein, the term “prophylactic agent” refers to any agent that can prevent or inhibit the development or onset of disease progression of RSV from the upper to the lower respiratory tract and/or prevent or inhibit LRI, acute RSV disease, otitis media, and/or a symptom or respiratory condition relating to RSV infection in a subject; the prevention or inhibition of an upper respiratory tract RSV infection, lower respiratory tract RSV infection, acute RSV disease, otitis media, or a respiratory condition relating thereto resulting from the administration of a therapy (e.g., a prophylactic or therapeutic agent). The term also refers to preventing or inhibiting the recurrence, spread or onset of a RSV infection (e.g., acute RSV disease or RSV URI and/or LRI), otitis media, and/or a symptom or respiratory condition relating thereto (including, but not limited to, asthma, wheezing, RAD, or a combination thereof), and/or prevent the progression of an upper respiratory tract RSV infection to a lower respiratory tract RSV infection, otitis media and/or a symptom or respiratory condition related thereto. In certain embodiments, the term “prophylactic agent” refers to an antibody of the invention. In certain other embodiments, the term “prophylactic agent” refers to an agent other than an antibody of the invention. Preferably, a prophylactic agent is an agent which is known to be useful to or has been or is currently being used to prevent acute RSV disease and/or LRI or impede the onset, development, progression and/or severity of a RSV infection (preferably a RSV URI and/or LRI) otitis media, and/or a symptom or respiratory condition related thereto. In some embodiments, the prophylactic agent is a modified antibody of the invention.

In certain embodiments of the invention, a “prophylactically effective serum titer” is the serum titer in a subject, preferably a human, that prevents RSV infection in the lungs and/or that reduces the incidence of a RSV infection (e.g., acute RSV disease, or RSV URI and/or LRI), otitis media and/or a symptom or respiratory condition related thereto in said subject. The term also refers to the serum titer in a subject that prevents or inhibits the recurrence, spread or onset of a RSV URI and/or LRI, otitis media, and/or a symptom or respiratory condition relating thereto (including, but not limited to, asthma, wheezing, RAD, or a combination thereof), and/or prevents or inhibits the progression of an upper respiratory tract RSV infection to a lower respiratory tract RSV infection, otitis media and/or a symptom or respiratory condition related thereto. In some embodiments, the prophylactically effective serum titer prevents the progression of an upper respiratory tract RSV infection to a lower respiratory tract RSV infection, otitis media and/or a symptom or respiratory condition related thereto. Preferably, the prophylactically effective serum titer reduces the incidence of RSV infections in humans with the greatest probability of complications resulting from RSV infection (e.g., a human with cystic fibrosis, bronchopulmonary dysplasia, congenital heart disease, congenital immunodeficiency or acquired immunodeficiency, a human who has had a bone marrow transplant, a human infant, or an elderly human). In certain other embodiments of the invention, a “prophylactically effective serum titer” is the serum titer in a cotton rat that results in a RSV titer 5 days after challenge with 10⁵ pfu that is 99% lower than the RSV titer 5 days after challenge with 10⁵ pfu of RSV in a cotton rat not administered an antibody that immunospecifically binds to a RSV antigen.

As used herein, the term “refractory” refers to a RSV infection (e.g., acute RSV disease and/or RSV URI and/or LRI), otitis media or a respiratory condition related thereto that is not responsive to one or more therapies (e.g., currently available therapies). In a certain embodiment, a RSV infection (e.g., acute RSV disease, or RSV URI and/or LRI), otitis media or a respiratory condition related thereto is refractory to a therapy means that at least some significant portion of the symptoms associated with said RSV infection (e.g., acute RSV disease or RSV URI and/or LRI), otitis media or a respiratory condition related thereto are not eliminated or lessened by that therapy. The determination of whether a RSV infection (e.g., acute RSV disease, or RSV URI and/or LRI), otitis media or a respiratory condition related thereto is refractory can be made either in vivo or in vitro by any method known in the art for assaying the effectiveness of therapy for the infection, otitis media or the respiratory condition related thereto.

The term “RSV antigen” refers to a RSV polypeptide to which an antibody immunospecifically binds. A RSV antigen also refers to an analog or derivative of a RSV polypeptide or fragment thereof to which an antibody immunospecifically binds. In some embodiments, a RSV antigen is a RSV F antigen, RSV G antigen or a RSV SH antigen.

The term “saccharide” as used herein refers to a class of molecules that are derivatives of polyhydric alcohols. Saccharides are commonly referred to as carbohydrates and may contain different amounts of sugar (saccharide) units, e.g., monosaccharides, disaccharides and polysaccharides.

The term “serum titer” as used herein refers to an average serum titer in a population of least 10, preferably at least 20, and most preferably at least 40 subjects up to about 100, 1000 or more.

As used herein, the term “side effects” encompasses unwanted and adverse effects of a therapy (e.g., a prophylactic or therapeutic agent). Unwanted effects are not necessarily adverse. An adverse effect from a therapy (e.g., a prophylactic or therapeutic agent) might be harmful or uncomfortable or risky. Examples of side effects include, but are not limited to, URI, otitis media, rhinitis, diarrhea, cough, gastroenteritis, wheezing, nausea, vomiting, anorexia, abdominal cramping, fever, pain, loss of body weight, dehydration, alopecia, dyspenea, insomnia, dizziness, mucositis, nerve and muscle effects, fatigue, dry mouth, and loss of appetite, rashes or swellings at the site of administration, fin-like symptoms such as fever, chills and fatigue, digestive tract problems and allergic reactions. Additional undesired effects experienced by patients are numerous and known in the art. Many are described in the Physician's Desk Reference (58^{th} ed., 2004).

The term “small molecule” and analogous terms include, but are not limited to, peptides, peptidomimetics, amino acids, amino acid analogues, polynucleotides, polynucleotide analogues, nucleotides, nucleotide analogues, organic or inorganic compounds (i.e., including heterorganic and/or ganometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The terms “stability” and “stable” as used herein in the context of a liquid formulation comprising an antibody that immuno specifically binds to a RSV antigen refer to the resistance of the antibody in the formulation to thermal and chemical unfolding, aggregation, degradation or fragmentation under given manufacture, preparation, transportation and storage conditions. The “stable” formulations of the invention retain biological activity equal to or more than 80%, 85%, 90%, 95%, 98%, 99%, or 99.5% under given manufacture, preparation, transportation and storage conditions. The stability of the antibody can be assessed by degrees of aggregation, degradation or fragmentation by methods known to those skilled in the art, including but not limited to reduced Capillary Gel Electrophoresis (rCGE), Sodium Dodecyl Sulfate Polyacrylamide Gel Electrophoresis (SDS-PAGE) and HPSEC, compared to a reference, that is, a commercially available lyophilized palivizumab reconstituted to 100 mg/ml in 50 mM histidine/3.2 mM glycine buffer with 6% mannitol at pH 6.0. The reference regularly gives a single peak (≧97% area) by HPSEC. The overall stability of a formulation comprising an antibody that immunospecifically binds to a RSV antigen can be assessed by various immunological assays including, for example, ELISA and radioimmunoassay using the specific epitope of RSV.

As used herein, the terms “subject” and “patient” are used interchangeably. As used herein, a subject is preferably a mammal such as a non-primate (e.g., cows, pigs, horses, cats, dogs, rats, etc.) and a primate (e.g., monkey and human), most preferably a human. In one embodiment, the subject is a mammal, preferably a human, with a RSV infection (e.g., acute RSV disease, or a RSV URI and/or LRI) or otitis media. In another embodiment, the subject is a mammal, preferably a human, at risk of developing a RSV infection (e.g., acute RSV disease, or a RSV URI and/or LRI) or otitis media (e.g., an immunocompromised or immunosuppressed mammal, or a genetically predisposed mammal). In one embodiment, the subject is a human with a respiratory condition (including, but not limited to asthma, wheezing or RAD) that stems from, is caused by or associated with a RSV infection. In some embodiments, the subject is 0-5 years old or is a human infant, preferably age 0-2 years old (e.g., 0-12 months old). In other embodiments, the subject is an elderly subject.

The term “substantially free of surfactant” as used herein refers to a formulation of an antibody that immunospecifically binds to a RSV antigen, said formulation containing less than 0.0005%, less than 0.0003%, or less than 0.0001% of surfactants and/or less than 0.0005%, less than 0.0003%, or less than 0.0001% of surfactants.

The term “substantially free of salt” as used herein refers to a formulation of an antibody that immunospecifically binds to a RSV antigen, said formulation containing less than 0.0005%, less than 0.0003%, or less than 0.0001% of inorganic salts.

The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials.

As used herein, the term “therapeutic agent” refers to any agent that can be used in the treatment, management or amelioration of a RSV infection (e.g., acute RSV disease or a RSV URI and/or LRI), otitis media or a symptom or a respiratory condition related thereto (e.g., asthma, wheezing and/or RAD). In certain embodiments, the term “therapeutic agent” refers to an antibody of the invention. In certain other embodiments, the term “therapeutic agent” refers to an agent other than an antibody of the invention. Preferably, a therapeutic agent is an agent which is known to be useful for, or has been or is currently being used for the treatment, management or amelioration of a RSV infection (e.g., acute RSV disease and/or a RSV URI and/or LRI), otitis media, or one or more symptoms or respiratory conditions related thereto. In certain embodiments, the therapeutic agent is a modified antibody of the invention.

The term “synergistic” as used herein refers to a combination of therapies (e.g., use of prophylactic or therapeutic agents) which is more effective than the additive effects of any two or more single therapy. For example, a synergistic effect of a combination of prophylactic or therapeutic agents permits the use of lower dosages of one or more of the agents and/or less frequent administration of said agents to a subject with a RSV infection. The ability to utilize lower dosages of prophylactic or therapeutic therapies and/or to administer said therapies less frequently reduces the toxicity associated with the administration of said therapies to a subject without reducing the efficacy of said therapies in the prevention, management, treatment or amelioration of a RSV infection (e.g., acute RSV disease, or a RSV URI and/or LRI), otitis media, or a symptom or respiratory condition relating thereto (including, but not limited to, asthma, wheezing, RAD, or a combination thereof). In addition, a synergistic effect can result in improved efficacy of therapies in the prevention, management, treatment or amelioration of a RSV infection (e.g., acute RSV disease, or a RSV URI and/or LRI), otitis media, or a symptom or respiratory condition relating thereto (including, but not limited to, asthma, wheezing, RAD, or a combination thereof). Finally, synergistic effect of a combination of therapies (e.g., prophylactic or therapeutic agents) may avoid or reduce adverse or unwanted side effects associated with the use of any single therapy.

In certain embodiments of the invention, a “therapeutically effective serum titer” is the serum titer in a subject, preferably a human, that reduces the severity, the duration and/or the symptoms associated with a RSV infection (e.g., acute RSV disease or RSV URI and/or LRI) in said subject. Preferably, the therapeutically effective serum titer reduces the severity, the duration and/or the number symptoms associated with a RSV infection (e.g., acute RSV disease or RSV URI and/or LRI) in humans with the greatest probability of complications resulting from the infection (e.g., a human with cystic fibrosis, bronchopulmonary dysplasia, congenital heart disease, congenital immunodeficiency or acquired immunodeficiency, a human who has had a bone marrow transplant, a human infant, or an elderly human). In certain other embodiments of the invention, a “therapeutically effective serum titer” is the serum titer in a cotton rat that results in a RSV titer 5 days after challenge with 10⁵ pfu that is 99% lower than the RSV titer 5 days after challenge with 10⁵ pfu of RSV in a cotton rat not administered an antibody that immunospecifically binds to a RSV antigen.

As used herein, the term “therapy” refers to any protocol, method and/or agent that can be used in the prevention, management, treatment and/or amelioration of a RSV infection (e.g., acute RSV disease, or a RSV URI and/or LRI), otitis media, or a symptom or respiratory condition relating thereto (including, but not limited to, asthma, wheezing, RAD, or a combination thereof). In certain embodiments, the terms “therapies” and “therapy” refer to a biological therapy, supportive therapy, and/or other therapies useful in the prevention, management, treatment and/or amelioration of a RSV infection (e.g., acute RSV disease, or a RSV URI and/or LRI), otitis media, or a symptom or respiratory condition relating thereto (including, but not limited to, asthma, wheezing, RAD, or a combination thereof) known to one of skill in the art such as medical personnel.

As used herein, the terms “treat,” “treatment” and “treating” refer to the reduction or amelioration of the progression, severity, and/or duration of a RSV infection (e.g., acute RSV disease, or a RSV URI and/or LRI), otitis media, or a symptom or respiratory condition relating thereto (including, but not limited to, asthma, wheezing, RAD, or a combination thereof) resulting from the administration of one or more therapies (including, but not limited to, the administration of one or more prophylactic or therapeutic agents, such as an antibody of the invention). In specific embodiments, such terms refer to the reduction or inhibition of the replication of RSV, the inhibition or reduction in the spread of RSV to other tissues or subjects (e.g., the spread to the lower respiratory tract), the inhibition or reduction of infection of a cell with a RSV, the inhibition or reduction of acute RSV disease, the inhibition or reduction of otitis media, the inhibition or reduction of the progression from a LRI to URI, the inhibition or reduction of a respiratory condition caused by or associated with RSV infection (e.g., asthma, wheezing and/or RAD), and/or the inhibition or reduction of one or more symptoms associated with a RSV infection.

The term “upper respiratory” tract refers to the major passages and structures of the upper respiratory tract including the nose or nostrils, nasal cavity, mouth, throat (pharynx), and voice box (larynx).

The term “very little to no loss of the biological activities” as used herein refers to antibody activities, including specific binding abilities of antibodies to a RSV antigen as measured by various immunological assays, including, but not limited to ELISAs and radioimmunoassays. In one embodiment, the antibodies of the formulations of the invention retain approximately 50%, preferably 55%, 60%, 65%, 70%, 75%, 80%, 85%, 90%, 95% or 98% of the ability to immunospecifically bind to a RSV antigen as compared to a reference antibody (e.g., palivizumab) as measured by an immunological assay known to one of skill in the art or described herein. For example, an ELISA based assay may be used to compare the ability of an antibody to immunospecifically bind to a RSV antigen to a palivizumab reference standard. In this assay, plates are coated with a RSV antigen and the binding signal of a set concentration of a palivizumab reference standard is compared to the binding signal of the same concentration of a test antibody.

### 4. DESCRIPTION OF THE FIGURES

FIG. 1A-1B show the amino acid sequences of the (A) light chain variable region and (B) heavy chain variable region of a monoclonal antibody that binds to a RSV antigen, the potency of which can be increased by methods described herein or in Applicants' copending application Ser. Nos. 60/168,426 and 60/186,252 and U.S. Pat. No. 6,656,467. For reference purposes, this is the amino acid sequence of the palivizumab antibody disclosed in Johnson et al., 1997, J. Infect. Dis. 176:1215-1224 and U.S. Pat. No. 5,824,307. Here, the CDR regions are underlined while non-underlined residues form the framework (FR) regions of the variable regions of the antibody. In this antibody, the CDRs are derived from a mouse antibody while the framework regions are derived from a human antibody. The constant regions (not shown) are also derived from a human antibody.

FIG. 2A-2B show the (A) light chain variable region and (B) heavy light chain variable region for an antibody sequence. CDR regions are underlined, and the non-underlined residues form the framework of the variable regions of the antibody. This sequence differs from the sequence disclosed in FIGS. 1A-1B in the first 4 residues of VH CDR1 of the light chain, residue 103 of the light chain FR4 and residue 112 of the heavy chain FR4. For reference purposes, these VL and VH sequences are identical to the VL and VH domains of IX-493L1FR (see Table 2).

FIG. 3 summarizes the results of a RSV microneutralization assay using the anti-RSV antibodies A4B4L1FR-S28R (MEDI-524) and palivizumab, comparing the ability of both antibodies to inhibit the in vitro replication of RSV (Long) in the assay.

FIG. 4 summarizes the results of a RSV microneutralization assay demonstrating the ability of A4B4L1FR-S28R (MEDI-524) to inhibit the in vitro replication of RSV (Long) in the microneutralization assay.

FIG. 5A-5B summarize the results of experiments demonstrating the ability of A4B4L1FR-S28R (MEDI-524) to inhibit the in vivo replication of RSV (Long) in the upper and/or lower respiratory tract of cotton rats, in significantly lower doses than a known anti-RSV antibody, palivizumab.

FIG. 6A-6B show an amino acid sequence comparison of the (A) VH and (B) VL regions of palivizumab, 493L1FR, AFFF(1), and A4b4. CDR regions, as indicated in Kabat et al. (1991) Sequences of proteins of immunological interest. (U.S. Department of Health and Human Services, Washington, D.C.) 5^{th} ed., are in italics. Mutations decreasing k_{off} are labeled in gray, and mutations increasing kₒₙ are underlined.

FIG. 7A-7B show beneficial k_{off} and kₒₙ mutations (highlighted in bold). (A) Single mutations in 493L1FR that result in increased affinity to F protein due to the reduction in k_{off}. (B) Single mutations in AFFF(1), the best k_{off}-improved palivizumab variant, that result in increased affinity to F protein due to the increase in kₒₙ. AFFF(1) contains four beneficial k_{off} mutations which are circled in gray.

FIG. 8A-8D show the results of palivizumab and its variants derived from (A)-(B) viral inhibition assays, and (C)-(D) ELISA assays. (A) Titration of 493L1FR and k_{off}-improved palivizumab Fab variants on immobilized RSV F protein. (B) Inhibition of the binding of k_{off}-improved Fab variants to F protein by palivizumab IgG. In both (A) and (B), bacterial periplasmic extracts containing Fab variants AFFF(1) (□), AFSF (▴), S32A (⋄), 493L1FR (▪), and an irrelevant Fab (◯) were tested as described in Materials and Methods. For the inhibition study, Fab molar ratio of the palivizumab IgG (two Fabs per molecule) to Fab variants was plotted at x-axis. (C) Titration of palivizumab Fab and its kₒₙ-improved Fab variants on immobilized RSV F protein. (D) Inhibition of the binding of kₒₙ-improved Fab variants to F protein by palivizumab IgG. In both (C) and (D), purified Fab variants A4b4 (Δ), A12a6 (), palivizumab Fab (♦), and an irrelevant Fab (◯) were tested.

FIG. 9A-9D show RSV neutralization curves of palivizumab and its variants derived from a microneutralization assay. Several k_{off}-improved variants in the Fab (A) or IgG (B) format were measured for their abilities to inhibit RSV replication in HEp-2 cells. Variants AFFF(1) (□), AFSF (▴), AFFG (Δ), palivizumab (▪), and BSA (◯) were titrated. Several kₒₙ-improved variants as Fab (C) or IgG (D) were also measured. Variant A1e9 (Δ), A13c4 (♦), A12a6 (□), A4b4 (⋄), and palivizumab (▪) were titrated.

FIG. 10A-10D show a summary of the beneficial effects of k_{off}, kₒₙ and bivalence of the antibody on RSV neutralization as indicated by the reduction in IC₅₀ as determined in a microneutralization assay. (A) Comparison of the IC₅₀ of palivizumab Fab with its k_{off}-improved Fab variants. In Fab format, a strong correlation was observed between the IC₅₀ and k_{off}. Combinatorial k_{off} variants with two log reduction in k_{off} have ˜300-fold improvements in the ability to neutralize virus compared with palivizumab. (B) Conversion to IgG of palivizumab and its k_{off}-improved variants. The bivalent binding effect has increased significantly the ability to neutralize virus for the palivizumab and its single k_{off} mutation variants, but not the combinatorial k_{off} variants. The IC₅₀ values of palivizumab IgG and all of its k_{off}-variants converge at ˜3 nM. (C) The IC₅₀ of the combinatorial kₒₙ Fab variants. These variants have ˜4- to 5-fold improvements in kₒₙ, which resulted in substantial enhancements in viral neutralization compared with palivizumab. The differences in IC₅₀ among these kₒₙ variants are in part due to their differences in k_{off}. One outlier with a k_{off} of 2.19×10⁻⁴ s^{-1 is not included. (D) Conversion to IgG of the combinatorial k}ₒₙ-improved variants. Upon conversion to IgG, the IC₅₀ values of all the combinatorial kₒₙ variants converge at ˜0.1-0.2 nM, despite their differences observed in Fab formats. This bivalent effect was similarly observed in k_{off} variants. Overall, the kₒₙ improvement resulted in a 15- to 30-fold enhancement in viral neutralization compared with palivizumab IgG.

FIG. 11A-11D show comparative binding of palivizumab and one each of its best k_{off} and kₒₙ variants to affinity-purified F protein and to F protein on RSV-infected cells. Purified palivizumab (▪), AFFF(1) (k_{off}-improved; □), A4b4 (kₒₙ-improved; ♦) and an irrelevant antibody (◯) in the (A) Fab or (B) IgG format were measured for their binding to purified F protein immobilized at 100 ng/ml on IMMULON-1 plates. The same antibodies in the (C) Fab or (D) IgG format were also measured for their binding to F protein on acetone-fixed HEp-2 cells (1×10³ cells/well) infected with RSV Long strain.

FIG. 12 shows binding of IgGs of palivizumab and one each of its best k_{off} and kₒₙ variants to F protein on the surface of RSV-infected cells as measured by flow cytometry. After infection, HEp-2 cells were stained for RSV F protein with palivizumab, AFFF(1) (k_{off} variant) and A4b4 (kₒₙ variant) at 3 μg/ml, respectively.

FIG. 13A-13B show the nucleotide and translated amino acid sequence of the MEDI-524 (A) VH domain (SEQ ID NO:48) and (B) VL domain (SEQ ID NO:11). CDR sequences are underlined. Where palivizumab differs from MEDI-524, the palivizumab amino acid is shown below the MEDI-524 sequence. Residues that were introduced on the IX-493L1FR template (see also FIG. 2) are indicated in bold.

FIG. 14 shows the mean serum levels after a single IV dose of 3 mg/kg, 15 mg/kg or 30 mg/kg in healthy adults.

FIG. 15 shows the mean serum MEDI-524 trough concentrations during monthly IM injections of 15 mg/kg in a human clinical trial. Concentrations ≧30 μg/mL were maintained throughout dosing in ≧90% of children and increased with continued dosing as expected.

FIG. 16 shows the pharmacokinetic profile of MEDI-524 in nasal secretions following a single IV dose of 3 mg/kg, 15 mg/kg or 30 mg/kg of MEDI-524 or a placebo in children with RSV lower respiratory tract infections. The percent of subjects with MEDI-524 in nasal washes was directly proportional to the amount of MEDI-524 received.

FIG. 17 shows RSV viral titers in nasal secretions of children treated with MEDI-524 or placebo with the indicated doses at days 0, 1 and 2 post-dose. Participants who received MEDI-524 (groups pooled) experienced a significant decrease in mean log₁₀ PFU/mL between Study Day 0 and 1 compared to placebo recipients (Mean=−2.6, SD=1.6, vs. −0.9, SD=1.7; p<0.05).

FIG. 18 shows the percentage of participants with RSV in nasal secretions recovered from tissue culture at days 0, 1, and 2 post-dose. There was a statistically significant decrease in RSV in nasal secretions recovered from tissue culture in MEDI-524 as compared to placebo-treated patients, which indicates biological activity of MEDI-524 in the upper respiratory tract.

FIG. 19 shows the structure of the IgG hinge-Fc region indicating the locations of the residues identified to be involved in the interaction with the FcRn receptor (Ghetie et al., Immunology Today, 18(12):592-598, 1997).

FIG. 20A shows the amino acid sequence of the human IgG1 hinge-Fc region (SEQ ID NO:342) containing a hinge region (SEQ ID NO:341), CH2 domain (SEQ ID NO:339), and CH3 domain (SEQ ID NO:340).

FIG. 20B is similar to FIG. 20A, except that the amino acid residues are renumbered according to the EU Index as in Kabat et al., supra. Bolded regions are preferred embodiment regions of amino acid modifications (see Section 5.1.1).

FIGS. 21A-21B show the amino acid sequences of (A) human FcRn (SEQ ID NO:337) and (B) mouse FcRn (SEQ ID NO:338), respectively.

FIG. 22 shows the amino acid sequence of the human IgG1 hinge-Fc region (SEQ ID NO:342), in which wild-type residues which are mutated by amino acid substitutions are indicated in underlined bold-face.

FIG. 23 shows a schematic diagram of panning process for the phage-displayed modified hinge-Fc library.

FIG. 24 shows a summary of the occurrence of selected mutant residues at the variant positions in the libraries screened.

FIGS. 25A-25D. (A) shows the binding of murine FcRn to immobilized IgG1 having M252Y/S254T/T256E substitutions. Murine FcRn was injected at 10 different concentrations ranging from 1 nM to 556 nM over a surface on which 4000 resonance units (RU) of IgG1 had been coupled. After equilibrium was reached, residual bound protein was eluted with a pulse of PBS, pH 7.4. (B) shows the binding of human FcRn to immobilized IgG1/M252Y/S254T/T256E. Murine FcRn was injected at 8 different concentrations ranging from 71 nM to 2.86 μM over a surface on which 1000 RU of IgG1 had been coupled. After equilibrium was reached, residual bound protein was eluted with a pulse of PBS, pH 7.4. (C) and (D) show scatchard analyses of the data in (A) and (B), respectively, after correction for nonspecific binding. R_{eq} is the corrected equilibrium response at a given concentration, C. The plots are linear with correlation coefficients of 0.97 and 0.998, respectively. The apparent K_{d} are 24 nM and 225 nM, respectively.

FIGS. 26A-26H. (A)-(D) show the results from BIAcore analysis of the binding of murine FcRn at pH 6.0 and pH 7.4 to (A) wild type human IgG1, (B) M252Y/S254T/T256E, (C) H433K/N434F/Y436H, and (D) G385D/G386P/N389S, respectively, after correction for nonspecific binding. Murine FcRn was injected at a concentration of 1.1 μm over a surface on which 1000 RU of wild type IgG1, 1000 RU of M252Y/S254T/T256E, 955 RU of H433K/N434F/Y436H, and 939 RU of G385D/Q386P/N389S had been coupled. (E)-(H) show the results from BIAcore analysis of the binding of human FcRn at pH 6.0 and pH 7.4 to (E) wild type human IgG1, (F) M252Y/S254T/T256E, (G) H433K/N434F/Y436H, and (H) G385D/Q386P/N389S, respectively, after correction for nonspecific binding. Human FcRn was injected at a concentration of 1.4 μm over a surface on which 1000 RU of wild type IgG1, 1000 RU of M252Y/S254T/T256E, 955 RU of H433K/N434F/Y436H, and 939 RU of G385D/Q386P/N389S had been coupled.

FIG. 27 shows the space-filling model of the surface of the Fc fragment of a human IgG1 based upon the human IgG1 structure of Deisenhofer, 1981, Biochemistry 20:2361-2370. Residues are color-coded according to the gain of free energy of stabilization of the Fc-FcRn complex: red, substitutions at these positions were found to increase affinity by a factor of at least 2.5 times in the Fc/human FcRn interaction and of at least 5 time in the Fc/mouse FcRn interaction; blue, substitutions at those positions were found to increase affinity by a factor of less than 2 times in both the Fc-human FcRn and Fc-mouse FcRn interaction. The figure was drawn using Swiss pdb viewer (Guex and Peitsch, 1997, Electrophoresis 18:2714-2723).

FIG. 28 shows the changes in serum concentration ([Mab] ng/ml) over time (in days) of antibody having a wild type constant domain (palivizumab) (open squares), or constant domains with the following mutations: M252Y/S254T/T256E (open circles), G385D/Q386P/N389S (solid squares), and H433K/N434F/Y436H (solid circles). Antibody concentration was determined using anti-human IgG ELISA.

FIGS. 29A-29D shows the nucleotide and amino acid sequences of the heavy chain of MEDI-524 and MEDI-524-YTE. (A) shows the nucleotide sequence of the heavy chain of MEDI-524. (B) shows the amino acid sequence of the heavy chain of MEDI-524. (C) shows the nucleotide sequence of the heavy chain of MEDI-524-YTE, wherein the nucleotide sequence corresponding to the M252Y/S254T/T256E modifications are underlined. (D) shows the amino acid sequence of the heavy chain of MEDI-524-YTE, wherein the M252Y/S254T/T256E modifications are underlined.

FIG. 30 shows BIAcore analysis of the binding of human and Cynomolgus Monkey FcRn at pH 6.0 and pH 7.4 to MEDI-524-YTE. Human and Cynomolgus Monkey FcRn were injected at a concentration of 243 nM over a surface on which ˜1220 RU of MEDI-524-YTE had been coupled.

FIG. 31 shows the results of a RSV microneutralization assay of MEDI-524 and MEDI-524-YTE.

FIG. 32 shows clearance curves of MEDI-524 and MEDI-524-YTE following intravenous injection at 30 mg/kg in Cynomolgus Monkeys. Each time point represents the average serum concentration for ten animals. Standard deviations are indicated by error bars.

FIG. 33 shows human skin and lung tissue cross-reactivity with A4B4 antibody but not with MEDI-524 or an isotype control antibody.

FIG. 34 is a schematic diagram showing the outline for preparing purified antibodies that immunospecifically bind to a RSV antigen.

FIG. 35 shows the amino acid sequence of the VH chain of A4B4L1FR-S28R (SEQ ID NO: 254) comprising M252Y/S254T/T256E modifications in the IgG1 constant domain (MEDI-524-YTE).

### 5. DETAILED DESCRIPTION OF THE INVENTION

The present invention provides antibodies with a high affinity and/or high avidity for a RSV antigen, such as RSV F antigen that are effective in reducing upper as well as lower respiratory tract RSV infections at dosages less than or about equal to the dosage of palivizumab used to prevent only lower respiratory tract infections.

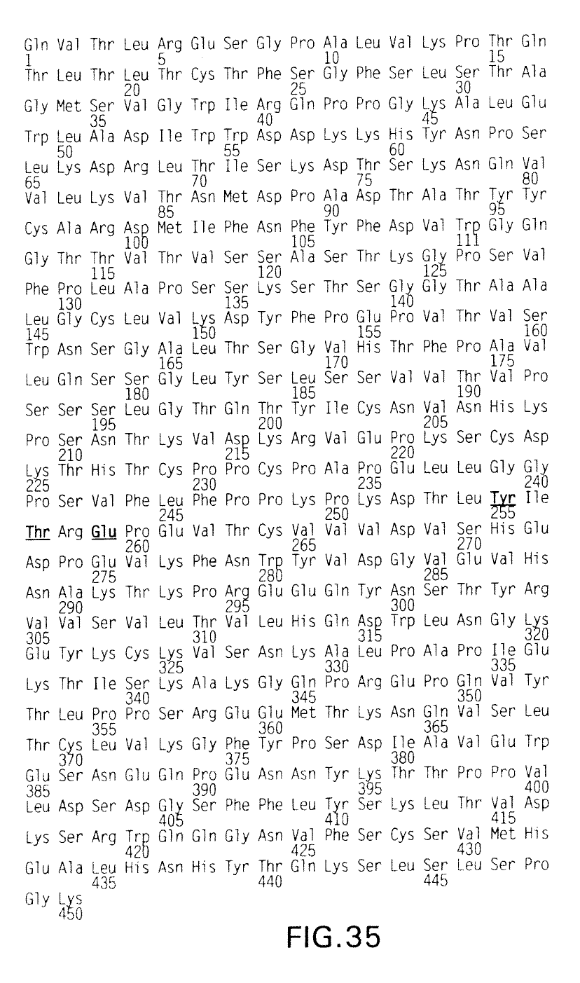

Additionally, the present invention provides an antibody with high affinity and/or high avidity for a RSV antigen (e.g., RSV F antigen) for the prevention, treatment and/or amelioration an upper respiratory tract RSV infection (URI) and/or lower respiratory tract RSV infection (LRI), wherein the antibody comprises one or more amino acid modifications in the IgG constant domain, or FcRn-binding fragment thereof (preferably a modified Fc domain or hinge-Fc domain) that increases the in vivo half-life of the IgG constant domain, or FcRn-binding fragment thereof (e.g., Fc or hinge-Fc domain), and any molecule attached thereto, and increases the affinity of the IgG, or FcRn-binding fragment thereof containing the modified region, for FcRn (i.e., a “modified antibody”). The amino acid modifications may be any modification of a residue (and, in some embodiments, the residue at a particular position is not modified but already has the desired residue), preferably at one or more of residues 251-256, 285-290, 308-314, 385-389, and 428-436, wherein the modification increases the affinity of the IgG, or FcRn-binding fragment thereof containing the modified region, for FcRn. In other embodiments, the antibody comprises a tyrosine at position 252 (252Y), a threonine at position 254 (254T), and/or a glutamic acid at position 256 (256E) (numbering of the constant domain according to the EU index in Kabat et al. (1991). Sequences of proteins of immunological interest. (U.S. Department of Health and Human Services, Washington, D.C.) 5^{th} ed. (“Kabat et al.”)) in the constant domain, or FcRn-binding fragment thereof. In other embodiments, the antibodies comprise 252Y, 254T, and 256E (see EU index in Kabat et al., supra) in the constant domain, or FcRn-binding fragment thereof (hereafter “YTE” see, e.g., FIG. 35).

The present invention provides methods of preventing, managing, treating, neutralizing, and/or ameliorating a RSV infection (e.g., acute RSV disease, or a RSV URI and/or LRI) in a subject comprising administering to said subject an effective amount of an antibody provided herein (a modified or unmodified antibody) which immunospecifically binds to a RSV antigen with high affinity and/or high avidity. Because a lower and/or longer-lasting serum titer of the antibodies of the invention will be more effective in the prevention, management, treatment and/or amelioration of a RSV infection (e.g., acute RSV disease, or a RSV URI and/or LRI) than the effective serum titer of known antibodies (e.g., palivizumab), lower and/or fewer doses of the antibody can be used to achieve a serum titer effective for the prevention, management, treatment and/or amelioration of a RSV infection (e.g., acute RSV disease, or a RSV URI and/or LRI), for example one or more doses per RSV season. The use of lower and/or fewer doses of an antibody of the invention that immunospecifically binds to a RSV antigen reduces the likelihood of adverse effects and are safer for administration to, e.g., infants, over the course of treatment (for example, due to lower serum titer, longer serum half-life and/or better localization to the upper respiratory tract and/or lower respiratory tract as compared to known antibodies (e.g., palivizumab). In certain embodiments, an antibody is administered once or twice per RSV season.

Accordingly, the invention provides antibodies, and methods of using the antibodies thereof, having an increased potency and/or that have increased affinity and/or increased avidity for a RSV antigen (preferably RSV F antigen) as compared to a known RSV antibody (e.g., palivizumab). In some embodiments, the antibody comprises a modified IgG constant domain, or FcRn-binding fragment thereof (preferably, Fc domain or hinge-Fc domain), which results in increased in vivo serum half-life, as compared to, for example, antibodies that do not comprise a modified IgG constant domain, or FcRn-binding fragment thereof (e.g., as compared to the same antibody that does not comprise one or more modifications in the IgG constant domain, or Fc-binding fragment thereof (i.e., the same, unmodified antibody), or as compared to another RSV antibody, such as palivizumab). In some embodiments, the antibodies are administered to a subject, wherein the subject is human subject. In certain embodiments, the subject is in need of therapy thereof. In some embodiments, the subject subjectively knows that he or she is in need or therapy. In other embodiments, the subject does not subjectively know that he or she is in need of therapy.

In a specific embodiment, the invention provides a method of preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD), the method comprising administering to a subject an effective amount of an antibody described herein, for example a modified or unmodified antibody (i.e., an antibody of the invention). In another embodiment, the invention provides a method of preventing, managing, treating and/or ameliorating an acute RSV disease, or progression to an acute RSV disease, the method comprising administering to a subject an effective amount of an antibody of the invention. In some embodiments, the symptom or respiratory condition relating to the RSV infection is asthma, wheezing, RAD, nasal congestion, nasal flaring, cough, tachypnea (rapid coughing), shortness of breath, fever, croupy cough, or a combination thereof. In some embodiments, both upper and lower respiratory tract RSV infections are prevented, treated, managed, and/or ameliorated. In preferred embodiments, the progression from an upper respiratory tract infection to a lower respiratory tract infection is prevented, treated, managed, and/or ameliorated. In other preferred embodiments, acute RSV disease, or the progression to an acute RSV disease, is prevented, treated, managed, and/or ameliorated.

In a specific embodiment, the invention provides a method of preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD), the method comprising administering to a subject an effective amount of an antibody of the invention. In another embodiment, the invention provides a method of preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD), the method comprising administering to a subject an effective amount of an antibody of the invention and an effective amount of a therapy other than an antibody of the invention. Preferably, such a therapy is useful in the prevention, management, treatment and/or amelioration of a RSV infection (preferably an acute RSV disease, or a RSV URI and/or LRI) or otitis media. In a preferred embodiment, the otitis media prevented, treated, managed and/or ameliorated in accordance with the methods of the invention stems from, is caused by or is associated with a RSV infection, preferably a RSV URI and/or LRI.

The present invention provides methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) in a subject, said methods comprising administering to said subject at least a first dose of an antibody of the invention so that said subject has a serum antibody titer of from about 0.1 μg/ml to about 800 μg/ml, such as between 0.1 μg/ml and 500 μg/ml, 0.1 μg/ml and 250 μg/ml, 0.1 μg/ml and 100 μg/ml, 0.1 μg/ml and 50 μg/ml, 0.1 μg/ml and 25 μg/ml or 0.1 μg/ml and 10 μg/ml. In certain embodiments, the serum antibody titer is at least 0.1 μg/ml, at least 0.2 μg/ml, at least 0.4 μg/ml, at least 0.6 μg/ml, at least 0.8 μg/ml, at least 1 μg/ml, at least 1.5 μg/ml, at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 45 μg/ml, at least 50 μg/ml, at least 55 μg/ml, at least 60 μg/ml, at least 65 μg/ml, at least 70 μg/ml, at least 75 μg/ml, at least 80 μg/ml, at least 85 μg/ml, at least 90 μg/ml, at least 95 μg/ml, at least 100 μg/ml, at least 105 μg/ml, at least 110 μg/ml, at least 115 μg/ml, at least 120 μg/ml, at least 125 μg/ml, at least 130 μg/ml, at least 135 μg/ml, at least 140 μg/ml, at least 145 μg/ml, at least 150 μg/ml, at least 155 μg/ml, at least 160 μg/ml, at least 165 μg/ml, at least 170 μg/ml, at least 175 μg/ml, at least 180 μg/ml, at least 185 μg/ml, at least 190 μg/ml, at least 195 μg/ml, or at least 200 μg/ml, at least 250 μg/ml, at least 300 μg/ml, at least 350 μg/ml, at least 400 μg/ml, at least 450 μg/ml, at least 500 μg/ml, at least 550 μg/ml, at least 600 μg/ml, at least 650 μg/ml, at least 700 μg/ml, at least 750 μg/ml, or at least 800 μg/ml. In one embodiment, a prophylactically or therapeutically effective dose results in a serum antibody titer of approximately 75 μg/ml or less, approximately 60 μg/ml or less, resulting in a serum antibody titer of approximately 50 μg/ml or less, approximately 45 μg/ml or less, approximately 30 μg/ml or less, and preferably at least 2 μg/ml, more preferably at least 4 μg/ml, and most preferably at least 6 μg/ml. The antibody of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the antibody comprises an IgG constant domain comprising YTE (e.g., MEDI-524 YTE).

In some embodiments the aforementioned serum antibody concentrations are present in the subject at about or for about 12 to 24 hours after the administration of the first dose of the antibody of the invention and prior to the optional administration of a subsequent dose. In some embodiments, the aforementioned serum antibody concentrations are present for a certain amount of days after the administration of the first dose of the antibody and prior to the optional administration of a subsequent dose, wherein said certain number of days is from about 20 days to about 180 days (or longer), such as between 20 days and 90 day, 20 days and 60 days, or 20 days and 30 days, and in certain embodiments is at least 20 days, at least 25 days, at least 30 days, at least 35 days, at least 40 days, at least 45 days, at least 50 days, at least 60 days, at least 75 days, at least 90 days, at least 105 days, at least 120 days, at least 135 days, at least 150 days, at least 165 days, at least 180 days or longer. In certain embodiments, the first dose of the antibody resulting in the aforementioned serum antibody concentrations is about 60 mg/kg or less, about 50 mg/kg or less, about 45 mg/kg or less, about 40 mg/kg or less, about 30 mg/kg or less, about 20 mg/kg or less, about 15 mg/kg or less, about 10 mg/kg or less, about 5 mg/kg or less, about 4 mg/kg or less, about 3 mg/kg, about 2 mg/kg or less, about 1.5 mg/kg or less, about 1.0 mg/kg or less, about 0.80 mg/kg or less, about 0.40 mg/kg or less, about 0.20 mg/kg or less, about 0.10 mg/kg or less, about 0.05 mg/kg or less, or about 0.025 mg/kg or less. In some embodiments, the first dose of an antibody of the invention is a prophylactically or therapeutically effective dose that results in any one of the aforementioned serum antibody concentrations. In one embodiment, the first dose of an antibody of the invention is administered in a sustained release formulation and/or by intranasal or pulmonary delivery. The antibody of the invention may or may not comprise a modified IgG (e.g., IgG1) constant domain, or FcRn-binding fragment thereof (e.g., Fc or hinge-Fc domain). In certain embodiments, the antibody is a modified antibody, and preferably comprises the YTE modification (e.g., MEDI-524 YTE).

The present invention also provides methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) in a subject, said methods comprising administering to said subject a first dose of an antibody of the invention so that said subject has a reduced RSV viral lung titer and/or RSV viral sputum titer (as determined using methods described herein (e.g., Example 6.9) or otherwise known in the art) as compared to a negative control, for example a subject receiving a placebo, as compared to the tiers in a subject prior to administration of the first dose of an antibody of the invention, or as compared to a subject receiving another RSV antibody (e.g., palivizumab). In embodiments, wherein the antibody is a modified antibody of the invention, the reduced RSV viral lung tier and/or RSV viral sputum titer may further be compared to a subject receiving the same antibody without the modifications in the IgG constant domain.

The present invention also provides methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) in a subject, said methods comprising administering to said subject a first dose of an antibody of the invention so that said subject has a nasal turbinate and/or nasal secretion antibody concentration of from about 0.01 μg/ml to about 2.5 μg/ml (or more). In certain embodiments, the nasal turbinate and/or nasal secretion antibody concentration is at least 0.01 μg/ml, at least 0.011 μg/ml, at least 0.012 μg/ml, at least 0.013 μg/ml, at least 0.014 μg/ml, at least 0.015 μg/ml, at least 0.016 μg/ml, at least 0.017 μg/ml, at least 0.018 μg/ml, at least 0.019 μg/ml, at least 0.02 μg/ml, at least 0.025 μg/ml, at least 0.03 μg/ml, at least 0.035 μg/ml, at least 0.04 μg/ml, at least 0.05 μg/ml, at least 0.06 μg/ml, at least 0.07 μg/ml, at least 0.08 μg/ml, at least 0.09 μg/ml, at least 0.1 μg/ml, at least 0.11 μg/ml, at least 0.115 μg/ml, at least 0.12 μg/ml, at least 0.125 μg/ml, at least 0.13 μg/ml, at least 0.135 μg/ml, at least 0.14 μg/ml, at least 0.145 μg/ml, at least 0.15 μg/ml, at least 0.155 μg/ml, at least 0.16 μg/ml, at least 0.165 μg/ml, at least 0.17 μg/ml, at least 0.175 μg/ml, at least 0.18 μg/ml, at least 0.185 μg/ml, at least 0.19 μg/ml, at least 0.195 μg/ml, at least 0.2 μg/ml, at least 0.3 μg/ml, at least 0.4 μg/ml, at least 0.5 μg/ml, at least 0.6 μg/ml, at least 0.7 μg/ml, at least 0.8 μg/ml, at least 0.9 μg/ml, at least 1.0 μg/ml, at least 1.1 μg/ml, at least 1.2 μg/ml, at least 1.3 μg/ml, at least 1.4 μg/ml, at least 1.5 μg/ml, at least 1.6 μg/ml, at least 1.7 μg/ml, at least 1.8 μg/ml, at least 1.9 μg/ml, at least 2.0 μg/ml, at least 2.1 μg/ml, at least 2.2 μg/ml, at least 2.3 μg/ml, at least 2.4 μg/ml, at least 2.5 μg/ml or more. The antibody of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the modified IgG constant domain comprises the YTE modification (e.g., MEDI-524 YTE).

In some embodiments the aforementioned nasal turbinate and/or nasal secretion antibody concentrations are present in the subject at about or for about 12 to 24 hours after the administration of the first dose of the antibody of the invention and prior to the optional administration of a subsequent dose. In some embodiments, the aforementioned nasal turbinate and/or nasal secretion antibody concentrations are present for a certain amount of days after the administration of the first dose of the antibody and prior to the optional administration of a subsequent dose, wherein said certain number of days is from about 20 days to about 180 days (or more), and in certain embodiments is at least 20 days, at least 25 days, at least 30 days, at least 35 days, at least 40 days, at least 45 days, at least 50 days, at least 60 days, at least 75 days, at least 90 days, at least 105 days, at least 120 days, at least 135 days, at least 150 days, at least 165 days, at least 180 days or more. In certain embodiments, the first dose of the antibody resulting in the aforementioned nasal turbinate and/or nasal secretion antibody concentrations is about 60 mg/kg or less, about 50 mg/kg or less, about 45 mg/kg or less, about 40 mg/kg or less, about 30 mg/kg or less, about 20 mg/kg or less, about 15 mg/kg or less, about 10 mg/kg or less, about 5 mg/kg or less, about 4 mg/kg or less, about 3 mg/kg, about 2 mg/kg or less, about 1.5 mg/kg or less, about 1.0 mg/kg or less, about 0.80 mg/kg or less, about 0.40 mg/kg or less, about 0.20 mg/kg or less, about 0.10 mg/kg or less, about 0.05 mg/kg or less, or about 0.025 mg/kg or less. In some embodiments, the first dose of an antibody of the invention is a prophylactically or therapeutically effective dose that results in any one of the aforementioned nasal turbinate and/or nasal secretion antibody concentrations. In one embodiment, the first dose of an antibody of the invention is administered in a sustained release formulation and/or by intranasal and/or pulmonary delivery. The antibody of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the modified IgG constant domain comprises the YTE modification (e.g., MEDI-524 YTE).

In specific embodiments, the present invention provides methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) in a subject, said methods comprising administering an effective amount of an antibody of the invention, wherein the effective amount results in a reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105-fold, about 110-fold, about 115-fold, about 120 fold, about 125-fold or higher in RSV titer in the nasal turbinate and/or nasal secretion. The fold-reduction in RSV titer in the nasal turbinate and/or nasal secretion may be as compared to a negative control (such as placebo), as compared to another therapy (including, but not limited to treatment with palivizumab), as compared to the titer in the patient prior to antibody administration or, in the case of modified antibodies, as compared to the same unmodified antibody (e.g., the same antibody prior to constant region modification). The antibody of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the modified IgG constant domain comprises the YTE modification (e.g., MEDI-524 YTE).

The present invention provides methods of neutralizing RSV in the upper and/or lower respiratory tract or in the middle ear using an antibody of the invention to achieve a prophylactically or therapeutically effective serum titer, wherein said effective serum titer is less than 30 μg/ml (and is preferably about 2 μg/ml, more preferably about 4 μg/ml, and most preferably about 6 μg/ml) for about 20, 25, 30, 35, 40, 45, 60, 75, 90, 105, 120, 135, 150, 165, 180 or more days after administration without any other dosage administration. The antibody of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the IgG constant domain comprises the YTE modification (e.g., MEDI-524 YTE).

In preferred embodiments, the antibodies used in accordance with the methods of the invention have a high affinity for RSV antigen. In one embodiment, the antibodies used in accordance with the methods of the invention have a higher affinity for a RSV antigen (e.g., RSV F antigen) than known antibodies, (e.g., palivizumab or other wild-type antibodies). The antibody used in accordance with the methods of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the IgG constant domain comprises the YTE modification (e.g., MEDI-524 YTE). In a specific embodiment, the antibodies used in accordance with the methods of the invention have a 20-fold, 25-fold, 30-fold, 35-fold, 40-fold, 45-fold, 50-fold, 55-fold, 60-fold, 65-fold, 70-fold, 75-fold, 80-fold, 90-fold, 100-fold or higher affinity for a RSV antigen than a known anti-RSV antibody as assessed by techniques described herein or known to one of skill in the art (e.g., a BIAcore assay). In a more specific embodiment, the antibodies used in accordance with the methods of the invention have a 20-fold, 25-fold, 30-fold, 35-fold, 40-fold, 45-fold, 50-fold, 55-fold, 60-fold, 65-fold, 70-fold, 75-fold, 80-fold, 90-fold, 100-fold or higher affinity for a RSV F antigen than palivizumab as assessed by techniques described herein or known to one of skill in the art (e.g., a BIAcore assay). In a preferred embodiment, the antibodies used in accordance with the methods of the invention have a 65-fold, preferably 70-fold, or higher affinity for a RSV F antigen than palivizumab as assessed by techniques described herein or known to one of skill in the art (e.g., a BIAcore assay). In accordance with these embodiments, the affinity of the antibodies are, in one embodiment, assessed by a BIAcore assay.

In one embodiment, the antibodies used in accordance with the methods of the invention immunospecifically bind to one or more RSV antigens and have an association rate constant or kₒₙ rate (antibody (Ab)+antigen (Ag)—kₒₙ—>Ab−Ag) of between about 10⁵ M⁻¹ s⁻¹ to about 10⁸ M⁻¹ s⁻¹ (or higher), and in certain embodiments is at least 10⁵ M⁻¹ s⁻¹, at least 2×10⁵ at least 4×10⁵ M⁻¹ s⁻¹, at least 5×10⁵ M⁻¹ s⁻¹, at least 10⁶ M⁻¹ s⁻¹, at least 5×10⁶ M⁻¹ s⁻¹, at least 10⁷ M⁻¹ s⁻¹, at least 5×10⁷ M⁻¹ s⁻¹, or at least 10⁸ M⁻¹ s⁻¹. In another embodiment, the antibodies used in accordance with the methods of the invention immunospecifically bind to a RSV antigen and have a kₒₙ rate that is 1-fold, 1.5-fold, 2-fold, 3-fold, 4-fold or 5-fold higher than a known anti-RSV antibody. In a preferred embodiment, the antibodies used in accordance with the methods of the invention immunospecifically bind to a RSV F antigen and have a kₒₙ rate that is 1-fold, 2-fold, 3-fold, 4-fold, 5-fold or higher than palivizumab. A more detailed explanation of individual rate constant and affinity calculations can be found in the BIAevaluation Software Handbook (BIAcore, Inc., Piscataway, N.J.) and Kuby (1994) Immunology, 2^{nd} Ed. (W.H. Freeman & Co., New York, N.Y.). The antibody used in accordance with the methods of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the IgG constant domain comprises the YTE modification (e.g., MEDI-524 YTE).

In a specific embodiment, the antibodies used in accordance with the methods of the invention immunospecifically bind to one or more RSV antigens and have a k_{off} rate (Ab−Ag—K_{off}—>Ab+Ag) of less than 5×10⁻¹ s⁻¹, less than 10⁻¹ s⁻¹, less than 5×10⁻² s⁻¹, less than 10⁻² s⁻¹, less than 5×10⁻³ s⁻¹, less than 10⁻³ s⁻¹, and preferably less than 5×10⁻⁴ s⁻¹, less than 10⁻⁴ s⁻¹, less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s⁻¹, less than 10⁻⁷ s⁻¹, less than 5×10⁻⁸ s⁻¹, less than 10⁻⁸ s⁻¹, less than 5×10⁻⁹ s¹, less than 10⁻⁹ s⁻¹, less than 5×10⁻¹⁰ s⁻¹, or less than 10⁻¹⁰ s⁻¹. In another embodiment, the antibodies used in accordance with the methods of the invention immunospecifically bind to a RSV antigen and have a k_{off} rate that is 1-fold, 1.5-fold, 2-fold, 3-fold, 4-fold, 5-fold, 10-fold, 20-fold, 30-fold, 40-fold, 50-fold, 60-fold, 70-fold, 80-fold, 90-fold, or 100-fold lower than a known anti-RSV antibody. In a preferred embodiment, the antibodies used in accordance with the methods of the invention immunospecifically bind to a RSV F antigen and have a k_{off} rate that is 1-fold, 2-fold, 3-fold, 4-fold, 5-fold, 10-fold, 15-fold, 20-fold, 30-fold, 40-fold, 50-fold, 60-fold, 70-fold, 80-fold, 90-fold, or 100-fold or lower than palivizumab. The antibody used in accordance with the methods of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the IgG constant domain comprises the YTE modification (e.g., MEDI-524 YTE).

In a specific embodiment, the antibodies used in accordance with the methods of the invention immunospecifically bind to one or more RSV antigens have a kₒₙ of between about 10⁵ M⁻¹ s⁻¹ and 10⁸ M⁻¹ s⁻¹ (or higher), and in certain embodiments is at least 10⁵ M⁻¹ s⁻¹, preferably at least 2×10⁵ M⁻¹ s⁻¹, at least 4×10⁵ M⁻¹ s⁻¹, at least 5×10⁵ M⁻¹ s⁻¹, at least 10⁶ M⁻¹ s⁻¹, at least 5×10⁶ M⁻¹ s⁻¹, at least 10⁷ M⁻¹ s⁻¹, at least 5×10⁷ M⁻¹ s⁻¹, or at least 10⁸ M⁻¹ s⁻¹ and also have a k_{off} rate of less than 5×10⁻¹ s⁻¹, less than 10⁻¹ s⁻¹, less than 5×10⁻² s⁻¹, less than 10⁻² s⁻¹, less than 5×10⁻³ s⁻¹, less than 10⁻³ s⁻¹, and preferably less than 5×10⁻⁴ s⁻¹, less than 10⁻⁴ s⁻¹, less than 7.5×10⁻⁵ s⁻¹, less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s⁻¹, less than 10⁻⁷ s⁻¹, less than 5×10⁻⁸ s⁻¹, less than 10⁻⁸ s⁻¹, less than 5×10⁻⁹ s⁻¹, less than 10⁻⁹ s⁻¹, less than 5×10⁻¹⁰ s⁻¹, or less than 10⁻¹⁰ s⁻¹. In one embodiment, an antibody of the invention has a kₒₙ that is about 2-fold, about 3-fold, about 4-fold, or about 5-fold, or higher than palivizumab. In another embodiment, an antibody of the invention has a k_{off} that is about 2-fold, about 3-fold, about 4-fold, or about 5-fold, or lower than palivizumab. The antibody used in accordance with the methods of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the IgG constant domain comprises the YTE modification (e.g., MEDI-524 YTE).

In a specific embodiment, the antibodies used in accordance with the methods of the invention immunospecifically bind to one or more RSV antigens and have an affinity constant or Kₐ (kₒₙ/k_{off}) of from about 10² M⁻¹ to about 5×10¹⁵ M⁻¹, and in certain embodiments is at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷M⁻¹, at least 10⁸ M⁻¹, and preferably at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹ at least 5×10¹¹ M⁻¹, at least 10¹² M⁻¹, at least 5×10¹² M⁻¹, at least 10¹³M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. The antibody used in accordance with the methods of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the IgG constant domain comprises the YTE modification (e.g., MEDI-524 YTE).

In one embodiment, an antibody used in accordance with the methods of the invention has a dissociation constant or Kₐ (k_{off}/kₒₙ) of less than 5×10⁻² M, less than 10⁻² M, less than 5×10⁻³ M, less than 10⁻³ M, less than 5×10⁻⁴ M, less than 10⁻⁴ M, less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹²M, less than 5×10⁻¹³ M, less than 10⁻¹³ M, less than 5×10⁻¹⁴ M, less than 10⁻¹⁴ M, less than 5×10⁻¹⁵ M, less than 10⁻¹⁵ M, or less than 5×10⁻¹⁶ M. The antibody used in accordance with the methods of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the IgG constant domain comprises the YTE modification (e.g., MEDI-524 YTE).

In a specific embodiment, the antibodies used in accordance with the methods of the invention immunospecifically bind to a RSV antigen and have a dissociation constant (K_{d}) of less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM as assessed using an described herein or known to one of skill in the art (e.g., a BIAcore assay). In another embodiment, the antibodies used in accordance with the methods of the invention immunospecifically bind to a RSV antigen and have a dissociation constant (K_{d}) of between 25 to 3400 pM, 25 to 3000 pM, 25 to 2500 pM, 25 to 2000 pM, 25 to 1500 pM, 25 to 1000 pM, 25 to 750 pM, 25 to 500 pM, 25 to 250 pM, 25 to 100 pM, 25 to 75 pM, 25 to 50 pM as assessed using an described herein or known to one of skill in the art (e.g., a BIAcore assay). In another embodiment, the antibodies used in accordance with the methods of the invention immunospecifically bind to a RSV antigen and have a dissociation constant (K_{d}) of 500 pM, preferably 100 pM, more preferably 75 pM and most preferably 50 pM as assessed using an described herein or known to one of skill in the art (e.g., a BIAcore assay). The antibody used in accordance with the methods of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the IgG constant domain comprises the YTE modification (e.g., MEDI-524 YTE).

The present invention also provides methods for preventing, managing, treating and/or ameliorating a RSV infection (e.g., acute RSV disease, or a RSV URI and/or LRI) and/or one or more symptoms associated with an upper and/or lower respiratory tract, middle ear RSV infection and/or RSV disease, said methods comprising administering to a subject a composition (for example, by pulmonary delivery or intranasal delivery) comprising one or more antibodies of the invention which immunospecifically bind to one or more RSV antigens (e.g., RSV F antigen) with higher affinity and/or higher avidity than known antibodies such as, e.g., palivizumab (e.g., antibodies or antibody fragments having an affinity of from about 2×10⁸ M⁻¹ to about 5×10¹² M⁻¹ (or higher), and preferably at least 2×10⁸ M⁻¹, at least 2.5×10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, or at least 5×10¹² M⁻¹ for one or more RSV antigens). The antibody used in accordance with the methods of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the IgG constant domain comprises the YTE modification (e.g., MEDI-524 YTE).

The IC₅₀ is the concentration of antibody that neutralizes 50% of the RSV in an in vitro microneutralization assay. In certain embodiments, the microneutralization assay is a microneutralization assay described herein (for example, as described in Examples 6.4, 6.8, and 6.18 herein) or as in Johnson et al., 1999, J. Infectious Diseases 180:35-40. In specific embodiments, the antibodies used in accordance with the methods of the invention immunospecifically bind to one or more RSV antigens and have a median inhibitory concentration (IC₅₀) of less than 6 nM, less than 5 nM, less than 4 nM, less than 3 nM, less than 2 nM, less than 1.75 nM, less than 1.5 nM, less than 1.25 nM, less than 1 nM, less than 0.75 nM, less than 0.5 nM, less than 0.25 nM, less than 0.1 nM, less than 0.05 nM, less than 0.025 nM, or less than 0.01 nM, in an in vitro microneutralization assay. The antibody used in accordance with the methods of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the IgG constant domain comprises the YTE modification (e.g., MEDI-524 YTE).

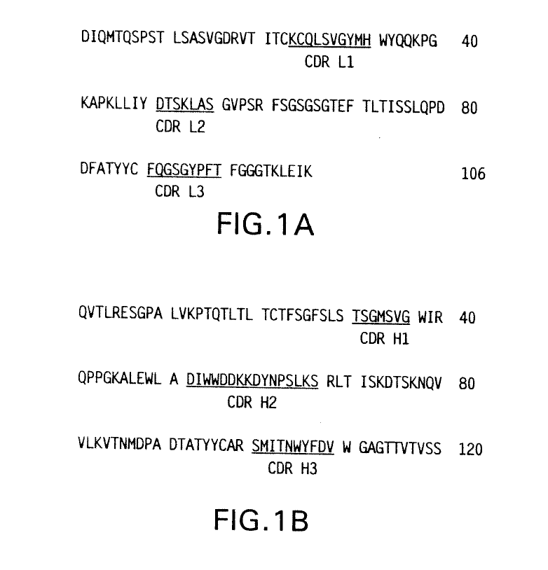

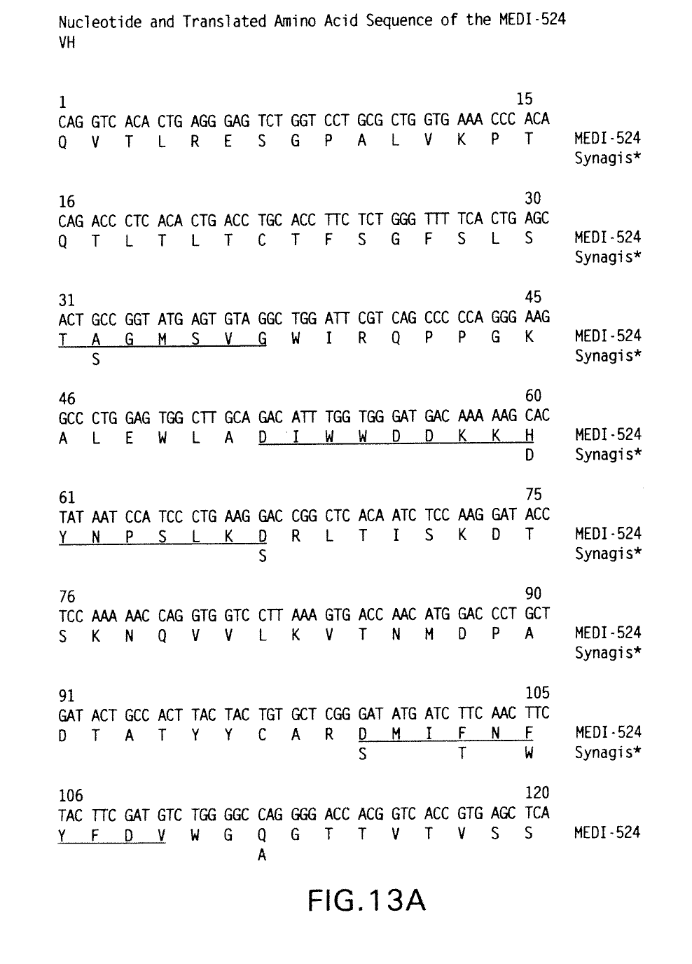

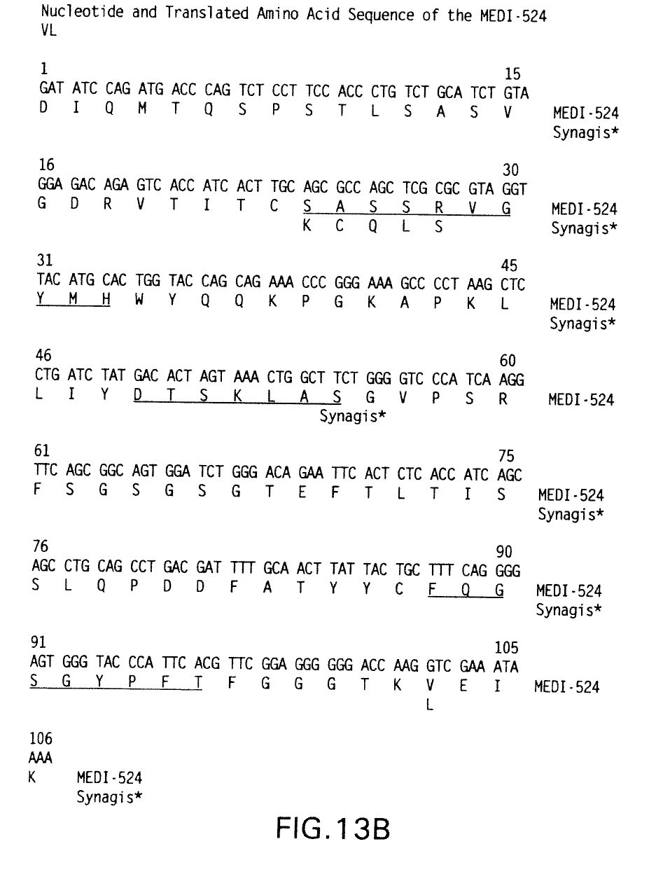

The methods of the invention also encompass the use of antibodies that immunospecifically bind to a RSV antigen (e.g., RSV F antigen), the antibodies comprising a heavy chain variable (VH) chain having the amino acid sequence of any VH chain used in Table 2. The methods of the invention also encompass the use of antibodies that immunospecifically bind to a RSV antigen (e.g., RSV F antigen), the antibodies comprising a VH domain having the amino acid sequence of any VH domain listed in Table 2. The methods of the invention further encompass the use of antibodies that immunospecifically bind to a RSV antigen (e.g., RSV F antigen), the antibodies comprising one or more (e.g., one, two or three) VH complementarity determining regions (CDRs) having the amino acid sequence of one or more VH CDRs listed in Table 2 and/or Tables 3A-3C. In preferred embodiments, the antibody comprises VH framework regions that are identical to those shown in FIG. 13A. In other embodiments, the antibody comprises VH framework regions that are identical to those of the VH framework region shown in FIG. 1B. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The methods of the invention also encompass the use of antibodies that immunospecifically bind to a RSV antigen (e.g., RSV F antigen), the antibodies comprising a light chain variable (VL) chain having the amino acid sequence of any VL chain used in Table 2. The methods of the invention also encompass the use of antibodies that immunospecifically bind to a RSV antigen (e.g., RSV F antigen), the antibodies comprising a light chain variable (VL) domain having the amino acid sequence of any VL domain listed in Table 2. The methods of the invention also encompass the use of antibodies that immunospecifically bind to a RSV antigen (e.g., RSV F antigen), the antibodies comprising one or more VL CDRs having the amino acid sequence of one or more VL CDRs listed in Table 2 and/or Tables 3D-3F. In preferred embodiments, the antibody comprises VL framework regions are identical to that shown in FIG. 13B. In other embodiments, the antibody comprises VL framework regions that are identical to that shown in FIG. 1A. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The methods of the invention also encompass the use of antibodies that immunospecifically bind to a RSV antigen (e.g., RSV F antigen), the antibodies comprising a VH chain having an amino acid sequence of any VH chain listed in Table 2 and a VL chain having an amino acid sequence of any VL chain listed in Table 2. The methods of the invention also encompass the use of antibodies that immunospecifically bind to a RSV antigen (e.g., RSV F antigen), the antibodies comprising a VH domain and a VL domain having the amino acid sequence of any VH domain and any VL domain listed in Table 2. The methods of the invention further encompass the use of antibodies that immunospecifically bind to a RSV antigen (e.g., RSV F antigen), the antibodies comprising any one or more (e.g., one, two, or three) VH CDRs and any one or more (e.g., one, two, or three) VL CDRs having an amino acid sequence of one or more VH CDRs and one or more VL CDRs listed in Table 2 and/or Tables 3A-3F. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In some embodiments, the methods of the invention encompass the use of an antibody listed in Table 2. In certain embodiments, the antibody listed in Table 2 comprises a modified IgG constant domain, or FcRn-binding fragment thereof (preferably, Fc domain or hinge-Fc domain). In preferred embodiments, the methods of the invention encompass the use of a A4B4L1FR-S28R (MEDI-524) (FIG. 13) antibody or a modified antibody thereof. In one embodiment, the antibody comprises a VH and/or VL domain(s) or chain(s) of the A4B4L1FR-S28R (MEDI-524) antibody. In certain embodiments, the A4B4L1FR-S28R (MEDI-524) antibody comprises a modified IgG constant domain, or FcRn-binding fragment thereof (preferably, Fc domain or hinge-Fc domain). In preferred embodiments, the A4B4L1FR-S28R (MEDI-524) antibody comprises a modified IgG, such as a modified IgG1, constant domain, or FcRn-binding fragment thereof, comprising YTE.

Thus, methods of the invention encompass the use of modified antibodies which have increased in vivo half-lives compared to known anti-RSV antibodies as a result of, e.g., one or more modifications in amino acid residues identified to be involved in the interaction between the Fc domain of said modified antibodies and the FcRn receptor. In one embodiment, the methods of the invention encompass the use of an antibody that immunospecifically binds to a RSV antigen (e.g., RSV F antigen) with a high affinity and/or high avidity (e.g., an antibody that has a higher affinity and/or avidity for a RSV F antigen than palivizumab, including but not limited to those described in Table 2), and which comprises a modified IgG constant domain, or FcRn-binding fragment thereof (preferably, Fc domain or hinge-Fc domain), wherein the modified IgG constant domain results in increased affinity of the modified IgG constant domain for the FcRn relative to the same antibody that does not comprise a modified IgG domain or another RSV-antibody, such as the Fc domain of palivizumab. In accordance with this embodiment, the increased affinity of the Fc domain of said modified antibodies results in an in vivo half-life of said modified antibodies of from about 20 days to about 180 days (or more) and in some embodiments is at least 20 days, at least 25 days, at least 30 days, at least 35 days, at least 40 days, at least 45 days, at least 50 days, at least 60 days, at least 75 days, at least 90 days, at least 105 days, at least 120 days, at least 135 days, at least 150 days, at least 165 days, at least 180 days or longer. In a preferred embodiment, the modified antibody comprises the VH and VL domain or chain of A4B4L1FR-S28R (MEDI-524) (FIG. 13), or an antigen-binding fragment thereof, and an Fc domain with increased affinity for the FcRn receptor relative to the Fc domain of, e.g., palivizumab. In certain embodiments, the modified antibody comprises the YTE modification.

The methods of the invention encompass the use of one or more antibodies (modified or unmodified) which immunospecifically bind to one or more RSV antigens (preferably, RSV F antigen) wherein said antibody is pegylated. In one embodiment, the methods of the invention encompass the use of one or more pegylated antibodies that immunospecifically bind to one or more RSV antigens (preferably, a RSV F antigen) with a high avidity and/or high affinity (e.g., a higher affinity for a RSV F antigen than palivizumab), including but not limited to those described in Table 2. In a preferred embodiment, the antibody is a pegylated A4B4L1FR-S28R (MEDI-524) antibody or an antigen-binding fragment thereof.

In one embodiment, the methods of the invention encompass the use of one or more pegylated antibodies which immunospecifically bind to a RSV antigen with a higher affinity and/or avidity (e.g., higher than palivizumab). In a specific embodiment, the pegylated antibody comprises a VH and/or VL domain or chain of an antibody described in Table 2. In a preferred embodiment, the pegylated antibody comprises a VH and/or VL domain or chain of A4B4L1FR-S28R (MEDI-524) (FIG. 13) or an antigen binding fragment thereof. In one embodiment, the antibody comprises a VH and/or VL domain or chain of an antibody listed in Table 2. In a preferred embodiment, the pegylated antibody comprises the VH and VL chain of A4B4L1FR-S28R (MEDI-524). In certain embodiments, the pegylated antibody is a modified pegylated antibody.

5.1 Antibodies

It should be recognized that antibodies that immunospecifically bind to a RSV antigen are known in the art. For example, palivizumab is a humanized monoclonal antibody presently used for the prevention of RSV infection in pediatric patients. The present invention provides methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) by administering to a subject an effective amount of an anti-RSV antibody of the invention (preferably, A4B4L1FR-S28R (MEDI-524) or an antigen-binding fragment thereof).

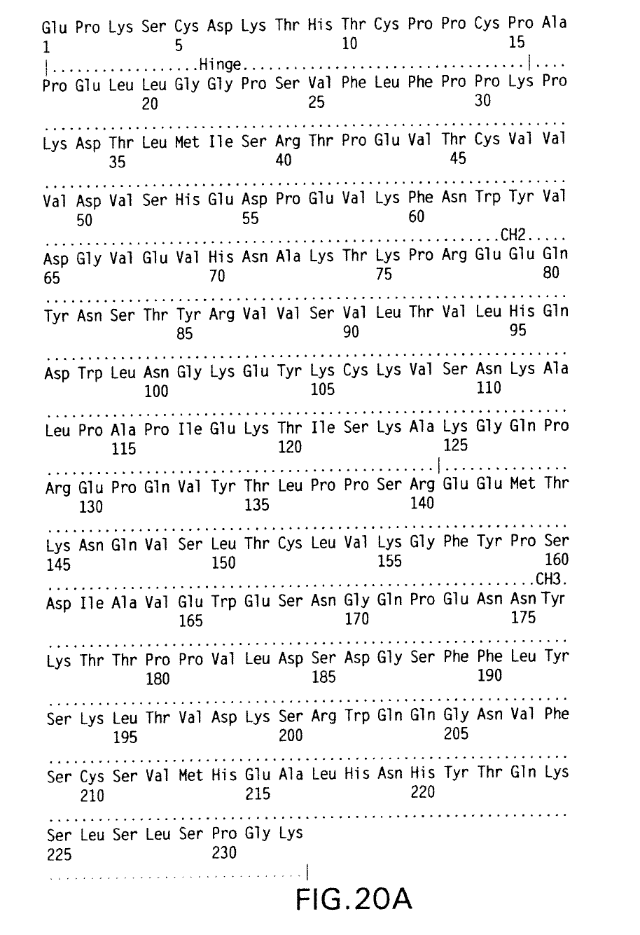

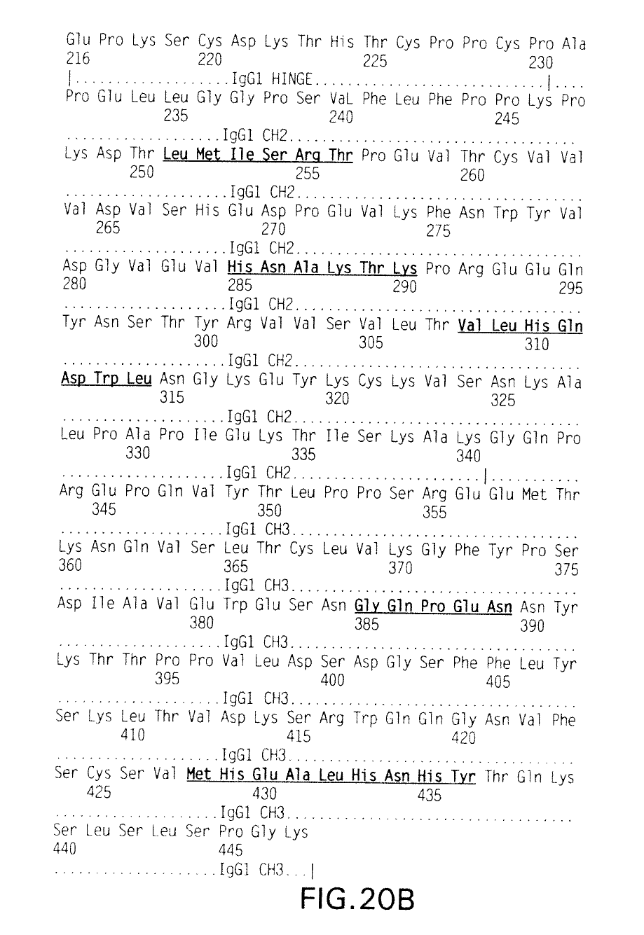

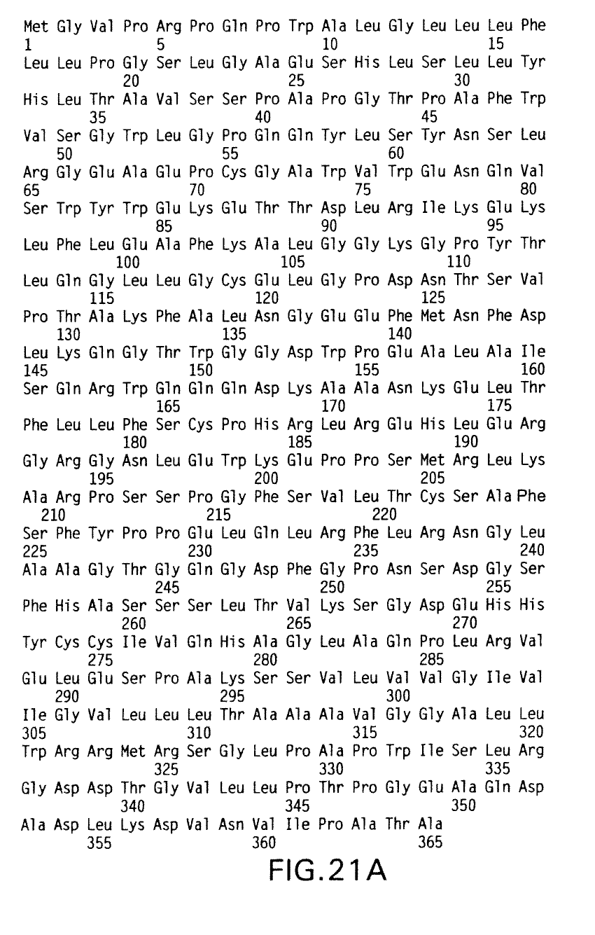

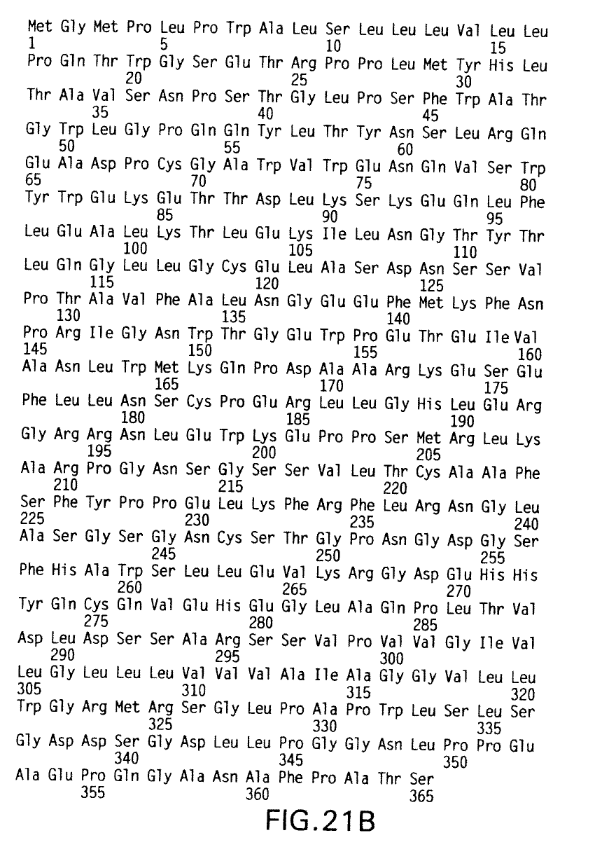

The present invention also provides methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) by administering to a subject an effective amount of an anti-RSV antibody of the invention, wherein the antibody comprises a modified IgG constant domain, or FcRn-binding fragment thereof (preferably, Fc domain or hinge-Fc domain). In preferred embodiments, the modified antibody is a modified A4B4L1FR-S28R (MEDI-524) antibody (e.g., MEDI-524-YTE). The amino acid modifications may be any modification of a residue (and, in some embodiments, the residue at a particular position is not modified but already has the desired residue), preferably at one or more of residues 251-256, 285-290, 308-314, 385-389, and 428-436, that increases the in vivo half-life of the IgG constant domain, or FcRn-binding fragment thereof (e.g., Fc or hinge-Fc domain), and any molecule attached thereto, and increases the affinity of the modified IgG, or fragment thereof, for FcRn. In preferred embodiment, the modified antibodies have one or more amino acid modifications in the second constant CH2 domain (residues 231-340 of human IgG1) (e.g., SEQ ID NO:339) (see, e.g., FIG. 20B) and/or the third constant CH3 domain (residues 341-447 of human IgG1) (e.g., SEQ ID NO:340) (see, e.g., FIG. 20B), with numbering according to the EU Index as in Kabat, supra. In certain embodiments, the antibody comprises a tyrosine at position 252 (252Y), a threonine at position 254 (254T), and/or a glutamic acid at position 256 (256E) (e.g., a M252Y, S254T and/or T256E mutation (see EU index in Kabat et al. (1991). Sequences of proteins of immunological interest. (U.S. Department of Health and Human Services, Washington, D.C.) 5^{th} ed.) in the constant domain, or FcRn-binding fragment thereof.

Set forth below, is a more detailed description of the antibodies encompassed within the various aspects of the invention.

The present invention provides antibodies (modified and unmodified) that immunospecifically bind to one or more RSV antigens. Preferably, the antibodies of the invention immunospecifically bind to one or more RSV antigens regardless of the strain of RSV. The present invention also provides antibodies that differentially or preferentially bind to RSV antigens from one strain of RSV versus another RSV strain. In a specific embodiment, the antibodies of the invention immunospecifically bind to the RSV F glycoprotein, G glycoprotein or SH protein. In a preferred embodiment, the antibodies present invention immunospecifically bind to the RSV F glycoprotein. In another preferred embodiment, the antibodies of the present invention bind to the A, B, or C antigenic sites of the RSV F glycoprotein.

Antibodies of the invention include, but are not limited to, monoclonal antibodies, multispecific antibodies, human antibodies, humanized antibodies, chimeric antibodies, single domain antibodies, camelised antibodies, single chain Fvs (scFv) single chain antibodies, Fab fragments, F(ab′) fragments, disulfide-linked Fvs (sdFv) intrabodies, and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to a RSV antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. In a specific embodiment, an antibody (modified or unmodified) of the invention is an IgG antibody, preferably an IgG1. In another specific embodiment, an antibody of the invention is not an IgA antibody.

The antibodies of the invention may be from any animal origin including birds and mammals (e.g., human, murine, donkey, sheep, rabbit, goat, guinea pig, camel, horse, or chicken). Preferably, the antibodies of the invention are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies of the present invention may be monospecific, bispecific, trispecific or of greater multispecificity. Multispecific antibodies may be specific for different epitopes of a RSV polypeptide or may be specific for both a RSV polypeptide as well as for a heterologous epitope, such as a heterologous polypeptide or solid support material. See, e.g., PCT publications WO 93/17715, WO 92/08802, WO 91/00360, and WO 92/05793; Tutt, et al., J. Immunol. 147:60-69 (1991); U.S. Pat. Nos. 4,474,893, 4,714,681, 4,925,648, 5,573,920, and 5,601,819; and Kostelny et al., J. Immunol. 148:1547-1553 (1992).

In a specific embodiment, antibodies for use in the methods of the invention are bispecific T cell engagers (BiTEs). Bispecific T cell engagers (BiTE) are bispecific antibodies that can redirect T cells for antigen-specific elimination of targets. A BiTE molecule has an antigen-binding domain that binds to a T cell antigen (e.g., CD3) at one end of the molecule and an antigen binding domain that will bind to an antigen on the target cell. A BiTE molecule was recently described in International Publication No. WO 99/54440, which is herein incorporated by reference. This publication describes a novel single-chain multifunctional polypeptide that comprises binding sites for the CD19 and CD3 antigens (CD19×CD3). This molecule was derived from two antibodies, one that binds to CD19 on the B cell and an antibody that binds to CD3 on the T cells. The variable regions of these different antibodies are linked by a polypeptide sequence, thus creating a single molecule. Also described, is the linking of the heavy chain (VH) and light chain (VL) variable domains with a flexible linker to create a single chain, bispecific antibody.

In an embodiment of this invention, an antibody or ligand that immunospecifically binds a RSV polypeptide will comprise a portion of the BiTE molecule. For example, the V_{H} and/or V_{L} of an antibody that binds a RSV polypeptide can be fused to an anti-CD3 binding portion such as that of the molecule described above, thus creating a BiTE molecule that targets the RSV polypeptide. In addition to the VH and/or VL domains of antibody against a RSV polypeptide, other molecules that bind the RSV polypeptide can comprise the BiTE molecule. In another embodiment, the BiTE molecule can comprise a molecule that binds to other T cell antigens (other than CD3). For example, ligands and/or antibodies that immunospecifically bind to T-cell antigens like CD2, CD4, CD8, CD11a, TCR, and CD28 are contemplated to be part of this invention. This list is not meant to be exhaustive but only to illustrate that other molecules that can immunospecifically bind to a T cell antigen can be used as part of a BiTE molecule. These molecules can include the VH and/or VL portions of the antibody or natural ligands (for example LFA3 whose natural ligand is CD3).

In certain embodiments, the antibody to be used with the invention binds to an intracellular epitope, i.e., is an intrabody. An intrabody comprises at least a portion of an antibody that is capable of immunospecifically binding an antigen and preferably does not contain sequences coding for its secretion. Such antibodies will bind antigen intracellularly. In one embodiment, the intrabody comprises a single-chain Fv (“scFv”). scFvs are antibody fragments comprising the VH and VL domains of an antibody, wherein these domains are present in a single polypeptide chain. Generally, the scFv polypeptide further comprises a polypeptide linker between the VH and VL domains which enables the scFv to form the desired structure for antigen binding. For a review of scFvs see Pluckthun in The Pharmacology of Monoclonal Antibodies, vol. 113, Rosenburg and Moore eds. Springer-Verlag, New York, pp. 269-315 (1994). In a further embodiment, the intrabody preferably does not encode an operable secretory sequence and thus remains within the cell (see generally Marasco, W A, 1998, Intrabodies: Basic Research and Clinical Gene Therapy Applications, Springer: New York).

The present invention provides for antibodies that exhibit a high potency in an assay described herein. High potency antibodies can be produced by methods disclosed in copending U.S. patent application Ser. Nos. 60/168,426, 60/186,252, U.S. Publication No. 2002/0098189, and U.S. Pat. No. 6,656,467 (which are incorporated herein by reference in their entirety) and methods described herein. For example, high potency antibodies can be produced by genetically engineering appropriate antibody gene sequences and expressing the antibody sequences in a suitable host. The antibodies produced can be screened to identify antibodies with, e.g., high kₒₙ values in a BIAcore assay.

In a specific embodiment, an antibody of the invention has approximately 20-fold, 25-fold, 30-fold, 35-fold, 40-fold, 45-fold, 50-fold, 55-fold, 60-fold, 65-fold, 70-fold, 75-fold, 80-fold, 90-fold, 100-fold or higher affinity for a RSV antigen (e.g., RSV F antigen) than palivizumab or an antibody-binding fragment thereof as assessed by an assay known in the art or described herein (e.g., a BIAcore assay). In another embodiment, an antibody of the invention has an approximately 1-fold, 1.5-fold, 2-fold, 3-fold, 4-fold, 5-fold, or more higher Kₐ than palivizumab or an antigen-binding fragment thereof as assessed by an assay known in the art or described herein. In another embodiment, an antibody of the invention has an approximately 1-fold, 2-fold, 3-fold, 4-fold, 5-fold, 6-fold, 7-fold, 8-fold, 9-fold, 10-fold, 11-fold 12-fold, 13-fold, 14-fold, 15-fold, 16-fold, 17-fold, 18-fold, 19-fold, or 20-fold or more potent than palivizumab or an antigen-binding fragment thereof in an in vitro microneutralization assay. In certain embodiments, the microneutralization assay is a microneutralization assay described herein (for example, as described in Examples 6.4, 6.8, and 6.18 herein) or as in Johnson et al., 1999, J. Infectious Diseases 180:35-40. The amino acid sequence of palivizumab is disclosed, e.g., in Johnson et al., 1997, J. Infectious Disease 176:1215-1224, and U.S. Pat. No. 5,824,307, each of which is incorporated herein by reference in its entirety. In some embodiments, an antibody of the invention is an antibody comprising a VH domain of SEQ ID NO:7 (or VH chain of SEQ ID NO:208) and/or a VL domain of SEQ ID NO:8 (or VL chain of SEQ ID NO:209). In some embodiments, an antibody of the invention is an antibody comprising a VH domain of SEQ ID NO:7 (or VH chain of SEQ ID NO:208) and/or a VL domain of SEQ ID NO:8 (or VL chain of SEQ ID NO:209). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE). In other embodiments, a modified antibody of the invention is a modified palivizumab antibody or a modified antibody comprising a VH domain of SEQ ID NO:7 (or VH chain of SEQ ID NO:208) and/or a VL domain of SEQ ID NO:8 (or VL chain of SEQ ID NO:209).

The present invention provides for antibodies that immunospecifically bind to one or more RSV antigens, said antibodies comprising the amino acid sequence of palivizumab with one or more amino acid residue substitutions in the variable light (VL) domain and/or variable heavy (VH) domain or chain depicted in FIG. 1. The present invention also provides for antibodies that immunospecifically bind to one or more RSV antigens, said antibodies comprising the amino acid sequence of palivizumab with one or more amino acid residue substitutions in one or more VL CDRs and/or one or more VH CDRs. In a specific embodiment, an antibody comprises the amino acid sequence of palivizumab with one or more amino acid residue substitutions of the amino acid residues indicated in bold face and underlining in Table 1. In another embodiment, an antibody comprises the amino sequence of palivizumab with one or more amino acid residue substitutions of the amino acid residues indicated in bold face and underlining in Table 1 and one or more amino acid residue substitutions of the framework regions of the variable domains of palivizumab (e.g., mutations in framework region 4 of the heavy and/or light variable domains). In accordance with these embodiments, the amino acid residue substitutions can be conservative or non-conservative. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE). The antibody generated by introducing substitutions in the VH domain, VH CDRs, VL domain and/or VL CDRs of palivizumab can be tested in vitro and in vivo, for example, for its ability to bind to RSV F antigen, for its ability to neutralize RSV, or for its ability to prevent, manage, treat and/or ameliorate a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). In certain embodiments, the antibody does not comprise the VH chain and/or VL chain of palivizumab. In some embodiments, the antibody does not comprise the VH domain and/or the VL domain of palivizumab. In other embodiments, the antibody does not comprise a VH CDR1, VH CDR2, and/or VH CDR3 of palivizumab. In yet other embodiments, the antibody does not comprise a VL CDR1, VL CDR2, and/or VL CDR3 of palivizumab. In specific embodiments, the antibody is not palivizumab.

TABLE 1
CDR Sequences of palivizumab

| CDR | Sequence* | SEQ ID NO: |
|---|---|---|
| VH1 | TSGMSVG | 1 |
| VH2 | DIWWDDKKDYNPSLKS | 2 |
| VH3 | SMITNWYFDV | 3 |
| VL1 | KCQLSVGYMH | 4 |
| VL2 | DTSKLAS | 5 |
| VL3 | FQGSGYPFT | 6 |

*Bold faced & underlined amino acid residues are preferred residues which should be substituted.

The antibodies of the present invention include those antibodies and antigen-binding fragments of the antibodies referenced in Table 2, the Examples Section, and elsewhere in the application. In all cases, the antibody may be a modified antibody (i.e., comprises a modified IgG constant domain or FcRn binding fragment thereof (e.g., the Fc domain or hinge-Fc domain)) or may be an unmodified antibody (i.e., does not comprise a modified IgG constant domain or FcRn binding fragment thereof). In a specific embodiment, an antibody of the present invention is AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4 antibody. In another embodiment, an antibody of the invention comprises an antigen-binding fragment (e.g., a Fab fragment of) AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4. In a preferred embodiment, an antibody of the invention is A4B4L1FR-S28R (MEDI-524) antibody or an antigen-binding fragment thereof. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

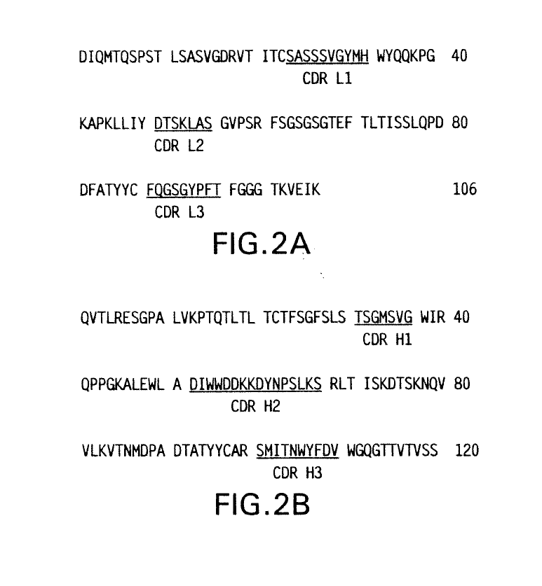

In some embodiments, a AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, and/or A17h4 antibody comprises the framework region of palivizumab (see FIG. 1). In preferred embodiments, a AFFF, P12f2, P12f4, P11d4, Ale9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, and/or A17h4 antibody comprises the framework region of palivizumab with the exception that there is an amino acid substitution of an A105Q in the heavy chain framework 4 (FR4) (Kabat et al. (1991) Sequences of proteins of immunological interest. (U.S. Department of Health and Human Services, Washington, D.C.) 5^{th} ed.) (i.e., position 112 in SEQ ID NO:7 (palivizumab VH domain)) and an L104V in the light chain FR4 (i.e., position 103 in SEQ ID NO:8 (palivizumab VL domain)). An example of an antibody that comprises a framework with these VH and VL single mutations is shown in FIG. 2 (1X-493L1FR) and in FIG. 13 (MEDI-524). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In a specific embodiment, the present invention provides for one or more antibodies that immunospecifically bind to one or more RSV F antigens, said antibodies comprising a VH chain and/or VL chain having the amino acid sequence of a VH chain and/or VL chain of AFFF, P12f2, P12f4, P11d4, Ale9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, and/or A17h4. In a preferred embodiment, an antibody of the invention immunospecifically binds to a RSV F antigen, and said antibody comprises a VH chain and/or a VL chain having the amino acid sequence of the VH and/or VL chain of A4B4L1FR-S28R (MEDI-524). In another embodiment, the present invention provides for one or more antibodies that immunospecifically bind to one or more RSV antigens, said antibodies comprising a VH domain and/or VL domain having the amino acid sequence of a VH domain and/or VL domain of AFFF, P12f2, P12f4, P11d4, Ale9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, and/or A17h4. In a preferred embodiment, an antibody of the invention immunospecifically binds to a RSV F antigen, and said antibody comprises a VH domain and/or VL domain having the amino acid sequence of the VH domain and/or VL domain of A4B4L1FR-S28R (MEDI-524). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, the present invention provides for antibodies that immunospecifically bind to one or more RSV antigens, said antibodies comprising one, two, three, or more CDRs having the amino acid sequence of one, two, three, or more CDRs of AFFF, P12f2, P12f4, P11d4, Ale9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, and/or A17h4. In a preferred embodiment, an antibody of the invention immunospecifically binds to a RSV antigen, and said antibody comprises one, two, three, or more CDRs having the amino acid sequence of one, two, three, or more CDRs of A4B4L1FR-S28R (MEDI-524). In yet another embodiment, the present invention provides for one or more antibodies that immunospecifically bind to one or more RSV F antigens, said antibodies comprising a combination of VH CDRs and/or VL CDRs having the amino acid sequence of VH CDRs and/or VL CDRs of AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, and/or A17h4. In a preferred embodiment, an antibody of the invention immunospecifically binds to a RSV F antigen and said antibody comprises a combination of VH CDRs and/or VL CDRs having the amino acid sequence of the VH CDRs and/or VL CDRs of A4B4L1FR-S28R (MEDI-524). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention provides antibodies that immunospecifically bind to one or more RSV antigens (e.g., RSV F antigen), said antibodies comprising a VH chain having an amino acid sequence of any one of the VH chains listed in Table 2. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The invention also provides antibodies that immunospecifically bind to one or more RSV antigens (e.g., RSV F antigen), said antibodies comprising a VH domain having an amino acid sequence of any one of the VH domains listed in Table 2. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention also provides antibodies that immunospecifically bind to one or more RSV antigens, said antibodies comprising one or more VH CDRs (e.g., VH CDR1, VH CDR2, and/or VH CDR3) having an amino acid sequence of any one of the VH CDRs listed in Table 2 and/or Tables 3A-3C. In certain embodiments of the invention, an antibody comprising a VH CDR having an amino acid sequence of any of one of the VH CDRs listed in Table 2 and/or Tables 3A-3C is not palivizumab. In some embodiments, the antibody comprises one, two or three of the VH CDRs listed in Table 2 and/or Tables 3A-3C. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE). In some embodiments, a modified antibody comprising a VH CDR having an amino acid sequence of any one of the VH CDRs listed in Table 2 and/or Tables 3A-3C is a modified palivizumab.

TABLE 2
Antibodies & Fragments Thereof

| Antibody | VH | VH | | | | VL | VL | | | |
|---|---|---|---|---|---|---|---|---|---|---|
| Name | Chain | Domain | VH CDR1 | VH CDR2 | VH CDR3 | Chain | Domain | VL CDR1 | VL CDR2 | VL CDR3 |
| **palivizumab | SEQ ID | SEQ ID | TSGMSVG | DIWWDDKKDYN | SMITNWYFDV | SEQ ID | SEQ ID | KCQLSVGYMH | DTSKLAS | FQGSGYPFT |
| | NO: 208 | NO: 7 | (SEQ ID NO: 1) | PSLKS | (SEQ ID NO: 3) | NO: 209 | NO: 8 | (SEQ ID NO: 4) | (SEQ ID NO: 5) | (SEQ ID NO: 6) |
| | | | | (SEQ ID NO: 2) | | | | | | |
| ***AFFF | SEQ ID | SEQ ID | TAGMSVG | DIWWDDKKDYN | SMITNFYFDV | SEQ ID | SEQ ID | SASSSVGYMH | DTFKLAS | FQFSGYPFT |
| | NO: 210 | NO: 9 | (SEQ ID NO: 10) | PSLKS | (SEQ ID NO: 12) | NO: 211 | NO: 13 | (SEQ ID NO: 14) | (SEQ ID | (SEQ ID |
| | | | | (SEQ ID NO: 2) | | | | | NO: 15) | NO: 16) |
| ***P12f2 | SEQ ID | SEQ ID | TPGMSVG | DIWWDDKKHYN | DMIFNFYFDV | SEQ ID | SEQ ID | SLSSRVGYMH | DTFYLSS | FQGSGYPFT |
| | NO: 212 | NO: 17 | (SEQ ID NO: 18) | PSLKD | (SEQ ID NO: 20) | NO: 213 | NO: 21 | (SEQ ID NO: 22) | (SEQ ID | (SEQ ID NO: 6) |
| | | | | (SEQ ID NO: 19) | | | | | NO: 23) | |
| ***P12f4 | SEQ ID | SEQ ID | TPGMSVG | DIWWDGKKHYN | DMIFNFYFDV | SEQ ID | SEQ ID | SLSSRVGYMH | DTRGLPS | FQGSGYPFT |
| | NO: 214 | NO: 24 | (SEQ ID NO: 18) | PSLKD | (SEQ ID NO: 20) | NO: 215 | NO: 26 | (SEQ ID NO: 22) | (SEQ ID | (SEQ ID NO: 6) |
| | | | | (SEQ ID NO: 25) | | | | | NO: 27) | |
| ***P11d4 | SEQ ID | SEQ ID | TPGMSVG | DIWWDGKKHYN | DMIFNWYFDV | SEQ ID | SEQ ID | SPSSRVGYMH | DTMRLAS | FQGSGYPFT |
| | NO: 216 | NO: 28 | (SEQ ID NO: 18) | PSLKD | (SEQ ID NO: 29) | NO: 217 | NO: 30 | (SEQ ID NO: 31) | (SEQ ID | (SEQ ID NO: 6) |
| | | | | (SEQ ID NO: 25) | | | | | NO: 32) | |
| ***Ale9 | SEQ ID | SEQ ID | TAGMSVG | DIWWDGKKHYN | DMIFNWYFDV | SEQ ID | SEQ ID | SLSSRVGYMH | DTFKLSS | FQGSGYPFT |
| | NO: 218 | NO: 33 | (SEQ ID NO: 10) | PSLKD | (SEQ ID NO: 29) | NO: 219 | NO: 34 | (SEQ ID NO: 22) | (SEQ ID | (SEQ ID NO: 6) |
| | | | | (SEQ ID NO: 25) | | | | | NO: 35) | |
| ***A12a6 | SEQ ID | SEQ ID | TAGMSVG | DIWWDGKKDYN | DMIFNFYFDV | SEQ ID | SEQ ID | SASSRVGYMH | DTFKLSS | FQGSGYPFT |
| | NO: 220 | NO: 36 | (SEQ ID NO: 10) | PSLKD | (SEQ ID NO: 20) | NO: 221 | NO: 38 | (SEQ ID NO: 39) | (SEQ ID | (SEQ ID NO: 6) |
| | | | | (SEQ ID NO: 37) | | | | | NO: 35) | |
| ***A13c4 | SEQ ID | SEQ ID | TAGMSVG | DIWWDGKKSYN | DMIFNFYFDV | SEQ ID | SEQ ID | SLSSRVGYMH | DTMYQSS | FQGSGYPFT |
| | NO: 222 | NO: 40 | (SEQ ID NO: 10) | PSLKD | (SEQ ID NO: 20) | NO: 223 | NO: 42 | (SEQ ID NO: 22) | (SEQ ID | (SEQ ID NO: 6) |
| | | | | (SEQ ID NO: 41) | | | | | NO: 43) | |
| ***A17d4 | SEQ ID | SEQ ID | TAGMSVG | DIWWDDKKSYN | DMIFNFYFDV | SEQ ID | SEQ ID | LPSSRVGYMH | DTMYQSS | FQGSGYPFT |
| | NO: 224 | NO: 44 | (SEQ ID NO: 10) | PSLKD | (SEQ ID NO: 20) | NO: 225 | NO: 46 | (SEQ ID NO: 47) | (SEQ ID | (SEQ ID NO: 6) |
| | | | | (SEQ ID NO: 45) | | | | | NO: 43) | |
| ***A4B4 | SEQ ID | SEQ ID | TAGMSVG | DIWWDDKKHYN | DMIFNFYFDV | SEQ ID | SEQ ID | SASSRVGYMH | DTFFLDS | FQGSGYPFT |
| | NO: 226 | NO: 48 | (SEQ ID NO: 10) | PSLKD | (SEQ ID NO: 20) | NO: 227 | NO: 49 | (SEQ ID NO: 39) | (SEQ ID | (SEQ ID NO: 6) |
| | | | | (SEQ ID NO: 19) | | | | | NO: 50) | |
| ****A8c7 | SEQ ID | SEQ ID | TAGMSVG | DIWWDDKKSYN | DMIFNWYFDV | SEQ ID | SEQ ID | SPSSRVGYMH | DTRYQSS | FQGSGYPFT |
| | NO: 228 | NO: 51 | (SEQ ID NO: 10) | PSLKD | (SEQ ID NO: 29) | NO: 229 | NO: 52 | (SEQ ID NO: 31) | (SEQ ID | (SEQ ID NO: 6) |
| | | | | (SEQ ID NO: 45) | | | | | NO: 53) | |
| *1X- | SEQ ID | SEQ ID | TSGMSVG | DIWWDDKKDYN | SMITNWYFDV | SEQ ID | SEQ ID | SASSSVGYMH | DTSKLAS | FQGSGYPFT |
| 493L1FR | NO: 230 | NO: 343 | (SEQ ID NO: 1) | PSLKS | (SEQ ID NO: 3) | NO: 231 | NO: 54 | (SEQ ID NO: 14) | (SEQ ID NO: 5) | (SEQ ID NO: 6) |
| | | | | (SEQ ID NO: 2) | | | | | | |
| *H3-3F4 | SEQ ID | SEQ ID | TAGMSVG | DIWWDDKKDYN | DMIFNWYFDV | SEQ ID | SEQ ID | SASSSVGYMH | DTFKLAS | FQGSGYPFT |
| | NO: 232 | NO: 55 | (SEQ ID NO: 10) | PSLKS | (SEQ ID NO: 29) | NO: 233 | NO: 56 | (SEQ ID NO: 14) | (SEQ ID | (SEQ ID NO: 6) |
| | | | | (SEQ ID NO: 2) | | | | | NO: 15) | |
| *M3H9 | SEQ ID | SEQ ID | TAGMSVG | DIWWDDKKDYN | DMIFNWYFDV | SEQ ID | SEQ ID | SASSSVGYMH | DTYKQTS | FQGSGYPFT |
| | NO: 234 | NO: 55 | (SEQ ID NO: 10) | PSLKS | (SEQ ID NO: 29) | NO: 235 | NO: 70 | (SEQ ID NO: 14) | (SEQ ID | (SEQ ID NO: 6) |
| | | | | (SEQ ID NO: 2) | | | | | NO: 57) | |
| *Y10H6 | SEQ ID | SEQ ID | TAGMSVG | DIWWDDKKDYN | DMIFNWYFDV | SEQ ID | SEQ ID | SASSSVGYMH | DTRYLSS | FQGSGYPFT |
| | NO: 236 | NO: 55 | (SEQ ID NO: 10) | PSLKS | (SEQ ID NO: 29) | NO: 237 | NO: 58 | (SEQ ID NO: 14) | (SEQ ID | (SEQ ID NO: 6) |
| | | | | (SEQ ID NO: 2) | | | | | NO: 59) | |
| *DG | SEQ ID | SEQ ID | TAGMSVG | DIWWDDKKDYN | DMITNFYFDV | SEQ ID | SEQ ID | SASSSVGYMH | DTFKLAS | FQGSGYPFT |
| (aka | NO: 238 | NO: 78 | (SEQ ID NO: 10) | PSLKS | (SEQ ID NO: 79) | NO: 239 | NO: 56 | (SEQ ID NO: 14) | (SEQ ID | (SEQ ID NO: 6) |
| D95/G93) | | | | (SEQ ID NO: 2) | | | | | NO: 15) | |
| AFFF(1) | SEQ ID | SEQ ID | TAGMSVG | DIWWDDKKDYN | SMITNFYFDV | SEQ ID | SEQ ID | SASSSVGYMH | DTFKLAS | FQGSFYPFT |
| | NO: 240 | NO: 9 | (SEQ ID NO: 10) | PSLKS | (SEQ ID NO: 12) | NO: 241 | NO: 60 | (SEQ ID NO: 14) | (SEQ ID | (SEQ ID |
| | | | | (SEQ ID NO: 2) | | | | | NO: 15) | NO: 61) |
| *6H8 | SEQ ID | SEQ ID | TAGMSVG | DIWWDDKKDYN | DMITNFYFDV | SEQ ID | SEQ ID | SASSSVGYMH | DTFKLTS | FQGSGYPFT |
| | NO: 242 | NO: 78 | (SEQ ID NO: 10) | PSLKS | (SEQ ID NO: 79) | NO: 243 | NO: 62 | (SEQ ID NO: 14) | (SEQ ID | (SEQ ID NO: 6) |
| | | | | (SEQ ID NO: 2) | | | | | NO: 63) | |
| *L1-7E5 | SEQ ID | SEQ ID | TAGMSVG | DIWWDDKKDYN | DMITNFYFDV | SEQ ID | SEQ ID | SASSRVGYMH | DTFKLAS | FQGSGYPFT |
| | NO: 244 | NO: 78 | (SEQ ID NO: 10) | PSLKS | (SEQ ID NO: 79) | NO: 245 | NO: 64 | (SEQ ID NO: 39) | (SEQ ID | (SEQ ID NO: 6) |
| | | | | (SEQ ID NO: 2) | | | | | NO: 15) | |
| *L2-15B10 | SEQ ID | SEQ ID | TAGMSVG | DIWWDDKKDYN | DMITNFYFDV | SEQ ID | SEQ ID | SASSSVGYMH | DTFRLAS | FQGSGYPFT |
| | NO: 246 | NO: 78 | (SEQ ID NO: 10) | PSLKS | (SEQ ID NO: 79) | NO: 247 | NO: 65 | (SEQ ID NO: 14) | (SEQ ID | (SEQ ID NO: 6) |
| | | | | (SEQ ID NO: 2) | | | | | NO: 66) | |
| *A13a11 | SEQ ID | SEQ ID | TAGMSVG | DIWWDDKKHYN | DMIFNWYFDV | SEQ ID | SEQ ID | SPSSRVGYMH | DTYRHSS | FQGSGYPFT |
| | NO: 248 | NO: 67 | (SEQ ID NO: 10) | PSLKD | (SEQ ID NO: 29) | NO: 249 | NO: 68 | (SEQ ID NO: 31) | (SEQ ID | (SEQ ID NO: 6) |
| | | | | (SEQ ID NO: 19) | | | | | NO: 69) | |
| *A1h5 | SEQ ID | SEQ ID | TAGMSVG | DIWWDGKKHYN | DMIFNWYFDV | SEQ ID | SEQ ID | SLSSSVGYMH | DTFFHRS | FQGSGYPFT |
| | NO: 250 | NO: 33 | (SEQ ID NO: 10) | PSLKD | (SEQ ID NO: 29) | NO: 251 | NO: 71 | (SEQ ID NO: 72) | (SEQ ID | (SEQ ID NO: 6) |
| | | | | (SEQ ID NO: 25) | | | | | NO: 73) | |
| A4B4(1) | SEQ ID | SEQ ID | TAGMSVG | DIWWDDKKHYN | DMIFNFYFDV | SEQ ID | SEQ ID | SASSRVGYMH | DTLLLDS | FQGSGYPFT |
| | NO: 252 | NO: 48 | (SEQ ID NO: 10) | PSLKD | (SEQ ID NO: 20) | NO: 253 | NO: 74 | (SEQ ID NO: 39) | (SEQ ID | (SEQ ID NO: 6) |
| | | | | (SEQ ID NO: 19) | | | | | NO: 75) | |
| ***A4B4L1F | SEQ ID | SEQ ID | TAGMSVG | DIWWDDKKHYN | DMIFNFYFDV | SEQ ID | SEQ ID | SASSRVGYMH | DTSKLAS | FQGSGYPFT |
| R-S28R | NO: 254 | NO: 48 | (SEQ ID NO: 10) | PSLKD | (SEQ ID NO: 20) | NO: 255 | NO: 11 | (SEQ ID NO: 39) | (SEQ ID NO: 5) | (SEQ ID NO: 6) |
| (aka | | | | (SEQ ID NO: 19) | | | | | | |
| MEDI-524) | | | | | | | | | | |
| ***A4B4- | SEQ ID | SEQ ID | TAGMSVG | DIWWDDKKHYN | DMIFNFYFDV | SEQ ID | SEQ ID | SASSRVGYMH | DTSFLDS | FQGSGYPFT |
| F52S | NO: 256 | NO: 48 | (SEQ ID NO: 10) | PSLKD | (SEQ ID NO: 20) | NO: 257 | NO: 76 | (SEQ ID NO: 39) | (SEQ ID | (SEQ ID NO: 6) |
| | | | | (SEQ ID NO: 19) | | | | | NO: 77) | |
| ***A17d4(1) | SEQ ID | SEQ ID | TAGMSVG | DIWWDGKKSYN | DMIFNFYFDV | SEQ ID | SEQ ID | LPSSRVGYMH | DTMYQSS | FQGSGYPFT |
| | NO: 222 | NO: 40 | (SEQ ID NO: 10) | PSLKD | (SEQ ID NO: 20) | NO: 225 | NO: 46 | (SEQ ID NO: 47) | (SEQ ID | (SEQ ID NO: 6) |
| | | | | (SEQ ID NO: 41) | | | | | NO: 43) | |
| ***A3e2 | SEQ ID | SEQ ID | TAGMSVG | DIWWGDKGHYN | DMIFNWYFDV | SEQ ID | SEQ ID | SASSSVGYMH | DTFYLHS | FQGSGYPFT |
| | NO: 303 | NO: 304 | (SEQ ID NO: 10) | PSLKD | (SEQ ID NO: 29) | NO: 306 | NO: 307 | (SEQ ID NO: 14) | (SEQ ID | (SEQ ID NO: 6) |
| | | | | (SEQ ID NO: 305) | | | | | NO: 308) | |
| ***A14a4 | SEQ ID | SEQ ID | TAGMSVG | DIWWDDKKSYN | DMITNWYFDV | SEQ ID | SEQ ID | LLSSRVGYMH | DTYYQTS | FQGSGYPFT |
| | NO: 309 | NO: 310 | (SEQ ID NO: 10) | PSLKD | (SEQ ID NO: 311) | NO: 312 | NO: 313 | (SEQ ID NO: 314) | (SEQ ID | (SEQ ID NO: 6) |
| | | | | (SEQ ID NO: 45) | | | | | NO: 315) | |
| ***A16b4 | SEQ ID | SEQ ID | TAGMSVG | DIWWDDKKHYN | DMIFNWYFDV | SEQ ID | SEQ ID | LLSSRVGYMH | DTMYQAS | FQGSGYPFT |
| | NO: 316 | NO: 317 | (SEQ ID NO: 10) | PSLKD | (SEQ ID NO: 29) | NO: 318 | NO: 319 | (SEQ ID NO: 320) | (SEQ ID | (SEQ ID NO: 6) |
| | | | | (SEQ ID NO: 19) | | | | | NO: 321) | |
| ***A17b5 | SEQ ID | SEQ ID | TAGMSVG | DIWWDDKKHYN | DMIFNWYFDV | SEQ ID | SEQ ID | SLSSRVGYMH | DTYYLPS | FQGSGYPFT |
| | NO: 322 | NO: 323 | (SEQ ID NO: 10) | PSLKD | (SEQ ID NO: 29) | NO: 324 | NO: 325 | (SEQ ID NO: 22) | (SEQ ID | (SEQ ID NO: 6) |
| | | | | (SEQ ID NO: 19) | | | | | NO: 326) | |
| ***A17f5 | SEQ ID | SEQ ID | TAGMSVG | DIWWDDKKDYN | DMIFNWYFDV | SEQ ID | SEQ ID | SLSSRVGYMH | DTFRHTS | FQGSGYPFT |
| | NO: 327 | NO: 328 | (SEQ ID NO: 10) | PSLKD | (SEQ ID NO: 29) | NO: 330 | NO: 331 | (SEQ ID NO: 22) | (SEQ ID | (SEQ ID NO: 6) |
| | | | | (SEQ ID NO: 329) | | | | | NO: 332) | |
| ***A17h4 | SEQ ID | SEQ ID | TAGMSVG | DIWWDGKKHYN | DMIFNWYFDV | SEQ ID | SEQ ID | SPSSSVGYMH | DTYYLAS | FQGSGYPFT |
| | NO: 218 | NO: 33 | (SEQ ID NO: 10) | PSLKD | (SEQ ID NO: 29) | NO: 333 | NO: 334 | (SEQ ID NO: 335) | (SEQ ID | (SEQ ID NO: 6) |
| | | | | (SEQ ID NO: 25) | | | | | NO: 336) | |

Bold faced and underlined amino acid residues are the residues which differ from the amino acid sequence in palivizumab;
Fab fragment produced (*);
Monoclonal antibody produced (**);
Fab fragment & monoclonal antibody produced (***)

TABLE 3A
VH CDR1 Sequences

| | |
|---|---|
| TSGMSVG | (SEQ ID NO: 1) |
| TAGMSVG | (SEQ ID NO: 10) |
| TPGMSVG | (SEQ ID NO: 18) |

Bold faced & underlined amino acid residues are the residues which differ from the amino acid sequence in palivizumab

TABLE 3B
VH CDR2 Sequences

| | |
|---|---|
| DIWWDDKKDYNPSLKS (SEQ ID NO: 2) | DIWWDGKKDYNPSLKS (SEQ ID NO: 100) |
| DIWWDDKKDYNPSLKD (SEQ ID NO: 86) | DIWWDGKKDYNPSLKD (SEQ ID NO: 103) |
| DIWWDDKKHYNPSLKS (SEQ ID NO: 82) | DIWWDGKKHYNPSLKS (SEQ ID NO: 106) |
| DIWWDDKKHYNPSLKD (SEQ ID NO: 19) | DIWWDGKKHYNPSLKD (SEQ ID NO: 25) |
| DIWWDDKKSYNPSLKS (SEQ ID NO: 109) | DIWWDGKKSYNPSLKS (SEQ ID NO: 114) |
| DIWWDDKKSYNPSLKD (SEQ ID NO: 111) | DIWWDGKKSYNPSLKD (SEQ ID NO: 41) |
| DIWWDDKGDYNPSLKS (SEQ ID NO: 384) | DIWWDGKGDYNPSLKS (SEQ ID NO: 390) |
| DIWWDDKGDYNPSLKD (SEQ ID NO: 385) | DIWWDGKGDYNPSLKD (SEQ ID NO: 391) |
| DIWWDDKGHYNPSLKS (SEQ ID NO: 386) | DIWWDGKGHYNPSLKS (SEQ ID NO: 392) |
| DIWWDDKGHYNPSLKD (SEQ ID NO: 387) | DIWWDGKGHYNPSLKD (SEQ ID NO: 393) |
| DIWWDDKGSYNPSLKS (SEQ ID NO: 388) | DIWWDGKGSYNPSLKS (SEQ ID NO: 394) |
| DIWWDDKGSYNPSLKD (SEQ ID NO: 389) | DIWWDGKGSYNPSLKD (SEQ ID NO: 395) |

Bold faced & underlined amino acid residues are the residues which differ from the amino acid sequence in palivizumab

TABLE 3C
VH CDR3 Sequences

| | |
|---|---|
| SMITNWYFDV (SEQ ID NO: 3) | DMITNWYFDV |
| | (SEQ ID NO: 83) |
| SMITNFYFDV (SEQ ID NO: 12) | DMITNFYFDV |
| | (SEQ ID NO: 29) |
| SMIFNWYFDV (SEQ ID NO: 94) | DMIFNWYFDV |
| | (SEQ ID NO: 79) |
| SMIFNFYFDV (SEQ ID NO: 97) | DMIFNFYFDV |
| | (SEQ ID NO: 20) |

Bold faced & underlined amino acid residues are the residues which differ from the amino acid sequence in palivizumab

TABLE 3D
VL CDR1 Sequences

| | | |
|---|---|---|
| KCQLSVGYMH (SEQ ID NO: 4) | SCQLSVGYMH (SEQ ID NO: 127) | LCQLSVGYMH (SEQ ID NO: 204) |
| KCQLRVGYMH (SEQ ID NO: 87) | SCQLRVGYMH (SEQ ID NO: 132) | LCQLRVGYMH (SEQ ID NO: 206) |
| KCQLFVGYMH (SEQ ID NO: 396) | SCQLFVGYMH (SEQ ID NO: 436) | LCQLFVGYMH (SEQ ID NO: 476) |
| KCQSSVGYMH (SEQ ID NO: 80) | SCQSSVGYMH (SEQ ID NO: 129) | LCQSSVGYMH (SEQ ID NO: 205) |
| KCQSRVGYMH (SEQ ID NO: 84) | SCQSRVGYMH (SEQ ID NO: 130) | LCQSRVGYMH (SEQ ID NO: 203) |
| KCQSFVGYMH (SEQ ID NO: 397) | SCQSFVGYMH (SEQ ID NO: 437) | LCQSFVGYMH (SEQ ID NO: 477) |
| KCQVSVGYMH (SEQ ID NO: 398) | SCQVSVGYMH (SEQ ID NO: 438) | LCQVSVGYMH (SEQ ID NO: 478) |
| KCQVRVGYMH (SEQ ID NO: 399) | SCQVRVGYMH (SEQ ID NO: 439) | LCQVRVGYMH (SEQ ID NO: 479) |
| KCQVFVGYMH (SEQ ID NO: 400) | SCQVFVGYMH (SEQ ID NO: 440) | LCQVFVGYMH (SEQ ID NO: 480) |
| KCSLSVGYMH (SEQ ID NO: 112) | SCSLSVGYMH (SEQ ID NO: 142) | LCSLSVGYMH (SEQ ID NO: 196) |
| KCSLRVGYMH (SEQ ID NO: 119) | SCSLRVGYMH (SEQ ID NO: 148) | LCSLRVGYMH (SEQ ID NO: 198) |
| KCSLFVGYMH (SEQ ID NO: 401) | SCSLFVGYMH (SEQ ID NO: 441) | LCSLFVGYMH (SEQ ID NO: 481) |
| KCSSSVGYMH (SEQ ID NO: 115) | SCSSSVGYMH (SEQ ID NO: 144) | LCSSSVGYMH (SEQ ID NO: 197) |
| KCSSRVGYMH (SEQ ID NO: 117) | SCSSRVGYMH (SEQ ID NO: 146) | LCSSRVGYMH (SEQ ID NO: 195) |
| KCSSFVGYMH (SEQ ID NO: 402) | SCSSFVGYMH (SEQ ID NO: 442) | LCSSFVGYMH (SEQ ID NO: 482) |
| KCSVSVGYMH (SEQ ID NO: 403) | SCSVSVGYMH (SEQ ID NO: 443) | LCSVSVGYMH (SEQ ID NO: 483) |
| KCSVRVGYMH (SEQ ID NO: 404) | SCSVRVGYMH (SEQ ID NO: 444) | LCSVRVGYMH (SEQ ID NO: 484) |
| KCSVFVGYMH (SEQ ID NO: 405) | SCSVFVGYMH (SEQ ID NO: 445) | LCSVFVGYMH (SEQ ID NO: 485) |
| KAQLSVGYMH (SEQ ID NO: 182) | SAQLSVGYMH (SEQ ID NO: 207) | LAQLSVGYMH (SEQ ID NO: 486) |
| KAQLRVGYMH (SEQ ID NO: 180) | SAQLRVGYMH (SEQ ID NO: 190) | LAQLRVGYMH (SEQ ID NO: 487) |
| KAQLFVGYMH (SEQ ID NO: 406) | SAQLFVGYMH (SEQ ID NO: 446) | LAQLFVGYMH (SEQ ID NO: 488) |
| KAQSSVGYMH (SEQ ID NO: 181) | SAQSSVGYMH (SEQ ID NO: 191) | LAQSSVGYMH (SEQ ID NO: 489) |
| KAQSRVGYMH (SEQ ID NO: 179) | SAQSRVGYMH (SEQ ID NO: 189) | LAQSRVGYMH (SEQ ID NO: 490) |
| KAQSFVGYMH (SEQ ID NO: 407) | SAQSFVGYMH (SEQ ID NO: 447) | LAQSFVGYMH (SEQ ID NO: 491) |
| KAQVSVGYMH (SEQ ID NO: 408) | SAQVSVGYMH (SEQ ID NO: 448) | LAQVSVGYMH (SEQ ID NO: 492) |
| KAQVRVGYMH (SEQ ID NO: 409) | SAQVRVGYMH (SEQ ID NO: 449) | LAQVRVGYMH (SEQ ID NO: 493) |
| KAQVFVGYMH (SEQ ID NO: 410) | SAQVFVGYMH (SEQ ID NO: 450) | LAQVFVGYMH (SEQ ID NO: 494) |
| KASLSVGYMH (SEQ ID NO: 186) | SASLSVGYMH (SEQ ID NO: 188) | LASLSVGYMH (SEQ ID NO: 495) |
| KASLRVGYMH (SEQ ID NO: 184) | SASLRVGYMH (SEQ ID NO: 187) | LASLRVGYMH (SEQ ID NO: 496) |
| KASLFVGYMH (SEQ ID NO: 411) | SASLFVGYMH (SEQ ID NO: 451) | LASLFVGYMH (SEQ ID NO: 497) |
| KASSSVGYMH (SEQ ID NO: 185) | SASSSVGYMH (SEQ ID NO: 14) | LASSSVGYMH (SEQ ID NO: 498) |
| KASSRVGYMH (SEQ ID NO: 183) | SASSRVGYMH (SEQ ID NO: 39) | LASSRVGYMH (SEQ ID NO: 499) |
| KASSFVGYMH (SEQ ID NO: 412) | SASSFVGYMH (SEQ ID NO: 452) | LASSFVGYMH (SEQ ID NO: 500) |
| KASVSVGYMH (SEQ ID NO: 413) | SASVSVGYMH (SEQ ID NO: 453) | LASVSVGYMH (SEQ ID NO: 501) |
| KASVRVGYMH (SEQ ID NO: 414) | SASVRVGYMH (SEQ ID NO: 454) | LASVRVGYMH (SEQ ID NO: 502) |
| KASVFVGYMH (SEQ ID NO: 415) | SASVFVGYMH (SEQ ID NO: 455) | LASVFVGYMH (SEQ ID NO: 503) |
| KLQLSVGYMH (SEQ ID NO: 89) | SLQLSVGYMH (SEQ ID NO: 134) | LLQLSVGYMH (SEQ ID NO: 504) |
| KLQLRVGYMH (SEQ ID NO: 98) | SLQLRVGYMH (SEQ ID NO: 140) | LLQLRVGYMH (SEQ ID NO: 505) |
| KLQLFVGYMH (SEQ ID NO: 416) | SLQLFVGYMH (SEQ ID NO: 456) | LLQLFVGYMH (SEQ ID NO: 506) |
| KLQSSVGYMH (SEQ ID NO: 92) | SLQSSVGYMH (SEQ ID NO: 136) | LLQSSVGYMH (SEQ ID NO: 507) |
| KLQSRVGYMH (SEQ ID NO: 95) | SLQSRVGYMH (SEQ ID NO: 138) | LLQSRVGYMH (SEQ ID NO: 508) |
| KLQSFVGYMH (SEQ ID NO: 417) | SLQSFVGYMH (SEQ ID NO: 457) | LLQSFVGYMH (SEQ ID NO: 509) |
| KLQVSVGYMH (SEQ ID NO: 418) | SLQVSVGYMH (SEQ ID NO: 458) | LLQVSVGYMH (SEQ ID NO: 510) |
| KLQVRVGYMH (SEQ ID NO: 419) | SLQVRVGYMH (SEQ ID NO: 459) | LLQVRVGYMH (SEQ ID NO: 511) |
| KLQVFVGYMH (SEQ ID NO: 420) | SLQVFVGYMH (SEQ ID NO: 460) | LLQVFVGYMH (SEQ ID NO: 512) |
| KLSLSVGYMH (SEQ ID NO: 101) | SLSLSVGYMH (SEQ ID NO: 120) | LLSLSVGYMH (SEQ ID NO: 513) |
| KLSLRVGYMH (SEQ ID NO: 110) | SLSLRVGYMH (SEQ ID NO: 125) | LLSLRVGYMH (SEQ ID NO: 514) |
| KLSLFVGYMH (SEQ ID NO: 421) | SLSLFVGYMH (SEQ ID NO: 461) | LLSLFVGYMH (SEQ ID NO: 515) |
| KLSSSVGYMH (SEQ ID NO: 104) | SLSSSVGYMH (SEQ ID NO: 122) | LLSSSVGYMH (SEQ ID NO: 516) |
| KLSSRVGYMH (SEQ ID NO: 107) | SLSSRVGYMH (SEQ ID NO: 22) | LLSSRVGYMH (SEQ ID NO: 517) |
| KLSSFVGYMH (SEQ ID NO: 422) | SLSSFVGYMH (SEQ ID NO: 462) | LLSSFVGYMH (SEQ ID NO: 518) |
| KLSVSVGYMH (SEQ ID NO: 423) | SLSVSVGYMH (SEQ ID NO: 463) | LLSVSVGYMH (SEQ ID NO: 519) |
| KLSVRVGYMH (SEQ ID NO: 424) | SLSVRVGYMH (SEQ ID NO: 464) | LLSVRVGYMH (SEQ ID NO: 520) |
| KLSVFVGYMH (SEQ ID NO: 425) | SLSVFVGYMH (SEQ ID NO: 465) | LLSVFVGYMH (SEQ ID NO: 521) |
| KPQLSVGYMH (SEQ ID NO: 163) | SPQLSVGYMH (SEQ ID NO: 177) | LPQLSVGYMH (SEQ ID NO: 200) |
| KPQLRVGYMH (SEQ ID NO: 159) | SPQLRVGYMH (SEQ ID NO: 173) | LPQLRVGYMH (SEQ ID NO: 202) |
| KPQLFVGYMH (SEQ ID NO: 426) | SPQLFVGYMH (SEQ ID NO: 466) | LPQLFVGYMH (SEQ ID NO: 522) |
| KPQSSVGYMH (SEQ ID NO: 161) | SPQSSVGYMH (SEQ ID NO: 176) | LPQSSVGYMH (SEQ ID NO: 201) |
| KPQSRVGYMH (SEQ ID NO: 157) | SPQSRVGYMH (SEQ ID NO: 171) | LPQSRVGYMH (SEQ ID NO: 199) |
| KPQSFVGYMH (SEQ ID NO: 427) | SPQSFVGYMH (SEQ ID NO: 467) | LPQSFVGYMH (SEQ ID NO: 523) |
| KPQVSVGYMH (SEQ ID NO: 428) | SPQVSVGYMH (SEQ ID NO: 468) | LPQVSVGYMH (SEQ ID NO: 524) |
| KPQVRVGYMH (SEQ ID NO: 429) | SPQVRVGYMH (SEQ ID NO: 469) | LPQVRVGYMH (SEQ ID NO: 525) |
| KPQVFVGYMH (SEQ ID NO: 430) | SPQVFVGYMH (SEQ ID NO: 470) | LPQVFVGYMH (SEQ ID NO: 526) |
| KPSLSVGYMH (SEQ ID NO: 155) | SPSLSVGYMH (SEQ ID NO: 169) | LPSLSVGYMH (SEQ ID NO: 192) |
| KPSLRVGYMH (SEQ ID NO: 152) | SPSLRVGYMH (SEQ ID NO: 166) | LPSLRVGYMH (SEQ ID NO: 194) |
| KPSLFVGYMH (SEQ ID NO: 431) | SPSLFVGYMH (SEQ ID NO: 471) | LPSLFVGYMH (SEQ ID NO: 527) |
| KPSSSVGYMH (SEQ ID NO: 153) | SPSSSVGYMH (SEQ ID NO: 168) | LPSSSVGYMH (SEQ ID NO: 193) |
| KPSSRVGYMH (SEQ ID NO: 150) | SPSSRVGYMH (SEQ ID NO: 31) | LPSSRVGYMH (SEQ ID NO: 47) |
| KPSSFVGYMH (SEQ ID NO: 432) | SPSSFVGYMH (SEQ ID NO: 472) | LPSSFVGYMH (SEQ ID NO: 528) |
| KPSVSVGYMH (SEQ ID NO: 433) | SPSVSVGYMH (SEQ ID NO: 473) | LPSVSVGYMH (SEQ ID NO: 529) |
| KPSVRVGYMH (SEQ ID NO: 434) | SPSVRVGYMH (SEQ ID NO: 474) | LPSVRVGYMH (SEQ ID NO: 530) |
| KPSVFVGYMH (SEQ ID NO: 435) | SPSVFVGYMH (SEQ ID NO: 475) | LPSVFVGYMH (SEQ ID NO: 531) |

Bold faced & underlined amino acid residues are the residues which differ from the amino acid sequence in palivizumab

TABLE 3E
VL CDR2 Sequences

| | | | | | | |
|---|---|---|---|---|---|---|
| DTSKLAS (SEQ | DTFKLAS (SEQ | DTYKLAS (SEQ | DTRKLAS(SEQ | DTMKLAS (SEQ | DTKKLAS (SEQ | DTLKLAS(SEQ |
| ID NO: 5) | ID NO: 15) | ID NO: 799) | ID | ID | ID NO: 1211) | ID NO: 135) |
| | | | NOS: 113&174) | NOS: 121&162) | | |
| DTSKLSS (SEQ | DTFKLSS (SEQ | DTYKLSS (SEQ | DTRKLSS(SEQ | DTMKLSS (SEQ | DTKKLSS (SEQ | DTLKLSS (SEQ |
| ID NO: 165) | ID NO: 96) | ID NO: 800) | ID NO: 175) | ID NO: 164) | ID NO: 1212) | ID NO: 1355) |
| DTSKLKS (SEQ | DTFKLKS (SEQ | DTYKLKS (SEQ | DTRKLKS (SEQ | DTMKLKS (SEQ | DTKKLKS (SEQ | DTLKLKS (SEQ |
| ID NO: 532) | ID NO: 660) | ID NO: 801) | ID NO: 943) | ID NO: 1076) | ID NO: 1213) | ID NO: 1356) |
| DTSKLRS (SEQ | DTFKLRS(SEQ | DTYKLRS (SEQ | DTRKLRS (SEQ | DTMKLRS (SEQ | DTKKLRS (SEQ | DTLKLRS (SEQ |
| ID NO: 533) | ID NO: 661) | ID NO: 802) | ID NO: 944) | ID NO: 1077) | ID NO: 1214) | ID NO: 1357) |
| DTSKLHS (SEQ | DTFKLHS(SEQ | DTYKLHS (SEQ | DTRKLHS (SEQ | DTMKLHS (SEQ | DTKKLHS (SEQ | DTLKLHS (SEQ |
| ID NO: 534) | ID NO: 662) | ID NO: 803) | ID NO: 945) | ID NO: 1078) | ID NO: 1215) | ID NO: 1358) |
| DTSKLPS (SEQ | DTFKLPS (SEQ | DTYKLPS (SEQ | DTRKLPS(SEQ | DTMKLPS (SEQ | DTKKLPS (SEQ | DTLKLPS (SEQ |
| ID NO: 102) | ID NO: 663) | ID NO: 804) | ID NO: 118) | ID NO: 1079) | ID NO: 1216) | ID NO: 1359) |
| DTSKLTS (SEQ | DTFKLTS (SEQ | DTYKLTS (SEQ | DTRKLTS (SEQ | DTMKLTS (SEQ | DTKKLTS (SEQ | DTLKLTS (SEQ |
| ID NO: 535) | ID NO: 664) | ID NO: 805) | ID NO: 946) | ID NO: 1080) | ID NO: 1217) | ID NO: 1360) |
| DTSKLDS (SEQ | DTFKLDS (SEQ | DTYKLDS (SEQ | DTRKLDS (SEQ | DTMKLDS (SEQ | DTKKLDS (SEQ | DTLKLDS(SEQ |
| ID NO: 128) | ID NO: 665) | ID NO: 806) | ID NO: 947) | ID NO: 1081) | ID NO: 1218) | ID NO: 131) |
| DTSKHAS (SEQ | DTFKHAS (SEQ | DTYKHAS (SEQ | DTRKHAS (SEQ | DTMKHAS (SEQ | DTKKHAS (SEQ | DTLKHAS (SEQ |
| ID NO: 536) | ID NO: 666) | ID NO: 807) | ID NO: 948) | ID NO: 1082) | ID NO: 1219) | ID NO: 1361) |
| DTSKHSS (SEQ | DTFKHSS (SEQ | DTYKHSS (SEQ | DTRKHSS (SEQ | DTMKHSS (SEQ | DTKKHSS (SEQ | DTLKHSS (SEQ |
| ID NO: 537) | ID NO: 667) | ID NO: 808) | ID NO: 949) | ID NO: 1083) | ID NO: 1220) | ID NO: 1362) |
| DTSKHKS (SEQ | DTFKHKS (SEQ | DTYKHKS (SEQ | DTRKHKS (SEQ | DTMKHKS (SEQ | DTKKHKS (SEQ | DTLKHKS (SEQ |
| ID NO: 538) | ID NO: 668) | ID NO: 809) | ID NO: 950) | ID NO: 1084) | ID NO: 1221) | ID NO: 1363) |
| DTSKHRS (SEQ | DTFKHRS(SEQ | DTYKHRS (SEQ | DTRKHRS (SEQ | DTMKHRS (SEQ | DTKKHRS (SEQ | DTLKHRS (SEQ |
| ID NO: 539) | ID NO: 669) | ID NO: 810) | ID NO: 951) | ID NO: 1085) | ID NO: 1222) | ID NO: 1364) |
| DTSKHHS (SEQ | DTFKHHS (SEQ | DTYKHHS (SEQ | DTRKHHS (SEQ | DTMKHHS (SEQ | DTKKHHS (SEQ | DTLKHHS (SEQ |
| ID NO: 540) | ID NO: 670) | ID NO: 811) | ID NO: 952) | ID NO: 1086) | ID NO: 1223) | ID NO: 1365) |
| DTSKHPS (SEQ | DTFKHPS (SEQ | DTYKHPS (SEQ | DTRKHPS (SEQ | DTMKHPS (SEQ | DTKKHPS (SEQ | DTLKHPS (SEQ |
| ID NO: 541) | ID NO: 671) | ID NO: 812) | ID NO: 953) | ID NO: 1087) | ID NO: 1224) | ID NO: 1366) |
| DTSKHTS (SEQ | DTFKHTS (SEQ | DTYKHTS (SEQ | DTRKHTS (SEQ | DTMKHTS (SEQ | DTKKHTS (SEQ | DTLKHTS (SEQ |
| ID NO: 542) | ID NO: 672) | ID NO: 813) | ID NO: 954) | ID NO: 1088) | ID NO: 1225) | ID NO: 1367) |
| DTSKHDS (SEQ | DTFKHDS (SEQ | DTYKHDS (SEQ | DTRKHDS (SEQ | DTMKHDS (SEQ | DTKKHDS (SEQ | DTLKHDS (SEQ |
| ID NO: 543) | ID NO: 673) | ID NO: 814) | ID NO: 955) | ID NO: 1089) | ID NO: 1226) | ID NO: 1368) |
| DTSKQAS (SEQ | DTFKQAS (SEQ | DTYKQAS (SEQ | DTRKQAS(SEQ | DTMKQAS (SEQ | DTKKQAS (SEQ | DTLKQAS (SEQ |
| ID NO: 139) | ID NO: 674) | ID NO: 815) | ID NO: 170) | ID NO: 154) | ID NO: 1227) | ID NO: 1369) |
| DTSKQSS (SEQ | DTFKQSS (SEQ | DTYKQSS (SEQ | DTRKQSS(SEQ | DTMKQSS (SEQ | DTKKQSS (SEQ | DTLKQSS (SEQ |
| ID NO: 141) | ID NO: 675) | ID NO: 816) | ID NO: 172) | ID NO: 156) | ID NO: 1228) | ID NO: 1370) |
| DTSKQKS (SEQ | DTFKQKS (SEQ | DTYKQKS (SEQ | DTRKQKS (SEQ | DTMKQKS (SEQ | DTKKQKS (SEQ | DTLKQKS (SEQ |
| ID NO: 544) | ID NO: 676) | ID NO: 817) | ID NO: 956) | ID NO: 1090) | ID NO: 1229) | ID NO: 1371) |
| DTSKQRS (SEQ | DTFKQRS (SEQ | DTYKQRS (SEQ | DTRKQRS (SEQ | DTMKQRS (SEQ | DTKKQRS (SEQ | DTLKQRS (SEQ |
| ID NO: 545) | ID NO: 677) | ID NO: 818) | ID NO: 957) | ID NO: 1091) | ID NO: 1230) | ID NO: 1372) |
| DTSKQHS (SEQ | DTFKQHS (SEQ | DTYKQHS (SEQ | DTRKQHS (SEQ | DTMKQHS (SEQ | DTKKQHS (SEQ | DTLKQHS (SEQ |
| ID NO: 546) | ID NO: 678) | ID NO: 819) | ID NO: 958) | ID NO: 1092) | ID NO: 1231) | ID NO: 1373) |
| DTSKQPS (SEQ | DTFKQPS (SEQ | DTYKQPS (SEQ | DTRKQPS (SEQ | DTMKQPS (SEQ | DTKKQPS (SEQ | DTLKQPS (SEQ |
| ID NO: 547) | ID NO: 679) | ID NO: 820) | ID NO: 959) | ID NO: 1093) | ID NO: 1232) | ID NO: 1374) |
| DTSKQTS (SEQ | DTFKQTS (SEQ | DTYKQTS (SEQ | DTRKQTS (SEQ | DTMKQTS (SEQ | DTKKQTS (SEQ | DTLKQTS (SEQ |
| ID NO: 548) | ID NO: 680) | ID NO: 821) | ID NO: 960) | ID NO: 1094) | ID NO: 1233) | ID NO: 1375) |
| DTSKQDS (SEQ | DTFKQDS (SEQ | DTYKQDS (SEQ | DTRKQDS (SEQ | DTMKQDS (SEQ | DTKKQDS (SEQ | DTLKQDS (SEQ |
| ID NO: 549) | ID NO: 681) | ID NO: 822) | ID NO: 961) | ID NO: 1095) | ID NO: 1234) | ID NO: 1376) |
| DTSGLAS (SEQ | DTFGLAS (SEQ | DTYGLAS (SEQ | DTRGLAS(SEQ | DTMGLAS (SEQ | DTKGLAS (SEQ | DTLGLAS (SEQ |
| ID NO: 105) | ID NO: 682) | ID NO: 823) | ID NO: 116) | ID NO: 1096) | ID NO: 1235) | ID NO: 1377) |
| DTSGLSS (SEQ | DTFGLSS (SEQ | DTYGLSS (SEQ | DTRGLSS (SEQ | DTMGLSS (SEQ | DTKGLSS (SEQ | DTLGLSS (SEQ |
| ID NO: 550) | ID NO: 683) | ID NO: 824) | ID NO: 962) | ID NO: 1097) | ID NO: 1236) | ID NO: 1378) |
| DTSGLKS (SEQ | DTFGLKS (SEQ | DTYGLKS (SEQ | DTRGLKS (SEQ | DTMGLKS (SEQ | DTKGLKS (SEQ | DTLGLKS (SEQ |
| ID NO: 551) | ID NO: 684) | ID NO: 825) | ID NO: 963) | ID NO: 1098) | ID NO: 1237) | ID NO: 1379) |
| DTSGLRS (SEQ | DTFGLRS (SEQ | DTYGLRS (SEQ | DTRGLRS (SEQ | DTMGLRS (SEQ | DTKGLRS (SEQ | DTLGLRS (SEQ |
| ID NO: 552) | ID NO: 685) | ID NO: 826) | ID NO: 964) | ID NO: 1099) | ID NO: 1238) | ID NO: 1380) |
| DTSGLHS (SEQ | DTFGLHS (SEQ | DTYGLHS (SEQ | DTRGLHS (SEQ | DTMGLHS (SEQ | DTKGLHS (SEQ | DTLGLHS (SEQ |
| ID NO: 553) | ID NO: 686) | ID NO: 827) | ID NO: 965) | ID NO: 1100) | ID NO: 1239) | ID NO: 1381) |
| DTSGLPS (SEQ | DTFGLPS (SEQ | DTYGLPS (SEQ | DTRGLPS(SEQ | DTMGLPS (SEQ | DTKGLPS (SEQ | DTLGLPS (SEQ |
| ID NO: 108) | ID NO: 687) | ID NO: 828) | ID NO: 27) | ID NO: 1101) | ID NO: 1240) | ID NO: 1382) |
| DTSGLTS (SEQ | DTFGLTS (SEQ | DTYGLTS (SEQ | DTRGLTS (SEQ | DTMGLTS (SEQ | DTKGLTS (SEQ | DTLGLTS (SEQ |
| ID NO: 554) | ID NO: 688) | ID NO: 829) | ID NO: 966) | ID NO: 1102) | ID NO: 1241) | ID NO: 1383) |
| DTSGLDS (SEQ | DTFGLDS (SEQ | DTYGLDS (SEQ | DTRGLDS (SEQ | DTMGLDS (SEQ | DTKGLDS (SEQ | DTLGLDS (SEQ |
| ID NO: 555) | ID NO: 689) | ID NO: 830) | ID NO: 967) | ID NO: 1103) | ID NO: 1242) | ID NO: 1384) |
| DTSGHAS (SEQ | DTFGHAS (SEQ | DTYGHAS (SEQ | DTRGHAS (SEQ | DTMGHAS (SEQ | DTKGHAS (SEQ | DTLGHAS (SEQ |
| ID NO: 556) | ID NO: 690) | ID NO: 831) | ID NO: 968) | ID NO: 1104) | ID NO: 1243) | ID NO: 1385) |
| DTSGHSS (SEQ | DTFGHS1S (SEQ | DTYGHSS (SEQ | DTRGHSS (SEQ | DTMGHSS (SEQ | DTKGHSS (SEQ | DTLGHSS (SEQ |
| ID NO: 557) | ID NO: 691) | ID NO: 832) | ID NO: 969) | ID NO: 1105) | ID NO: 1244) | ID NO: 1386) |
| DTSGHKS (SEQ | DTFGHKS (SEQ | DTYGHKS (SEQ | DTRGHKS (SEQ | DTMGHKS (SEQ | DTKGHKS (SEQ | DTLGHKS (SEQ |
| ID NO: 558) | ID NO: 692) | ID NO: 833) | ID NO: 970) | ID NO: 1106) | ID NO: 1245) | ID NO: 1387) |
| DTSGHRS (SEQ | DTFGHRS (SEQ | DTYGHRS (SEQ | DTRGHRS (SEQ | DTMGHRS (SEQ | DTKGHRS (SEQ | DTLGHRS (SEQ |
| ID NO: 559) | ID NO: 693) | ID NO: 834) | ID NO: 971) | ID NO: 1107) | ID NO: 1246) | ID NO: 1388) |
| DTSGHHS (SEQ | DTFGHHS (SEQ | DTYGHHS (SEQ | DTRGHHS (SEQ | DTMGHHS (SEQ | DTKGHHS (SEQ | DTLGHHS (SEQ |
| ID NO: 560) | ID NO: 694) | ID NO: 835) | ID NO: 972) | ID NO: 1108) | ID NO: 1247) | ID NO: 1389) |
| DTSGHPS (SEQ | DTFGHPS (SEQ | DTYGHPS (SEQ | DTRGHPS (SEQ | DTMGHPS (SEQ | DTKGHPS (SEQ | DTLGHPS (SEQ |
| ID NO: 561) | ID NO: 695) | ID NO: 836) | ID NO: 973) | ID NO: 1109) | ID NO: 1248) | ID NO: 1390) |
| DTSGHTS (SEQ | DTFGHTS (SEQ | DTYGHTS (SEQ | DTRGHTS (SEQ | DTMGHTS (SEQ | DTKGHTS (SEQ | DTLGHTS (SEQ |
| ID NO: 562) | ID NO: 696) | ID NO: 837) | ID NO: 974) | ID NO: 1110) | ID NO: 1249) | ID NO: 1391) |
| DTSGHDS (SEQ | DTFGHDS (SEQ | DTYGHDS (SEQ | DTRGHDS (SEQ | DTMGHDS (SEQ | DTKGHDS (SEQ | DTLGHDS (SEQ |
| ID NO: 563) | ID NO: 697) | ID NO: 838) | ID NO: 975) | ID NO: 1111) | ID NO: 1250) | ID NO: 1392) |
| DTSGQAS (SEQ | DTFGQAS (SEQ | DTYGQAS (SEQ | DTRGQAS (SEQ | DTMGQAS (SEQ | DTKGQAS (SEQ | DTLGQAS (SEQ |
| ID NO: 564) | ID NO: 698) | ID NO: 839) | ID NO: 976) | ID NO: 1112) | ID NO: 1251) | ID NO: 1393) |
| DTSGQSS (SEQ | DTFGQSS (SEQ | DTYGQSS (SEQ | DTRGQSS (SEQ | DTMGQSS (SEQ | DTKGQSS (SEQ | DTLGQSS (SEQ |
| ID NO: 565) | ID NO: 699) | ID NO: 840) | ID NO: 977) | ID NO: 1113) | ID NO: 1252) | ID NO: 1394) |
| DTSGQKS (SEQ | DTFGQKS (SEQ | DTYGQKS (SEQ | DTRGQKS (SEQ | DTMGQKS (SEQ | DTKGQKS (SEQ | DTLGQKS (SEQ |
| ID NO: 566) | ID NO: 700) | ID NO: 841) | ID NO: 978) | ID NO: 1114) | ID NO: 1253) | ID NO: 1395) |
| DTSGQRS (SEQ | DTFGQRS (SEQ | DTYGQRS (SEQ | DTRGQRS (SEQ | DTMGQRS (SEQ | DTKGQRS (SEQ | DTLGQRS (SEQ |
| ID NO: 567) | ID NO: 701) | ID NO: 842) | ID NO: 979) | ID NO: 1115) | ID NO: 1254) | ID NO: 1396) |
| DTSGQHS (SEQ | DTFGQHS (SEQ | DTYGQHS (SEQ | DTRGQHS (SEQ | DTMGQHS (SEQ | DTKGQHS (SEQ | DTLGQHS (SEQ |
| ID NO: 568) | ID NO: 702) | ID NO: 843) | ID NO: 980) | ID NO: 1116) | ID NO: 1255) | ID NO: 1397) |
| DTSGQPS (SEQ | DTFGQPS (SEQ | DTYGQPS (SEQ | DTRGQPS (SEQ | DTMGQPS (SEQ | DTKGQPS (SEQ | DTLGQPS (SEQ |
| ID NO: 569) | ID NO: 703) | ID NO: 844) | ID NO: 981) | ID NO: 1117) | ID NO: 1256) | ID NO: 1398) |
| DTSGQTS (SEQ | DTFGQTS (SEQ | DTYGQTS (SEQ | DTRGQTS (SEQ | DTMGQTS (SEQ | DTKGQTS (SEQ | DTLGQTS (SEQ |
| ID NO: 570) | ID NO: 704) | ID NO: 845) | ID NO: 982) | ID NO: 1118) | ID NO: 1257) | ID NO: 1399) |
| DTSGQDS (SEQ | DTFGQDS (SEQ | DTYGQDS (SEQ | DTRGQDS (SEQ | DTMGQDS (SEQ | DTKGQDS (SEQ | DTLGQDS (SEQ |
| ID NO: 571) | ID NO: 705) | ID NO: 846) | ID NO: 983) | ID NO: 1119) | ID NO: 1258) | ID NO: 1400) |
| DTSRLAS (SEQ | DTFRLAS (SEQ | DTYRLAS (SEQ | DTRRLAS (SEQ | DTMRLAS (SEQ | DTKRLAS (SEQ | DTLRLAS (SEQ |
| ID NO: 123) | ID NO: 706) | ID NO: 847) | ID NO: 984) | ID NO: 32) | ID NO: 1259) | ID NO: 1401) |
| DTSRLSS (SEQ | DTFRLSS (SEQ | DTYRLSS (SEQ | DTRRLSS (SEQ | DTMRLSS (SEQ | DTKRLSS (SEQ | DTLRLSS (SEQ |
| ID NO: 572) | ID NO: 707) | ID NO: 848) | ID NO: 985) | ID NO: 1120) | ID NO: 1260) | ID NO: 1402) |
| DTSRLKS (SEQ | DTFRLKS (SEQ | DTYRLKS (SEQ | DTRRLKS (SEQ | DTMRLKS (SEQ | DTKRLKS (SEQ | DTLRLKS (SEQ |
| ID NO: 573) | ID NO: 708) | ID NO: 849) | ID NO: 986) | ID NO: 1121) | ID NO: 1261) | ID NO: 1403) |
| DTSRLRS (SEQ | DTFRLRS (SEQ | DTYRLRS (SEQ | DTRRLRS (SEQ | DTMRLRS (SEQ | DTKRLRS (SEQ | DTLRLRS (SEQ |
| ID NO: 574) | ID NO: 709) | ID NO: 850) | ID NO: 987) | ID NO: 1122) | ID NO: 1262) | ID NO: 1404) |
| DTSRLHS (SEQ | DTFRLHS (SEQ | DTYRLHS (SEQ | DTRRLHS (SEQ | DTMRLHS (SEQ | DTKRLHS (SEQ | DTLRLHS (SEQ |
| ID NO: 575) | ID NO: 710) | ID NO: 851) | ID NO: 988) | ID NO: 1123) | ID NO: 1263) | ID NO: 1405) |
| DTSRLPS (SEQ | DTFRLPS (SEQ | DTYRLPS (SEQ | DTRRLPS (SEQ | DTMRLPS (SEQ | DTKRLPS (SEQ | DTLRLPS (SEQ |
| ID NO: 576) | ID NO: 711) | ID NO: 852) | ID NO: 989) | ID NO: 1124) | ID NO: 1264) | ID NO: 1406) |
| DTSRLTS (SEQ | DTFRLTS (SEQ | DTYRLTS (SEQ | DTRRLTS (SEQ | DTMRLTS (SEQ | DTKRLTS (SEQ | DTLRLTS (SEQ |
| ID NO: 577) | ID NO: 712) | ID NO: 853) | ID NO: 990) | ID NO: 1125) | ID NO: 1265) | ID NO: 1407) |
| DTSRLDS (SEQ | DTFRLDS (SEQ | DTYRLDS (SEQ | DTRRLDS (SEQ | DTMRLDS (SEQ | DTKRLDS (SEQ | DTLRLDS (SEQ |
| ID NO: 578) | ID NO: 713) | ID NO: 854) | ID NO: 991) | ID NO: 1126) | ID NO: 1266) | ID NO: 1408) |
| DTSRHAS (SEQ | DTFRHAS (SEQ | DTYRHAS (SEQ | DTRRHAS (SEQ | DTMRHAS (SEQ | DTKRHAS (SEQ | DTLRHAS (SEQ |
| ID NO: 579) | ID NO: 714) | ID NO: 855) | ID NO: 992) | ID NO: 1127) | ID NO: 1267) | ID NO: 1409) |
| DTSRHSS (SEQ | DTFRHSS (SEQ | DTYRHSS (SEQ | DTRRHSS (SEQ | DTMRHSS (SEQ | DTKRHSS (SEQ | DTLRHSS (SEQ |
| ID NO: 580) | ID NO: 715) | ID NO: 856) | ID NO: 993) | ID NO: 1128) | ID NO: 1268) | ID NO: 1410) |
| DTSRHKS (SEQ | DTFRHKS (SEQ | DTYRHKS (SEQ | DTRRHKS (SEQ | DTMRHKS (SEQ | DTKRHKS (SEQ | DTLRHKS (SEQ |
| ID NO: 581) | ID NO: 716) | ID NO: 857) | ID NO: 994) | ID NO: 1129) | ID NO: 1269) | ID NO: 1411) |
| DTSRHRS (SEQ | DTFRHRS (SEQ | DTYRHRS (SEQ | DTRRHRS (SEQ | DTMRHRS (SEQ | DTKRHRS (SEQ | DTLRHRS (SEQ |
| ID NO: 582) | ID NO: 717) | ID NO: 858) | ID NO: 995) | ID NO: 1130) | ID NO: 1270) | ID NO: 1412) |
| DTSRHHS (SEQ | DTFRHHS (SEQ | DTYRHHS (SEQ | DTRRHHS (SEQ | DTMRHHS (SEQ | DTKRHHS (SEQ | DTLRHHS (SEQ |
| ID NO: 583) | ID NO: 718) | ID NO: 859) | ID NO: 996) | ID NO: 1131) | ID NO: 1271) | ID NO: 1413) |
| DTSRHPS (SEQ | DTFRHPS (SEQ | DTYRHPS (SEQ | DTRRHPS (SEQ | DTMRHPS (SEQ | DTKRHPS (SEQ | DTLRHPS (SEQ |
| ID NO: 584) | ID NO: 719) | ID NO: 860) | ID NO: 997) | ID NO: 1132) | ID NO: 1272) | ID NO: 1414) |
| DTSRHTS (SEQ | DTFRHTS (SEQ | DTYRHTS (SEQ | DTRRHTS (SEQ | DTMRHTS (SEQ | DTKRHTS (SEQ | DTLRHTS (SEQ |
| ID NO: 585) | ID NO: 720) | ID NO: 861) | ID NO: 998) | ID NO: 1133) | ID NO: 1273) | ID NO: 1415) |
| DTSRHDS (SEQ | DTFRHDS (SEQ | DTYRHDS (SEQ | DTRRHDS (SEQ | DTMRHDS (SEQ | DTKRHDS (SEQ | DTLRHDS (SEQ |
| ID NO: 586) | ID NO: 721) | ID NO: 862) | ID NO: 999) | ID NO: 1134) | ID NO: 1274) | ID NO: 1416) |
| DTSRQAS (SEQ | DTFRQAS (SEQ | DTYRQAS (SEQ | DTRRQAS (SEQ | DTMRQAS (SEQ | DTKRQAS (SEQ | DTLRQAS (SEQ |
| ID NO: 587) | ID NO: 722) | ID NO: 863) | ID NO: 1000) | ID NO: 1135) | ID NO: 1275) | ID NO: 1417) |
| DTSRQSS (SEQ | DTFRQSS (SEQ | DTYRQSS (SEQ | DTRRQSS (SEQ | DTMRQSS (SEQ | DTKRQSS (SEQ | DTLRQSS (SEQ |
| ID NO: 588) | ID NO: 723) | ID NO: 864) | ID NO: 1001) | ID NO: 1136) | ID NO: 1276) | ID NO: 1418) |
| DTSRQKS (SEQ | DTFRQKS (SEQ | DTYRQKS (SEQ | DTRRQKS (SEQ | DTMRQKS (SEQ | DTKRQKS (SEQ | DTLRQKS (SEQ |
| ID NO: 589) | ID NO: 724) | ID NO: 865) | ID NO: 1002) | ID NO: 1137) | ID NO: 1277) | ID NO: 1419) |
| DTSRQRS (SEQ | DTFRQRS (SEQ | DTYRQRS (SEQ | DTRRQRS (SEQ | DTMRQRS (SEQ | DTKRQRS (SEQ | DTLRQRS (SEQ |
| ID NO: 590) | ID NO: 725) | ID NO: 866) | ID NO: 1003) | ID NO: 1138) | ID NO: 1278) | ID NO: 1420) |
| DTSRQHS (SEQ | DTFRQHS (SEQ | DTYRQHS (SEQ | DTRRQHS (SEQ | DTMRQHS (SEQ | DTKRQHS (SEQ | DTLRQHS (SEQ |
| ID NO: 591) | ID NO: 726) | ID NO: 867) | ID NO: 1004) | ID NO: 1139) | ID NO: 1279) | ID NO: 1421) |
| DTSRQPS (SEQ | DTFRQPS (SEQ | DTYRQPS (SEQ | DTRRQPS (SEQ | DTMRQPS (SEQ | DTKRQPS (SEQ | DTLRQPS (SEQ |
| ID NO: 592) | ID NO: 727) | ID NO: 868) | ID NO: 1005) | ID NO: 1140) | ID NO: 1280) | ID NO: 1422) |
| DTSRQTS (SEQ | DTFRQTS (SEQ | DTYRQTS (SEQ | DTRRQTS (SEQ | DTMRQTS (SEQ | DTKRQTS (SEQ | DTLRQTS (SEQ |
| ID NO: 593) | ID NO: 728) | ID NO: 869) | ID NO: 1006) | ID NO: 1141) | ID NO: 1281) | ID NO: 1423) |
| DTSRQDS (SEQ | DTFRQDS (SEQ | DTYRQDS (SEQ | DTRRQDS (SEQ | DTMRQDS (SEQ | DTKRQDS (SEQ | DTLRQDS (SEQ |
| ID NO: 594) | ID NO: 729) | ID NO: 870) | ID NO: 1007) | ID NO: 1142) | ID NO: 1282) | ID NO: 1424) |
| DTSYLAS (SEQ | DTFYLAS (SEQ | DTYYLAS (SEQ | DTRYLAS(SEQ | DTMYLAS (SEQ | DTKYLAS (SEQ | DTLYLAS (SEQ |
| ID | ID NO: 99) | ID NO: 871) | ID NO: 178) | ID NO: 158) | ID NO: 1283) | ID NO: 1425) |
| NOS: 81&143) | | | | | | |
| DTSYLSS (SEQ | DTFYLSS (SEQ | DTYYLSS (SEQ | DTRYLSS(SEQ | DTMYLSS (SEQ | DTKYLSS (SEQ | DTLYLSS (SEQ |
| ID | ID NO: 90) | ID NO: 872) | ID NO: 59) | ID NO: 160) | ID NO: 1284) | ID NO: 1426) |
| NOS: 85&145) | | | | | | |
| DTSYLKS (SEQ | DTFYLKS (SEQ | DTYYLKS (SEQ | DTRYLKS (SEQ | DTMYLKS (SEQ | DTKYLKS (SEQ | DTLYLKS (SEQ |
| ID NO: 595) | ID NO: 730) | ID NO: 873) | ID NO: 1008) | ID NO: 1143) | ID NO: 1285) | ID NO: 1427) |
| DTSYLRS (SEQ | DTFYLRS (SEQ | DTYYLRS (SEQ | DTRYLRS (SEQ | DTMYLRS (SEQ | DTKYLRS (SEQ | DTLYLRS (SEQ |
| ID NO: 596) | ID NO: 731) | ID NO: 874) | ID NO: 1009) | ID NO: 1144) | ID NO: 1286) | ID NO: 1428) |
| DTSYLHS (SEQ | DTFYLHS (SEQ | DTYYLHS (SEQ | DTRYLHS (SEQ | DTMYLHS (SEQ | DTKYLHS (SEQ | DTLYLHS (SEQ |
| ID NO: 597) | ID NO: 732) | ID NO: 875) | ID NO: 1010) | ID NO: 1145) | ID NO: 1287) | ID NO: 1429) |
| DTSYLPS (SEQ | DTFYLPS (SEQ | DTYYLPS (SEQ | DTRYLPS (SEQ | DTMYLPS (SEQ | DTKYLPS (SEQ | DTLYLPS (SEQ |
| ID NO: 598) | ID NO: 733) | ID NO: 876) | ID NO: 1011) | ID NO: 1146) | ID NO: 1288) | ID NO: 1430) |
| DTSYLTS (SEQ | DTFYLTS (SEQ | DTYYLTS (SEQ | DTRYLTS (SEQ | DTMYLTS (SEQ | DTKYLTS (SEQ | DTLYLTS (SEQ |
| ID NO: 599) | ID NO: 734) | ID NO: 877) | ID NO: 1012) | ID NO: 1147) | ID NO: 1289) | ID NO: 1431) |
| DTSYLDS (SEQ | DTFYLDS (SEQ | DTYYLDS (SEQ | DTRYLDS (SEQ | DTMYLDS (SEQ | DTKYLDS (SEQ | DTLYLDS (SEQ |
| ID NO: 600) | ID NO: 735) | ID NO: 878) | ID NO: 1013) | ID NO: 1148) | ID NO: 1290) | ID NO: 1432) |
| DTSYHAS (SEQ | DTFYHAS (SEQ | DTYYHAS (SEQ | DTRYHAS (SEQ | DTMYHAS | DTKYHAS (SEQ | DTLYHAS (SEQ |
| ID NO: 601) | ID NO: 736) | ID NO: 879) | ID NO: 1014) | (SEQ ID | ID NO: 1291) | ID NO: 1433) |
| | | | | NO: 1149) | | |
| DTSYHSS (SEQ | DTFYHSS (SEQ | DTYYHSS (SEQ | DTRYHSS (SEQ | DTMYHSS (SEQ | DTKYHSS (SEQ | DTLYHSS (SEQ |
| ID NO: 602) | ID NO: 737) | ID NO: 880) | ID NO: 1015) | ID NO: 1150) | ID NO: 1292) | ID NO: 1434) |
| DTSYHKS (SEQ | DTFYHKS (SEQ | DTYYHKS (SEQ | DTRYHKS (SEQ | DTMYHKS | DTKYHKS (SEQ | DTLYHKS (SEQ |
| ID NO: 603) | ID NO: 738) | ID NO: 881) | ID NO: 1016) | (SEQ ID | ID NO: 1293) | ID NO: 1435) |
| | | | | NO: 1151) | | |
| DTSYHRS (SEQ | DTFYHRS (SEQ | DTYYHRS (SEQ | DTRYHRS (SEQ | DTMYHRS (SEQ | DTKYHRS (SEQ | DTLYHRS (SEQ |
| ID NO: 604) | ID NO: 739) | ID NO: 882) | ID NO: 1017) | ID NO: 1152) | ID NO: 1294) | ID NO: 1436) |
| DTSYHHS (SEQ | DTFYHHS (SEQ | DTYYHHS (SEQ | DTRYHHS (SEQ | DTMYHHS (SEQ | DTKYHHS (SEQ | DTLYHHS (SEQ |
| ID NO: 605) | ID NO: 740) | ID NO: 883) | ID NO: 1018) | ID NO: 1153) | ID NO: 1295) | ID NO: 1437) |
| DTSYHPS (SEQ | DTFYHPS (SEQ | DTYYHPS (SEQ | DTRYHPS (SEQ | DTMYHPS (SEQ | DTKYHPS (SEQ | DTLYHPS (SEQ |
| ID NO: 606) | ID NO: 741) | ID NO: 884) | ID NO: 1019) | ID NO: 1154) | ID NO: 1296) | ID NO: 1438) |
| DTSYHTS (SEQ | DTFYHTS (SEQ | DTYYHTS (SEQ | DTRYHTS (SEQ | DTMYHTS (SEQ | DTKYHTS (SEQ | DTLYHTS (SEQ |
| ID NO: 607) | ID NO: 742) | ID NO: 885) | ID NO: 1020) | ID NO: 1155) | ID NO: 1297) | ID NO: 1439) |
| DTSYHDS (SEQ | DTFYHDS (SEQ | DTYYHDS (SEQ | DTRYHDS (SEQ | DTMYHDS (SEQ | DTKYHDS (SEQ | DTLYHDS (SEQ |
| ID NO: 608) | ID NO: 743) | ID NO: 886) | ID NO: 1021) | ID NO: 1156) | ID NO: 1298) | ID NO: 1440) |
| DTSYQAS (SEQ | DTFYQAS (SEQ | DTYYQAS (SEQ | DTRYQAS(SEQ | DTMYQAS (SEQ | DTKYQAS (SEQ | DTLYQAS (SEQ |
| ID NO: 147) | ID NO: 744) | ID NO: 887) | ID NO: 167) | ID NO: 151) | ID NO: 1299) | ID NO: 1441) |
| DTSYQSS (SEQ | DTFYQSS (SEQ | DTYYQSS (SEQ | DTRYQSS(SEQ | DTMYQSS (SEQ | DTKYQSS (SEQ | DTLYQSS (SEQ |
| ID NO: 149) | ID NO: 745) | ID NO: 888) | ID NO: 53) | ID NO: 43) | ID NO: 1300) | ID NO: 1442) |
| DTSYQKS (SEQ | DTFYQKS (SEQ | DTYYQKS (SEQ | DTRYQKS (SEQ | DTMYQKS (SEQ | DTKYQKS (SEQ | DTLYQKS (SEQ |
| ID NO: 609) | ID NO: 746) | ID NO: 889) | ID NO: 1022) | ID NO: 1157) | ID NO: 1301) | ID NO: 1443) |
| DTSYQRS (SEQ | DTFYQRS (SEQ | DTYYQRS (SEQ | DTRYQRS (SEQ | DTMYQRS (SEQ | DTKYQRS (SEQ | DTLYQRS (SEQ |
| ID NO: 610) | ID NO: 747) | ID NO: 890) | ID NO: 1023) | ID NO: 1158) | ID NO: 1302) | ID NO: 1444) |
| DTSYQHS (SEQ | DTFYQHS (SEQ | DTYYQHS (SEQ | DTRYQHS (SEQ | DTMYQHS (SEQ | DTKYQHS (SEQ | DTLYQHS (SEQ |
| ID NO: 611) | ID NO: 748) | ID NO: 891) | ID NO: 1024) | ID NO: 1159) | ID NO: 1303) | ID NO: 1445) |
| DTSYQPS (SEQ | DTFYQPS (SEQ | DTYYQPS (SEQ | DTRYQPS (SEQ | DTMYQPS (SEQ | DTKYQPS (SEQ | DTLYQPS (SEQ |
| ID NO: 612) | ID NO: 749) | ID NO: 892) | ID NO: 1025) | ID NO: 1160) | ID NO: 1304) | ID NO: 1446) |
| DTSYQTS (SEQ | DTFYQTS (SEQ | DTYYQTS (SEQ | DTRYQTS (SEQ | DTMYQTS (SEQ | DTKYQTS (SEQ | DTLYQTS (SEQ |
| ID NO: 613) | ID NO: 750) | ID NO: 893) | ID NO: 1026) | ID NO: 1161) | ID NO: 1305) | ID NO: 1447) |
| DTSYQDS (SEQ | DTFYQDS (SEQ | DTYYQDS (SEQ | DTRYQDS (SEQ | DTMYQDS (SEQ | DTKYQDS (SEQ | DTLYQDS (SEQ |
| ID NO: 614) | ID NO: 751) | ID NO: 894) | ID NO: 1027) | ID NO: 1162) | ID NO: 1306) | ID NO: 1448) |
| DTSFLAS (SEQ | DTFFLAS (SEQ | DTYFLAS (SEQ | DTRFLAS (SEQ | DTMFLAS (SEQ | DTKFLAS (SEQ | DTLFLAS (SEQ |
| ID NO: 615) | ID NO: 752) | ID NO: 895) | ID NO: 1028) | ID NO: 1163) | ID NO: 1307) | ID NO: 1449) |
| DTSFLSS (SEQ | DTFFLSS (SEQ | DTYFLSS (SEQ | DTRFLSS (SEQ | DTMFLSS (SEQ | DTKFLSS (SEQ | DTLFLSS (SEQ |
| ID NO: 616) | ID NO: 753) | ID NO: 896) | ID NO: 1029) | ID NO: 1164) | ID NO: 1308) | ID NO: 1450) |
| DTSFLKS (SEQ | DTFFLKS (SEQ | DTYFLKS (SEQ | DTRFLKS (SEQ | DTMFLKS (SEQ | DTKFLKS (SEQ | DTLFLKS (SEQ |
| ID NO: 617) | ID NO: 754) | ID NO: 897) | ID NO: 1030) | ID NO: 1165) | ID NO: 1309) | ID NO: 1451) |
| DTSFLRS (SEQ | DTFFLRS (SEQ | DTYFLRS (SEQ | DTRFLRS (SEQ | DTMFLRS (SEQ | DTKFLRS (SEQ | DTLFLRS (SEQ |
| ID NO: 618) | ID NO: 755) | ID NO: 898) | ID NO: 1031) | ID NO: 1166) | ID NO: 1310) | ID NO: 1452) |
| DTSFLHS (SEQ | DTFFLHS (SEQ | DTYFLHS (SEQ | DTRFLHS (SEQ | DTMFLHS (SEQ | DTKFLHS (SEQ | DTLFLHS (SEQ |
| ID NO: 619) | ID NO: 756) | ID NO: 899) | ID NO: 1032) | ID NO: 1167) | ID NO: 1311) | ID NO: 1453) |
| DTSFLPS (SEQ | DTFFLPS (SEQ | DTYFLPS (SEQ | DTRFLPS (SEQ | DTMFLPS (SEQ | DTKFLPS (SEQ | DTLFLPS (SEQ |
| ID NO: 620) | ID NO: 757) | ID NO: 900) | ID NO: 1033) | ID NO: 1168) | ID NO: 1312) | ID NO: 1454) |
| DTSFLTS (SEQ | DTFFLTS (SEQ | DTYFLTS (SEQ | DTRFLTS (SEQ | DTMFLTS (SEQ | DTKFLTS (SEQ | DTLFLTS (SEQ |
| ID NO: 621) | ID NO: 758) | ID NO: 901) | ID NO: 1034) | ID NO: 1169) | ID NO: 1313) | ID NO: 1455) |
| DTSFLDS (SEQ | DTFFLDS (SEQ | DTYFLDS (SEQ | DTRFLDS (SEQ | DTMFLDS (SEQ | DTKFLDS (SEQ | DTLFLDS (SEQ |
| ID NO: 77) | ID NO: 50) | ID NO: 902) | ID NO: 1035) | ID NO: 1170) | ID NO: 1314) | ID NO: 1456) |
| DTSFHAS (SEQ | DTFFHAS (SEQ | DTYFHAS (SEQ | DTRFHAS (SEQ | DTMFHAS (SEQ | DTKFHAS (SEQ | DTLFHAS (SEQ |
| ID NO: 622) | ID NO: 759) | ID NO: 903) | ID NO: 1036) | ID NO: 1171) | ID NO: 1315) | ID NO: 1457) |
| DTSFHSS (SEQ | DTFFHSS (SEQ | DTYFHSS (SEQ | DTRFHSS (SEQ | DTMFHSS (SEQ | DTKFHSS (SEQ | DTLFHSS (SEQ |
| ID NO: 623) | ID NO: 760) | ID NO: 904) | ID NO: 1037) | ID NO: 1172) | ID NO: 1316) | ID NO: 1458) |
| DTSFHKS (SEQ | DTFFHKS (SEQ | DTYFHKS (SEQ | DTRFHKS (SEQ | DTMFHKS (SEQ | DTKFHKS (SEQ | DTLFHKS (SEQ |
| ID NO: 624) | ID NO: 761) | ID NO: 905) | ID NO: 1038) | ID NO: 1173) | ID NO: 1317) | ID NO: 1459) |
| DTSFHRS (SEQ | DTFFHRS (SEQ | DTYFHRS (SEQ | DTRFHRS (SEQ | DTMFHRS (SEQ | DTKFHRS (SEQ | DTLFHRS (SEQ |
| ID NO: 625) | ID NO: 762) | ID NO: 906) | ID NO: 1039) | ID NO: 1174) | ID NO: 1318) | ID NO: 1460) |
| DTSFHHS (SEQ | DTFFHHS (SEQ | DTYFHHS (SEQ | DTRFHHS (SEQ | DTMFHHS (SEQ | DTKFHHS (SEQ | DTLFHHS (SEQ |
| ID NO: 626) | ID NO: 763) | ID NO: 907) | ID NO: 1040) | ID NO: 1175) | ID NO: 1319) | ID NO: 1461) |
| DTSFHPS (SEQ | DTFFHPS (SEQ | DTYFHPS (SEQ | DTRFHPS (SEQ | DTMFHPS (SEQ | DTKFHPS (SEQ | DTLFHPS (SEQ |
| ID NO: 627) | ID NO: 764) | ID NO: 908) | ID NO: 1041) | ID NO: 1176) | ID NO: 1320) | ID NO: 1462) |
| DTSFHTS (SEQ | DTFFHTS (SEQ | DTYFHTS (SEQ | DTRFHTS (SEQ | DTMFHTS (SEQ | DTKFHTS (SEQ | DTLFHTS (SEQ |
| ID NO: 628) | ID NO: 765) | ID NO: 909) | ID NO: 1042) | ID NO: 1177) | ID NO: 1321) | ID NO: 1463) |
| DTSFHDS (SEQ | DTFFHDS (SEQ | DTYFHDS (SEQ | DTRFHDS (SEQ | DTMFHDS (SEQ | DTKFHDS (SEQ | DTLFHDS (SEQ |
| ID NO: 629) | ID NO: 766) | ID NO: 910) | ID NO: 1043) | ID NO: 1178) | ID NO: 1322) | ID NO: 1464) |
| DTSFQAS (SEQ | DTFFQAS (SEQ | DTYFQAS (SEQ | DTRFQAS (SEQ | DTMFQAS (SEQ | DTKFQAS (SEQ | DTLFQAS (SEQ |
| ID NO: 630) | ID NO: 767) | ID NO: 911) | ID NO: 1044) | ID NO: 1179) | ID NO: 1323) | ID NO: 1465) |
| DTSFQSS (SEQ | DTFFQSS (SEQ | DTYFQSS (SEQ | DTRFQSS (SEQ | DTMFQSS (SEQ | DTKFQSS (SEQ | DTLFQSS (SEQ |
| ID NO: 631) | ID NO: 768) | ID NO: 912) | ID NO: 1045) | ID NO: 1180) | ID NO: 1324) | ID NO: 1466) |
| DTSFQKS (SEQ | DTFFQKS (SEQ | DTYFQKS (SEQ | DTRFQKS (SEQ | DTMFQKS (SEQ | DTKFQKS (SEQ | DTLFQKS (SEQ |
| ID NO: 632) | ID NO: 769) | ID NO: 913) | ID NO: 1046) | ID NO: 1181) | ID NO: 1325) | ID NO: 1467) |
| DTSFQRS (SEQ | DTFFQRS (SEQ | DTYFQRS (SEQ | DTRFQRS (SEQ | DTMFQRS (SEQ | DTKFQRS (SEQ | DTLFQRS (SEQ |
| ID NO: 633) | ID NO: 770) | ID NO: 914) | ID NO: 1047) | ID NO: 1182) | ID NO: 1326) | ID NO: 1468) |
| DTSFQHS (SEQ | DTFFQHS (SEQ | DTYFQHS (SEQ | DTRFQHS (SEQ | DTMFQHS (SEQ | DTKFQHS (SEQ | DTLFQHS (SEQ |
| ID NO: 634) | ID NO: 771) | ID NO: 915) | ID NO: 1048) | ID NO: 1183) | ID NO: 1327) | ID NO: 1469) |
| DTSFQPS (SEQ | DTFFQPS (SEQ | DTYFQPS (SEQ | DTRFQPS (SEQ | DTMFQPS (SEQ | DTKFQPS (SEQ | DTLFQPS (SEQ |
| ID NO: 635) | ID NO: 772) | ID NO: 916) | ID NO: 1049) | ID NO: 1184) | ID NO: 1328) | ID NO: 1470) |
| DTSFQTS (SEQ | DTFFQTS (SEQ | DTYFQTS (SEQ | DTRFQTS (SEQ | DTMFQTS (SEQ | DTKFQTS (SEQ | DTLFQTS (SEQ |
| ID NO: 636) | ID NO: 773) | ID NO: 917) | ID NO: 1050) | ID NO: 1185) | ID NO: 1329) | ID NO: 1471) |
| DTSFQDS (SEQ | DTFFQDS (SEQ | DTYFQDS (SEQ | DTRFQDS (SEQ | DTMFQDS (SEQ | DTKFQDS (SEQ | DTLFQDS (SEQ |
| ID NO: 637) | ID NO: 774) | ID NO: 918) | ID NO: 1051) | ID NO: 1186) | ID NO: 1330) | ID NO: 1472) |
| DTSLLAS (SEQ | DTFLLAS (SEQ | DTYLLAS (SEQ | DTRLLAS (SEQ | DTMLLAS (SEQ | DTKLLAS (SEQ | DTLLLAS (SEQ |
| ID NO: 124) | ID NO: 775) | ID NO: 919) | ID NO: 1052) | ID NO: 1187) | ID NO: 1331) | ID NO: 133) |
| DTSLLSS (SEQ | DTFLLSS (SEQ | DTYLLSS (SEQ | DTRLLSS (SEQ | DTMLLSS (SEQ | DTKLLSS (SEQ | DTLLLSS (SEQ |
| ID NO: 638) | ID NO: 776) | ID NO: 920) | ID NO: 1053) | ID NO: 1188) | ID NO: 1332) | ID NO: 1473) |
| DTSLLKS (SEQ | DTFLLKS (SEQ | DTYLLKS (SEQ | DTRLLKS (SEQ | DTMLLKS (SEQ | DTKLLKS (SEQ | DTLLLKS (SEQ |
| ID NO: 639) | ID NO: 777) | ID NO: 921) | ID NO: 1054) | ID NO: 1189) | ID NO: 1333) | ID NO: 1474) |
| DTSLLRS (SEQ | DTFLLRS (SEQ | DTYLLRS (SEQ | DTRLLRS (SEQ | DTMLLRS (SEQ | DTKLLRS (SEQ | DTLLLRS (SEQ |
| ID NO: 640) | ID NO: 778) | ID NO: 922) | ID NO: 1055) | ID NO: 1190) | ID NO: 1334) | ID NO: 1475) |
| DTSLLHS (SEQ | DTFLLHS (SEQ | DTYLLHS (SEQ | DTRLLHS (SEQ | DTMLLHS (SEQ | DTKLLHS (SEQ | DTLLLHS (SEQ |
| ID NO: 641) | ID NO: 779) | ID NO: 923) | ID NO: 1056) | ID NO: 1191) | ID NO: 1335) | ID NO: 1476) |
| DTSLLPS (SEQ | DTFLLPS (SEQ | DTYLLPS (SEQ | DTRLLPS (SEQ | DTMLLPS (SEQ | DTKLLPS (SEQ | DTLLLPS (SEQ |
| ID NO: 642) | ID NO: 780) | ID NO: 924) | ID NO: 1057) | ID NO: 1192) | ID NO: 1336) | ID NO: 1477) |
| DTSLLTS (SEQ | DTFLLTS (SEQ | DTYLLTS (SEQ | DTRLLTS (SEQ | DTMLLTS (SEQ | DTKLLTS (SEQ | DTLLLTS (SEQ |
| ID NO: 643) | ID NO: 781) | ID NO: 925) | ID NO: 1058) | ID NO: 1193) | ID NO: 1337) | ID NO: 1478) |
| DTSLLDS (SEQ | DTFLLDS (SEQ | DTYLLDS (SEQ | DTRLLDS (SEQ | DTMLLDS (SEQ | DTKLLDS (SEQ | DTLLLDS (SEQ |
| ID NO: 126) | ID NO: 782) | ID NO: 926) | ID NO: 1059) | ID NO: 1194) | ID NO: 1338) | ID NO: 75) |
| DTSLHAS (SEQ | DTFLHAS (SEQ | DTYLHAS (SEQ | DTRLHAS (SEQ | DTMLHAS (SEQ | DTKLHAS (SEQ | DTLLHAS (SEQ |
| ID NO: 644) | ID NO: 783) | ID NO: 927) | ID NO: 1060) | ID NO: 1195) | ID NO: 1339) | ID NO: 1479) |
| DTSLHSS (SEQ | DTFLHSS (SEQ | DTYLHSS (SEQ | DTRLHSS (SEQ | DTMLHSS (SEQ | DTKLHSS (SEQ | DTLLHSS (SEQ |
| ID NO: 645) | ID NO: 784) | ID NO: 928) | ID NO: 1061) | ID NO: 1196) | ID NO: 1340) | ID NO: 1480) |
| DTSLHKS (SEQ | DTFLHKS (SEQ | DTYLHKS (SEQ | DTRLHKS (SEQ | DTMLHKS (SEQ | DTKLHKS (SEQ | DTLLHKS (SEQ |
| ID NO: 646) | ID NO: 785) | ID NO: 929) | ID NO: 1062) | ID NO: 1197) | ID NO: 1341) | ID NO: 1481) |
| DTSLHRS (SEQ | DTFLHRS (SEQ | DTYLHRS (SEQ | DTRLHRS (SEQ | DTMLHRS (SEQ | DTKLHRS (SEQ | DTLLHRS (SEQ |
| ID NO: 647) | ID NO: 786) | ID NO: 930) | ID NO: 1063) | ID NO: 1198) | ID NO: 1342) | ID NO: 1482) |
| DTSLHHS (SEQ | DTFLHHS (SEQ | DTYLHHS (SEQ | DTRLHHS (SEQ | DTMLHHS (SEQ | DTKLHHS (SEQ | DTLLHHS (SEQ |
| ID NO: 648) | ID NO: 787) | ID NO: 931) | ID NO: 1064) | ID NO: 1199) | ID NO: 1343) | ID NO: 1483) |
| DTSLHPS (SEQ | DTFLHPS (SEQ | DTYLHPS (SEQ | DTRLHPS (SEQ | DTMLHPS (SEQ | DTKLHPS (SEQ | DTLLHPS (SEQ |
| ID NO: 649) | ID NO: 788) | ID NO: 932) | ID NO: 1065) | ID NO: 1200) | ID NO: 1344) | ID NO: 1484) |
| DTSLHTS (SEQ | DTFLHTS (SEQ | DTYLHTS (SEQ | DTRLHTS (SEQ | DTMLHTS (SEQ | DTKLHTS (SEQ | DTLLHTS (SEQ |
| ID NO: 650) | ID NO: 789) | ID NO: 933) | ID NO: 1066) | ID NO: 1201) | ID NO: 1345) | ID NO: 1485) |
| DTSLHDS (SEQ | DTFLHDS (SEQ | DTYLHDS (SEQ | DTRLHDS (SEQ | DTMLHDS (SEQ | DTKLHDS (SEQ | DTLLHDS (SEQ |
| ID NO: 651) | ID NO: 790) | ID NO: 934) | ID NO: 1067) | ID NO: 1202) | ID NO: 1346) | ID NO: 1486) |
| DTSLQAS (SEQ | DTFLQAS (SEQ | DTYLQAS (SEQ | DTRLQAS (SEQ | DTMLQAS (SEQ | DTKLQAS (SEQ | DTLLQAS (SEQ |
| ID NO: 652) | ID NO: 791) | ID NO: 935) | ID NO: 1068) | ID NO: 1203) | ID NO: 1347) | ID NO: 1487) |
| DTSLQSS (SEQ | DTFLQSS (SEQ | DTYLQSS (SEQ | DTRLQSS (SEQ | DTMLQSS (SEQ | DTKLQSS (SEQ | DTLLQSS (SEQ |
| ID NO: 653) | ID NO: 792) | ID NO: 936) | ID NO: 1069) | ID NO: 1204) | ID NO: 1348) | ID NO: 1488) |
| DTSLQKS (SEQ | DTFLQKS (SEQ | DTYLQKS (SEQ | DTRLQKS (SEQ | DTMLQKS (SEQ | DTKLQKS (SEQ | DTLLQKS (SEQ |
| ID NO: 654) | ID NO: 793) | ID NO: 937) | ID NO: 1070) | ID NO: 1205) | ID NO: 1349) | ID NO: 1489) |
| DTSLQRS (SEQ | DTFLQRS (SEQ | DTYLQRS (SEQ | DTRLQRS (SEQ | DTMLQRS (SEQ | DTKLQRS (SEQ | DTLLQRS (SEQ |
| ID NO: 655) | ID NO: 794) | ID NO: 938) | ID NO: 1071) | ID NO: 1206) | ID NO: 1350) | ID NO: 1490) |
| DTSLQHS (SEQ | DTFLQHS (SEQ | DTYLQHS (SEQ | DTRLQHS (SEQ | DTMLQHS (SEQ | DTKLQHS (SEQ | DTLLQHS (SEQ |
| ID NO: 656) | ID NO: 795) | ID NO: 939) | ID NO: 1072) | ID NO: 1207) | ID NO: 1351) | ID NO: 1491) |
| DTSLQPS (SEQ | DTFLQPS (SEQ | DTYLQPS (SEQ | DTRLQPS (SEQ | DTMLQPS (SEQ | DTKLQPS (SEQ | DTLLQPS (SEQ |
| ID NO: 657) | ID NO: 796) | ID NO: 940) | ID NO: 1073) | ID NO: 1208) | ID NO: 1352) | ID NO: 1492) |
| DTSLQTS (SEQ | DTFLQTS (SEQ | DTYLQTS (SEQ | DTRLQTS (SEQ | DTMLQTS (SEQ | DTKLQTS (SEQ | DTLLQTS (SEQ |
| ID NO: 658) | ID NO: 797) | ID NO: 941) | ID NO: 1074) | ID NO: 1209) | ID NO: 1353) | ID NO: 1493) |
| DTSLQDS (SEQ | DTFLQDS (SEQ | DTYLQDS (SEQ | DTRLQDS (SEQ | DTMLQDS (SEQ | DTKLQDS (SEQ | DTLLQDS (SEQ |
| ID NO: 659) | ID NO: 798) | ID NO: 942) | ID NO: 1075) | ID NO: 1210) | ID NO: 1354) | ID NO: 1494) |

Bold faced & underlined amino acid residues are the residues which differ from the amino acid sequence in palivizumab

TABLE 3F
VL CDR3 Sequences

| | |
|---|---|
| FQGSGYPFT | (SEQ ID NO: 6) |
| FQGSFYPFT | (SEQ ID NO: 61) |
| FQGSYYPFT | (SEQ ID NO: 1495) |
| FQGSWYPFT | (SEQ ID NO: 1496) |

Bold faced and underlined amino acid residues are the residues which differ from the amino acid sequence in palivizumab

In one embodiment, antibodies of the invention comprise a VH CDR1 having the amino acid sequence of SEQ ID NO:1, SEQ ID NO:10 or SEQ ID NO:18. In another embodiment, antibodies of the invention comprise a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329. In another embodiment, antibodies of the invention comprise a VH CDR3 having the amino acid sequence of SEQ ID NO:3, SEQ ID NO:12, SEQ ID NO:20, SEQ ID NO:29, SEQ ID NO:79, or SEQ ID NO:311. In another embodiment, antibodies of the invention comprise a VH CDR1 having the amino acid sequence of SEQ ID NO:1, SEQ ID NO:10 or SEQ ID NO:18, a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329, and a VH CDR3 having the amino acid sequence of SEQ ID NO:3, SEQ ID NO:12, SEQ ID NO:20, SEQ ID NO:29, SEQ ID NO:79, or SEQ ID NO:311. In a preferred embodiment, antibodies of the invention comprise a VH CDR1 having the amino acid sequence of SEQ ID NO:10, a VH CDR2 having the amino acid sequence of SEQ ID NO:19, and a VH CDR3 having the amino acid sequence of SEQ ID NO:20. In accordance with these embodiments, the antibodies immunospecifically bind to a RSV F antigen. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In one embodiment, the amino acid sequence of the VH domain of an antibody of the invention is:

Q V T L R E S G P A L V K P T
Q T L T L T C T F S G F S L S
T A G M S V G W I R Q P P G K
A L E W L A D I W W D D K K H
Y N P S L K D R L T I S K D T
S K N Q V V L K V T N M D P A
D T A T Y Y C A R D M I F N F
Y F D V W G Q* G T T V T V S S

(SEQ ID NO:48), wherein the three underlined regions indicate the VH CDR1, CDR2, and CDR3 regions, respectively; the four non-underlined regions correlate with the VH FR1, FR2, FR3, FR4, respectively; and the asterisk indicates the position of an A→Q mutation in VH FR4 as compared to the VH FR4 of palivizumab shown in FIG. 1B (SEQ ID NO:7). This VH domain (SEQ ID NO:48) is identical to that of the MEDI-524 (and MEDI-524-YTE) antibody described elsewhere herein and shown in FIG. 13A. In some embodiments, this VH FR can be used in combination with any of the VH CDRs identified in Table 1 and/or Tables 3A-C. In one embodiment, the MEDI-524 antibody comprises the VH domain of FIG. 13A (SEQ ID NO:48) and the C-gamma-1 (nG1m) constant domain described in Johnson et al. (1997), J. Infect. Dis. 176, 1215-1224 and U.S. Pat. No. 5,824,307. In certain embodiments, said antibody comprises a modified IgG, such as a modified IgG1, constant domain, or FcRn-binding fragment thereof. In one embodiment, an antibody of the invention comprises a VH chain having the amino acid sequence of SEQ ID NO:208 and/or a VH domain having the amino acid sequence of SEQ ID NO:7. In another embodiment, an antibody of the invention comprises a VH chain having the amino acid sequence SEQ ID NO:254. In another embodiment, a modified antibody of the invention comprises a VH domain having the amino acid sequence SEQ ID NO:48. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention provides antibodies that immunospecifically bind to one or more RSV antigens (e.g., RSV F antigen), said antibodies comprising a VL chain having an amino acid sequence of any one of the VL chains listed in Table 2. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention also provides antibodies that immunospecifically bind to one or more RSV antigens (e.g., RSV F antigens), said antibodies comprising a VL domain having an amino acid sequence of any one of the VL domains listed in Table 2. The present invention also provides antibodies that immunospecifically bind to one or more RSV antigens (e.g., RSV F antigens), said antibodies comprising one or more VL CDRs having an amino acid sequence of any one of the VL CDRs listed in Table 2 and/or Tables 3D-3F. In some embodiments, the antibody comprises one, two or three of the VL CDRs listed in Table 2 and/or Tables 3D-3F. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In one embodiment of the present invention, the antibodies comprise a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:72, SEQ ID NO:314, SEQ ID NO:320, or SEQ ID NO:335. In another embodiment, antibodies of the invention comprise a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336. In another embodiment, antibodies of the invention comprise a VL CDR3 having the amino acid sequence of SEQ ID NO:6, SEQ ID NO:16 or SEQ ID NO:61. In another embodiment, antibodies of the invention comprise a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:72, SEQ ID NO:314, SEQ ID NO:320, or SEQ ID NO:335, a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336, and a VL CDR3 having the amino acid sequence of SEQ ID NO:6, SEQ ID NO:16 or SEQ ID NO:61. In a preferred embodiment, antibodies of the invention comprise a VL CDR1 having the amino acid sequence of SEQ ID NO:39, a VLCDR2 having the amino acid sequence of SEQ ID NO:5, and a VLCDR3 having the amino acid sequence of SEQ ID NO:6. In a specific embodiment, the antibodies have a high affinity for RSV antigen (e.g., RSV F antigen). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In one embodiment the amino acid sequence of the VL domain of an antibody of the invention is:

D I Q M T Q S P S T L S A S V
G D R V T I T C S A S S R V G
Y M H W Y Q Q K P G K A P K L
L I Y D T S K L A S G V P S R
F S G S G S G T E F T L T I S
S L Q P D D F A T Y Y C F Q G
S G Y P F T F G G G T K V* E I
K

(SEQ ID NO:11), wherein the three underlined regions indicate the VL CDR1, CDR2, and CDR3 regions, respectively; the four non-underlined regions correlate with the VL FR1, FR2, FR3, FR4, respectively; the asterisk indicates the position of an L→V mutation in VL FR4 as compared to the VL FR4 of palivizumab shown in FIG. 1A. This VL domain (SEQ ID NO:11) is identical to that of the MEDI-524 antibody described elsewhere herein and shown in FIG. 13B. In some embodiments, this VL framework can be used in combination with any of the VL CDRs identified in Table 1 and/or Tables 3D-3F. In one embodiment, the MEDI-524 antibody comprises the VL domain of FIG. 13B (SEQ ID NO:209) and the C-kappa constant domain described in Johnson et al. (1997) J. Infect. Dis. 176, 1215-1224 and U.S. Pat. No. 5,824,307, wherein said antibody comprises a modified IgG, such as a modified IgG1, constant domain, or FcRn-binding fragment thereof. In one embodiment, an antibody of the invention comprises a VL chain having the amino acid sequence of SEQ ID NO:209 and/or a VL domain having the amino acid sequence of SEQ ID NO:8. In another embodiment, an antibody of the invention comprises a VL chain having the amino acid sequence SEQ ID NO:255 and/or a VL domain having the amino acid sequence SEQ ID NO:11. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention further provides antibodies that immunospecifically bind to one or more RSV antigens (e.g., RSV F antigen), wherein the antibody comprises any VH chain disclosed herein combined with any VL chain disclosed herein, or any other VL chain. The present invention also provides antibodies that immunospecifically bind to one or more RSV antigens (e.g., RSV F antigen), wherein the antibody comprises any VL chain disclosed herein combined with any VH chain disclosed herein, or any other VH chain. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention also provides antibodies that immunospecifically bind to one or more RSV antigens (e.g., RSV F antigens), said antibodies comprising any VH domain disclosed herein combined with any VL domain disclosed herein, or any other VL domain. The present invention further provides antibodies that immunospecifically bind to one or more RSV antigens (e.g., RSV F antigens), said antibodies comprising any VL domain disclosed herein combined with any VH domain disclosed herein, or any other VH domain. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In a specific embodiment, antibodies that immunospecifically bind to a RSV antigen (e.g., RSV F antigens) comprise a VH domain having the amino acid sequence of SEQ ID NO:7, SEQ ID NO:9, SEQ ID NO:17, SEQ ID NO:24, SEQ ID NO:28, SEQ ID NO:33, SEQ ID NO:36, SEQ ID NO:40, SEQ ID NO:44, SEQ ID NO:48, SEQ ID NO:51, SEQ ID NO:55, SEQ ID NO:67, SEQ ID NO:78, SEQ ID NO:304, SEQ ID NO:310, SEQ ID NO:317, SEQ ID NO:323, or SEQ ID NO:328, and a VL domain having the amino acid sequence of SEQ ID NO:8, SEQ ID NO:13, SEQ ID NO:21, SEQ ID NO:26, SEQ ID NO:30, SEQ ID NO:34, SEQ ID NO:38, SEQ ID NO:42, SEQ ID NO:46, SEQ ID NO:49, SEQ ID NO:52, SEQ ID NO:54, SEQ ID NO:56, SEQ ID NO:58, SEQ ID NO:60, SEQ ID NO:62, SEQ ID NO:64, SEQ ID NO:65, SEQ ID NO:68, SEQ ID NO:70, SEQ ID NO:71, SEQ ID NO:74, SEQ ID NO:76, SEQ ID NO:307, SEQ ID NO:313, SEQ ID NO:319, SEQ ID NO:325, SEQ ID NO:331, or SEQ ID NO:334. In a preferred embodiment, antibodies that immunospecifically bind to a RSV F antigen comprise a VH domain having the amino acid sequence of SEQ ID NO:48 and a VL domain comprising the amino acid sequence of SEQ ID NO:11. In another specific embodiment, the antibodies of the invention have a high affinity and/or high avidity for a RSV antigen (e.g., RSV F antigen). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention further provides antibodies that specifically bind to a RSV antigen (e.g., RSV F antigen), wherein the antibody comprises any VH CDR1 disclosed herein, optionally in combination with any VH CDR2 disclosed herein (or other VH CDR2), and/or optionally in combination with any VH CDR3 disclosed herein (or other VH CDR3)), and/or optionally in combination with any VL CDR1 disclosed herein (or other VL CDR1), and/or optionally in combination with any VL CDR2 disclosed herein (or other VL CDR2), and/or optionally in combination with any VL CDR3 disclosed herein (or other VL CDR3). The present invention also provides antibodies that specifically bind to a RSV antigen (e.g., RSV F antigen), wherein the antibody comprises any VH CDR2 disclosed herein, optionally in combination with any VH CDR1 disclosed herein (or other VH CDR1), and/or optionally in combination with any VH CDR3 disclosed herein (or other VH CDR3)), and/or optionally in combination with any VL CDR1 disclosed herein (or other VL CDR1), and/or optionally in combination with any VL CDR2 disclosed herein (or other VL CDR2), and/or optionally in combination with any VL CDR3 disclosed herein (or other VL CDR3). The present invention also provides antibodies that specifically bind to a RSV antigen (e.g., RSV F antigen), wherein the antibody comprises any VH CDR3 disclosed herein, optionally in combination with any VH CDR1 disclosed herein (or other VH CDR1), and/or optionally in combination with any VH CDR2 disclosed herein (or other VH CDR3)), and/or optionally in combination with any VL CDR1 disclosed herein (or other VL CDR1), and/or optionally in combination with any VL CDR2 disclosed herein (or other VL CDR2), and/or optionally in combination with any VL CDR3 disclosed herein (or other VL CDR3). The present invention also provides antibodies that specifically bind to a RSV antigen (e.g., RSV F antigen), wherein the antibody comprises any VL CDR1 disclosed herein, optionally in combination with any VH CDR1 disclosed herein (or other VH CDR1), and/or optionally in combination with any VH CDR2 disclosed herein (or other VH CDR2)), and/or optionally in combination with any VH CDR3 disclosed herein (or other VH CDR3), and/or optionally in combination with any VL CDR2 disclosed herein (or other VL CDR2), and/or optionally in combination with any VL CDR3 disclosed herein (or other VL CDR3). The present invention further provides antibodies that specifically bind to a RSV antigen (e.g., RSV F antigen), wherein the antibody comprises any VL CDR2 disclosed herein, optionally in combination with any VH CDR1 disclosed herein (or other VH CDR1), and/or optionally in combination with any VH CDR2 disclosed herein (or other VH CDR2)), and/or optionally in combination with any VH CDR3 disclosed herein (or other VH CDR3), and/or optionally in combination with any VL CDR1 disclosed herein (or other VL CDR1), and/or optionally in combination with any VL CDR3 disclosed herein (or other VL CDR3). The present invention also provides antibodies that specifically bind to a RSV antigen (e.g., RSV F antigen), wherein the antibody comprises any VL CDR3 disclosed herein, optionally in combination with any VH CDR1 disclosed herein (or other VH CDR1), and/or optionally in combination with any VH CDR2 disclosed herein (or other VH CDR2)), and/or optionally in combination with any VH CDR3 disclosed herein (or other VH CDR3), and/or optionally in combination with any VL CDR1 disclosed herein (or other VL CDR1), and/or optionally in combination with any VL CDR2 disclosed herein (or other VL CDR2). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention also provides antibodies comprising one or more VH CDRs and one or more VL CDRs listed in Table 2 and/or Tables 3A-3F. In particular, the invention provides for an antibody comprising a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 2 and/or Tables 3A-3F. In a specific embodiment, the antibodies of the invention have a high affinity and/or high avidity for a RSV antigen (e.g., RSV F antigen). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The invention also provides for an antibody that immuno specifically binds to a RSV F antigen, comprising a VH CDR1 and a VL CDR1, a VH CDR1 and a VL CDR2, a VH CDR1 and a VL CDR3, a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 2 and/or Tables 3A-3F, supra. In another specific embodiment, the antibodies of the invention have a high affinity and/or high avidity for a RSV antigen (e.g., RSV F antigen). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In one embodiment, an antibody of the invention comprises a VH CDR1 having the amino acid sequence of SEQ ID NO:1, SEQ ID NO:10 or SEQ ID NO:18 and a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:314, SEQ ID NO:320, or SEQ ID NO:335. In another embodiment, an antibody of the invention comprises a VH CDR1 having the amino acid sequence of SEQ ID NO:1, SEQ ID NO:10 or SEQ ID NO:18 and a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336. In another embodiment, an antibody of the invention comprises a VH CDR1 having the amino acid sequence of SEQ ID NO:1, SEQ ID NO:10 or SEQ ID NO:18 and a VL CDR3 having the amino acid sequence of SEQ ID NO:6, SEQ ID NO:16 or SEQ ID NO:61. In accordance with these embodiments, the antibody immunospecifically binds to a RSV F antigen. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, an antibody of the invention comprises a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329, and a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:314, SEQ ID NO:320, or SEQ ID NO:335. In another embodiment, an antibody of the invention comprises a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329, and a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336. In another embodiment, an antibody of the invention comprises a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329, and a VL CDR3 having the amino acid sequence of SEQ ID NO:6, SEQ ID NO:16, or SEQ ID NO:61. In accordance with these embodiments, the antibody immunospecifically binds to a RSV F antigen. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, an antibody of the invention comprises a VH CDR3 having the amino acid sequence of SEQ ID NO:3, SEQ ID NO:12, SEQ ID NO:20, SEQ ID NO:29, SEQ ID NO:79, or SEQ ID NO:311, and a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:314, SEQ ID NO:320, or SEQ ID NO:335. In another embodiment, an antibody of the invention comprises a VH CDR3 having the amino acid sequence of SEQ ID NO:3, SEQ ID NO:12, SEQ ID NO:20, SEQ ID NO:29, SEQ ID NO:79, or SEQ ID NO:311, and a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336. In a preferred embodiment, an antibody of the invention comprises a VH CDR3 having the amino acid sequence of SEQ ID NO:3, SEQ ID NO:12, SEQ ID NO:20, SEQ ID NO:29, SEQ ID NO:79, or SEQ ID NO:311, and a VL CDR3 having the amino acid sequence of SEQ ID NO:6, SEQ ID NO:16, or SEQ ID NO:61. In accordance with these embodiments, the antibody immunospecifically binds to a RSV F antigen. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In some embodiments, modified antibody is a modified MEDI-524 antibody comprising the VH domain of FIG. 13A (SEQ ID NO:48), the VL domain of FIG. 13B, and the C-gamma-1 (nG1m) constant domain described in Johnson et al. (1997), J. Infect. Dis. 176, 1215-1224 and U.S. Pat. No. 5,824,307, wherein said antibody comprises a modified IgG, such as a modified IgG1, constant domain, or FcRn-binding fragment thereof. In other embodiments, modified antibody is a modified MEDI-524 antibody comprising the VH domain of FIG. 13A (SEQ ID NO:48), the VL domain of FIG. 13B, and the C-gamma-1 (nG1m) constant domain described in Johnson et al. (1997), J. Infect. Dis. 176, 1215-1224 and U.S. Pat. No. 5,824,307, wherein said antibody comprises one or more of a tyrosine at position 252, a threonine at position 254, and a glutamic acid at position 256 (numbered according to the EU index as in Kabat, supra), and preferably comprises the YTE modification (i.e., a tyrosine at position 252, a threonine at position 254, and a glutamic acid at position 256). In certain embodiments, modified antibody is a modified MEDI-524 antibody comprising the VH domain of FIG. 13A (SEQ ID NO:48), the VL domain of FIG. 13B, and the C-gamma-1 (nG1m) constant domain described in Johnson et al. (1997), J. Infect. Dis. 176, 1215-1224 and U.S. Pat. No. 5,824,307 wherein said antibody comprises a tyrosine at position 252, a threonine at position 254, and a glutamic acid at position 256 (numbered according to the EU index as in Kabat, supra) (hereafter “MEDI-524-YTE”).

The present invention also provides for a nucleic acid molecule(s) encoding an antibody (modified or unmodified) of the invention. In some embodiments, the nucleic acid molecule(s) encoding the antibody of the invention is isolated. In other embodiments, the nucleic acid molecule(s) encoding the antibody of the invention is not isolated. In yet other embodiments, the nucleic acid molecule(s) encoding the antibody of the invention is integrated, e.g., into chromosomal DNA or an expression vector. In a specific embodiment, nucleic acid molecules of the invention encode for the antibodies or antigen-binding fragments of the antibodies referenced in Table 2, and modified antibodies thereof. In one embodiment, a nucleic acid molecule(s) of the invention encode for AFFF, P12f2, P12f4, P11d4, Ale9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4 antibody. In another embodiment, nucleic acid molecule(s) of the invention encode for an antigen-binding fragment of AFFF, P12f2, P12f4, P11d4, Ale9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4 antibody. In one embodiment, nucleic acid molecule(s) of the invention encode for A4B4L1FR-S28R (MEDI-524) or an antigen-binding fragment thereof. In an embodiment, nucleic acid molecule(s) of the invention encode for MEDI-524-YTE. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, a nucleic acid molecule(s) of the invention encodes an antibody that immuno specifically binds to a RSV antigen (e.g., RSV F antigen), the antibody comprising a VH chain having an amino acid sequence of any one of the VH chains listed in Table 2. In another embodiment, a nucleic acid molecule(s) of the invention encodes an antibody that immunospecifically binds a RSV antigen (e.g., RSV F antigen), the antibody comprising a VH domain having an amino acid sequence of any one of the VH domains listed in Table 2. In another embodiment, a nucleic acid molecule(s) of the invention encodes an antibody that immunospecifically binds to a RSV antigen (e.g., RSV F antigen), the antibody comprising a VH CDR1 having an amino acid sequence of any one of the VH CDR1s listed in Table 2 and/or Table 3A. In another embodiment, a nucleic acid molecule(s) of the invention encodes an antibody that immunospecifically binds a RSV antigen (e.g., RSV F antigen), the antibody comprising a VH CDR2 having an amino acid sequence of any one of the VH CDR2s listed in Table 2 and/or Table 3B. In yet another embodiment, a nucleic acid molecule(s) of the invention encodes an antibody that immunospecifically binds a RSV antigen (e.g., RSV F antigen), the antibody comprising a VH CDR3 having an amino acid sequence of any one of the VH CDR3s listed in Table 2 and/or Table 3C. In some embodiments, the nucleic acid encodes a MEDI-524-YTE antibody. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, a nucleic acid molecule(s) of the invention encodes an antibody that immunospecifically binds to a RSV antigen (e.g., RSV F antigen), the antibody comprising a VL chain having an amino acid sequence of any one of the VL chains listed in Table 2. In one embodiment, a nucleic acid molecule(s) of the invention encodes an antibody that immunospecifically binds a RSV antigen (e.g., RSV F antigen), the antibody comprising a VL domain having an amino acid sequence of any one of the VL domains listed in Table 2. In another embodiment, a nucleic acid molecule(s) of the present invention encodes an antibody that immunospecifically binds a RSV antigen (e.g., RSV F antigen), the antibody comprising a VL CDR1 having amino acid sequence of any one of the VL CDR1s listed in Table 2 and/or Table 3D. In another embodiment, a nucleic acid molecule(s) of the present invention encodes an antibody that immunospecifically binds a RSV antigen (e.g., RSV F antigen), the antibody comprising a VL CDR2 having an amino acid sequence of any one of the VL CDR2s listed in Table 2 and/or Table 3E. In yet another embodiment, a nucleic acid molecule(s) of the present invention encodes an antibody that immunospecifically binds a RSV antigen (e.g., RSV F antigen), the antibody comprising a VL CDR3 having an amino acid sequence of any one of the VL CDR3s listed in Table 2 and/or Table 3F. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, a nucleic acid molecule(s) comprises a nucleotide sequence encoding a VH domain of an antibody that immunospecifically binds to a RSV antigen (e.g., RSV F antigen), where the VH domain comprises one, two or three VH CDRs having the amino acid sequence of one, two or three of the VH CDRs listed in Table 2 and/or Table 3A-3C. In one embodiment, a nucleic acid molecule(s) comprises a nucleotide sequence encoding a VL domain of an antibody that immunospecifically binds to a RSV antigen (e.g., RSV F antigen), where the VL domain comprises one, two or three VL CDRs having the amino acid sequence of one, two or three of the VL CDRs listed in Table 2 and/or Table 3D-3F. In another embodiment, a nucleic acid molecule(s) comprises a nucleotide sequence encoding a VH chain of an antibody that immunospecifically binds to a RSV antigen (e.g., RSV F antigen), where the VH chain comprises one, two or three VH CDRs having the amino acid sequence of one, two or three of the VH CDRs listed in Table 2 and/or Table 3A-3C. In another embodiment, a nucleic acid molecule(s) comprises a nucleotide sequence encoding a VL chain of an antibody that immunospecifically binds to a RSV antigen (e.g., RSV F antigen), where the VL chain comprises one, two or three VL CDRs having the amino acid sequence of one, two or three of the VL CDRs listed in Table 2 and/or Table 3D-3F. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, a nucleic acid molecule(s) of the invention encodes an antibody that immunospecifically binds to a RSV antigen (e.g., RSV F antigen), the antibody comprising a VH domain comprising an amino acid sequence of any one of the VH chains listed in Table 2. In another embodiment, a nucleic acid molecule(s) of the invention encodes an antibody that immunospecifically binds to a RSV antigen (e.g., RSV F antigen), the antibody comprising a VL domain having an amino acid sequence of any one of the VH chains listed in Table 2. In another embodiment, a nucleic acid molecule(s) of the invention encodes an antibody that immunospecifically binds to a RSV antigen (e.g., RSV F antigen), the antibody comprising a VH domain having an amino acid sequence of any one of the VH domains listed in Table 2 and a VL domain having an amino acid sequence of any one of the VL domains listed in Table 2 and/or Tables 3D-3F. In another embodiment, a nucleic acid molecule(s) of the invention encodes a modified antibody that immunospecifically binds a RSV antigen (e.g., RSV F antigen), the antibody comprising a VH CDR1, a VL CDR1, a VH CDR2, a VL CDR2, a VH CDR3, a VL CDR3, or any combination thereof having an amino acid sequence listed in Table 2 and/or Tables 3A-3F. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, the invention provides a nucleic acid molecule(s) encoding an antibody that immunospecifically binds to a RSV antigen, the antibody comprising a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 2 and/or Tables 3A-3F. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention also provides antibodies that immunospecifically bind to a RSV antigen (e.g., RSV F antigen), the antibodies comprising derivatives of the VH domains, VH CDRs, VL domains, and VL CDRs described herein that immunospecifically bind to a RSV antigen. The present invention also provides antibodies comprising derivatives of palivizumab, AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4, wherein said antibodies immunospecifically bind to one or more RSV antigens (e.g., RSV F antigen). Standard techniques known to those of skill in the art can be used to introduce mutations in the nucleotide sequence encoding a molecule of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention provides antibodies that immunospecifically bind to a RSV antigen (e.g., RSV F antigen), said antibodies comprising the amino acid sequence of the variable heavy domain and/or variable light domain or an antigen-binding fragment thereof of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4 with one or more amino acid residue substitutions in the variable heavy domain and/or variable light domain or antigen-binding fragment. The present invention also provides for antibodies that immunospecifically bind to a RSV antigen (e.g., RSV F antigen), said antibodies comprising the amino acid sequence of the variable heavy domain and/or variable light domain or an antigen-binding fragment thereof of AFFF, P12f2, P12f4, P11d4, Ale9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4 with one or more amino acid residue substitutions in one or more VH CDRs and/or one or more VL CDRs. Non-limiting examples of amino acid residues in the VH CDRs and VL CDRs of AFFF, P12f2, P12f4, P11d4, Ale9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4, which may be substituted, are shown in bold in Table 2. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention also provides antibodies that immunospecifically bind to a RSV antigen, said antibodies comprising the amino acid sequence of the VH domain and/or VL domain or an antigen-binding fragment thereof of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4 with one or more amino acid residue substitutions in one or more VH frameworks and/or one or more VL frameworks. The antibody generated by introducing substitutions in the VH domain, VH CDRs, VL domain, VL CDRs and/or frameworks of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4 can be tested in vitro and/or in vivo, for example, for its ability to bind to a RSV antigen, or for its ability to prevent, manage, treat and/or ameliorate a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In a specific embodiment, an antibody that immunospecifically binds to a RSV antigen (e.g., RSV F antigen) comprises an amino acid sequence encoded by a nucleotide sequence that hybridizes to the nucleotide sequence(s) encoding palivizumab, AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, A17h4, or an antigen-binding fragment thereof under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3).

In another embodiment, an antibody that immunospecifically binds to a RSV antigen (e.g., RSV F antigen) comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4, or an antigen-binding fragment thereof. In preferred embodiment, an antibody that immunospecifically binds to a RSV F antigen comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence of A4B4L1FR-S28R (MEDI-524), or an antigen-binding fragment thereof.

In a specific embodiment, an antibody that immunospecifically binds to a RSV antigen (e.g., RSV F antigen) comprises an amino acid sequence of a VH domain and/or an amino acid sequence a VL domain encoded by a nucleotide sequence that hybridizes to the nucleotide sequence encoding any one of the VH and/or VL domains listed in Table 2 under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3). In another embodiment, an antibody that immunospecifically binds to a RSV antigen comprises an amino acid sequence of a VH CDR or an amino acid sequence of a VL CDRs encoded by a nucleotide sequence that hybridizes to the nucleotide sequence encoding any one of the VH CDRs or VL CDRs listed in Table 2 and/or Tables 3A-3F under stringent conditions e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art. In yet another embodiment, an antibody that immunospecifically binds to a RSV antigen (e.g., RSV F antigen) comprises an amino acid sequence of a VH CDR and an amino acid sequence of a VL CDR encoded by nucleotide sequences that hybridizes to the nucleotide sequences encoding any one of the VH CDRs and VL CDRs, respectively, listed in Table 2 and/or Tables 3A-3F under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In another embodiment, an antibody that immunospecifically binds to a RSV antigen (e.g., RSV F antigen) comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to any one of the VH domains listed in Table 2. In another embodiment, an antibody that immunospecifically binds to a RSV antigen comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VH CDRs listed in Table 2 and/or Tables 3A-3C. In another embodiment, an antibody that immunospecifically binds to a RSV F antigen comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to any one of the VL domains listed in Table 2. In another embodiment, an antibody that immunospecifically binds to a RSV antigen (e.g., RSV F antigen) comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs listed in Table 2 and/or Tables 3D-3F.

The present invention also encompasses antibodies that compete with an antibody or Fab fragment listed in Table 2 for binding to a RSV antigen (e.g., RSV F antigen). The present invention also encompasses polypeptides, proteins and peptides comprising VL domains and/or VH domains that compete with a polypeptide, protein or peptide comprising a VL domain and/or a VH domain listed in Table 2 for binding to a RSV F antigen. Further, the present invention encompasses polypeptides, proteins and peptides comprising VL CDRs and/or VH CDRs that compete with a polypeptide, protein or peptide comprising a VL CDR and/or VH CDR listed in Table 2 and/or Tables 3A-3F for binding to a RSV F antigen.

The antibodies of the invention include derivatives that are chemically modified, i.e., by the covalent attachment of any type of molecule to the antibody. For example, but not by way of limitation, the antibody derivatives include antibodies that have been chemically modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The present invention also provides antibodies that immunospecifically bind to a RSV antigen (e.g., RSV F antigen) which comprise a framework region known to those of skill in the art (e.g., a human or non-human fragment). The framework region may be naturally occurring or consensus framework regions. Preferably, the framework region of an antibody of the invention is human (see, e.g., Chothia et al., 1998, J. Mol. Biol. 278:457-479 for a listing of human framework regions, which is incorporated by reference herein in its entirety). In a specific embodiment, an antibody of the invention comprises the framework region of A4B4L1FR-S28R (MEDI-524).

In a specific embodiment, the present invention provides for antibodies that immunospecifically bind to a RSV F antigen, said antibodies comprising the amino acid sequence of one or more of the CDRs of an antibody listed in Table 2 (i.e., AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4) and/or one or more of the CDRs in Table 3A-3F, and human framework regions with one or more amino acid substitutions at one, two, three or more of the following residues: (a) rare framework residues that differ between the murine antibody framework (i.e., donor antibody framework) and the human antibody framework (i.e., acceptor antibody framework); (b) Venier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention encompasses antibodies that immunospecifically bind to a RSV F antigen, said antibodies comprising the amino acid sequence of the VH domain and/or VL domain or an antigen-binding fragment thereof of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4 with mutations (e.g., one or more amino acid substitutions) in the framework regions. In certain embodiments, antibodies that immunospecifically bind to a RSV antigen comprise the amino acid sequence of the VH domain and/or VL domain or an antigen-binding fragment thereof of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4 with one or more amino acid residue substitutions in the framework regions of the VH and/or VL domains. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention also encompasses antibodies which immunospecifically bind to one or more RSV antigens (e.g., RSV F antigens), said antibodies comprising the amino acid sequence of A4B4L1FR-S28R (MEDI-524) with mutations (e.g., one or more amino acid substitutions) in the framework regions. In certain embodiments, antibodies which immunospecifically bind to one or more RSV F antigens comprise the amino acid sequence of A4B4L1FR-S28R (MEDI-524) with one or more amino acid residue substitutions in the framework regions of the VH and/or VL domains and one or more modifications in the constant domain, or FcRn-binding fragment thereof (preferably the Fc domain or hinge-Fdc domain). In a specific embodiment, modified antibodies which immunospecifically bind to one or more RSV F antigens comprise the framework regions depicted in FIG. 2 or FIG. 13. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention also encompasses antibodies that immunospecifically bind to a RSV antigen, said antibodies comprising the amino acid sequence of the VH domain and/or VL domain of an antibody in Table 2 (i.e., AFFF, P12f2, P12f4, P11d4, Ale9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4) with mutations (e.g., one or more amino acid residue substitutions) in the hypervariable and framework regions. Preferably, the amino acid substitutions in the hypervariable and framework regions improve binding of the antibody to a RSV antigen. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention also encompasses antibodies which immunospecifically bind to one or more RSV F antigens, said antibodies comprising the amino acid sequence of A4B4L1FR-S28R (MEDI-524) with mutations (e.g., one or more amino acid residue substitutions) in the variable and framework regions. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The present invention also provides antibodies of the invention that immunospecifically bind to a RSV antigen (e.g., RSV F antigen) which comprise constant regions known to those of skill in the art (e.g., the C-gamma-1 (G1m) constant domain described in Johnson et al. (1997), J. Infect. Dis. 176:1215-1224 and U.S. Pat. No. 5,824,307). Preferably, the constant regions of a modified or unmodified antibody of the invention provided herein are human. In a specific embodiment, an antibody of the invention comprises the constant regions of A4B4L1FR-S28R (MEDI-524). In other embodiments, the modified antibodies of the invention comprise a modified IgG constant domain, or FcRn-binding fragment thereof (preferably, Fc domain or hinge-Fc domain). In certain embodiments, the modified antibodies of the invention comprise a modified IgG, such as a modified IgG1, constant domain, or FcRn binding fragment thereof. In a preferred embodiment, the above-referenced modified antibodies comprise a modified IgG, such as a modified IgG1, constant domain, or FcRn binding fragment thereof, comprising YTE.

The present invention also provides for fusion proteins comprising an antibody provided herein that immunospecifically binds to a RSV antigen and a heterologous polypeptide. Preferably, the heterologous polypeptide that the antibody are fused to is useful for targeting the antibody to respiratory epithelial cells.

The present invention also provides for panels of antibodies that immunospecifically bind to a RSV antigen. In specific embodiments, the invention provides for panels of antibodies having different association rate constants different dissociation rate constants, different affinities for a RSV antigen, and/or different specificities for a RSV antigen. The invention provides panels of about 10, preferably about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well plates for assays such as ELISAs. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

### 5.1.1 Modifications of Antibodies to Increase Half-Lives

The present invention provides for modified antibodies that immunospecifically bind to a RSV antigen which have an extended (or increased) half-life in vivo. In particular, the present invention provides modified antibodies that immunospecifically bind to a RSV antigen which have a half-life in a subject, preferably a mammal and most preferably a human, of from about 3 days to about 180 days (or more), and in some embodiments greater than 3 days, greater than 7 days, greater than 10 days, greater than 15 days, greater than 20 days, greater than 25 days, greater than 30 days, greater than 35 days, greater than 40 days, greater than 45 days, greater than 50 days, at least about 60 days, greater than 75 days, greater than 90 days, greater than 105 days, greater than 120 days, greater than 135 days, greater than 150 days, greater than 165 days, or greater than 180 days. In preferred embodiments, the modified antibodies comprise a modified IgG constant domain, or FcRn-binding fragment thereof (preferably, Fc domain or hinge-Fc domain), resulting in an extended in vivo half-life. In preferred embodiments, the modified antibodies comprise a modified IgG, such as a modified IgG1, constant domain, or FcRn binding fragment thereof, comprising YTE. In some embodiments, the modified antibody is MEDI-524-YTE.

In certain embodiments, the in vivo half-life of the modified antibody is increased as compared to as compared to the same antibody that does not comprise one or more modifications in the IgG constant domain, or FcRn-binding fragment thereof, as determined using methods described herein or known in the art (see Example 6.17). In some embodiments, the half-life of the modified antibody is increased by about 2-fold, about 3-fold, about 4-fold, about 5-fold, about 6-fold, about 7-fold, about 8-fold, about 9-fold, about 10-fold, about 20-fold or more as compared to the same antibody that does not comprise one or more modifications in the IgG constant domain, or FcRn-binding fragment thereof. In certain embodiments, the half-life of the modified antibody is increased by 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, 30 days or more as compared to the same antibody that does not comprise one or more modifications in the IgG constant domain, or FcRn-binding fragment thereof.

In a specific embodiment, modified antibodies having an increased half-life in vivo are be generated by introducing one or more amino acid modifications (i.e., substitutions, insertions or deletions) into an IgG constant domain, or FcRn-binding fragment thereof (preferably a Fc or hinge-Fc domain fragment). See, e.g., International Publication Nos. WO 02/060919; WO 98/23289; and WO 97/34631; and U.S. Pat. No. 6,277,375; each of which is incorporated herein by reference in its entirety. In a preferred embodiment, the modified antibodies have one or more amino acid modifications in the second constant CH2 domain (residues 231-340 of human IgG1) (e.g., SEQ ID NO:339) and/or the third constant CH3 domain (residues 341-447 of human IgG1) (e.g., SEQ ID NO:340), with numbering according to the EU Index as in Kabat, supra. (See, e.g., FIG. 20B).

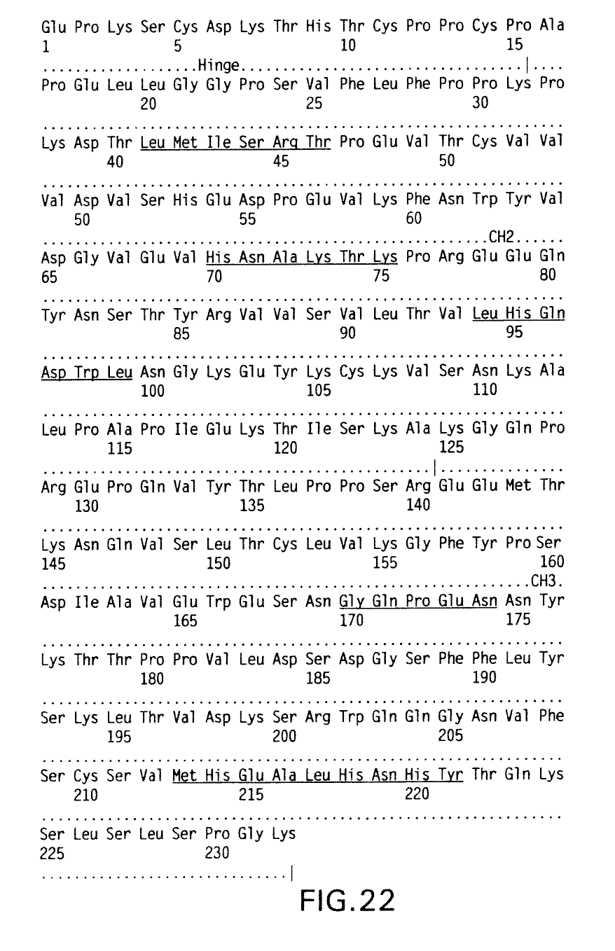

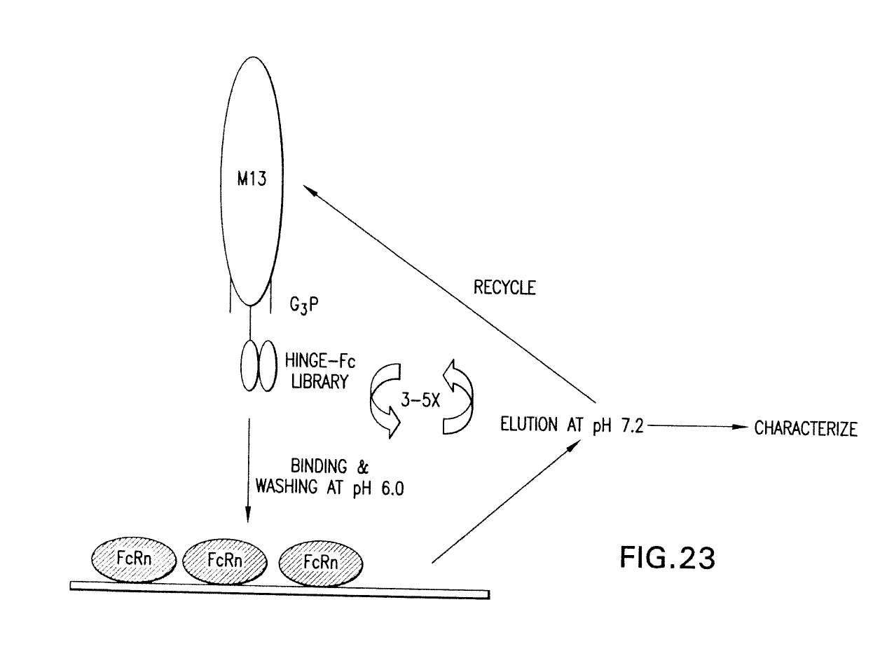

The present invention provides amino acid residues and/or modifications in particular portions of the constant domain (e.g., of an IgG molecule) that interact with the FcRn, which modifications increase the affinity of the IgG, or fragment thereof, for the FcRn. Accordingly, the invention provides molecules, preferably proteins, more preferably immunoglobulins (including any antibody disclosed in Section 5.1 or elsewhere in this application), that comprise an IgG (e.g., IgG1) constant domain, or FcRn-binding fragment thereof (preferably a Fc or hinge-Fc domain fragment), having one or more amino acid modifications (i.e., substitutions, insertions, deletions, and/or naturally occurring residues) in one or more regions that interact with the FcRn, which modifications increase the affinity of the IgG or fragment thereof, for the FcRn, and also increase the in vivo half-life of the molecule. In certain embodiments, the one or more amino acid modifications are made in one or more of residues 251-256, 285-290, 308-314, 385-389, and 428-436 of the IgG hinge-Fc region (for example, as in the human IgG1 hinge-Fc region depicted in FIG. 20B, FIG. 22, or SEQ ID NO:342), or analogous residues thereof, as determined by amino acid sequence alignment, in other IgG hinge-Fc regions. Numbering of residues are according to the EU index in Kabat et al. (1991). Sequences of proteins of immunological interest. (U.S. Department of Health and Human Services, Washington, D.C.) 5^{th} ed. (“Kabat et al.”). An exemplary human IgG1 constant domain hinge-Fc region is depicted in FIG. 20B with numbering according to the EU Index as in Kabat et al., supra. Due to natural variations in IgG constant domain sequences (see, e.g., Kabat et al., supra), in certain instances, a first amino acid residue may be substituted with a second amino acid residue at a given position (for example, in the sequence shown in FIG. 20B, the Met at position 252 may be substituted with a Tyr) or, alternatively, the second residue may be already present in antibody at the given position, in which case substitution is not necessary (for example, the Met at position 252 remains a Met). Antibody modifications are described in co-owned and co-pending U.S. Ser. No. 10/020,354 which is incorporated herein by reference in its entirety.

In a preferred embodiment, the amino acid modifications are made in a human IgG constant domain such as a human IgG1 constant domain (e.g., those described in Kabat et al., supra), or FcRn-binding fragment thereof (preferably, Fc domain or hinge-Fc domain). In a certain embodiment, the modifications are not made at residues 252, 254, or 256 (i.e., all are made at one or more of residues 251, 253, 255, 285-290, 308-314, 385-389, or 428-436) of the IgG constant domain. In one embodiment, the amino acid modifications are not the substitution with leucine at residue 252, with serine at 254, and/or with phenylalanine at position 256. In particular, in certain embodiments, such modifications are not made when the IgG constant domain, hinge-Fc domain, hinge-Fc domain or other FcRn-binding fragment thereof is derived from a mouse.

The amino acid modifications may be any modification, for example, at one or more of residues 251-256, 285-290, 308-314, 385-389, and 428-436 (see, e.g., FIG. 20B), that increases the in vivo half-life of the IgG constant domain, or FcRn-binding fragment thereof (e.g., Fc or hinge-Fc domain), and any molecule attached thereto, and increases the affinity of the IgG, or fragment thereof, for FcRn. In some embodiments, the modified antibodies comprise one or more amino acid substitutions, naturally occurring amino acids, or combinations thereof, at the indicated amino acid positions. Preferably, the one or more modifications also result in a higher binding affinity of the constant domain, or FcRn-binding fragment thereof, for FcRn at pH 6.0 than at pH 7.4. In other embodiments, the modifications alter (i.e., increase or decrease) bioavailability of the molecule, in particular, alters (i.e., increases or decreases) transport (or concentration or half-life) of the molecule to mucosal surfaces (e.g., of the lungs) or other portions of a target tissue. In a preferred embodiment, the amino acid modifications alter (preferably, increase) transport or concentration or half-life of the molecule to the lungs. In other embodiments, the amino acid modifications alter (preferably, increase) transport (or concentration or half-life) of the molecule to the heart, pancreas, liver, kidney, bladder, stomach, large or small intestine, respiratory tract, lymph nodes, nervous tissue (central and/or peripheral nervous tissue), muscle, epidermis, bone, cartilage, joints, blood vessels, bone marrow, prostate, ovary, uterine, tumor or cancer tissue, etc. In a preferred embodiment, the amino acid modifications do not abolish, or, more preferably, do not alter, other immune effector or receptor binding functions of the constant domain, for example, but not limited to complement fixation, ADCC and binding to FcγRI, FcγRII, and FcγRIII, as can be determined by methods well-known and routine in the art. In another preferred embodiment, the modified FcRn-binding fragment of the constant domain does not contain sequences that mediate immune effector functions or other receptor binding. Such fragments may be particularly useful for conjugation to a non-IgG or non-immunoglobulin molecule to increase the in vivo half-life thereof. In yet another embodiment, the effector functions are selectively altered (e.g., to reduce or increase effector functions).

In certain embodiments, the IgG constant domain comprises a modification at one or more of residues 308, 309, 311, 312 and 314. In some embodiments, a modified antibody comprises a threonine at position 308, proline at position 309, serine at position 311, aspartic acid at position 312, and/or leucine at position 314. In other embodiments, a modified antibody comprises an isoleucine at position 308, proline at position 309, and/or a glutamic acid at position 311. In yet another embodiment, a modified antibody comprises a threonine at position 308, a proline at position 309, a leucine at position 311, an alanine at position 312, and/or an alanine at position 314. Accordingly, in certain embodiments a modified antibody comprises a constant domain, wherein the residue at position 308 is a threonine or isoleucine, the residue at position 309 is proline, the residue at position 311 is serine, glutamic acid or leucine, the residue at position 312 is alanine, and/or the residue at position 314 is leucine or alanine. In one embodiment, a modified antibody comprises threonine at position 308, proline at position 309, serine at position 311, aspartic acid at position 312, and/or leucine at position 314.

In some embodiments, a modified antibody comprises a constant domain, wherein one or more of residues 251, 252, 254, 255, and 256 is modified. In specific embodiments, residue 251 is leucine or arginine, residue 252 is tyrosine, phenylalanine, serine, tryptophan or threonine, residue 254 is threonine or serine, residue 255 is arginine, leucine, glycine, or isoleucine, and/or residue 256 is serine, arginine, glutamine, glutamic acid, aspartic acid, alanine, asparagine or threonine. In a more specific embodiment, residue 251 is leucine, residue 252 is tyrosine, residue 254 is threonine or serine, residue 255 is arginine, and/or residue 256 is glutamic acid. In certain embodiments, the residue at position 252 is a tyrosine, the residue at position 254 is a threonine, or the residue at position 256 is a glutamic acid. In preferred embodiments, modified IgG, such as a modified IgG1, constant domain, or FcRn binding fragment thereof, comprises the YTE modification, i.e., the residue at position 252 is a tyrosine (Y), the residue at position 254 is a threonine (T), and the residue at position 256 is a glutamic acid (E). In preferred embodiments, the modified antibody is MEDI-524-YTE.

In specific embodiments, the amino acid modifications are substitutions at one or more of residues 428, 433, 434, and 436. In some embodiments, residue 428 is threonine, methionine, leucine, phenylalanine, or serine, residue 433 is lysine, arginine, serine, isoleucine, proline, glutamine or histidine, residue 434 is phenylalanine, tyrosine, or histidine, and/or residue 436 is histidine, asparagine, arginine, threonine, lysine, or methionine. In a more specific embodiment, residues at position 428 and/or 434 are substituted with methionine, and/or histidine respectively.

In other embodiments, the amino acid sequence comprises modifications at one or more of residues 385, 386, 387, and 389. In specific embodiments, residue 385 is arginine, aspartic acid, serine, threonine, histidine, lysine, alanine or glycine, residue 386 is threonine, proline, aspartic acid, serine, lysine, arginine, isoleucine, or methionine, residue 387 is arginine, proline, histidine, serine, threonine, or alanine, and/or residue 389 is proline, serine or asparagine. In more specific embodiments, one or more of positions 385, 386, 387, and 389 are arginine, threonine, arginine, and proline, respectively. In yet another specific embodiment, one or more of positions 385, 386, and 389 are aspartic acid, proline, and serine, respectively.

In some embodiments, amino acid modifications are made at one or a combination of residues 251, 252, 254, 255, 256, 308, 309, 311, 312, 314, 385, 386, 387, 389, 428, 433, 434, and/or 436, particularly where the modifications are amino acid residues described immediately above for these residues.

In some embodiments, the molecule of the invention contains a Fc region, or FcRn-binding fragment thereof, having one or more of the following: leucine at residue 251, tyrosine at residue 252, threonine or serine at residue 254, arginine at residue 255, threonine at residue 308, proline at residue 309, serine at residue 311, aspartic acid at residue 312, leucine at residue 314, arginine at residue 385, threonine at residue 386, arginine at residue 387, proline at residue 389, methionine at residue 428, and/or tyrosine at residue 434.

In certain embodiments, the FcRn-binding fragment has a modification at 1, 2, 3, 4, 5, 6, 7, 8, 9, 10, 11, 12, 13, 14, 15, 16, 17, or all 18 of residues 251, 252, 254, 255, 256, 308, 309, 311, 312, 314, 385, 386, 387, 389, 428, 433, 434, and/or 436.

Due to natural variations in IgG constant domain sequences (see, e.g., Kabat et al., supra), in certain instances, a first amino acid residue may be substituted (or otherwise modified) with a second amino acid residue at a given position (for example, in the sequence shown in FIG. 20B, the Met at position 252 may be substituted with a Tyr) or, alternatively, the second residue may be already present in antibody at the given position, in which case substitution is not necessary (for example, the Met at position 252 remains a Met). Amino acid modifications can be made by any method known in the art and many such methods are well known and routine for the skilled artisan. For example, but not by way of limitation, amino acid substitutions, deletions and insertions may be accomplished using any well-known PCR-based technique. Amino acid substitutions may be made by site-directed mutagenesis (see, for example, Zoller and Smith, Nucl. Acids Res. 10:6487-6500, 1982; Kunkel, Proc. Natl. Acad. Sci. USA 82:488, 1985, which are hereby incorporated by reference in their entireties). Mutants that result in increased affinity for FcRn and increased in vivo half-life may readily be screened using well-known and routine assays, such as those described in Sections 5.5 and 5.6, infra. In a preferred method, amino acid substitutions are introduced at one or more residues in the IgG constant domain or FcRn-binding fragment thereof and the mutated constant domains or fragments are expressed on the surface of bacteriophage which are then screened for increased FcRn binding affinity (see, in particular, Sections 5.5 and 5.6, infra).

Preferably, the modified amino acid residues are surface exposed residues. Additionally, in making amino acid substitutions, preferably the amino acid residue to be substituted is a conservative amino acid substitution, for example, a polar residue is substituted with a polar residue, a hydrophilic residue with a hydrophilic residue, hydrophobic residue with a hydrophobic residue, a positively charged residue with a positively charged residue, or a negatively charged residue with a negatively charged residue. Moreover, preferably, the modified amino acid residue is not highly or completely conserved across species and/or is critical to maintain the constant domain tertiary structure or to FcRn binding. For example, but not by way of limitation, modification of the histidine at residue 310 is not preferred.

Specific mutants of the Fc domain that have increased affinity for FcRn were isolated after the third-round panning (as described in Section 6.17) from a library of mutant human IgG1 molecules having mutations at residues 308-314 (histidine at position 310 and tryptophan at position 313 are fixed), those isolated after the fifth-round panning of the library for residues 251-256 (isoleucine at position 253 is fixed), those isolated after fourth-round panning of the library for residues 428-436 (histidine at position 429, glutamic acid at position 430, alanine at position 431, leucine at position 432, and histidine at position 435 are fixed), and those isolated after sixth-round panning of the library for residues 385-389 (glutamic acid at position 388 is fixed) are listed in Table 33, infra. The wild type human IgG1 has a sequence Val-Leu-His-Gln-Asp-Trp-Leu (SEQ ID NO:344) at positions 308-314, Leu-Met-Ile-Ser-Arg-Thr (SEQ ID NO:345) at positions 251-256, Met-His-Glu-Ala-Leu-His-Asn-His-Tyr (SEQ ID NO:346) at positions 428-436, and Gly-Gln-Pro-Glu-Asn (SEQ ID NO:347) at positions 386-389.

In some embodiments, an antibody of the invention contains a Fc region, or FcRn-binding fragment thereof, having one or more particular amino acid residues among the amino acid residues at positions 251-256 of the Fc region selected from the group consisting of the following residues: residue 252 is tyrosine, phenylalanine, serine, tryptophan or threonine; residue 254 is threonine; residue 255 is arginine, leucine, glycine, or isoleucine; and residue 256 is serine, arginine, glutamine, glutamic acid, aspartic acid, or threonine. In a particular embodiment, at least one amino acid modification is selected from the group consisting of the following: residue 251 is leucine, residue 252 is tyrosine, residue 254 is threonine, residue 255 is arginine, and residue 256 is glutamic acid. In certain embodiments, residue 252 is not leucine, alanine, or valine; residue 253 is not alanine; residue 254 is not serine or alanine; residue 255 is not alanine; and/or residue 256 is not alanine, histidine, phenylalanine, glycine, or asparagine.

In another embodiment, a modified antibody of the invention contains a Fc region, or FcRn-binding fragment thereof, having one or more particular amino acid residues among the amino acid residues at positions 285-290 of the Fc region. In particular embodiments, residue 285 is not alanine; residue 286 is not alanine, glutamine, serine, or aspartic acid; residue 288 is not alanine; residue 289 is not alanine; and/or residue 290 is not alanine, glutamine, serine, glutamic acid, arginine, or glycine.

In some embodiments, a modified antibody of the invention contains a Fc region, or FcRn-binding fragment thereof, having one or more particular amino acid residues among the amino acid residues at positions 308-314 of the Fc region selected from the group consisting of the following residues: a threonine at position 308, a proline at position 309, a serine at position 311, and an aspartic acid at position 312. In another embodiment, an antibody of the invention comprises one or more specific modifications selected from the group consisting of an isoleucine at position 308, a proline at position 309, and a glutamic acid at position 311. In another embodiment, a modified antibody comprises one or more specific amino acid residues selected from the group consisting of a threonine at position 308, a proline at position 309, and a leucine at position 311. In certain embodiments, position 309 is not an alanine; position 310 is not an alanine; position 311 is not an alanine or an asparagine; position 312 is not an alanine; and/or position 314 is not an arginine.

Accordingly, in certain embodiments a modified antibody comprises a constant domain having one or more particular amino acid residues in the Fc region selected from the group consisting of the following residues: the residue at position 308 is threonine or isoleucine; the residue at position 309 is proline; the residue at position 311 is serine, glutamic acid or leucine; the residue at position 312 is aspartic acid; and the residue at position 314 is leucine or alanine. In an embodiment, the modified antibody comprises a constant domain having one or more particular amino acid residues in the Fc region selected from the group consisting of the following residues: threonine at position 308, proline at position 309, serine at position 311, aspartic acid at position 312, and leucine at position 314.

In some embodiments, an antibody of the invention contains a Fc region, or FcRn-binding fragment thereof, having one or more particular amino acid residues among the amino acid residues at positions 385-389 of the Fc region selected from the group consisting of the following residues: residue 385 is arginine, aspartic acid, serine, threonine, histidine, lysine, alanine or glycine; residue 386 is threonine, proline, aspartic acid, serine, lysine, arginine, isoleucine, or methionine; residue 387 is arginine, proline, histidine, serine, threonine, or alanine; and residue 389 is proline, serine or asparagine. In particular embodiments, one or more of the amino acid residue at positions 385, 386, 387, and 389 is arginine, threonine, arginine, and proline, respectively. In another specific embodiment, one or more of the amino acid residues at positions 385, 386, and 389 is aspartic acid, proline, and serine, respectively. In particular embodiments, the amino acid at any one of positions 386, 388, and 389 is not an alanine.

In some embodiments, the amino acid modifications are at one or more of residues 428-436. In specific embodiments, residue 428 is threonine, methionine, leucine, phenylalanine, or serine, residue 433 is arginine, serine, isoleucine, proline, glutamine or histidine, residue 434 is phenylalanine, tyrosine, or histidine, and/or residue 436 is histidine, asparagine, arginine, threonine, lysine, or methionine. In a more specific embodiment, residues at position 428 and/or 434 are substituted with methionine, and/or histidine respectively. In some embodiments, the amino acid residue at position 430 is not alanine; the amino acid residue at position 433 is not alanine or lysine; the amino acid at position 434 is not alanine or glutamine; the amino acid at position 435 is not alanine, arginine, or tyrosine; and/or the amino acid at position 436 is not alanine or tyrosine.

In another embodiment, an antibody of the invention contains a Fc region, or FcRn-binding fragment thereof, having one or more particular amino acid residues in the Fc region selected from the group consisting of a leucine at residue 251, a tyrosine at residue 252, a threonine at residue 254, an arginine at residue 255, a threonine at residue 308, a proline at residue 309, a serine at residue 311, an aspartic acid at residue 312, a leucine at residue 314, an arginine at residue 385, a threonine at residue 386, an arginine at residue 387, a proline at residue 389, a methionine at residue 428, and a tyrosine at residue 434.

In one embodiment, the invention provides modified immunoglobulin molecules that have increased in vivo half-life and affinity for FcRn relative to unmodified molecules (and, in some embodiments, altered bioavailability such as increased or decreased transport to mucosal surfaces or other target tissues). Such immunoglobulin molecules include IgG molecules that naturally contain an FcRn-binding fragment and other non-IgG immunoglobulins (e.g., IgE, IgM, IgD, IgA and IgY) or fragments of immunoglobulins that have been engineered to contain an FcRn-binding fragment (i.e., fusion proteins comprising non-IgG immunoglobulin or a portion thereof and an FcRn-binding fragment). In both cases the FcRn-binding fragment has one or more amino acid modifications that increase the affinity of the constant domain fragment for FcRn, such as those provided above.

The modified immunoglobulins include any immunoglobulin molecule that binds (preferably, immuno specifically, i.e., competes off non-specific binding), as determined by immunoassays well known in the art and described herein for assaying specific antigen-antibody binding an antigen and contains an FcRn-binding fragment. Such antibodies include, but are not limited to, polyclonal, monoclonal, bi-specific, multi-specific, human, humanized, and chimeric antibodies, single chain antibodies, Fab fragments, F(ab′)₂ fragments, disulfide-linked Fvs, and fragments containing either a VL or VH domain or even a CDR that specifically binds an antigen, that, in certain cases, are engineered to contain or to be fused to an FcRn-binding fragment.

The IgG molecules of the invention, and FcRn-binding fragments thereof, are preferably IgG1 subclass of IgGs, but may also be any other IgG subclasses of given animals. For example, in humans, the IgG class includes IgG1, IgG2, IgG3, and IgG4; and mouse IgG includes IgG1, IgG2a, IgG2b, IgG2c and IgG3. It is known that certain IgG subclasses, for example, mouse IgG2b and IgG2c, have higher clearance rates than, for example, IgG1 (Medesan et al., Eur. J. Immunol., 28:2092-2100, 1998). Thus, when using IgG subclasses other than IgG1, it may be advantageous to substitute one or more of the residues, particularly in the CH2 and CH3 domains, that differ from the IgG1 sequence with those of IgG1, thereby increasing the in vivo half-life of the other types of IgG.

The immunoglobulins (and other proteins used herein) may be from any animal origin including birds and mammals. Preferably, the antibodies are human, rodent (e.g., mouse and rat), donkey, sheep, rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example, in U.S. Pat. No. 5,939,598 by Kucherlapati et al.

Modification of any of the antibodies of the invention (e.g., those with increased affinity and/or avidity for a RSV antigen) and/or other therapeutic antibodies to increase the in vivo half-life permits administration of lower effective dosages and/or less frequent dosing of the therapeutic antibody. Such modification to increase in vivo half-life can also be useful to improve diagnostic immunoglobulins as well, for example, permitting administration of lower doses to achieve sufficient diagnostic sensitivity.

In some embodiments, to prolong the in vivo serum circulation of antibodies of the invention, inert polymer molecules such as high molecular weight polyethyleneglycol (PEG) are attached to the antibodies with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein.

In another embodiment, antibodies of the invention are conjugated to albumin in order to make the antibody more stable in vivo or have a longer half-life in vivo. The techniques are well-known in the art, see, e.g., International Publication Nos. WO 93/15199, WO 93/15200, and WO 01/77137; and European Patent No. EP 413,622, all of which are incorporated herein by reference.

One or more modifications in amino acid residues 251-256, 285-290, 308-314, 385-389, and 428-436 of the constant domain may be introduced utilizing any technique known to those of skill in the art. The constant domain or fragment thereof having one or more modifications in amino acid residues 251-256, 285-290, 308-314, 385-389, and 428-436 may be screened by, for example, a binding assay to identify the constant domain or fragment thereof with increased affinity for the FcRn receptor (e.g., as described in Sections 5.5 and 5.6, infra). Those modifications in the hinge-Fc domain or the fragments thereof which increase the affinity of the constant domain or fragment thereof for the FcRn receptor can be introduced into antibodies to increase the in vivo half-lives of said antibodies. Further, those modifications in the constant domain or the fragment thereof which increase the affinity of the constant domain or fragment thereof for the FcRn can be fused to bioactive molecules to increase the in vivo half-lives of said bioactive molecules (and, preferably alter (increase or decrease) the bioavailability of the molecule, for example, to increase or decrease transport to mucosal surfaces (or other target tissue) (e.g., the lungs).

### 5.1.2 Antibody Conjugates and Fusion Proteins

In some embodiments, antibodies of the invention are conjugated or recombinantly fused to a diagnostic, detectable or therapeutic agent or any other molecule. When in vivo half-life is desired to be increased, said antibodies can be modified antibodies. The conjugated or recombinantly fused antibodies can be useful, e.g., for monitoring or prognosing the onset, development, progression and/or severity of a RSV URI and/or LRI or otitis media as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I,), carbon (¹⁴C), sulfur (³⁵S), tritium (³H), indium (¹¹⁵In, ¹¹³In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, 90Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Sn; and positron emitting metals using various positron emission tomographies, and non-radioactive paramagnetic metal ions.

The present invention further encompasses uses of the antibodies of the invention conjugated or recombinantly fused to a therapeutic moiety (or one or more therapeutic moieties). The antibody may be conjugated or recombinantly fused to a therapeutic moiety, such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine); alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP), and cisplatin); anthracyclines (e.g., daunorubicin (formerly daunomycin) and doxorubicin); antibiotics (e.g., d actinomycin (formerly actinomycin), bleomycin, mithramycin, and anthramycin (AMC)); Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference); hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)); cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucorticoids, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN 1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709); adenosine deaminase inhibitors (e.g., Fludarabine phosphate and 2-Chlorodeoxyadenosine); ibritumomab tiuxetan (Zevalin®); tositumomab (Bexxar®)) and pharmaceutically acceptable salts, solvates, clathrates, and prodrugs thereof.

Further, an antibody of the invention may be conjugated or recombinantly fused to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, γ-interferon, α-interferon, nerve growth factor, platelet derived growth factor, tissue plasminogen activator, an apoptotic agent, e.g., TNF-γ, TNF-γ, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), an anti-angiogenic agent, e.g., angiostatin, endostatin or a component of the coagulation pathway (e.g., tissue factor); or, a biological response modifier such as, for example, a lymphokine (e.g., interferon gamma, interleukin-1 (“IL-1”), interleukin-2 (“IL-2”), interleukin-5 (“IL-5”), interleukin-6 (“IL-6”), interleukin-7 (“IL-7”), interleukin 9 (“IL-9”), interleukin-10 (“IL-10”), interleukin-12 (“IL-12”), interleukin-15 (“IL-15”), interleukin-23 (“IL-23”), granulocyte macrophage colony stimulating factor (“GM-CSF”), and granulocyte colony stimulating factor (“G-CSF”)), or a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid, and fibrin monomer).

The present invention encompasses antibodies of the invention (e.g., modified antibodies) recombinantly fused or chemically conjugated (including both covalent and non-covalent conjugations) to a heterologous protein or polypeptide (or fragment thereof, preferably to a polypeptide of about 10, about 20, about 30, about 40, about 50, about 60, about 70, about 80, about 90 or about 100 amino acids) to generate fusion proteins. In particular, the invention provides fusion proteins comprising an antigen-binding fragment of an antibody of the invention (e.g., a Fab fragment, Fd fragment, Fv fragment, F(ab)₂ fragment, a VH domain, a VH CDR, a VL domain or a VL CDR) and a heterologous protein, polypeptide, or peptide. Preferably, the heterologous protein, polypeptide, or peptide that the antibody is fused to is useful for targeting the antibody to a particular cell type. For example, an antibody that immunospecifically binds to a cell surface receptor expressed by a particular cell type (e.g., an immune cell) may be fused or conjugated to a modified antibody of the invention.

In one embodiment, a fusion protein of the invention comprises AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4 antibody and a heterologous polypeptide. In another embodiment, a fusion protein of the invention comprises an antigen-binding fragment of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4 and a heterologous polypeptide. In another embodiment, a fusion protein of the invention comprises one or more VH domains having the amino acid sequence of any one of the VH domains listed in Table 2 or one or more VL domains having the amino acid sequence of any one of the VL domains listed in Table 2 and a heterologous polypeptide. In another embodiment, a fusion protein of the present invention comprises one or more VH CDRs having the amino acid sequence of any one of the VH CDRs listed in Table 2 and/or Tables 3A-3C and a heterologous polypeptide. In another embodiment, a fusion protein comprises one or more VL CDRs having the amino acid sequence of any one of the VL CDRs listed in Table 2 and/or Tables 3D-3F and a heterologous polypeptide. In another embodiment, a fusion protein of the invention comprises at least one VH domain and at least one VL domain listed in Table 2 and a heterologous polypeptide. In yet another embodiment, a fusion protein of the invention comprises at least one VH CDR and at least one VL CDR domain listed in Table 2 and/or Tables 3A-3F and a heterologous polypeptide. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In addition, an antibody of the invention can be conjugated to therapeutic moieties such as a radioactive metal ion, such as alpha-emitters such as ²¹³Bi or macrocyclic chelators useful for conjugating radiometal ions, including but not limited to, ¹³¹In, ¹³¹Lu, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Moreover, antibodies of the invention can be fused to marker sequences, such as a peptide to facilitate purification. In preferred embodiments, the marker amino acid sequence is a hexa-histidine peptide, such as the tag provided in a pQE vector (QIAGEN, Inc.), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

Methods for fusing or conjugating therapeutic moieties (including polypeptides) to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), Thorpe et al., 1982, Immunol. Rev. 62:119-58; —C—U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-10539, 1991; Traunecker et al., Nature, 331:84-86, 1988; Zheng et al., J. Immunol., 154:5590-5600, 1995; Vil et al., Proc. Natl. Acad. Sci. USA, 89:11337-11341, 1992; and U.S. Provisional Application No. 60/727,043 (Attorney Docket No. 10271-165-888) filed Oct. 14, 2005 entitled “Methods of Preventing and Treating RSV Infections and Related Conditions;” and U.S. Provisional No. 60/727,042 (Attorney Docket No. 10271-174-888) filed Oct. 14, 2005 by Genevieve Losonsky entitled “Methods of Administering/Dosing Anti-RSV Antibodies for Prophylaxis and Treatment of RSV Infections and Respiratory Conditions;” which are incorporated herein by reference in their entireties.

In particular, fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of antibodies of the invention (e.g., antibodies with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies, or the encoded antibodies, may be altered by being subjected to random mutagenesis by error-prone PCR, random nucleotide insertion or other methods prior to recombination. A polynucleotide encoding an antibody of the invention may be recombined with one or more components, motifs, sections, parts, domains, fragments, etc. of one or more heterologous molecules.

An antibody of the invention can also be conjugated to a second antibody to form an antibody heteroconjugate as described by Segal in U.S. Pat. No. 4,676,980, which is incorporated herein by reference in its entirety.

The therapeutic moiety or drug conjugated or recombinantly fused to an antibody of the invention that immunospecifically binds to a RSV antigen should be chosen to achieve the desired prophylactic or therapeutic effect(s). In certain embodiments, the antibody is a modified antibody. A clinician or other medical personnel should consider the following when deciding on which therapeutic moiety or drug to conjugate or recombinantly fuse to an antibody of the invention: the nature of the disease, the severity of the disease, and the condition of the subject.

Antibodies of the invention may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### 5.1.3 Intrabody Proteins as Therapeutics

In some embodiments, an antibody of the invention is an intrabody. Methods of producing intrabodies are discussed in Section 5.7, infra. In one embodiment, a recombinantly expressed intrabody protein is administered to a patient. Such an intrabody polypeptide must be intracellular to mediate a prophylactic or therapeutic effect. In this embodiment of the invention, the intrabody polypeptide is associated with a “membrane permeable sequence.” Membrane permeable sequences are polypeptides capable of penetrating through the cell membrane from outside of the cell to the interior of the cell. When linked to another polypeptide, membrane permeable sequences can also direct the translocation of that polypeptide across the cell membrane as well.

In one embodiment, the membrane permeable sequence is the hydrophobic region of a signal peptide (see, e.g., Hawiger, 1999, Curr. Opin. Chem. Biol. 3:89-94; Hawiger, 1997, Curr. Opin. Immunol. 9:189-94; U.S. Pat. Nos. 5,807,746 and 6,043,339, which are incorporated herein by reference in their entireties). The sequence of a membrane permeable sequence can be based on the hydrophobic region of any signal peptide. The signal peptides can be selected, e.g., from the SIGPEP database (see e.g., von Heijne, 1987, Prot. Seq. Data Anal. 1:41-2; von Heijne and Abrahmsen, 1989, FEBS Lett. 224:439-46). When a specific cell type is to be targeted for insertion of an intrabody polypeptide, the membrane permeable sequence is preferably based on a signal peptide endogenous to that cell type. In another embodiment, the membrane permeable sequence is a viral protein (e.g., Herpes Virus Protein VP22) (see e.g., Phelan et al., 1998, Nat. Biotechnol. 16:440-3). A membrane permeable sequence with the appropriate properties for a particular intrabody and/or a particular target cell type can be determined empirically by assessing the ability of each membrane permeable sequence to direct the translocation of the intrabody across the cell membrane. Examples of membrane permeable sequences include, but are not limited to, those sequences disclosed in Table 4.

TABLE 4

| Sequence | SEQ ID NO. |
|---|---|
| Ala Ala Val Ala Leu Lue Pro Ala Val | SEQ ID NO: 37 |
| Leu Leu Ala Leu Leu Ala Pro | |
| Ala Ala Val Leu Leu Pro Val Leu | SEQ ID NO: 38 |
| Leu Ala Ala Pro | |
| Val Thr Val Leu Ala Leu Gly Ala Leu | SEQ ID NO: 39 |
| Ala Gly Val Gly Val Gly | |

In another embodiment, the membrane permeable sequence can be a derivative. In this embodiment, the amino acid sequence of a membrane permeable sequence has been altered by the introduction of amino acid residue substitutions, deletions, additions, and/or modifications. For example, but not by way of limitation, a polypeptide may be modified, e.g., by glycosylation, acetylation, pegylation, phosphorylation, amidation, derivatization by known protecting/blocking groups, proteolytic cleavage, linkage to a cellular ligand or other protein, etc. A derivative of a membrane permeable sequence polypeptide may be modified by chemical modifications using techniques known to those of skill in the art, including, but not limited to specific chemical cleavage, acetylation, formylation, metabolic synthesis of tunicamycin, etc. Further, a derivative of a membrane permeable sequence polypeptide may contain one or more non-classical amino acids. In one embodiment, a polypeptide derivative possesses a similar or identical function as an unaltered polypeptide. In another embodiment, a derivative of a membrane permeable sequence polypeptide has an altered activity when compared to an unaltered polypeptide. For example, a derivative membrane permeable sequence polypeptide can translocate through the cell membrane more efficiently or be more resistant to proteolysis.

The membrane permeable sequence can be attached to the intrabody in a number of ways. In one embodiment, the membrane permeable sequence and the intrabody are expressed as a fusion protein. In this embodiment, the nucleic acid encoding the membrane permeable sequence is attached to the nucleic acid encoding the intrabody using standard recombinant DNA techniques (see e.g., Rojas et al., 1998, Nat. Biotechnol. 16:370-5). In a further embodiment, there is a nucleic acid sequence encoding a spacer peptide placed in between the nucleic acids encoding the membrane permeable sequence and the intrabody. In another embodiment, the membrane permeable sequence polypeptide is attached to the intrabody polypeptide after each is separately expressed recombinantly (see e.g., Zhang et al., 1998, PNAS 95:9184-9). In this embodiment, the polypeptides can be linked by a peptide bond or a non peptide bond (e.g., with a crosslinking reagent such as glutaraldehyde or a thiazolidino linkage see e.g., Hawiger, 1999, Curr. Opin. Chem. Biol. 3:89-94) by methods standard in the art.

The administration of the membrane permeable sequence-intrabody polypeptide can be by parenteral administration, e.g., by intravenous injection including regional perfusion through a blood vessel supplying the tissues(s) or organ(s) having the target cell(s), or by inhalation of an aerosol, subcutaneous or intramuscular injection, intranasal administration, topical administration such as to skin wounds and lesions, direct transfection into, e.g., bone marrow cells prepared for transplantation and subsequent transplantation into the subject, and direct transfection into an organ that is subsequently transplanted into the subject. Further administration methods include oral administration, particularly when the complex is encapsulated, or rectal administration, particularly when the complex is in suppository form. A pharmaceutically acceptable carrier includes any material that is not biologically or otherwise undesirable, i.e., the material may be administered to an individual along with the selected complex without causing any undesirable biological effects or interacting in a deleterious manner with any of the other components of the pharmaceutical composition in which it is contained.

Conditions for the administration of the membrane permeable sequence-intrabody polypeptide can be readily be determined, given the teachings in the art (see e.g., Remington's Pharmaceutical Sciences, 18^{th} Ed., E. W. Martin (ed.), Mack Publishing Co., Easton, Pa. (1990)). If a particular cell type in vivo is to be targeted, for example, by regional perfusion of an organ or tumor, cells from the target tissue can be biopsied and optimal dosages for import of the complex into that tissue can be determined in vitro to optimize the in vivo dosage, including concentration and time length. Alternatively, culture cells of the same cell type can also be used to optimize the dosage for the target cells in vivo.

### 5.2 Prophylactic and Therapeutic Uses of Antibodies

The present invention is directed to antibody-based therapies which involve administering antibodies of the invention to a subject, preferably a human, (e.g., to a subject in need thereof) for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). Prophylactic and therapeutic agents of the invention include, but are not limited to, antibodies of the invention (including analogs and derivatives thereof as described herein) and nucleic acids encoding the antibodies of the invention (including analogs and derivatives thereof and anti-idiotypic antibodies as described herein). Antibodies of the invention may be provided in pharmaceutically acceptable compositions as known in the art or as described herein (see, e.g., Sections 5.1 and 5.3). The antibody used in accordance with the methods of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the IgG constant domain comprises the YTE modification (e.g., MEDI-524 YTE).

Antibodies of the present invention that function as antagonists of a RSV infection can be administered to a subject, preferably a human, to treat, prevent or ameliorate a RSV URI and/or LRI, otitis media (preferably, stemming from, caused by, or associated with a RSV infection), or a symptom or respiratory condition relating thereto (including, but not limited to, asthma, wheezing, RAD, or a combination thereof). For example, antibodies that disrupt or prevent the interaction between a RSV antigen and its host cell receptor may be administered to subject, preferably a human, to prevent, manage, treat and/or ameliorate a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD).

In a specific embodiment, an antibody of the invention prevents or inhibits RSV from binding to its host cell receptor by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV binding to its host cell receptor in the absence of said antibody or in the presence of a negative control in an assay known to one of skill in the art or described herein, such as by a competition assay (see, e.g., Example 6.8) or microneutralization assay (see, e.g., Example 6.6). In another embodiment, a combination of antibodies of the invention prevents or inhibits RSV from binding to its host cell receptor by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV binding to its host cell receptor in the absence of said antibodies or in the presence of a negative control in an assay known to one of skill in the art or described herein. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE). In certain embodiments, one or more modified and/or unmodified antibodies of the invention can be administered either alone or in combination. In some embodiments, a combination of antibodies of the invention act synergistically to prevent or inhibit RSV from binding to its host and receptor and/or in preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD).

In a specific embodiment, an antibody of the invention (modified or unmodified) prevents or inhibits RSV-induced fusion by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV-induced fusion in the absence of said antibody or in the presence of a negative control in an assay known to one of skill in the art or described herein (see, e.g., Example 6.6). In another embodiment, a combination of antibodies of the invention prevents or inhibits RSV-induced fusion by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV-induced fusion in the absence of said antibodies or in the presence of a negative control in an assay known to one of skill in the art or described herein. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE). Thus, in some embodiments, the antibody is a modified antibody, and in other embodiments, the antibody is not a modified antibody.

In a specific embodiment, an antibody of the invention prevents or inhibits RSV-induced fusion after viral attachment to cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV-induced fusion after viral attachment to cells in the absence of said antibody or in the presence of a negative control in an assay known to one of skill in the art or described herein (see, e.g., Example 6.6). In another embodiment, a combination of antibodies of the invention prevents or inhibits RSV-induced fusion after viral attachment to cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV-induced fusion after viral attachment to cells in the absence of said antibodies or in the presence of a negative control in an assay known to one of skill in the art or described herein. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE). Thus, in some embodiments, the antibody is a modified antibody, and in other embodiments, the antibody is not a modified antibody.

Antibodies of the invention that do not prevent RSV from binding its host cell receptor but inhibit or downregulate RSV replication or inhibit RSV fusion to a cell can also be administered to a subject to treat, prevent or ameliorate a RSV URI and/or LRI, otitis media (stemming from, caused by, or associated with a RSV infection), or a symptom or respiratory condition relating thereto (including, but not limited to, asthma, wheezing, RAD, or a combination thereof). The ability of an antibody of the invention to inhibit or downregulate RSV replication may be determined by techniques described herein or otherwise known in the art (see, e.g., Example 6.4). For example, the inhibition or downregulation of RSV replication can be determined by detecting the RSV titer in the lungs of a subject, preferably a human. In further embodiments, the inhibition or downregulation of RSV replication can be determined by detecting the amount of RSV in the nasal passages or in the middle ear by methods known in the art (e.g., Northern blot analysis, RT-PCR, Western Blot analysis, etc.). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE). Thus, in some embodiments, the antibody is a modified antibody, and in other embodiments, the antibody is not a modified antibody.

In some embodiments, an antibody of the invention results in reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105 fold, about 110-fold, about 115-fold, about 120 fold, about 125-fold or higher in RSV titer in the lung. The fold-reduction in RSV titer may be as compared to a negative control (such as placebo), as compared to another treatment (including, but not limited to treatment with palivizumab), or as compared to the titer in the patient prior to antibody administration. In certain embodiments, the above-referenced antibody comprises a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE). Thus, in some embodiments, the antibody is a modified antibody, and in other embodiments, the antibody is not a modified antibody. In embodiments, wherein the antibody is a modified antibody of the invention, the reduction may further be compared to a subject receiving the same antibody without the modifications in the IgG constant domain.

In a specific embodiment, an antibody of the present invention inhibits or downregulates RSV replication by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV replication in absence of said antibody or in the presence of a negative control in an assay known in the art or described herein (see, e.g., Example 6.4). In another embodiment, a combination of antibodies of the invention inhibits or downregulates RSV replication by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV replication in absence of said antibodies or in the presence of a negative control in an assay known in the art or described herein. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE). Thus, in some embodiments, the antibody is a modified antibody, and in other embodiments, the antibody is not a modified antibody.

In some embodiments, an antibody of the invention results in reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105 fold, about 110-fold, about 115-fold, about 120 fold, about 125-fold or higher in RSV titer in the upper respiratory tract. The fold-reduction in RSV titer may be as compared to a negative control (such as placebo), as compared to another treatment (including, but not limited to treatment with palivizumab), or as compared to the titer in the patient prior to antibody administration. In certain embodiments, the above-referenced antibody comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE). Thus, in some embodiments, the antibody is a modified antibody, and in other embodiments, the antibody is not a modified antibody. In embodiments, wherein the antibody is a modified antibody of the invention, the reduction may further be compared to a subject receiving the same antibody without the modifications in the IgG constant domain.

In other embodiments, an antibody of the invention results in reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105 fold, about 110-fold, about 115-fold, about 120 fold, about 125-fold or higher in RSV titer in the lower respiratory tract. The fold-reduction in RSV titer may be as compared to a negative control (such as placebo), as compared to another treatment (including, but not limited to treatment with palivizumab), or as compared to the titer in the patient prior to antibody administration. In certain embodiments, the above-referenced antibody comprises a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE). Thus, in some embodiments, the antibody is a modified antibody, and in other embodiments, the antibody is not a modified antibody. In embodiments, wherein the antibody is a modified antibody of the invention, the reduction may further be compared to a subject receiving the same antibody without the modifications in the IgG constant domain.

One or more antibodies of the present invention (e.g., a MEDI-524 antibody or a modified MEDI-524 antibody, such as MEDI-524-YTE) have reduced or no cross-reactivity with human, rat (e.g., cotton rat), and/or monkey (e.g., cynomolgus monkey, or chimpanzee) tissue samples as compared to another anti-RSV antibody, as determined by techniques described herein or otherwise known in the art (see, e.g., Example 6.19). In some embodiments, an antibody of the invention has reduced or no cross-reactivity as compared to A4b4 (see, e.g., Example 6.19). In some embodiments, the antibody of the invention has reduced or no cross reactivity as that seen with a negative control antibody (e.g., an anti-human IgG antibody, such as a human monoclonal IgG1 kappa antibody, with different antigen specificity than the antibody of the invention). In certain embodiments, the tissue sample is skin or lung. In other embodiments, the tissue sample is adrenal gland, blood leukocytes, blood vessel (e.g., endothelium), bone marrow, brain (e.g., cerebrum or cerebellum), breast (mammary gland), eye, colon, large intestine, small intestine, esophagus, stomach, heart, kidney (e.g., glomerulus or tubule), liver, lung, lymph node, ovary, fallopian tube (e.g., oviduct), pancreas, parathyroid, peripheral nerve, pituitary, placenta, prostate, salivary gland, skin, spinal cord, spleen, striated (e.g., skeletal) muscle, testis, thymus, thyroid, tonsil, ureter, urinary bladder, and/or uterus (e.g., endometrium or cervix) tissue. In certain embodiments, the antibody (e.g., a MEDI-524 antibody or a modified MEDI-524 antibody, such as MEDI-524-YTE) has a reduction in cross-reactivity with a human tissue sample (e.g., skin or lung) of about 100-fold, 90-fold, 80-fold, 70-fold, 60-fold, 50-fold, 40-fold, 30-fold, 20-fold, 10-fold, 5-fold, or 2-fold as compared to another anti-RSV antibody (e.g., A4b4). In preferred embodiments, the tissue is skin or lung and the antibody (e.g., a MEDI-524 or a modified MEDI-524 antibody, such as MEDI-524-YTE) has reduced or no cross-reactivity with the tissue as compared to A4b4, as determined by techniques described herein or otherwise known in the art (see, e.g., Example 6.19).

One or more antibodies of the present invention that immunospecifically bind to one or more RSV antigens may be used locally or systemically in the body as a prophylactic or therapeutic agent. The antibodies of the invention may also be advantageously utilized in combination with other antibodies (e.g., monoclonal or chimeric antibodies), or with lymphokines or hematopoietic growth factors (such as, e.g., IL-2, IL-3 and IL-7), which, for example, serve to increase the number or activity of effector cells which interact with the antibodies. The antibodies of this invention may also be advantageously utilized in combination with other antibodies (e.g., monoclonal or chimeric antibodies), or with lymphokines or hematopoietic growth factors (such as, e.g., IL-2, IL-3 and IL-7), which, for example, serve to increase the immune response. The antibodies of this invention may also be advantageously utilized in combination with one or more drugs used to treat RSV infection such as, for example anti-viral agents. Antibodies of the invention may be used in combination with one or more of the following drugs: ribavirin (Valent Pharmaceuticals International), NIH-351 (Gemini Technologies), recombinant RSV vaccine (MedImmune Vaccines), RSVf-2 (Intracel), F-50042 (Pierre Fabre), T-786 (Trimeris), VP-36676 (ViroPharma), RFI-641 (American Home Products), VP-14637 (ViroPharma), PFP-1 and PFP-2 (American Home Products), RSV vaccine (Avant Immunotherapeutics), F-50077 (Pierre Fabre), and any one of the anti-viral polycyclic compounds taught in WO 05/061513 (Biota Scientific Management Pty Ltd.). In a specific embodiment, an effective amount of an antibody of the invention and an effective amount of another therapy is used.

The antibodies of the invention may be administered alone or in combination with other types of therapies (e.g., hormonal therapy, immunotherapy, and anti-inflammatory agents). In some embodiments, the antibodies of the invention act synergistically with the other therapies. Generally, administration of products of a species origin or species reactivity (in the case of antibodies) that is the same species as that of the patient is preferred. Thus, in a preferred embodiment, human or humanized antibodies, derivatives, analogs, or nucleic acids, are administered to a human patient for therapy or prophylaxis.

In specific embodiments, an antibody of the invention is administered in combination with one or more anti-IL-9 antibodies (such as those disclosed in U.S. Publication No. 2005/0002934) either alone or in combination with one or more modified antibodies of the invention and/or other types of therapies or other agents (e.g., hormone therapy, immunotherapy, and anti-inflammatory agents, such as those disclosed in U.S. Publication No. 2005/0002934, which is herein incorporated by reference in its entirety).

It is preferred to use high affinity and/or potent in vivo inhibiting antibodies and/or neutralizing antibodies that immunospecifically bind to a RSV antigen, for both immunoassays directed to RSV, and the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). It is also preferred to use polynucleotides encoding high affinity and/or potent in vivo inhibiting antibodies and/or neutralizing antibodies that immunospecifically bind to a RSV antigen, for both immunoassays directed to RSV and therapy for a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). Such antibodies will preferably have an affinity for the RSV F glycoprotein and/or fragments of the F glycoprotein.

The methods of the invention comprise the administration of one or more antibodies of the invention to patients suffering from or expected to suffer from (e.g., patients with a genetic predisposition for or patients that have previously suffered from) a RSV infection (e.g., acute RSV disease or RSV URI and/or LRI), otitis media (preferably, stemming from, caused by, or associated with a RSV infection), or a symptom or respiratory condition relating thereto (including, but not limited to, asthma, wheezing, RAD, or a combination thereof). Such patients may have been previously treated or are currently being treated for the RSV infection, otitis media, or a symptom or respiratory condition related thereto, e.g., with a therapy other than a modified antibody of the invention. In one embodiment, the methods of the invention comprise the administration of one or more antibodies of the invention to patients that are immunocompromised or immunosuppressed. In another embodiment, an antibody of the invention is administered to a human with cystic fibrosis, bronchopulmonary dysplasia, congenital heart disease, congenital immunodeficiency or acquired immunodeficiency, or to a human who has had a bone marrow transplant to prevent, manage, treat and/or ameliorate a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). In another embodiment, an antibody of the invention is administered to a human infant, preferably a human infant born prematurely or a human infant at risk of hospitalization for RSV infection, to prevent, manage, treat and/or ameliorate a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). In yet another embodiment, an antibody of the invention is administered to the elderly or people in group homes (e.g., nursing homes or rehabilitation centers). In accordance with the invention, an antibody of the invention may be used as any line of therapy, including, but not limited to, a first, second, third, fourth and/or fifth line of therapy. Further, in accordance with the invention, an antibody of the invention can be used before or after any adverse effects or intolerance of the therapies other than an antibody of the invention occurs. The invention encompasses methods for administering one or more antibodies of the invention to prevent the onset of an acute RSV disease and/or to treat or lessen the recurrence of a RSV URI and/or LRI or otitis media.

In one embodiment, the invention also provides methods of prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) as alternatives to current therapies. In a specific embodiment, the current therapy has proven or may prove to be too toxic (i.e., results in unacceptable or unbearable side effects) for the patient. In another embodiment, an antibody of the invention decreases the side effects as compared to the current therapy. In another embodiment, the patient has proven refractory to a current therapy. In such embodiments, the invention provides for the administration of one or more antibodies of the invention without any other anti-infection therapies. In certain embodiments, a patient with a RSV infection (e.g., acute RSV disease or RSV URI and/or LRI), is refractory to a therapy when the infection has not significantly been eradicated and/or the symptoms have not been significantly alleviated. The determination of whether a patient is refractory can be made either in vivo or in vitro by any method known in the art for assaying the effectiveness of a therapy for infections, using art-accepted meanings of “refractory” in such a context. In various embodiments, a patient with a RSV infection (e.g., acute RSV disease or RSV URI and/or LRI) is refractory when viral replication has not decreased or has increased following therapy.

In certain embodiments, one or more antibodies of the invention can be administered to a patient instead of another therapy to treat a RSV infection (e.g., acute RSV disease or RSV URI and/or LRI), otitis media or a symptom or respiratory condition related thereto (including, but not limited to, asthma, wheezing, RAD, or a combination thereof). In one embodiment, the invention provides methods of preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). The invention also encompasses methods of preventing the onset or reoccurrence of a RSV infection (e.g., acute RSV disease, or a RSV URI and/or LRI) or otitis media (preferably, stemming from, caused by or associated with a RSV infection, such as a RSV URI and/or LRI) in patients at risk of developing such infections or otitis media.

In certain embodiments, an effective amount of one or more modified antibodies of the invention is administered in combination with one or more supportive measures to a subject to prevent, manage, treat and/or ameliorate a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). Non-limiting examples of supportive measures include humidification of the air by an ultrasonic nebulizer, aerolized racemic epinephrine, oral dexamethasone, intravenous fluids, intubation, fever reducers (e.g., ibuprofen, acetometaphin), and antibiotic and/or anti-fungal therapy (i.e., to prevent or treat secondary bacterial and/or fungal infections).

In a specific embodiment, the invention provides methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD), said methods comprising administering to a subject an effective amount of one or more antibodies of the invention alone or in combination with one or more anti-viral agents such as, but not limited to, amantadine, rimantadine, oseltamivir, znamivir, ribavarin, RSV-IVIG (i.e., intravenous immune globulin infusion) (RESPIGAM™), EphA2/EphrinA1 Modulators, and/or an anti-IL-9 antibody (see, e.g., U.S. Publication No. 2005/0002934), and/or any one of the anti-viral polycyclic compounds disclosed in WO 05/061513.

In a specific embodiment, the invention provides methods for preventing, managing, treating, and/or ameliorating one or more secondary responses to a primary viral infection, said methods comprising administering an effective amount of one or more antibodies of the invention alone or in combination with an effective amount of other therapies (e.g., other prophylactic or therapeutic agents). Examples of secondary responses to a primary viral infection include, but are not limited to, asthma-like responsiveness to mucosal stimula, elevated total respiratory resistance, increased susceptibility to secondary viral, bacterial, and fungal infections, and development of conditions such as, but not limited to, bronchiolitis, pneumonia, croup, and febrile bronchitis.

In a specific embodiment, the invention provides methods of preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD), said methods comprising administering to a subject an effective amount of one or more antibodies of the invention in combination with an effective amount of an EphA2/EphrinA1 Modulator (U.S. Provisional Appn. Ser. No. 60/622,489, filed Oct. 27, 2004, entitled “Use of Modulators of EphA2 and EphrinA1 for the Treatment and Prevention of Infections,” which is incorporated by reference herein in its entirety). In another specific embodiment, the invention provides methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD), said methods comprising administering to a subject an effective amount of one or more antibodies of the invention in combination with an effective amount of siplizumab (MedImmune, Inc., International Pub. No. WO 02/069904, which is incorporated herein by reference in its entirety). In another embodiment, the invention provides methods of preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD), said methods comprising administering to a subject an effective amount of one or more antibodies in combination with an effective amount of one or more anti-IL-9 antibodies, such as those disclosed in U.S. Publication No. 2005/0002934, which is incorporated herein by reference in its entirety. In yet another embodiment, the invention provides methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD), said methods comprising administering to a subject an effective amount of one or more antibodies of the invention in combination with an effective amount of two or more of the following: EphA2/EphrinA1 modulators, an anti-IL-9 antibody and/or siplizumab.

The invention also encompasses methods of preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) in patients who are susceptible to adverse reactions to conventional therapies. The invention further encompasses methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) for which no other anti-viral therapy is available.

The invention encompasses methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) in a patient who has proven refractory to therapies other than modified antibodies of the invention but are no longer on these therapies. In certain embodiments, the patients being treated in accordance with the methods of this invention are patients already being treated with antibiotics, anti-virals, anti-fungals, or other biological therapy/immunotherapy. Among these patients are refractory patients, patients who are too young for conventional therapies, and patients with reoccurring RSV URI and/or LRI or otitis media (preferably, stemming from, caused by or associated with a RSV infection, such as a RSV URI and/or LRI) or a symptom or respiratory condition relating thereto (including, but not limited to, asthma, wheezing, RAD, or a combination thereof) despite treatment with existing therapies.

The present invention encompasses methods for preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) as an alternative to other conventional therapies. In specific embodiments, the patient being or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, treat, and/or ameliorate a RSV URI and/or LRI, otitis media (preferably, stemming from, caused by or associated with a RSV infection, such as a RSV URI and/or LRI) or a symptom or respiratory condition relating thereto (including, but not limited to, asthma, wheezing, RAD, or a combination thereof).

The dosage amounts and frequencies of administration provided herein are encompassed by the terms “effective amount”, “therapeutically effective” and “prophylactically effective.” The dosage and frequency further will typically vary according to factors specific for each patient depending on the specific therapeutic or prophylactic agents administered, the severity and type of infection, the route of administration, as well as age, body weight, response, and the past medical history of the patient. Suitable regimens can be selected by one skilled in the art by considering such factors and by following, for example, dosages reported in the literature and recommended in the Physician's Desk Reference (58^{th} ed., 2004). See Section 5.3 for exemplary dosage amounts and frequencies of administration of the prophylactic and therapeutic agents provided by the invention.

In specific embodiments, antibodies of the invention are administered to an animal are of a species origin or species reactivity that is the same species as that of the animal. Thus, in a preferred embodiment, human or humanized antibodies, or nucleic acids encoding human or human, are administered to a human patient for therapy or prophylaxis.

In preferred embodiments, a modified antibody of the invention having an extended in vivo half-life can be used in passive immunotherapy (for either therapy or prophylaxis). Because of the extended half-life, passive immunotherapy or prophylaxis can be accomplished using lower doses and/or less frequent administration of the antibody resulting in fewer side effects, better patient compliance, less costly therapy/prophylaxis, etc. In a preferred embodiment, the therapeutic/prophylactic is an antibody that binds RSV, for example, any one or more of the anti-RSV antibodies described in Section 5.1, supra, (or elsewhere herein), wherein said antibody is a modified antibody. In certain embodiments, unmodified antibodies of the invention can be used in passive immunotherapy, either alone or in combination with a modified antibody of the invention.

### 5.3 Methods of Administration, Frequency, and Dosing of Antibodies

The present invention further provides for compositions comprising one or more antibodies of the invention (including modified antibodies) for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). In a specific embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises an AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, and/or A17h4 antibody. In another specific embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises an antigen-binding fragment of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), or A4B4-F52S, A17d4(1), A3e2, A14a4, A16b4, A17b5, A17f5, or A17h4. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH domains having an amino acid sequence of any one of the VH domains listed in Table 2. In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH CDR1s having an amino acid sequence of any one of the VH CDR1s listed in Table 2 and/or Table 3A. In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH CDR2s having an amino acid sequence of any one of the VH CDR2s listed in Table 2 and/or Table 3B. In a preferred embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH CDR3s having an amino acid sequence of any one of the VH CDR3s listed in Table 2 and/or Table 3C. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VL domains having an amino acid sequence of any one of the VL domains listed in Table 2. In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VL CDR1s having an amino acid sequence of any one of the VL CDR1s listed in Table 2 or Table 3D. In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VL CDR2s having an amino acid sequence of any one of the VL CDR2s listed in Table 2 and/or Table 3E. In a preferred embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VL CDR3s having an amino acid sequence of any one of the VL CDR3s listed in Table 2 and/or Table 3F. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH domains having an amino acid sequence of any one of the VH domains listed in Table 2 and one or more VL domains having an amino acid sequence of any one of the VL domains listed in Table 2. In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH CDR1s having an amino acid sequence of any one of the VH CDR1s listed in Table 2 and/or Table 3A and one or more VL CDR1s having an amino acid sequence of any one of the VL CDR1s listed in Table 2 and/or Table 3D. In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH CDR1s having an amino acid sequence of any one of the VH CDR1s listed in Table 2 and/or Table 3A and one or more VL CDR2s having an amino acid sequence of any one of the VL CDR2s listed in Table 2 and/or Table 3E. In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH CDR1s having an amino acid sequence of any one of the VH CDR1s listed in Table 2 and/or Table 3A and one or more VL CDR3s having an amino acid sequence of any one of the VL CDR3s listed in Table 2 and/or Table 3F. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH CDR2s having an amino acid sequence of any one of the VH CDR2s listed in Table 2 and/or Table 3B and one or more VL CDR1s having an amino acid sequence of any one of the VL CDR1s listed in Table 2 and/or Table 3D. In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH CDR2s having an amino acid sequence of any one of the VH CDR2s listed in Table 2 and/or Table 3B and one or more VL CDR2s having an amino acid sequence of any one of the VL CDR2s listed in Table 2 and/or Table 3E. In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH CDR2s having an amino acid sequence of any one of the VH CDR2s listed in Table 2 and/or Table 3B and one or more VL CDR3s having an amino acid sequence of any one of the VL CDR3s listed in Table 2 and/or Table 3F. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH CDR3s having an amino acid sequence of any one of the VH CDR3s listed in Table 2 and/or Table 3C and one or more VL CDR1s having an amino acid sequence of any one of the VL CDR1s listed in Table 2 and/or Table 3D. In another embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH CDR3s having an amino acid sequence of any one of the VH CDR3s listed in Table 2 and/or Table 3C and one or more VL CDR2s having an amino acid sequence of any one of the VL CDR2s listed in Table 2 and/or Table 3E. In a preferred embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises one or more antibodies comprising one or more VH CDR3s having an amino acid sequence of any one of the VH CDR3s listed in Table 2 and/or Table 3C and one or more VL CDR3s having an amino acid sequence of any one of the VL CDR3s listed in Table 2 and/or Table 3F. In a preferred embodiment, a composition for use in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) comprises A4B4L1FR-S28R (MEDI-524) or an antigen-binding fragment thereof. In yet another embodiment, a composition of the present invention comprises one or more fusion proteins of the invention. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

As discussed in more detail below, a composition of the invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionucleotides, or toxins. See, e.g., PCT publications WO 92/08495; WO 91/14438; WO 89/12624; U.S. Pat. No. 5,314,995; and EP 396,387.

Antibodies of the present invention may be used, for example, to purify, detect, and target RSV antigens, in both in vitro and in vivo diagnostic and therapeutic methods. For example, the modified antibodies have use in immunoassays for qualitatively and quantitatively measuring levels of the RSV in biological samples such as sputum. See, e.g., Harlow et al., Antibodies: A Laboratory Manual, (Cold Spring Harbor Laboratory Press, 2nd ed. 1988) (incorporated by reference herein in its entirety).

The invention also provides methods of preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) by administrating to a subject of an effective amount of an antibody, or pharmaceutical composition comprising an antibody of the invention. In a preferred aspect, an antibody is substantially purified (i.e., substantially free from substances that limit its effect or produce undesired side-effects). The subject administered a therapy is preferably a mammal such as non-primate (e.g., cows, pigs, horses, cats, dogs, rats etc.) or a primate (e.g., a monkey, such as a cynomolgous monkey, or a human). In a preferred embodiment, the subject is a human. In another preferred embodiment, the subject is a human infant or a human infant born prematurely. In another embodiment, the subject is a human with a RSV URI and/or LRI, otitis media stemming from, caused by or associated with a RSV infection, cystic fibrosis, bronchopulmonary dysplasia, congenital heart disease, congenital immunodeficiency or acquired immunodeficiency, a human who has had a bone marrow transplant, or an elderly human.

Various delivery systems are known and can be used to administer a prophylactic or therapeutic agent (e.g., a modified antibody of the invention), including, but not limited to, encapsulation in liposomes, microparticles, microcapsules, recombinant cells capable of expressing the antibody, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent (e.g., an antibody of the invention), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antibody of the present invention), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. In a specific embodiment, an antibody of the invention, or composition of the invention is administered using Alkermes AIR™ pulmonary drug delivery technology (Alkermes, Inc., Cambridge, Mass.).

In a specific embodiment, it may be desirable to administer a prophylactic or therapeutic agent, or a pharmaceutical composition of the invention locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. Preferably, when administering an antibody of the invention, care must be taken to use materials to which the antibody does not absorb.

In another embodiment, a prophylactic or therapeutic agent, or a composition of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, a prophylactic or therapeutic agent, or a composition of the invention can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of a prophylactic or therapeutic agent (e.g., an antibodies of the invention) or a composition of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the nasal passages or lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the invention. See, e.g., U.S. Pat. No. 4,526,938, PCT publication WO 91/05548, PCT publication WO 96/20698, Ning et al., 1996, “Intratumoral Radioimmunotherapy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment, where the composition of the invention is a nucleic acid encoding a prophylactic or therapeutic agent (e.g., an antibody of the invention), the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

In a specific embodiment, a composition of the invention comprises one, two or more antibodies of the invention. In another embodiment, a composition of the invention comprises one, two or more antibodies of the invention and a prophylactic or therapeutic agent other than an antibody of the invention. Preferably, the agents are known to be useful for or have been or are currently used for the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). In addition to prophylactic or therapeutic agents, the compositions of the invention may also comprise a carrier.

The compositions of the invention include bulk drug compositions useful in the manufacture of pharmaceutical compositions (e.g., compositions that are suitable for administration to a subject or patient) that can be used in the preparation of unit dosage forms. In a preferred embodiment, a composition of the invention is a pharmaceutical composition. Such compositions comprise a prophylactically or therapeutically effective amount of one or more prophylactic or therapeutic agents (e.g., a modified antibody of the invention or other prophylactic or therapeutic agent), and a pharmaceutically acceptable carrier. Preferably, the pharmaceutical compositions are formulated to be suitable for the route of administration to a subject.

In a specific embodiment, the term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of the antibody, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Such compositions, however, may be administered by a route other than intravenous.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides that an antibody of the invention is packaged in a hermetically sealed container such as an ampoule or sachette indicating the quantity of antibody. In one embodiment, the antibody is supplied as a dry sterilized lyophilized powder or water free concentrate in a hermetically sealed container and can be reconstituted, e.g., with water or saline to the appropriate concentration for administration to a subject. Preferably, the antibody is supplied as a dry sterile lyophilized powder in a hermetically sealed container at a unit dosage of at least 0.5 mg, at least 1 mg, at least 2 mg, or at least 3 mg, and more preferably at least 5 mg, at least 10 mg, at least 15 mg, at least 25 mg, at least 30 mg, at least 35 mg, at least 45 mg, at least 50 mg, at least 60 mg, or at least 75 mg. The lyophilized antibody can be stored at between 2 and 8° C. in its original container and the antibody can be administered within 12 hours, preferably within 6 hours, within 5 hours, within 3 hours, or within 1 hour after being reconstituted. In an alternative embodiment, a modified antibody is supplied in liquid form in a hermetically sealed container indicating the quantity and concentration of the antibody. Preferably, the liquid form of the antibody is supplied in a hermetically sealed container at least 0.1 mg/ml, at least 0.5 mg/ml, or at least 1 mg/ml, and more preferably at least 2.5 mg/ml, at least 3 mg/ml, at least 5 mg/ml, at least 8 mg/ml, at least 10 mg/ml, at least 15 mg/ml, at least 25 mg/ml, at least 30 mg/ml, or at least 60 mg/ml.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a prophylactic or therapeutic agent (e.g., an antibody of the invention), or a composition of the invention that will be effective in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) can be determined by standard clinical techniques. For example, the dosage of a prophylactic or therapeutic agent, or a composition comprising an antibody of the invention that will be effective in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) can be determined by administering the composition to a cotton rat, measuring the RSV titer after challenging the cotton rat with 10⁵ pfu of RSV and comparing the RSV titer to that obtain for a cotton rat not administered the prophylactic or therapeutic agent, or the composition. Accordingly, a dosage that results in a 2 log decrease or a 99% reduction in RSV titer in the cotton rat challenged with 10⁵ pfu of RSV relative to the cotton rat challenged with 10⁵ pfu of RSV but not administered the prophylactic or therapeutic agent, or the composition is the dosage of the composition that can be administered to a human for the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD).

The dosage of a composition which will be effective in the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) can be determined by administering the composition to an animal model (e.g., a cotton rat or monkey) and measuring the serum titer, lung concentration or nasal turbinate and/or nasal secretion concentration of a modified antibody that immunospecifically bind to a RSV antigen. Accordingly, a dosage of an antibody or a composition that results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, and preferably at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). In addition, in vitro assays may optionally be employed to help identify optimal dosage ranges. In some embodiments, the antibody is a modified antibody (e.g., MEDI-524-YTE).

The precise dose to be employed in the formulation will also depend on the route of administration, and the seriousness of the RSV URI and/or LRI or otitis media, and should be decided according to the judgment of the practitioner and each patient's circumstances. Effective doses may be extrapolated from dose-response curves derived from in vitro or animal model (e.g., the cotton rat or Cynomolgous monkey) test systems.

For the antibodies of the invention, the dosage administered to a patient is typically 0.0.25 mg/kg to 100 mg/kg of the patient's body weight. In some embodiments, the dosage administered to the patient is about 3 mg/kg to about 60 mg/kg of the patient's body weight. Preferably, the dosage administered to a patient is between 0.025 mg/kg and 20 mg/kg of the patient's body weight, more preferably 1 mg/kg to 15 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of the antibodies of the invention may be reduced by enhancing uptake and tissue penetration (e.g., into the nasal passages and/or lung) of the antibodies by modifications such as, for example, lipidation. In a preferred embodiment, the dosage of A4B4L1FR-S28R (MEDI-524) or antigen-binding fragment thereof (including a modified A4B4L1FR-S28R antibody, such as MEDI-524-YTE) to be administered to is about 60 mg/kg, about 50 mg/kg, about 40 mg/kg, about 30 mg/kg, about 15 mg/kg, about 10 mg/kg, about 5 mg/kg, about 3 mg/kg, about 2 mg/kg, about 1 mg/kg, about 0.80 mg/kg, about 0.50 mg/kg, about 0.40 mg/kg, about 0.20 mg/kg, about 0.10 mg/kg, about 0.05 mg/kg, or about 0.025 mg/kg of the patient's body weight.

In a specific embodiment, antibodies of the invention, or compositions comprising antibodies of the invention are administered once a month just prior to (e.g., within three months, within two months, within one month) or during the RSV season. In another embodiment, antibodies of the invention, or compositions comprising modified antibodies of the invention are administered every two months just prior to or during the RSV season. In another embodiment, antibodies of the invention, or compositions comprising antibodies of the invention are administered every three months just prior to or during the RSV season. In a preferred embodiment, antibodies of the invention, or compositions comprising antibodies of the invention are administered once just prior to or during the RSV season. In preferred embodiment, antibodies of the invention are administered twice, and most preferably once, during a RSV season. In some embodiments, antibodies of the invention are administered just prior to the RSV season and can optionally administered once during the RSV season. In some embodiments, antibodies of the invention, or compositions comprising antibodies of the invention, are administered every 24 hours for at least three days, at least four days, at least five days, at least six days up to one week just prior to or during an RSV season. In specific embodiments, the daily administration of antibodies of the invention, or compositions comprising antibodies of the invention, occur soon after RSV infection is first recognized (i.e., when the patient has nasal congestion and/or other upper respiratory symptoms), but prior to presentation of clinically significant disease in the lungs (i.e., prior to lower respiratory disease manifestation) such that lower respiratory disease is prevented. In another embodiment, modified antibodies of the invention, or compositions comprising modified antibodies of the invention are administered intranasally once a day for about three (3) days while the patient presents with symptoms of RSV URI during the RSV season. Alternatively, in another embodiment, modified antibodies of the invention, or compositions comprising modified antibodies of the invention are administered intranasally once every other day for at least one week while the patient presents with symptoms of RSV URI during the RSV season. The term “RSV season” refers to the season when RSV infection is most likely to occur. Typically, the RSV season in the northern hemisphere commences in November and lasts through April. Preferably, the antibody comprises the VH and VL domain of A4B4L1FR-S28R (MEDI-524) (FIG. 13) or an antigen-binding fragment thereof. In preferred embodiments, the above referenced antibody is A4B4L1FR-S28R (MEDI-524). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In one embodiment, approximately 60 mg/kg or less, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, or approximately 1.5 mg/kg or less of an antibody the invention is administered 5 times, 4 times, 3 times, 2 times or, preferably, 1 time during a RSV season to a subject, preferably a human. In some embodiments, an antibody of the invention is administered about 1-12 times during the RSV season to a subject, wherein the doses may be administered as necessary, e.g., weekly, biweekly, monthly, bimonthly, trimonthly, etc., as determined by a physician. In some embodiments, a lower dose (e.g., 5-15 mg/kg) can be administered more frequently (e.g., 3-6 times) during a RSV season. In other embodiments, a higher dose (e.g., 30-60 mg/kg) can be administered less frequently (e.g., 1-3 times) during a RSV season. However, as will be apparent to those in the art, other dosing amounts and schedules are easily determinable and within the scope of the invention. In preferred embodiments, an antibody of the invention comprises one or more VH domains or chains and/or one or more VL domains or chains ion Table 2, and comprises a modified constant domain described, such as modifications at those residues in the IgG constant domain identified herein (see Section 5.1.1). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In one embodiment, approximately 60 mg/kg or less, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of an antibody the invention is administered to a patient five times during a RSV season to a subject, preferably a human, intramuscularly or intranasally. In another embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of an antibody the invention is administered to a patient three times during a RSV season to a subject, preferably a human, intramuscularly or intranasally. In yet another embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of an antibody the invention is administered two times and most preferably one time during a RSV season to a subject, preferably a human, intramuscularly or intranasally. In another embodiment, approximately 1 mg/kg or less, approximately 0.1 mg/kg or less, approximately 0.05 mg/kg or less or approximately 0.025 mg/kg of a modified antibody of the invention is administered once a day for at least three days or alternatively, every other day for at least one week during a RSV season to a subject, preferably human, intranasally. Preferably, the modified antibody comprises the VH and VL domain of A4B4L1 FR-S28R (MEDI-524) (FIG. 13) or an antigen-binding fragment thereof. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In a specific embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of an antibody the invention in a sustained release formulation is administered to a subject, preferably a human, to prevent, manage, treat and/or ameliorate a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). In another specific embodiment, an approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less bolus of an antibody the invention not in a sustained release formulation is administered to a subject, preferably a human, to prevent, manage, treat and/or ameliorate a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD), and after a certain period of time, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of the invention in a sustained release is administered to said subject (e.g., intranasally or intramuscularly) two, three or four times (preferably one time) during a RSV season. In accordance with this embodiment, a certain period of time can be 1 to 5 days, a week, two weeks, or a month. In another embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of a modified antibody of the invention in a sustained release formulation is administered to a subject, preferably a human, intramuscularly or intranasally two, three or four times (preferably one time) during a RSV season to prevent, manage, treat and/or ameliorate a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). Preferably, the antibody is A4B4L1FR-S28 or an antigen-binding fragment thereof. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of one or more antibodies of the invention is administered intranasally to a subject to prevent, manage, treat and/or ameliorate a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). In one embodiment, antibodies of the invention are administered intranasally to a subject to treat URI and to prevent lower respiratory tract infection and/or RSV disease. Preferably, the antibody is A4B4L1FR-S28 or an antigen-binding fragment thereof. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In certain embodiments, a single dose of a modified antibody of the invention (preferably a MEDI-524 or a modified MEDI-524 antibody, such as MEDI-524-YTE) is administered to a patient, wherein the dose is selected from the group consisting of about 0.025 mg/kg, about 0.05 mg/kg, about 0.10 mg/kg, about 0.20 mg/kg, about 0.40 mg/kg, about 0.50 mg/kg, about 0.80 mg/kg, or about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, or about 75 mg/kg. In specific embodiments, a single dose of a modified antibody of the invention (preferably a MEDI-524 or modified MDI-524 antibody, such as MEDI-524-YTE) is administered once per year or once during the course of a RSV season, or once within 3 months, 2 months, or 1 month prior to a RSV season. In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In some embodiments, a single dose of an antibody of the invention (preferably a MEDI-524 or a modified MDI-524 antibody, such as MEDI-524-YTE) is administered to a patient two, three, four, five, six, seven, eight, nine, ten, eleven, twelve times, thirteen, fourteen, fifteen, sixteen, seventeen, eighteen, nineteen, twenty, twenty-one, twenty-two, twenty-three, twenty-four, twenty five, or twenty six at bi-weekly (e.g., about 14 day) intervals over the course of a year (or alternatively over the course of a RSV season), wherein the dose is selected from the group consisting of about 0.025 mg/kg, about 0.05 mg/kg, about 0.10 mg/kg, about 0.20 mg/kg, about 0.40 mg/kg, about 0.50 mg/kg, about 0.80 mg/kg, or about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or a combination thereof (i.e., each dose monthly dose may or may not be identical). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In another embodiment, a single dose of an antibody of the invention (preferably a MEDI-524 or a modified MDI-524 antibody, such as MEDI-524-YTE) is administered to patient two, three, four, five, six, seven, eight, nine, ten, eleven, or twelve times at about monthly (e.g., about 30 day) intervals over the course of a year (or alternatively over the course of a RSV season), wherein the dose is selected from the group consisting of about 0.025 mg/kg, about 0.05 mg/kg, about 0.10 mg/kg, about 0.20 mg/kg, about 0.40 mg/kg, about 0.50 mg/kg, about 0.80 mg/kg, or about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or a combination thereof (i.e., each dose monthly dose may or may not be identical). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In one embodiment, a single dose of an antibody of the invention (preferably a MEDI-524 or a modified MDI-524 antibody, such as MEDI-524-YTE) is administered to a patient two, three, four, five, or six times at about bi-monthly (e.g., about 60 day) intervals over the course of a year (or alternatively over the course of a RSV season), wherein the dose is selected from the group consisting of about 0.025 mg/kg, about 0.05 mg/kg, about 0.10 mg/kg, about 0.20 mg/kg, about 0.40 mg/kg, about 0.50 mg/kg, about 0.80 mg/kg, or about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or a combination thereof (i.e., each bi-monthly dose may or may not be identical). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In some embodiments, a single dose of an antibody of the invention (preferably a MEDI-524 or a modified MDI-524 antibody, such as MEDI-524-YTE) is administered to a patient two, three, or four times at about tri-monthly (e.g., about 120 day) intervals over the course of a year (or alternatively over the course of a RSV season), wherein the dose is selected from the group consisting of about 0.025 mg/kg, about 0.05 mg/kg, about 0.10 mg/kg, about 0.20 mg/kg, about 0.40 mg/kg, about 0.50 mg/kg, about 0.80 mg/kg, or about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or a combination thereof (i.e., each tri-monthly dose may or may not be identical). In certain embodiments, the above-referenced antibodies comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

In certain embodiments, the route of administration for a dose of an antibody of the invention to a patient is intranasal, intramuscular, intravenous, or a combination thereof, but other routes described herein are also acceptable. Each dose may or may not be administered by an identical route of administration). In some embodiments, an antibody of the invention may be administered via multiple routes of administration simultaneously or subsequently to other doses of the same or a different antibody of the invention.

In certain embodiments, antibodies of the invention are administered prophylactically to a subject (e.g., an infant, an infant born prematurely, an immunocompromised subject, a medical worker, or an elderly subject). Antibodies of the invention can be prophylactically administered to a subject so as to prevent a RSV infection from being transmitted from one individual to another, or to lessen the infection that is transmitted. In some embodiments, the subject has been exposed to (and may or may not be asymptomatic) or is likely to be exposed to another individual having RSV infection (e.g., acute RSV disease, or a RSV URI and/or LRI). For example, said subjects include, but are not limited to, a child in the same school or daycare as another RSV-infected child or other RSV-infected individual, an elderly person in a nursing home as an other RSV-infected individual, or an individual in the same household as a RSV infected child or other RSV-infected individual, medical staff at a hospital working with RSV-infected patients, etc. Preferably, the antibody administered prophylactically to the subject is administered intranasally, but other routes of administration described herein are acceptable. In certain preferred embodiments, the antibody of the invention is MEDI-524 or MEDI-524-YTE. In some embodiments, the antibody of the invention is administered (e.g., intranasally) at a dose of about 0.025 mg/kg, about 0.05 mg/kg, about 0.10 mg/kg, about 0.20 mg/kg, about 0.40 mg/kg, about 0.50 mg/kg, about 0.80 mg/kg, about 1 mg/kg, about 2 mg/kg, about 3 mg/kg, about 5 mg/kg, about 10 mg/kg, about 15 mg/kg, about 30 mg/kg, about 40 mg/kg, or about 50 mg/kg. Lower dosages and less frequent administration is preferred, for example, intranasal administration (or other route) once every 2-4 hours, 4-6 hours, 6-8 hours, 8-10 hours, 10-12 hours, 12-14 hours, 14-16 hours, 16-18 hours, 18-20 hours, 20-22 hours, 22-24 hours (preferably once or twice per day) for about 3 days, about 5 days or about 7 days or as otherwise needed after potential or actual exposure to the RSV-infected individual. Any antibody of the invention described herein may be used, and in certain embodiments the antibody comprises a modified IgG (e.g., IgG1) constant domain, or FcRn binding fragment thereof (e.g., the Fc domain or hinge-Fc domain), and preferably the modified IgG constant domain comprises the YTE modification (e.g., MEDI-524-YTE). In certain embodiments, the antibody is administered as a liquid formulation composition, preferably intranasally.

### 5.3.1 Liquid Formulations Comprising Antibodies of the Invention

The present invention provides liquid formulations of antibodies of the invention, which formulations exhibit, in the absence of surfactant, inorganic salts, and/or other excipients, stability and low to undetectable levels of antibody fragmentation and/or aggregation, and very little to no loss of biological activities of the antibody or antibody fragment during manufacture, preparation, transportation, and storage. The liquid formulations of the present invention facilitate the administration of the antibodies of the invention for the prevention, management, treatment and/or amelioration of a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). In particular, the liquid formulations of the present invention enable a healthcare professional to quickly administer a sterile dosage of an antibody of the invention without having to accurately and aseptically reconstitute the antibody prior to administration as required for the lyophilized dosage form. Such liquid formulations can be manufactured more easily and cost effectively than lyophilized formulations since liquid formulations do not require a prolonged drying step, such as lyophilization, freeze-drying, etc. In a preferred embodiment, the liquid formulations are made by a process in which the antibody being formulated is in an aqueous phase throughout the purification and formulation process. Preferably, the liquid formulations are made by a process that does not include a drying step, for example, but not by way of limitation, a lyophilization, freeze-drying, spray-drying, or air-drying step. Liquid formulations that can be used in the methods of the invention are described in co-owned and co-pending U.S. Ser. No. 10/461,863, which is herein incorporated by reference in its entirety.

All liquid formulations of antibodies of the invention that immunospecifically bind to a RSV antigen described herein collectively referred to as “liquid formulations of the invention,” “antibody liquid formulations of the invention,” “liquid formulations of antibodies of the invention,” “liquid formulations of anti-RSV antibodies,” and analogous terms.

The present invention provides liquid antibody formulations which are substantially free of surfactants and/or inorganic salts. The present invention also provides liquid antibody formulations which are substantially free of surfactants and other excipients. The present invention also provides liquid antibody formulations which are substantially free of surfactants, inorganic salts and other excipients. The present invention further provides liquid antibody formulations which do not comprise other ingredients except for water or suitable solvents and an antibody of the invention. In a specific embodiment, such antibody formulations are homogeneous.

In one embodiment, a liquid formulation of the invention comprises, in an aqueous carrier, about 15 mg/ml of an antibody of the invention and histidine, wherein the liquid formulation is substantially free of surfactants and inorganic salts. In accordance with this embodiment, the liquid formulation may further comprises glycine and/or other excipients. In another embodiment, a liquid formulation of the invention comprises, in an aqueous carrier, about 15 mg/ml of an antibody of the invention and histidine, wherein the liquid formulation is substantially free of surfactants, inorganic salts and other excipients.

In one embodiment, the concentration of an antibody of the invention which is included in the liquid formulations of the invention is about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 150 mg/ml, about 200 mg/ml, about 250 mg/ml, or about 300 mg/ml. In another embodiment, the concentration of an antibody of the invention which is included in the liquid formulations of the invention is about 15 mg/ml to about 300 mg/ml, about 40 mg/ml to about 300 mg/ml, about 50 mg/ml to about 300 mg/ml, about 75 mg/ml to about 300 mg/ml, or about 100 mg/ml to about 300 mg/ml.

The liquid formulations of the invention can be used to prevent, manage, treat and/or ameliorate a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD). In one embodiment, a liquid formulation of the invention comprises an antibody listed in Table 2 or Table 3, or a derivative, analogue, or fragment thereof that immunospecifically binds to a RSV antigen. In a preferred embodiment, a liquid formulation of the invention comprises A4B4-L1S28R (MEDI-524). In another preferred embodiment, a liquid formulation of the invention comprises an antibody of the invention that comprises a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

The liquid formulations of the invention can also be used for diagnostic purposes to detect, diagnose, or monitor a RSV infection. Accordingly, the invention includes liquid formulations comprising antibodies or fragments thereof that immunospecifically bind to a RSV antigen conjugated or fused to a detectable agent or label can be used to detect, diagnose, or monitor a RSV infection.

In one embodiment, the concentration of histidine which is included in the liquid formulations of the invention ranges from about 1 mM to about 100 mM, about 10 mM to about 50 mM, about 20 mM to about 30 mM, or about 23 mM to about 27 mM. In another embodiment, the concentration of histidine which is included in the liquid formulations of the invention is 1 mM or more, 10 mM or more, 15 mM or more, 20 mM or more, 25 mM or more, 30 mM or more, 35 mM or more, 40 mM or more, 45 mM or more, 50 mM or more, 55 mM or more, 60 mM or more, 65 mM or more, 70 mM or more, 75 mM or more, 80 mM or more, 85 mM or more, 90 mM or more, 95 mM or more or 100 mM or more. In a preferred embodiment, the concentration of histidine that is included in the liquid formulation of the invention is about 25 mM. Histidine can be in the form of L-histidine, D-histidine, or a mixture thereof, but L-histidine is the most preferable. Histidine can be also in the form of hydrates. Histidine may be used in a form of pharmaceutically acceptable salt, such as hydrochloride (e.g., monohydrochloride and dihydrochloride), hydrobromide, sulfate, acetate, etc. The purity of histidine should be at least 98%, preferably at least 99%, and most preferably at least 99.5%.

The pH of the formulation should not be equal to the isoelectric point of the particular antibody to be used in the formulation and may range from about 5.0 to about 7, preferably about 5.5 to about 6.5, more preferably about 5.8 to about 6.2, and most preferably about 6.0.

In addition to histidine and an antibody of the invention, the liquid formulations of the present invention may further comprise glycine. In one embodiment, the concentration of glycine which is included in a liquid formulation of the invention is about 0.1 mM to about 100 mM. In another embodiment, the concentration of glycine which is included in a liquid formulation of the invention is less than 100 mM, less than 50 mM, less than 3.0 mM, less than 2.0 mM, or less than 1.8 mM. In a preferred embodiment, the concentration of glycine which is included in a liquid formulation of the invention is 1.6 mM. The amount of glycine in the formulation should not cause a significant buffering effect so that antibody precipitation at its isoelectric point can be avoided. Glycine may be also used in a form of pharmaceutically acceptable salt, such as hydrochloride, hydrobromide, sulfate, acetate, etc. The purity of glycine should be at least 98%, preferably at least 99%, and most preferably 99.5%. In a specific embodiment, glycine is included in the liquid formulations of the present invention.

Optionally, the liquid formulations of the present invention may further comprise other excipients, such as saccharides (e.g., sucrose, mannose, trehalose, etc.) and polyols (e.g., mannitol, sorbitol, etc.). In one embodiment, the other excipient is a saccharide. In a specific embodiment, the saccharide is sucrose, which is at a concentration ranging from between about 1% to about 20%, preferably about 5% to about 15%, and more preferably about 8% to 10%. In another embodiment, the other excipient is a polyol. Preferably, however, the liquid formulations of the present invention do not contain mannitol. In a specific embodiment, the polyol is polysorbate (e.g., Tween 20), which is at a concentration ranging from between about 0.001% to about 1%, preferably, about 0.01% to about 0.1%.

The liquid formulations of the present invention exhibit stability at the temperature ranges of 38° C.-42° C. for at least 60 days and, in some embodiments, not more than 120 days, of 20° C.-24° C. for at least 1 year, of 2° C.-8° C. (in particular, at 4° C.) for at least 3 years, at least 4 years, or at least 5 years and at −20° C. for at least 3 years, at least 4 years, or at least 5 years, as assessed by high performance size exclusion chromatography (HPSEC). Namely, the liquid formulations of the present invention have low to undetectable levels of aggregation and/or fragmentation, as defined herein, after the storage for the defined periods as set forth above. Preferably, no more than 5%, no more than 4%, no more than 3%, no more than 2%, no more than 1%, and most preferably no more than 0.5% of the antibody or antibody fragment forms an aggregate as measured by HPSEC, after the storage for the defined periods as set forth above. Furthermore, liquid formulations of the present invention exhibit almost no loss in biological activities of the antibody or antibody fragment during the prolonged storage under the condition described above, as assessed by various immunological assays including, but not limited to, enzyme-linked immunosorbent assay (ELISA) and radioimmunoassay to measure the ability of an antibody or antibody fragment to immunospecifically bind to a RSV antigen, and by a C3a/C4a assay to measure the complement activating ability of the antibody. In a specific embodiment, the liquid formulations exhibit very little to no loss of the biological activity(ies) of the antibodies or antibody fragments of the formulation compared to the reference antibodies as measured by antibody binding assays such as, e.g., ELISAs. The liquid formulations of the present invention retain after the storage for the above-defined periods more than 80%, more than 85%, more than 90%, more than 95%, more than 98%, more than 99%, or more than 99.5% of the initial biological activities of the formulation prior to the storage.

The liquid formulations of the present invention can be prepared as unit dosage forms. For example, a unit dosage per vial may contain 0.1 ml, 0.25 ml, 0.5 ml, 1 ml, 2 ml, 3 ml, 4 ml, 5 ml, 6 ml, 7 ml, 8 ml, 9 ml, 10 ml, 15 ml, or 20 ml of different concentrations of an antibody of the invention ranging from about 15 mg/ml to about 300 mg/ml. If necessary, these preparations can be adjusted to a desired concentration by adding a sterile diluent to each vial.

The invention encompasses stable liquid formulations comprising a single antibody of the invention, with the proviso that said antibody is not palivizumab. The invention also encompasses stable liquid formulations comprising two or more antibodies of the invention. In one embodiment, a stable liquid formulation of the invention comprises two or more antibodies of the invention, wherein one of the antibodies is palivizumab or a fragment thereof. In an alternative embodiment, a stable liquid formulation of the invention comprises two or more antibodies of the invention, with the proviso that the antibodies do not include palivizumab or a fragment thereof.

The present invention also provides kits comprising the liquid formulations of antibodies of the invention for use by, e.g., a healthcare professional. The present invention also provides methods of preventing, managing, treating and/or ameliorating a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) by administering the liquid formulations of the present invention. The liquid formulations of the present invention can also be used to diagnose, detect or monitor a RSV infection, such as an acute RSV disease, a RSV URI, or a RSV LRI).

In certain embodiments, a liquid formulation of the invention and one or more other therapies (e.g., one or more other prophylactic or therapeutic agents) useful for prevention, management, treatment and/or amelioration of a RSV infection (e.g., acute RSV disease, or a RSV URI and/or LRI) are administered in a cycle of less than about 3 weeks, about once every two weeks, about once every 10 days or about once every week. One cycle can comprise the administration of a therapy (e.g., a therapeutic or prophylactic agent) by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles. In certain embodiments, the liquid formulation of the invention is in a cycle of hours (e.g., about every 1 to 6 hours, 6 to 12 hours, 12 to 18 hours, or 18-24 hours) to days (e.g., daily, every other day, every third day, every fourth day, every fifth day, every sixth day or every seventh day). In certain embodiments, the liquid formulations of the invention are delivered intranasally. In some embodiments the antibody is an unmodified antibody of the invention. In other embodiments, the antibody comprise a modified IgG (e.g., IgG1) constant domain, or FcRn binding fragment thereof (e.g., the Fc domain or hinge-Fc domain), described herein, and preferably the modified IgG constant domain comprises the YTE modification (e.g., MEDI-524-YTE).

### 5.3.2 Methods of Preparing Liquid Formulations of the Invention

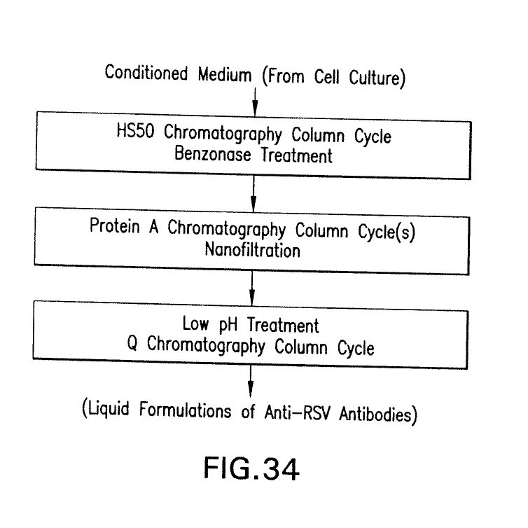

The present invention also provides methods for preparing liquid formulations of antibodies, in particular, those listed in Table 2 or Table 3 (or other antibodies of the invention described herein), or derivatives, analogues, or fragments thereof that immunospecifically bind to a RSV antigen. FIG. 34 is a schematic diagram showing the outline for preparing purified anti-RSV antibodies. The methods for preparing liquid formulations of the present invention comprise: concentrating a fraction containing the purified antibody or a fragment to a final antibody or fragment concentration of from about 15 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 110 mg/ml, about 125 mg/ml, about 150 mg/ml, about 200 mg/ml, about 250 mg/ml, or about 300 mg/ml using a semipermeable membrane with an appropriate molecular weight (MW) cutoff (e.g., 30 kD cutoff for whole antibody molecules and F(ab′)₂ fragments; and 10 kD cutoff for antibody fragments, such as Fab fragments) and difiltrating the concentrated antibody fraction into the formulation buffer using the same membrane. Conditioned medium containing antibody or a fragment thereof that immunospecifically binds to a RSV antigen is subjected to CUNO filtration and the filtered antibody is subjected to HS50 cation exchange chromatography. The fraction from the HS50 cation exchange chromatography is then subjected to rProtein A affinity chromatography followed by low pH treatment. Following low pH treatment, the antibody fraction is subject to super Q 650 anion exchange chromatography and then nanofiltration. The fraction of the antibody obtained after nanofiltration is then subjected to diafiltration to concentrate the antibody fraction into the formulation buffer using the same membrane.

The formulation buffer of the present invention comprises histidine at a concentration ranging from about 1 mM to about 100 mM, about 10 mM to about 50 mM, about 20 mM to about 30 mM, or about 23 mM to about 27 mM. Preferably, the formulation buffer of the present invention comprises histidine at a concentration of about 25 mM. The formulations may further comprise glycine at a concentration of less than 100 mM, less than 50 mM, less than 3.0 mM, less than 2.0 mM, or less than 1.8 mM. Preferably, the formulations comprise glycine at a concentration of 1.6 mM. The amount of glycine in the formulation should not cause a significant buffering in order to avoid antibody precipitation at its isoelectric point. The pH of the formulation may range from about 5.0 to about 7.0, preferably about 5.5 to about 6.5, more preferably about 5.8 to about 6.2, and most preferably about 6.0. To obtain an appropriate pH for a particular antibody, it is preferable that histidine (and glycine, if added) is first dissolved in water to obtain a buffer solution with higher pH than the desired pH and then the pH is brought down to the desired level by adding HCl. This way, the formation of inorganic salts (e.g., formation of NaCl when, for example, histidine hydrochloride is used as histidine and pH is raised to a desired level by adding NaOH) can be avoided.

The liquid formulations of the present invention can be prepared as unit dosage forms by preparing a vial containing an aliquot of the liquid formulation for a one-time use. For example, a unit dosage per vial may contain 0.1 ml, 0.25 ml, 0.5 ml, 1 ml, 2 ml, 3 ml, 4 ml, 5 ml, 6 ml, 7 ml, 8 ml, 9 ml, 10 ml, 15 ml, or 20 ml of different concentrations of an antibody of the invention ranging from about 15 mg/ml to about 300 mg/ml. If necessary, these preparations can be adjusted to a desired concentration by adding a sterile diluent to each vial.

The liquid formulations of the present invention may be sterilized by various sterilization methods, including sterile filtration, radiation, etc. In a most preferred embodiment, the difiltrated antibody formulation is filter-sterilized with a presterilized 0.2 or 0.22-micron filter. Sterilized liquid formulations of the present invention may be administered to a subject to prevent, treat, manage or ameliorate a RSV infection, one or more symptoms thereof, or a respiratory condition associated with, potentiated by, potentiating a RSV infection.

Preferably, the liquid formulations of the present invention are prepared by maintaining the antibodies in an aqueous solution at any time during the preparation. In other words, the liquid formulations are prepared without involving any step of drying the antibodies or the formulations themselves by, for example, lyophilization, vacuum drying, etc.

Although the invention is directed to liquid non-lyophilized formulations, it should be noted for the purpose of equivalents that the formulations of the invention may be lyophilized if desired. Thus, the invention encompasses lyophilized forms of the formulations of the invention although such lyophilized formulations are not necessary and, thus, not preferred.

### 5.3.3 Methods of Monitoring the Stability And Aggregation of Antibody Formulations

There are various methods available for assessing the stability of the liquid formulations of the present invention, based on the physical and chemical structures of the proteins (e.g., antibodies or fragments thereof) as well as on their biological activities. For example, to study denaturation of proteins, methods such as charge-transfer absorption, thermal analysis, fluorescence spectroscopy, circular dichroism, NMR, and HPSEC, are available. See, for example, Wang et al., 1988, J. of Parenteral Science & Technology 42(Suppl):S4-S26.

The rCGE and HPSEC are the most common and simplest methods to assess the formation of protein aggregates, protein degradation, and protein fragmentation. Accordingly, the stability of the liquid formulations of the present invention may be assessed by these methods.

For example, the stability of the liquid formulations of the present invention may be evaluated by HPSEC or rCGE, wherein the percent area of the peaks represents the non-degraded antibody or non-degraded antibody fragments. In particular, approximately 250 μg of the antibody or antibody fragment that immunospecifically binds to a RSV antigen (approximately 25 μl of a liquid formulation comprising 10 mg/ml said antibody or antibody fragment) is injected onto a TosoH Biosep TSK G3000SW_{XL} column (7.8 mm×30 cm) fitted with a TSK SW×1 guard column (6.0 mm CX 4.0 cm). The antibody or antibody fragment is eluted isocratically with 0.1 M disodium phosphate containing 0.1 M sodium sulfate and 0.05% sodium azide, at a flow rate of 0.8 to 1.0 ml/min. Eluted protein is detected using UV absorbance at 280 nm. palivizumab reference standard is run in the assay as a control, and the results are reported as the area percent of the product monomer peak compared to all other peaks excluding the included volume peak observed at approximately 12 to 14 minutes. Peaks eluting earlier than the monomer peak are recorded as percent aggregate.

The liquid formulations of the present invention exhibit low to undetectable levels of aggregation as measured by HPSEC or rCGE, that is, no more than 5%, no more than 4%, no more than 3%, no more than 2%, no more than 1%, and most preferably no more than 0.5% aggregate by weight protein, and low to undetectable levels of fragmentation, that is, 80% or higher, 85% or higher, 90% or higher, 95% or higher, 98% or higher, or 99% or higher, or 99.5% or higher of the total peak area in the peak(s) representing intact antibodies or fragments thereof. In the case of SDS-PAGE, the density or the radioactivity of each band stained or labeled with radioisotope can be measured and the % density or % radioactivity of the band representing non-degraded antibodies or fragments thereof can be obtained.

The stability of the liquid formulations of the present invention can be also assessed by any assays which measures the biological activity of the antibody or fragments thereof in the formulation. The biological activities of antibodies include, but are not limited to, antigen-binding activity, complement-activation activity, Fc-receptor binding activity, and so forth. Antigen-binding activity of the antibodies can be measured by any method known to those skilled in the art, including but not limited to ELISA, radioimmunoassay, Western blot, and the like. Complement-activation activity can be measured by a C3a/C4a assay in the system where the antibody which immunospecifically binds to a RSV antigen is reacted in the presence of the complement components with the cells expressing the RSV antigen. Also see Harlow et al., Antibodies: A Laboratory Manual, (Cold Spring Harbor Laboratory Press, 2nd ed. 1988) (incorporated by reference herein in its entirety). An ELISA based assay, e.g., may be used to compare the ability of an antibody or fragment thereof to immunospecifically bind to a RSV antigen to a palivizumab reference standard. In this assay, plates are coated with a RSV antigen and the binding signal of a set concentration of a palivizumab reference standard is compared to the binding signal of the same concentration of a test antibody or antibody fragment.

The purity of the liquid antibody formulations of the invention may be measured by any method well-known to one of skill in the art such as, e.g., HPSEC. The sterility of the liquid antibody formulations may be assessed as follows: sterile soybean-casein digest medium and fluid thioglycollate medium are inoculated with a test liquid antibody formulation by filtering the liquid antibody formulation through a sterile filter having a nominal porosity of 0.45 μm. When using the Sterisure™ or Steritest™ method, each filter device is aseptically filled with approximately 100 ml of sterile soybean-casein digest medium or fluid thioglycollate medium. When using the conventional method, the challenged filter is aseptically transferred to 100 ml of sterile soybean-casein digest medium or fluid thioglycollate medium. The media are incubated at appropriate temperatures and observed three times over a 14 day period for evidence of bacterial or fungal growth.

### 5.4 Gene Therapy

In a specific embodiment, nucleic acids comprising sequences encoding antibodies of the invention or functional derivatives thereof, are administered to prevent, manage, treat and/or ameliorate a RSV infection (e.g., acute RSV disease, or a RSV URI and/or LRI), otitis media (preferably, stemming from, caused by or associated with a RSV infection, such as a RSV URI and/or LRI), and/or a symptom or respiratory condition relating thereto (e.g., asthma, wheezing, and/or RAD) by way of gene therapy. Gene therapy refers to therapy performed by the administration to a subject of an expressed or expressible nucleic acid. In an embodiment of the invention, the nucleic acids produce their encoded antibody, and the antibody mediates a prophylactic or therapeutic effect.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred embodiment, a composition of the invention comprises nucleic acids encoding an antibody of the invention, said nucleic acids being part of an expression vector that expresses the antibody or chimeric proteins or heavy or light chains thereof in a suitable host. In particular, such nucleic acids have promoters, preferably heterologous promoters, operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438). In some embodiments, the expressed antibody molecule is a single chain antibody; alternatively, the nucleic acid sequences include sequences encoding both the heavy and light chains, or fragments thereof, of the antibody.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where the sequences are expressed to produce the encoded product. This can be accomplished by any of numerous methods known in the art, e.g., by constructing them as part of an appropriate nucleic acid expression vector and administering the vector so that the sequences become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO 92/20316; WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody of the invention are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy can be cloned into one or more vectors, which facilitates delivery of the gene into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the subject.

In an embodiment in which recombinant cells are used in gene therapy, nucleic acid sequences encoding an antibody of the invention are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### 5.5 Diagnostic Uses of Antibodies

Labeled antibodies of the invention (modified or unmodified) and derivatives and analogs thereof, which immunospecifically bind to a RSV antigen can be used for diagnostic purposes to detect, diagnose, or monitor a RSV URI and/or LRI or otitis media (preferably, stemming from, caused by or associated with a RSV infection, such as a RSV URI and/or LRI). The invention provides methods for the detection of a RSV infection (e.g., a RSV URI and/or LRI), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof) comprising: (a) assaying the expression of a RSV antigen in cells or a tissue sample of a subject using one or more antibodies of the invention that immunospecifically bind to the RSV antigen; and (b) comparing the level of the RSV antigen with a control level, e.g., levels in normal tissue samples not infected with RSV, whereby an increase in the assayed level of RSV antigen compared to the control level of the RSV antigen is indicative of a RSV infection (e.g., a RSV URI and/or LRI), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof).

The invention provides a diagnostic assay for diagnosing a RSV infection (e.g., a RSV URI and/or LRI), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof) comprising: (a) assaying for the level of a RSV antigen in cells or a tissue sample of an individual using one or more antibodies of the invention that immunospecifically bind to a RSV antigen; and (b) comparing the level of the RSV antigen with a control level, e.g., levels in normal tissue samples not infected with RSV, whereby an increase in the assayed RSV antigen level compared to the control level of the RSV antigen is indicative of a RSV infection (e.g., a RSV URI and/or LRI), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof). A more definitive diagnosis of a RSV infection (e.g., a RSV URI and/or LRI), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof) may allow health professionals to employ preventative measures or aggressive treatment earlier thereby preventing the development or further progression of the RSV infection or otitis media.

Antibodies of the invention can be used to assay RSV antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I) carbon (¹⁴C), sulfur (³⁵S), tritium (³H), indium (121In), and technetium (⁹⁹Tc); luminescent labels, such as luminol; and fluorescent labels, such as fluorescein and rhodamine, and biotin.

One aspect of the invention is the detection and diagnosis of a RSV infection (e.g., a RSV URI and/or LRI), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof) in a human. In one embodiment, diagnosis comprises: a) administering (for example, parenterally, subcutaneously, or intraperitoneally) to a subject an effective amount of a labeled antibody that immunospecifically binds to a RSV antigen; b) waiting for a time interval following the administering for permitting the labeled antibody to preferentially concentrate at sites in the subject (e.g., the nasal passages, lungs, mouth and ears) where the RSV antigen is expressed (and for unbound labeled molecule to be cleared to background level); c) determining background level; and d) detecting the labeled antibody in the subject, such that detection of labeled antibody above the background level indicates that the subject has a RSV infection (e.g., a RSV URI and/or LRI), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof). Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled antibody to preferentially concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

In one embodiment, monitoring of a RSV URI and/or LRI is carried out by repeating the method for diagnosing the RSV URI and/or LRI, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### 5.6 Biological Activity and Assays for Extended Half-Life of Modified Antibodies

Antibodies of the present invention may be characterized in a variety of ways. In particular, antibodies of the invention may be assayed for the ability to immunospecifically bind to a RSV antigen. Such an assay may be performed in solution (e.g., Houghten, 1992, Bio/Techniques 13:412-421), on beads (Lam, 1991, Nature 354:82-84), on chips (Fodor, 1993, Nature 364:555-556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310) (each of these references is incorporated herein in its entirety by reference). Antibodies that have been identified to immunospecifically bind to a RSV antigen (e.g., a RSV F antigen) can then be assayed for their specificity and affinity for a RSV antigen.

The modified antibodies of the invention may be assayed for immunospecific binding to a RSV antigen and cross-reactivity with other antigens by any method known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, incubating the membrane in blocking solution (e.g., PBS with 3% BSA or non-fat milk), washing the membrane in washing buffer (e.g., PBS-Tween 20), incubating the membrane with primary antibody (the antibody of interest) diluted in blocking buffer, washing the membrane in washing buffer, incubating the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of the present invention for a RSV antigen and the binding off-rates can be determined from the data by scatchard plot analysis. Competition with a second antibody can also be determined using radioimmunoassays. In this case, a RSV antigen is incubated with an antibody of the present invention conjugated to a labeled compound (e.g., ³H or ¹²⁵I) in the presence of increasing amounts of an unlabeled second antibody.

In a preferred embodiment, BIAcore kinetic analysis is used to determine the binding on and off rates of antibodies to a RSV antigen. BIAcore kinetic analysis comprises analyzing the binding and dissociation of a RSV antigen from chips with immobilized antibodies on their surface.

The antibodies of the invention can also be assayed for their ability to inhibit the binding of RSV to its host cell receptor using techniques known to those of skill in the art. For example, cells expressing the receptor for RSV can be contacted with RSV in the presence or absence of an antibody and the ability of the antibody to inhibit RSV's binding can measured by, for example, flow cytometry or a scintillation assay. RSV (e.g., a RSV antigen such as F glycoprotein or G glycoprotein) or the antibody can be labeled with a detectable compound such as a radioactive label (e.g., 32P, 35S, and 125I) or a fluorescent label (e.g., fluorescein isothiocyanate, rhodamine, phycoerythrin, phycocyanin, allophycocyanin, o-phthaldehyde and fluorescamine) to enable detection of an interaction between RSV and its host cell receptor. Alternatively, the ability of antibodies to inhibit RSV from binding to its receptor can be determined in cell-free assays. For example, RSV or a RSV antigen such as G glycoprotein can be contacted with an antibody and the ability of the antibody to inhibit RSV or the RSV antigen from binding to its host cell receptor can be determined. Preferably, the antibody is immobilized on a solid support and RSV or a RSV antigen is labeled with a detectable compound. Alternatively, RSV or a RSV antigen is immobilized on a solid support and the antibody is labeled with a detectable compound. RSV or a RSV antigen may be partially or completely purified (e.g., partially or completely free of other polypeptides) or part of a cell lysate. Further, a RSV antigen may be a fusion protein comprising the RSV antigen and a domain such as glutathionine S transferase. Alternatively, a RSV antigen can be biotinylated using techniques well known to those of skill in the art (e.g., biotinylation kit, Pierce Chemicals; Rockford, Ill.).

The antibodies of the invention can also be assayed for their ability to inhibit or downregulate RSV replication using techniques known to those of skill in the art. For example, RSV replication can be assayed by a plaque assay such as described, e.g., by Johnson et al., 1997, Journal of Infectious Diseases 176:1215-1224. The modified antibodies of the invention can also be assayed for their ability to inhibit or down-regulate the expression of RSV polypeptides. Techniques known to those of skill in the art, including, but not limited to, Western blot analysis, Northern blot analysis, and RT-PCR can be used to measure the expression of RSV polypeptides. Further, the antibodies of the invention can be assayed for their ability to prevent the formation of syncytia.

The antibodies of the invention are preferably tested in vitro, and then in vivo for the desired therapeutic or prophylactic activity, prior to use in humans. For example, in vitro assays which can be used to determine whether administration of a specific antibody or composition of the present invention is indicated, include in vitro cell culture assays in which a subject tissue sample is grown in culture, and exposed to or otherwise administered an antibody or composition of the present invention, and the effect of such an antibody or composition of the present invention upon the tissue sample is observed. In various specific embodiments, in vitro assays can be carried out with representative cells of cell types involved in a RSV infection (e.g., respiratory epithelial cells), to determine if an antibody or composition of the present invention has a desired effect upon such cell types. Preferably, the antibodies or compositions of the invention are also tested in in vitro assays and animal model systems prior to administration to humans. In a specific embodiment, cotton rats are administered an antibody the invention, or a composition of the invention, challenged with 10⁵ pfu of RSV, and four or more days later the rats are sacrificed and RSV titer and anti-RSV antibody serum titer is determined. Further, in accordance with this embodiment, the tissues (e.g., the lung tissues) from the sacrificed rats can be examined for histological changes.

In accordance with the invention, clinical trials with human subjects need not be performed in order to demonstrate the prophylactic and/or therapeutic efficacy of modified antibodies of the invention. In vitro and animal model studies using the antibodies can be extrapolated to humans and are sufficient for demonstrating the prophylactic and/or therapeutic utility of said antibodies.

Antibodies or compositions of the present invention for use in therapy can be tested for their toxicity in suitable animal model systems, including but not limited to rats, mice, cows, monkeys, and rabbits. For in vivo testing of an antibody or composition's toxicity any animal model system known in the art may be used.

Efficacy in preventing, managing, treating and/or ameliorating a RSV infection (e.g., acute RSV disease, or a RSV URI and/or LRI) may be demonstrated by determining the ability of an antibody or composition of the invention to inhibit the replication of the virus, to inhibit transmission or prevent the virus from establishing itself in its host, to reduce the incidence of a RSV URI and/or LRI, to prevent or reduce the progression of an upper respiratory tract RSV infection to a lower respiratory tract RSV infection, or to prevent, ameliorate or alleviate one or more symptoms associated with a RSV URI and/or LRI. Efficacy in treating, preventing or otherwise managing otitis media may be demonstrated by determining the ability of an antibody or composition of the invention to reduce the incidence or otitis media, to reduce the duration of otitis media, to prevent or reduce the progression of a RSV URI and/or LRI to otitis media, or to ameliorate one or more symptoms of otitis media. A therapy is considered therapeutic if there is, for example, a reduction is viral load, amelioration of one or more symptoms of a RSV URI and/or LRI or otitis media, or a respiratory condition relating thereto (including, but not limited to asthma, wheezing, RAD or a combination thereof), a reduction in the duration of a RSV URI and/or LRI or otitis media, a reduction in lower respiratory tract RSV infections, or a decrease in mortality and/or morbidity following administration of an antibody or composition of the invention. Further, the treatment is considered therapeutic if there is an increase in the immune response following the administration of one or more antibodies which immunospecifically bind to one or more RSV antigens.

Antibodies or compositions of the invention can be tested in vitro and in vivo for the ability to induce the expression of cytokines such as IFN-α, IFN-β, IFN-γ, IL-2, IL-3, IL-4, IL-5, IL-6, IL-7, IL-8, IL-9, IL-10, IL-12 and IL-15. Techniques known to those of skill in the art can be used to measure the level of expression of cytokines. For example, the level of expression of cytokines can be measured by analyzing the level of RNA of cytokines by, for example, RT-PCR and Northern blot analysis, and by analyzing the level of cytokines by, for example, immunoprecipitation followed by western blot analysis and ELISA. In a preferred embodiment, an antibody or composition of the invention is tested for its ability to induce the expression of IFN-γ.

Antibodies or compositions of the invention can be tested in vitro and in vivo for their ability to modulate the biological activity of immune cells, preferably human immune cells (e.g., T-cells, B-cells, and Natural Killer cells). The ability of an antibody or composition of the invention to modulate the biological activity of immune cells can be assessed by detecting the expression of antigens, detecting the proliferation of immune cells, detecting the activation of signaling molecules, detecting the effector function of immune cells, or detecting the differentiation of immune cells. Techniques known to those of skill in the art can be used for measuring these activities. For example, cellular proliferation can be assayed by ³H thymidine incorporation assays and trypan blue cell counts. Antigen expression can be assayed, for example, by immunoassays including, but are not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and FACS analysis. The activation of signaling molecules can be assayed, for example, by kinase assays and electrophoretic shift assays (EMSAs). Antibodies or compositions of the invention can also be tested for their ability to inhibit viral replication or reduce viral load in in vitro, ex vivo and in vivo assays. Antibodies or compositions of the invention can also be tested for their ability to decrease the time course of a RSV infection (e.g., a RSV URI and/or LRI), otitis media (preferably stemming from, caused by or associated with a RSV infection, such as an upper and/or lower respiratory tract infection), or a symptom or respiratory condition relating thereto (including, but not limited to, asthma, wheezing, RAD, or a combination thereof). Antibodies or compositions of the invention can also be tested for their ability to increase the survival period of humans suffering from a RSV infection (preferably, a RSV URI and/or LRI) by at least 25%, preferably at least 50%, at least 60%, at least 75%, at least 85%, at least 95%, or at least 99%. Further, antibodies or compositions of the invention can be tested for their ability reduce the hospitalization period of humans suffering from a RSV infection (preferably, a RSV URI and/or LRI) by at least 60%, preferably at least 75%, at least 85%, at least 95%, or at least 99%. Techniques known to those of skill in the art can be used to analyze the function of the antibodies or compositions of the invention in vivo.

The binding ability of IgGs and molecules comprising an IgG constant domain of FcRn fragment thereof to FcRn can be characterized by various in vitro assays. PCT publication WO 97/34631 by Ward discloses various methods in detail and is incorporated herein in its entirety by reference.

For example, in order to compare the ability of a modified antibody of the invention or fragments thereof to bind to FcRn with that of the unmodified or wild type IgG, the modified IgG or fragments thereof and the unmodified or wild type IgG can be radio-labeled and reacted with FcRn-expressing cells in vitro. The radioactivity of the cell-bound fractions can be then counted and compared. The cells expressing FcRn to be used for this assay are preferably endothelial cell lines including mouse pulmonary capillary endothelial cells (B10, D2.PCE) derived from lungs of B10.DB A/2 mice and SV40 transformed endothelial cells (SVEC) (Kim et al., J. Immunol., 40:457-465, 1994) derived from C3H/HeJ mice. However, other types of cells, such as intestinal brush borders isolated from 10- to 14-day old suckling mice, which express sufficient number of FcRn can be also used. Alternatively, mammalian cells which express recombinant FcRn of a species of choice can be also utilized. After counting the radioactivity of the bound fraction of modified IgG or that of the unmodified or wild type, the bound molecules can be then extracted with the detergent, and the percent release per unit number of cells can be calculated and compared.

Affinity of modified IgGs for FcRn can be measured by surface plasmon resonance (SPR) measurement using, for example, a BIAcore 2000 (BIAcore Inc.) as described previously (Popov et al., Mol. Immunol., 33:493-502, 1996; Karlsson et al., J. Immunol. Methods, 145:229-240, 1991, both of which are incorporated by reference in their entireties). In this method, FcRn molecules are coupled to a BIAcore sensor chip (e.g., CM5 chip by Pharmacia) and the binding of modified IgG to the immobilized FcRn is measured at a certain flow rate to obtain sensorgrams using BIA evaluation 2.1 software, based on which on- and off-rates of the modified IgG, constant domains, or fragments thereof, to FcRn can be calculated.

Relative affinities of modified IgGs or fragments thereof, and the unmodified or wild type IgG for FcRn can be also measured by a simple competition binding assay. Unlabeled modified IgG or unmodified or wild type IgG is added in different amounts to the wells of a 96-well plate in which FcRn is immobilize. A constant amount of radio-labeled unmodified or wild type IgG is then added to each well. Percent radioactivity of the bound fraction is plotted against the amount of unlabeled modified IgG or unmodified or wild type IgG and the relative affinity of the modified hinge-Fc can be calculated from the slope of the curve.

Furthermore, affinities of modified IgGs or fragments thereof, and the wild type IgG for FcRn can be also measured by a saturation study and the Scatchard analysis.

Transfer of modified IgG or fragments thereof across the cell by FcRn can be measured by in vitro transfer assay using radiolabeled IgG or fragments thereof and FcRn-expressing cells and comparing the radioactivity of the one side of the cell monolayer with that of the other side. Alternatively, such transfer can be measured in vivo by feeding 10- to 14-day old suckling mice with radiolabeled, modified IgG and periodically counting the radioactivity in blood samples which indicates the transfer of the IgG through the intestine to the circulation (or any other target tissue, e.g., the lungs). To test the dose-dependent inhibition of the IgG transfer through the gut, a mixture of radiolabeled and unlabeled IgG at certain ratio is given to the mice and the radioactivity of the plasma can be periodically measured (Kim et al., Eur. J. Immunol., 24:2429-2434, 1994).

The half-life of modified IgG or fragments thereof can be measured by pharmacokinetic studies according to the method described by Kim et al. (Eur. J. of Immuno. 24:542, 1994), which is incorporated by reference herein in its entirety. According to this method, radiolabeled modified IgG or fragments thereof is injected intravenously into mice and its plasma concentration is periodically measured as a function of time, for example, at 3 minutes to 72 hours after the injection. The clearance curve thus obtained should be biphasic, that is, α-phase and β-phase. For the determination of the in vivo half-life of the modified IgGs or fragments thereof, the clearance rate in 3-phase is calculated and compared with that of the unmodified or wild type IgG.

### 5.7 Methods of Producing Antibodies

Antibodies of the invention that immunospecifically bind to an antigen can be produced by any method known in the art for the synthesis of antibodies, in particular, by chemical synthesis or preferably, by recombinant expression techniques. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

Polyclonal antibodies that immunospecifically bind to an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563 681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a RSV antigen and once an immune response is detected, e.g., antibodies specific for a RSV antigen (preferably, RSV F antigen) are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Additionally, a RIMMS (repetitive immunization multiple sites) technique can be used to immunize an animal (Kilptrack et al., 1997 Hybridoma 16:381-9, incorporated by reference in its entirety). The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating antibodies by culturing a hybridoma cell secreting a modified antibody of the invention wherein, preferably, the hybridoma is generated by fusing splenocytes isolated from a mouse immunized with a RSV antigen with myeloma cells and then screening the hybridomas resulting from the fusion for hybridoma clones that secrete an antibody able to bind to a RSV antigen (preferably, RSV F antigen).

Antibody fragments which recognize specific RSV antigens (preferably, RSV F antigen) may be generated by any technique known to those of skill in the art. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain. Further, the antibodies of the present invention can also be generated using various phage display methods known in the art.

For example, antibodies can also be generated using various phage display methods. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/O1 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415, which are incorporated herein by reference in their entirety.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)₂, Fabc, Fv) in which all or substantially all of the CDR regions correspond to those of a non human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. Examples of VL and VH constant domains that can be used in certain embodiments of the invention include, but are not limited to, C-kappa and C-gamma-1 (nG1m) described in Johnson et al. (1997) J. Infect. Dis. 176, 1215-1224 and those described in U.S. Pat. No. 5,824,307. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental FR and CDR sequences, more often 90%, and most preferably greater than 95%. Humanized antibodies can be produced using variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), which is incorporated by reference herein in its entirety. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Reichmann et al., 1988, Nature 332:323, which are incorporated herein by reference in their entireties.)

Single domain antibodies, for example, antibodies lacking the light chains, can be produced by methods well-known in the art. See Riechmann et al., 1999, J. Immunol. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301, each of which is incorporated herein by reference in its entirety.

Further, the antibodies that immunospecifically bind to a RSV antigen (e.g., a RSV F antigen) can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250, which references are incorporated herein by reference in their entireties. Recombinant molecular biological techniques such as those described for recombinant production of antibodies may also be used in the generation of intrabodies.

In one embodiment, intrabodies of the invention retain about 75% of the binding effectiveness of the complete antibody (i.e., having the entire constant domain as well as the variable regions) to the antigen. More preferably, the intrabody retains at least 85% of the binding effectiveness of the complete antibody. Still more preferably, the intrabody retains at least 90% of the binding effectiveness of the complete antibody. Even more preferably, the intrabody retains at least 95% of the binding effectiveness of the complete antibody.

In producing intrabodies, polynucleotides encoding variable region for both the V_{H} and V_{L} chains of interest can be cloned by using, for example, hybridoma mRNA or splenic mRNA as a template for PCR amplification of such domains (Huse et al., 1989, Science 246:1276). In one preferred embodiment, the polynucleotides encoding the V_{H} and V_{L} domains are joined by a polynucleotide sequence encoding a linker to make a single chain antibody (scFv). The scFv typically comprises a single peptide with the sequence V_{H}-linker-V_{L} or V_{L}-linker-V_{H}. The linker is chosen to permit the heavy chain and light chain to bind together in their proper conformational orientation (see for example, Huston et al., 1991, Methods in Enzym. 203:46-121, which is incorporated herein by reference). In a further embodiment, the linker can span the distance between its points of fusion to each of the variable domains (e.g., 3.5 nm) to minimize distortion of the native Fv conformation. In such an embodiment, the linker is a polypeptide of at least 5 amino acid residues, at least 10 amino acid residues, at least 15 amino acid residues, or greater. In a further embodiment, the linker should not cause a steric interference with the V_{H} and V_{L} domains of the combining site. In such an embodiment, the linker is 35 amino acids or less, 30 amino acids or less, or 25 amino acids or less. Thus, in a most preferred embodiment, the linker is between 15-25 amino acid residues in length. In a further embodiment, the linker is hydrophilic and sufficiently flexible such that the V_{H} and V_{L} domains can adopt the conformation necessary to detect antigen. Intrabodies can be generated with different linker sequences inserted between identical V_{H} and V_{L} domains. A linker with the appropriate properties for a particular pair of V_{H} and V_{L} domains can be determined empirically by assessing the degree of antigen binding for each. Examples of linkers include, but are not limited to, those sequences disclosed in Table 5

TABLE 5

| Sequence | SEQ ID NO. |
|---|---|
| (Gly Gly Gly Gly Ser)₃ | SEQ ID NO: 260 |
| Glu Ser Gly Arg Ser Gly Gly Gly Gly | SEQ ID NO: 261 |
| Ser Gly Gly Gly Gly Ser | |
| Glu Gly Lys Ser Ser Gly Ser Gly Ser | SEQ ID NO: 262 |
| Glu Ser Lys Ser Thr | |
| Glu Gly Lys Ser Ser Gly Ser Gly Ser | SEQ ID NO: 263 |
| Glu Ser Lys Ser Thr Gln | |
| Glu Gly Lys Ser Ser Gly Ser Gly Ser | SEQ ID NO: 264 |
| Glu Ser Lys Val Asp | |
| Gly Ser Thr Ser Gly Ser Gly Lys Ser | SEQ ID NO: 265 |
| Ser Glu Gly Lys Gly | |
| Lys Glu Ser Gly Ser Val Ser Ser | SEQ ID NO: 266 |
| Glu Gln Leu Ala Gln Phe Arg Ser | |
| Leu Asp | |
| Glu Ser Gly Ser Val Ser Ser Glu Glu | SEQ ID NO: 267 |
| Leu Ala Phe Arg Ser Leu Asp | |

In one embodiment, intrabodies are expressed in the cytoplasm. In other embodiments, the intrabodies are localized to various intracellular locations. In such embodiments, specific localization sequences can be attached to the intrabody polypeptide to direct the intrabody to a specific location. Intrabodies can be localized, for example, to the following intracellular locations: endoplasmic reticulum (Munro et al., 1987, Cell 48:899-907; Hangejorden et al., 1991, J. Biol. Chem. 266:6015); nucleus (Lanford et al., 1986, Cell 46:575; Stanton et al., 1986, PNAS 83:1772; Harlow et al., 1985, Mol. Cell. Biol. 5:1605; Pap et al., 2002, Exp. Cell Res. 265:288-93); nucleolar region (Seomi et al., 1990, J. Virology 64:1803; Kubota et al., 1989, Biochem. Biophys. Res. Comm. 162:963; Siomi et al., 1998, Cell 55:197); endosomal compartiment (Bakke et al., 1990, Cell 63:707-716); mitochondrial matrix (Pugsley, A. P., 1989, “Protein Targeting”, Academic Press, Inc.); Golgi apparatus (Tang et al., 1992, J. Bio. Chem. 267:10122-6); liposomes (Letourneur et al., 1992, Cell 69:1183); peroxisome (Pap et al., 2002, Exp. Cell Res. 265:288-93); trans Golgi network (Pap et al., 2002, Exp. Cell Res. 265:288-93); and plasma membrane (Marchildon et al., 1984, PNAS 81:7679-82; Henderson et al., 1987, PNAS 89:339-43; Rhee et al., 1987, J. Virol. 61:1045-53; Schultz et al., 1984, J. Virol. 133:431-7; Ootsuyama et al., 1985, Jpn. J. Can. Res. 76:1132-5; Ratner et al., 1985, Nature 313:277-84). Examples of localization signals include, but are not limited to, those sequences disclosed in Table 6.

TABLE 6

| | | |
|---|---|---|
| Localization | Sequence | SEQ ID NO. |

| | | |
|---|---|---|
| endoplasmic reticulum | Lys Asp Glu Leu | SEQ ID NO: 268 |
| endoplasmic reticulum | Asp Asp Glu Leu | SEQ ID NO: 269 |
| endoplasmic reticulum | Asp Glu Glu Leu | SEQ ID NO: 270 |
| endoplasmic reticulum | Gln Glu Asp Leu | SEQ ID NO: 271 |
| endoplasmic reticulum | Arg Asp Glu Leu | SEQ ID NO: 272 |
| Nucleus | Pro Lys Lys Lys Arg Lys Val | SEQ ID NO: 273 |
| Nucleus | Pro Gln Lys Lys Ile Lys Ser | SEQ ID NO: 274 |
| Nucleus | Gln Pro Lys Lys Pro | SEQ ID NO: 275 |
| Nucleus | Arg Lys Lys Arg | SEQ ID NO: 276 |
| Nucleus | Lys Lys Lys Arg Lys | SEQ ID NO: 277 |
| nucleolar region | Arg Lys Lys Arg Arg Gln Arg Arg Arg Ala | SEQ ID NO: 278 |
| | His Gln | |
| nucleolar region | Arg Gln Ala Arg Arg Asn Arg Arg Arg Arg | SEQ ID NO: 279 |
| | Trp Arg Glu Arg Gln Arg | |
| nucleolar region | Met Pro Leu Thr Arg Arg Arg Pro Ala Ala | SEQ ID NO: 280 |
| | Ser Gln Ala Leu Ala Pro Thr Pro | |
| endosomal compartment | Met Asp Asp Gln Arg Asp Leu Ile Ser Asn | SEQ ID NO: 281 |
| | Asn Glu Gln Leu Pro | |
| mitochondrial matrix | Met Leu Phe Asn Leu Arg Xaa Xaa Leu Asn | SEQ ID NO: 282 |
| | Asn Ala Ala Phe Arg His Gly His Asn Phe | |
| | Met Val Arg Asn Phe Arg Cys Gly Gln Pro | |
| | Leu Xaa | |
| Peroxisome | Ala Lys Leu | SEQ ID NO: 283 |
| trans Golgi network | Ser Asp Tyr Gln Arg Leu | SEQ ID NO: 284 |
| plasma membrane | Gly Cys Val Cys Ser Ser Asn Pro | SEQ ID NO: 285 |
| plasma membrane | Gly Gln Thr Val Thr Thr Pro Leu | SEQ ID NO: 286 |
| plasma membrane | Gly Gly Glu Leu Ser Gln His Glu | SEQ ID NO: 287 |
| plasma membrane | Gly Asn Ser Pro Ser Tyr Asn Pro | SEQ ID NO: 288 |
| plasma membrane | Gly Val Ser Gly Ser Lys Gly Gln | SEQ ID NO: 289 |
| plasma membrane | Gly Gln Thr Ile Thr Thr Pro Leu | SEQ ID NO: 290 |
| plasma membrane | Gly Gln Thr Leu Thr Thr Pro Leu | SEQ ID NO: 291 |
| plasma membrane | Gly Gln Ile Phe Ser Arg Ser Ala | SEQ ID NO: 292 |
| plasma membrane | Gly Gln Ile His Gly Leu Ser Pro | SEQ ID NO: 293 |
| plasma membrane | Gly Ala Arg Ala Ser Val Leu Ser | SEQ ID NO: 294 |
| plasma membrane | Gly Cys Thr Leu Ser Ala Glu Glu | SEQ ID NO: 295 |

VH and VL domains are made up of the immunoglobulin domains that generally have a conserved structural disulfide bond. In embodiments where the intrabodies are expressed in a reducing environment (e.g., the cytoplasm), such a structural feature cannot exist. Mutations can be made to the intrabody polypeptide sequence to compensate for the decreased stability of the immunoglobulin structure resulting from the absence of disulfide bond formation. In one embodiment, the VH and/or VL domains of the intrabodies contain one or more point mutations such that their expression is stabilized in reducing environments (see Steipe et al., 1994, J. Mol. Biol. 240:188-92; Wirtz and Steipe, 1999, Protein Science 8:2245-50; Ohage and Steipe, 1999, J. Mol. Biol. 291:1119-28; Ohage et al., 1999, J. Mol. Biol. 291:1129-34).

### 5.7.1 Polynucleotides Encoding an Antibody

The invention provides polynucleotides comprising a nucleotide sequence encoding an antibody (modified or unmodified) of the invention that immunospecifically binds to a RSV antigen (e.g., RSV F antigen). The invention also encompasses polynucleotides that hybridize under high stringency, intermediate or lower stringency hybridization conditions, e.g., as defined supra, to polynucleotides that encode a modified antibody of the invention.

The polynucleotides may be obtained, and the nucleotide sequence of the polynucleotides determined, by any method known in the art. Since the amino acid sequences of AFFF, P12f2, P12f4, P11d4, Ale9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), A4B4L1FR-S28R (MEDI-524), A4B4-F52S, A17d4(1), A3e2, A14a4, A16b4, A17b5, A17f5, or A17h4 are known (see, e.g., Table 2), nucleotide sequences encoding these antibodies and modified versions of these antibodies can be determined using methods well known in the art, i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, fragments, or variants thereof, annealing and ligating of those oligonucleotides, and then amplification of the ligated oligonucleotides by PCR.

Alternatively, a polynucleotide encoding an antibody of the invention may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library or a cDNA library generated from, or nucleic acid, preferably poly A+RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

### 5.7.2 Mutagenesis

Once the nucleotide sequence of the antibody is determined (see, e.g., Section 5.7.4 below), the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions. In certain embodiments, amino acid substitutions, deletions and/or insertions are introduced into the epitope-binding domain regions of the antibodies and/or into the hinge-Fc regions of the antibodies which are involved in the interaction with the FcRn. In a preferred embodiment, antibodies having one or more modifications in the hinge-Fc domain at one or more of amino acid residues 251-256, 285-290, 308-314, 385-389, and 428-436 are generated.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278:457-479 for a listing of human framework regions). Preferably, the polynucleotide sequence generated by the combination of the framework regions and CDRs encodes an antibody that immunospecifically binds to a particular antigen (e.g., an IL-9 polypeptide). Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

Mutagenesis may be performed in accordance with any of the techniques known in the art including, but not limited to, synthesizing an oligonucleotide having one or more modifications within the sequence of the constant domain of an antibody or a fragment thereof (e.g., the CH2 or CH3 domain) to be modified. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered. A number of such primers introducing a variety of different mutations at one or more positions may be used to generated a library of mutants.

The technique of site-specific mutagenesis is well known in the art, as exemplified by various publications (see, e.g., Kunkel et al., Methods Enzymol., 154:367-82, 1987, which is hereby incorporated by reference in its entirety). In general, site-directed mutagenesis is performed by first obtaining a single-stranded vector or melting apart of two strands of a double stranded vector which includes within its sequence a DNA sequence which encodes the desired peptide. An oligonucleotide primer bearing the desired mutated sequence is prepared, generally synthetically. This primer is then annealed with the single-stranded vector, and subjected to DNA polymerizing enzymes such as T7 DNA polymerase, in order to complete the synthesis of the mutation-bearing strand. Thus, a heteroduplex is formed wherein one strand encodes the original non-mutated sequence and the second strand bears the desired mutation. This heteroduplex vector is then used to transform or transfect appropriate cells, such as E. coli cells, and clones are selected which include recombinant vectors bearing the mutated sequence arrangement. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art. Double stranded plasmids are also routinely employed in site directed mutagenesis which eliminates the step of transferring the gene of interest from a plasmid to a phage.

Alternatively, the use of PCR™ with commercially available thermostable enzymes such as Taq DNA polymerase may be used to incorporate a mutagenic oligonucleotide primer into an amplified DNA fragment that can then be cloned into an appropriate cloning or expression vector. See, e.g., Tomic et al., Nucleic Acids Res., 18(6):1656, 1987, and Upender et al., Biotechniques, 18(1):29-30, 32, 1995, for PCR™-mediated mutagenesis procedures, which are hereby incorporated in their entireties. PCR™ employing a thermostable ligase in addition to a thermostable polymerase may also be used to incorporate a phosphorylated mutagenic oligonucleotide into an amplified DNA fragment that may then be cloned into an appropriate cloning or expression vector (see e.g., Michael, Biotechniques, 16(3):410-2, 1994, which is hereby incorporated by reference in its entirety).

Other methods known to those of skill in art of producing sequence variants of the Fc domain of an antibody or a fragment thereof can be used. For example, recombinant vectors encoding the amino acid sequence of the constant domain of an antibody or a fragment thereof may be treated with mutagenic agents, such as hydroxylamine, to obtain sequence variants.

### 5.7.3 Panning

Vectors, in particular, phage, expressing constant domains or fragments thereof having one or more modifications in amino acid residues 251-256, 285-290, 308-314, 385-389, and/or 428-436 can be screened to identify constant domains or fragments thereof having increased affinity for FcRn to select out the highest affinity binders from a population of phage. Immunoassays which can be used to analyze binding of the constant domain or fragment thereof having one or more modifications in amino acid residues 251-256, 285-290, 308-314, 385-389, and/or 428-436 to the FcRn include, but are not limited to, radioimmunoassays, ELISA (enzyme linked immunosorbent assay), “sandwich” immunoassays, and fluorescent immunoassays. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly herein below (but are not intended by way of limitation). BIAcore kinetic analysis can also be used to determine the binding on and off rates of a constant domain or a fragment thereof having one or more modifications in amino acid residues 251-256, 285-290, 308-314, 385-389, and/or 428-436 to the FcRn. BIAcore kinetic analysis comprises analyzing the binding and dissociation of a constant domain or a fragment thereof having one or more modifications in amino acid residues 251-256, 285-290, 308-314, 385-389, and/or 428-436 from chips with immobilized FcRn on their surface (see Sections 5.1 and 6 herein).

### 5.7.4 Sequencing

Any of a variety of sequencing reactions known in the art can be used to directly sequence the nucleotide sequence encoding, e.g., variable regions and/or constant domains or fragments thereof having one or more modifications in amino acid residues 251-256, 285-290, 308-314, 385-389, and/or 428-436. Examples of sequencing reactions include those based on techniques developed by Maxim and Gilbert (Proc. Natl. Acad. Sci. USA, 74:560, 1977) or Sanger (Proc. Natl. Acad. Sci. USA, 74:5463, 1977). It is also contemplated that any of a variety of automated sequencing procedures can be utilized (Bio/Techniques, 19:448, 1995), including sequencing by mass spectrometry (see, e.g., PCT Publication No. WO 94/16101, Cohen et al., Adv. Chromatogr., 36:127-162, 1996, and Griffin et al., Appl. Biochem. Biotechnol., 38:147-159, 1993).

### 5.7.5 Recombinant Expression of an Antibody

Recombinant expression of an antibody of the invention (e.g., a heavy or light chain of an antibody of the invention or a single chain antibody of the invention) that immunospecifically binds to a RSV antigen (e.g., RSV F antigen) requires construction of an expression vector containing a polynucleotide that encodes the antibody. Once a polynucleotide encoding an antibody molecule, heavy or light chain of an antibody, or fragment thereof (preferably, but not necessarily, containing the heavy and/or light chain variable domain) of the invention has been obtained, the vector for the production of the antibody molecule may be produced by recombinant DNA technology using techniques well-known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a fragment thereof, or a heavy or light chain CDR, operably linked to a promoter. Such vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or fragment thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies of the invention which immunospecifically bind to a RSV antigen (preferably, RSV F antigen) is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2197-2199). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention may be fused to heterologous polypeptide sequences described herein or otherwise known in the art to facilitate purification.

### 5.8 Kits

The invention also provides a pharmaceutical pack or kit comprising one or more containers filled with one or more of the ingredients of the pharmaceutical compositions of the invention, such as one or more modified or unmodified antibodies provided herein. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises an antibody of the invention, preferably a purified antibody, in one or more containers. In a specific embodiment, the kits of the present invention contain a substantially isolated RSV antigen as a control. Preferably, the kits of the present invention further comprise a control antibody which does not react with the RSV antigen. In another specific embodiment, the kits of the present invention contain a means for detecting the binding of a modified antibody to a RSV antigen (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate). In specific embodiments, the kit may include a recombinantly produced or chemically synthesized RSV antigen. The RSV antigen provided in the kit may also be attached to a solid support. In a more specific embodiment the detecting means of the above described kit includes a solid support to which RSV antigen is attached. Such a kit may also include a non-attached reporter-labeled anti-human antibody. In this embodiment, binding of the antibody to the RSV antigen can be detected by binding of the said reporter-labeled antibody.

### 6. EXAMPLES

### 6.1 Example

### Kinetic Analysis of Humanized RSV mAbs by BIACORE™

A typical kinetic study involved the injection of 250 μl of monoclonal antibody (“mAb”) at varying concentrations (25-300 nM) in PBS buffer containing 0.05% Tween-20 (PBS/Tween). The flow rate was maintained at 75 μl/min, giving a 15 minute dissociation time. Following the injection of mAb, the flow was exchanged with PBS/Tween buffer for 30 min for determining the rate of dissociation. The sensor chip was regenerated between cycles with a 1 min pulse of 100 mM HCl. The regeneration step caused a minimal loss of binding capacity of the immobilized F-protein (4% loss per cycle). This small decrease did not change the calculated values of the rate constants for binding and dissociation (also called the kₒₙ and k_{off}, respectively).

More specifically, for measurement of k_{assoc} (or kₒₙ), F protein was directly immobilized by the EDC/NHS method (EDC=N-ethyl-N′-[3-diethylaminopropyl)-carbodimide). Briefly, 25 μg/ml of F protein in 10 mM NaoAc, pH 5.0 was prepared and about a 5-10 μl injection gives about 30-50 RU (response units) of immobilized F protein under the above referenced conditions. The blank was subtracted for kinetic analysis. The column could be regenerated using 100 mM HCl (with 60 seconds of contact time being required for full regeneration). This treatment removed bound Fab completely without damaging the immobilized antigen and could be used for over 40 regenerations. For kₒₙ measurements, Fab concentrations were 0.39 nM, 0.75 nM, 1.56 nM, 3.13 nM, 12.5 nM, 25 nM, 50 nM, and 100 nM. The dissociation phase was analyzed for approximately 900 seconds. Kinetics were analyzed by 1:1 Langmuir fitting (global fitting). Measurements were done in HBS-EP buffer (10 mM HEPES, pH 7.4, 150 mM NaCl, 3 mM EDTA, 0.005% (v/v) Surfactant P20.

For measurements of combinatorial clones, as disclosed herein, the kₒₙ and k_{off} were measured separately. The kₒₙ was measured at conditions that were the same as those for the single mutation clones and was analyzed similarly.

For measuring k_{off}, the following conditions were employed. Briefly, 4100 RU of F protein were immobilized (as above) with CM-dextran used as the blank. Here, 3000 RU of Fab was bound (with dissociated Fab high enough to offset machine fluctuation). HBS plus 5 nM F protein (about 350-2000 times higher than the K_{d}—the dissociation equilibrium constant) was used as buffer. The dissociation phase was 6-15 hours at a flow rate of 5 μl/min. Under the conditions used herein, re-binding of the dissociated Fab was minimal. For further details, see the manual with the biosensor.

The binding of the high affinity anti-RSV antibodies to the F protein, or other epitopic sites on RSV, disclosed herein was calculated from the ratio of the first order rate constant for dissociation to the second order rate constant for binding or association (K_{d}=k_{off}/kₒₙ). The value for kₒₙ was calculated based on the following rate equation:

dR/dt=kₒₙ[mAb]Rₘₐₓ−(kₒₙ[mAb]+k_{off})R

where R and Rₘₐₓ are the response units at time t and infinity, respectively. A plot of dr/dt as a function of R gives a slope of (kₒₙ [mAb]+/k_{off})—since these slopes are linearly related to the [mAb], the value kₒₙ can be derived from a replot of the slopes versus [mAb]. The slope of the new line is equal to kₒₙ. Although the value of k_{off} can be extrapolated from the Y-intercept, a more accurate value was determined by direct measurement of k_{off}. Following the injection phase of the mAb, PBS/Tween buffer flows across the sensor chip. From this point, [mAb]=0. The above stated equation for dR/dt thus reduces to:

dr/dt=k or dR/R=k_{off}dt

Integration of this equation then gives:

In(R₀/Rₜ)=k_{off}t

where R₀/Rₜ) are the response units at time 0 (start of dissociation phase) and t, respectively. Lastly, plotting In(R₀/R_{E}) as a function of t gives a slope of k_{off}.

The numerical values from such antibody variants were as shown in Tables 7-10 below.

TABLE 7
Summary of Kinetic Constants for High Potency Antibodies

| | | | IC₅₀ |
|---|---|---|---|
| ANTIBODY | kₒₙ × 10⁵ (M⁻¹s⁻¹) | k_{off} × 10⁻⁴ (s⁻¹) | (nM) |
| **palivizumab | 2.04; 1.89; 2.18 | 7.64; 7.38; 7.02 | 3.57 |
| **AFFF(1) | 1.08; 0.96; 1.24 | 2.74; 2.66; 2.06 | |
| *1X-493L1FR | 1.85 | 6.5 | |
| *H3-3F4 | 4.59; 4.67; 5.72; 6.25; 5.33 | 4.45; 4.02 | |
| *M3H | 6.05 | 3.38 | |
| *Y10H6 | 7.57 | 4.62 | |
| *DG | 2.65; 2.83; 4.16; 3.18; 2.88 | 1.67; 4.44 | |
| *AFFF(1) | 2.12; 1.56; 1.86 | 2.45; 4.46; 2.68 | |
| *6H8 | 3.14; 4.44 | 1.78; 4.73 | |
| *L1-7E5 | 3.29; 3.57; 4.05; 3.35; 4.26 | 1.92; 3.31; 2.29 | |
| *L2-15B10 | 3.69; 2.82; 3.12; 5.33; 3.78 | 1.34; 4.16; 2.70 | |
| *P12f2 | 6.63 | 2.82 | 0.65 |
| *P12f4 | 5.27 | 2.99 | 0.70 |
| *P11d4 | 5.70; 5.72 | 7.17 | >20 |
| *A1e9 | 7.9 | 4.53 | 2.5 |
| *A12a6 | 7.43 | 2.30 | 0.62 |
| *A13a11 | 7.35 | 2.50 | 2.04 |
| *A13c4 | 7.81; 7.35 | 2.80 | 0.52 |

TABLE 8
Monoclonal Antibodies vs. Bac-F (1:1)

| | kon (×E+5) | koff (×E−5) | K_{D} (nM) | Chi2 |
|---|---|---|---|---|
| P12f2 | 4.07 | 12.8 | 0.31 (13) | 0.9 |
| P12f4 | 4.95 | 5.55 | 0.11 (35) | 0.6 |
| A13c4 | 3.00 | 3.96 | 0.13 (30) | 1.2 |
| A12a6 | 4.60 | 1.65 | 0.04 (98) | 1.2 |
| A1e9 | 4.33 | 14.3 | 0.33 (12) | 2.5 |
| A8c7 | 4.17 | 8.75 | 0.21 (19) | 1.8 |
| P11d4 | 4.66 | 28.9 | 0.62 (6) | 1.0 |
| A17d4(1) | 4.56 | 4.07 | 0.09 (43) | 0.5 |
| A4B4 | 4.34 | 1.06 | 0.02 (195) | 1.5 |
| Palivizumab | 1.32 | 51.5 | 3.90 (1) | 0.6 |

TABLE 9
Monoclonal Antibodies vs. NUF4 (1:1)

| | kon (×E+5) | koff (×E−5) | K_{D} (nM) | Chi2 |
|---|---|---|---|---|
| P12f2 | 5.41 | 17.8 | 0.33 (26) | 1.2 |
| P12f4 | 9.43 | 22.9 | 0.24 (36) | 0.9 |
| A13c4 | 3.65 | 27.2 | 0.75 (12) | 1.8 |
| A12a6 | 4.00 | 29.1 | 0.73 (12) | 1.9 |
| A1e9 | 8.43 | 58.4 | 0.69 (13) | 0.9 |
| A8c7 | 8.25 | 53.5 | 0.65 (13) | 0.7 |
| P11d4 | 9.04 | 76.6 | 0.85 (10) | 2.5 |
| A17d4(1) | 4.99 | 36.2 | 0.73 (12) | 2.0 |
| A4B4 | 4.96 | 28.2 | 0.57 (15) | 1.9 |
| Palivizumab | 3.04 | 265 | 8.70 (1) | 0.4 |

TABLE 10
Monoclonal Antibodies vs. NUF4 (2:1)

| | kon (×E+5) | koff (×E−5) | K_{D} (nM) | Chi2 |
|---|---|---|---|---|
| P12f2 | 2.82 | 23.6 | 0.84 (371) | 1.5 |
| P12f4 | 2.73 | 63.6 | 2.33 (134) | 4.9 |
| A13c4 | 3.20 | 22.5 | 0.70 (446) | 1.7 |
| A12a6 | 2.18 | 40.8 | 1.87 (167) | 1.9 |
| A1e9 | 3.29 | 139 | 4.22 (74) | 2.8 |
| A8c7 | 4.30 | 114 | 2.65 (118) | 2.0 |
| P11d4 | 3.66 | 313 | 8.55 (36) | 3.6 |
| A17d4(1) | 2.64 | 29.2 | 1.11 (281) | 1.7 |
| A4B4 | 2.03 | 40.06 | 2.00 (156) | 1.4 |
| Palivizumab | 0.78 | 2420 | 312 (1) | 1.3 |

The bold and underlined amino acid residues of the indicated CDRs in Table 1 represent the amino acid residues located at the key locations within the CDRs of the high potency antibodies produced by the methods described herein and in copending applications Ser. Nos. 60/168,426 and 60/186,252. For example, to increase the potency of an antibody by producing a higher kₒₙ value, the amino acids located at the key positions as taught herein by the bold and underlined residues in Table 1 for the reference antibody would be replaced by the amino acids listed under CDRs in Table 2 and/or Table 3. Thus, these one letter codes represent the amino acids replacing the reference amino acids at the key positions (or critical positions) of the CDRs shown in FIG. 2 (residues in bold in the sequences of Table 2) for a reference antibody whose potency is to be increased.

### 6.2 Kinetic Analysis of Binding of A4B4L1FR-S28R (MEDI-524) by BIACORE™

The kinetics of the interactions of A4B4L1FR-S28R (MEDI-524) and palivizumab with RSV F-protein were determined by surface plasmon resonance (see, e.g., Jonsson et al., 1991, Biotechniques 11(5):620-627 and Johne, B. (1989). Epitope mapping by surface plasmon resonance in the BIAcore. Molecular Biotechnology 9(1):65-71) using a BIAcore 3000 instrument (BIAcore, Inc., Piscataway, N.J.). A recombinantly produced, C-terminally truncated RSV (A2 strain) F protein (Wathen et al., 1989, J Infect Dis 159(2):255-264) was used as the antigen for these studies. The truncated F protein, lacking the membrane anchor, was produced as a secreted product using a recombinant baculovirus expression system and was purified by successive chromatography steps on concanavalin-A and Q-sepharose columns. Purified F protein was covalently coupled to an N-hydroxysuccinimide-N-ethyl-N′-[3-diethylaminopropyl]-carbodiimide (EDC/NHS) activated CM5 sensor chip at a low protein density according to the manufacturer's protocol; unreacted active ester groups were blocked with 1 M ethanolamine. For reference purposes, a blank surface, containing no antigen, was prepared under identical immobilization conditions.

For kinetic measurements, a serial 2-fold dilution series of each mAb from 100 nm-0.2 nm, made in instrument buffer (HBS/Tween-20, BIAcore, Inc.), was injected over the F-protein and reference cell surfaces, which are connected in series. In each analysis, following the dissociation phase, the remaining bound antibody was removed from the sensor chip by passing a brief pulse of 100 mM HCl over the surface. Once an entire data set was collected, the resulting binding curves were globally fitted to a 1:1 Langmuir binding model using BIAevaluation software (BIAcore, Inc., Piscataway, N.J.). This algorithm calculates both the association rate (kₒₙ) and the dissociation rate (k_{off}), from which the apparent equilibrium binding constant, K_{d}, for each antibody was deduced as the ratio of the two rate constants, k_{off}/kₒₙ. A more detailed explanation of how the individual rate constants are derived can be found in the BIAevaluation Software Handbook (BIAcore, Inc., Piscataway, N.J.).

Kinetic analysis of binding by BIAcore evaluation (Table 11) revealed that, under the conditions of a low-density surface that were employed, A4B4L1FR-S28R (MEDI-524) had an approximately 70-fold greater affinity for RSV F protein than palivizumab. The increased affinity of MEDI-524 for the RSV F protein is attributed to a 4-fold increase in the association rate and an approximately 17-fold decrease in the dissociation rate. Since the rate at which MEDI-524 dissociates from the F protein surface approaches the detection limits of the BIAcore 3000 instrument, the dissociation rate generated for MEDI-524 is an estimation.

TABLE 11
Kinetic Analysis of Binding

| mAb | kₒₙ (M⁻¹s⁻¹) | k_{off} (s⁻¹) | K_{d} (pM) |
|---|---|---|---|
| palivizumab | 1.14E+05 | 3.95E−04 | 3460 |
| MEDI-524 | 4.73E+05 | 2.35E−05 | 50 |

### 6.3 Example

### Microneutralization Assay

Neutralization of the antibodies of the present invention were determined by microneutralization assay. This microneutralization assay is a modification of the procedures described by Anderson et al. (1985, J. Clin. Microbiol. 22:1050-1052, the disclosure of which is hereby incorporated by reference in its entirety). The procedure used here is described in Johnson et al., 1999, J. Infectious Diseases 180:35-40, the disclosure of which is hereby incorporated by reference in its entirety. Antibody dilutions were made in triplicate using a 96-well plate. Ten TCID₅₀ of respiratory syncytial virus (RSV—Long strain) were incubated with serial dilutions of the antibody (or Fabs) to be tested for 2 hours at 37° C. in the wells of a 96-well plate. RSV susceptible HEp-2 cells (2.5×10⁴) were then added to each well and cultured for 5 days at 37° C. in 5% CO₂. After 5 days, the medium was aspirated and cells were washed and fixed to the plates with 80% methanol and 20% PBS. RSV replication was then determined by F protein expression. Fixed cells were incubated with a biotin-conjugated anti-F protein monoclonal antibody (pan F protein, C-site-specific mAb 133-1H) washed and horseradish peroxidase conjugated avidin was added to the wells. The wells were washed again and turnover of substrate TMB (3,3′,5,5′-tetramethylbenzidine) was measured at 450 nm. The neutralizing titer was expressed as the antibody concentration that caused at least 50% reduction in absorbency at 450 nm (the OD₄₅₀) from virus-only control cells. The results from the assay for the monoclonal antibodies and Fab fragments listed in Table 2 are shown in Table 11, supra, and Table 12,

TABLE 12
End Point RSV Microneutralization Titer Of High On Rate Mutant IgG
and Fab

| | Mean | | Fold | Mean | | Fold | |
|---|---|---|---|---|---|---|---|
| | IC50 | STDEV | Difference | IC50 | STDEV | Difference | n |
| | (Curve) | Curve | (Curve | (Control) | Control | (Control | (assay |
| Molecule | μg/ml | IC50 | IC50) | μg/ml | IC50 | IC50) | repeat) |
| **palivizumab | 0.4527 | 0.208 | — | 0.5351 | 0.238 | — | 8 |
| **A1e9 | 0.0625 | 0.0268 | 7 | 0.0645 | 0.223 | 8 | 3 |
| **A17d4(1) | 0.0342 | 0.022 | 13 | 0.0354 | 0.0187 | 15 | 4 |
| **P11d4 | 0.0217 | 0.0331 | 21 | 0.0289 | 0.0110 | 19 | 5 |
| **P12f2 | 0.0231 | 0.0141 | 20 | 0.0223 | 0.0083 | 24 | 6 |
| **A8c7 | 0.0337 | 0.0309 | 13 | 0.0383 | 0.0283 | 14 | 5 |
| **A12a6 | 0.0357 | 0.0316 | 13 | 0.0354 | 0.0261 | 15 | 7 |
| **P12f4 | 0.0242 | 0.0163 | 19 | 0.0235 | 0.0076 | 23 | 7 |
| *A13c4 | 0.0376 | 0.0268 | 12 | 0.0375 | 0.0213 | 14 | 6 |
| **A4B4 | 0.0171 | 0.0018 | 27 | 0.0154 | 0.00417 | 35 | 2 |
| *A1e9 | 0.157 | — | 3 | 0.125 | — | 4 | 1 |
| *A17d4(1) | 0.0179 | — | 25 | 0.0171 | — | 31 | 1 |
| *P11d4 | >1.00 | — | — | >1.00 | — | — | 1 |
| *P12f2 | 0.0407 | 0.0112 | 11 | 0.0326 | 0.00905 | 16 | 2 |
| *A8c7 | 0.177 | — | 3 | 0.157 | — | 34 | 1 |
| *A12a6 | 0.0287 | 0.00417 | 16 | 0.0310 | 0.00982 | 17 | 2 |
| *P12f4 | 0.0464 | 0.00791 | 10 | 0.0351 | 0.0126 | 15 | 2 |
| *A13c4 | 0.0264 | 0.00141 | 17 | 0.0258 | 0.00071 | 21 | 2 |
| *A4B4 | 0.0414 | — | 11 | 0.0411 | — | 13 | 1 |
| *A13a11 | 0.120 | 0.0222 | 4 | 0.1022 | 0.0260 | 5 | 2 |
| *A1h5 | 0.194 | 0.462 | 2 | 0.176 | 0.0625 | 3 | 2 |

**Monoclonal Antibody
*Fab Fragment

### 6.4 RSV Microneutralization Assay

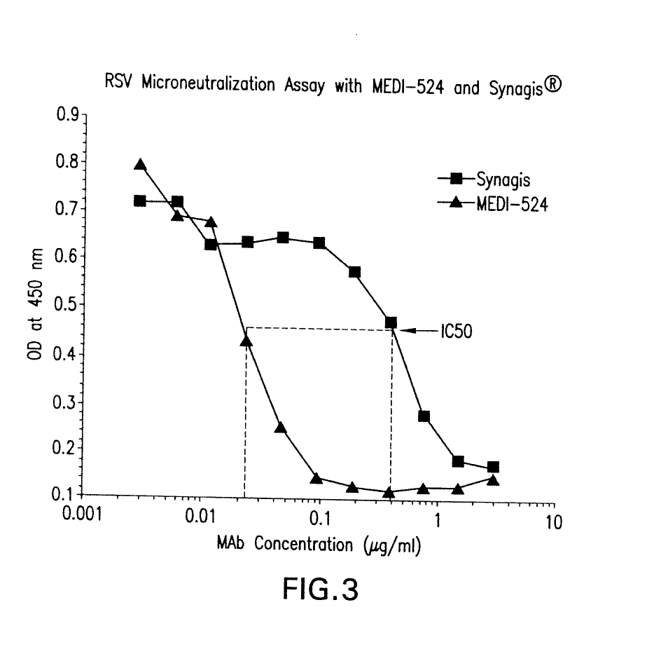

The ability of A4B4L1FR-S28R (MEDI-524) and palivizumab to inhibit the in vitro replication of RSV (Long strain) was evaluated using a RSV microneutralization assay. This assay is a modification of the procedure of Anderson et al. (Anderson et al., 1985, J Clin Microbiol 22: 1050-1052) as described by Johnson et al. (Johnson et al., 1997, J Infect Dis 176: 1215-1224). Antibody dilutions were made in duplicate to quadruplicate wells of a 96-well plate. Approximately 100-1000 TCID₅₀ of RSV (Long) were added to each dilution well and incubated for two hours at 37° C. Low passage, RSV susceptible HEp-2 cells (2.5×10⁴) were then added to each well and cultured for five days at 37° C. in a humidified 5% CO₂ incubator. After four or five days the cells were washed with PBS −0.1% Tween 20 and fixed to the plate with 80% acetone with 20% PBS. RSV replication was determined by quantitation of F protein expression using an F protein-specific ELISA. Fixed cells were incubated with the C-site specific, pa RSV F protein mAb 133-1H (Chemicon, Inc.), washed, and then incubated with horseradish peroxidase-conjugated goat anti-mouse IgG and washed again. The peroxidase substrate TMB (3,3′,5,5′-tetramethylbenzidine) was added to each well and the reaction was stopped after twenty minutes by the addition of 2 M H₂SO₄. Substrate turnover was measured at 450 nm (OD450) using a microplate reader. The neutralizing titer is expressed as the antibody concentration resulting in at least a 50% reduction in the OD450 value from control wells with virus only (IC₅₀). The results of this assay, shown in FIG. 3, indicate that MEDI-524 (average IC₅₀=18 ng/ml) is approximately 18-fold more potent than palivizumab (average IC₅₀=315 ng/ml).

### 6.5 RSV Microneutralization Assay with Cynomolgus BAL Samples

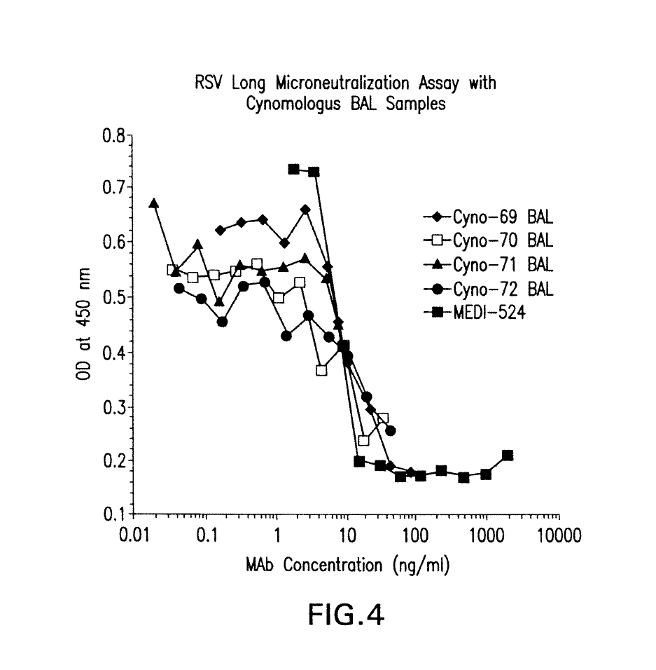

The ability of MEDI-524 present in the lungs of treated animals to inhibit the in vitro replication of RSV was evaluated using the RSV microneutralization assay. Four juvenile female cynomolgus monkeys (average weight 2.0 kg) were sedated with Telazol and dosed intravenously (i.v.) with MEDI-524 at 30 mg/kg body weight via the saphenous vein using an external infusion pump. Four days later, the animals were anesthetized with Telazol and a bronchial alveolar lavage (BAL) was performed on one lobe of the right lung with phosphate buffered saline (PBS). Titers of MEDI-524 in the BAL fluid were determined using a MEDI-524-specific ELISA. The BAL samples were tested undiluted and at serial 2-fold dilutions in the RSV microneutralization assay as above with purified MEDI-524 included as a control. The results of this assay, shown in FIG. 4, show that MEDI-524 retains full RSV neutralizing activity in the lungs of cynomolgus monkeys four days after infusion.

### 6.6 RSV Fusion Inhibition Assay

The ability of the antibodies of the invention to block RSV-induced fusion after viral attachment to the cells is determined in a fusion inhibition assay. This assay is identical to the microneutralization assay, except that the cells are infected with RSV (Long) for four hours prior to addition of antibody (Taylor et al., 1992, J. Gen. Virol. 73:2217-2223).

### 6.7 Isothermal Titration Calorimetry

Thermodynamic binding affinities and enthalpies were determined from isothermal titration calorimetry (ITC) measurements on the interaction of antibodies with RSV F glycoprotein (NUF4), an antigen which mimics the binding site of the RSV virus.

### Methods & Materials

### Antibodies & Antigen

A13c4, A17d4(1), A4B4, and palivizumab were diluted in dialysate and the concentrations were determined by UV spectroscopic absorption measurements with a Perkin-Elmer Lambda 4B Spectrophotometer using an extinction coefficient of 217,000 M cm⁻¹ at the peak maximum at 280 nm. The diluted NUF4 concentrations were calculated from the ratio of the mass of the original sample to that of the diluted sample since its extinction coefficient was too low to determine an accurate concentration without employing and losing a large amount of sample.

### ITC Measurements

The binding thermodynamics of the antibodies were determined from ITC measurements using a Microcal, Inc. VP Titration Calorimeter. The VP titration calorimeter consists of a matched pair of sample and reference vessels (1.409 ml) enclosed in an adiabatic enclosure and a rotating stirrer-syringe for titrating ligand solutions into the sample vessel. The ITC measurements were performed at 25° C. and 35° C. The sample vessel contained the antibody in the phosphate buffer while the reference vessel contained just the buffer solution. The phosphate buffer solution was saline 67 mM PO₄ at pH 7.4 from HyClone, Inc. Five or ten μl aliquots of the 0.05 to 0.1 mM NUF4 solution were titrated 3 to 4 minutes apart into the antibody sample solution until the binding was saturated as evident by the lack of a heat exchange signal. With some antibody sample solutions, additional constant amounts of heat with the addition of each aliquot were observed following binding saturation of the antibody. This was attributed to a heat of dilution of the NUF4 titrant and was subtracted from the titrant heats obtained during the titration prior to analysis of the data.

A non-linear, least square minimization software program from Microcal, Inc., Origin 5.0, was used to fit the incremental heat of the ith titration (ΔQ (i)) of the total heat, Qₜ, to the total titrant concentration, Xₜ, according to the following equations (I),

Qₜ=nCₜΔHb° V{1+Xₜ/nCₜ+1/nK_{b}Cₜ−[(1+Xₜ/nCₜ+1/nK_{b}Cₜ)²−4Xₜ/nCₜ]^{1/2}}/2 (1a)

ΔQ(i)=Q(i)+dVi/2V{Q(i)+Q(i−1)}−Q(i−1) (1b)

where Cₜ is the initial antibody concentration in the sample vessel, V is the volume of the sample vessel, and n is the stoichiometry of the binding reaction, to yield values of K_{b}, ΔH_{b}°, and n. The optimum range of sample concentrations for the determination of K_{b} depends on the value of K_{b} and is defined by the following relationship.

CₜK_{b}n>500 (2)

so that at 1 μM the maximum K_{b} that can be determined is less than 2.5×10⁸ M⁻¹. If the first titrant addition did not fit the binding isotherm, it was neglected in the final analysis since it may reflect release of an air bubble at the syringe opening-solution interface.

### Results

The ITC results are summarized in Table 13. The higher than 2 stoichiometries in Table 9 indicate that either the concentration determination of the antibody or NUF4 was incorrect. Since the same NUF4 sample was used as a titrant with antibodies having the amino acid sequence of A13c4 at 35° C. and A17d4(1) at 35° C., which exhibit in at least one of the titrations the correct stoichiometry of 2, it is assumed that the titrant concentration was correct and that the large values of n result from incorrectly determined antibody concentrations. However, it can be shown that the binding constants are critically dependent on the titrant concentration and, thus, despite the 2-3 disparity in n, the binding constants are correct. Since the binding constants of antibodies having the amino acid sequence of A4B4 and A13c4 at 25° C. were near the upper determination limit by ITC (equation 2) and with the limited amount of available NUF4, it was decided to use 35° C. as the reference temperature for comprising the binding affinities. The results summarized in Table 13 show that the binding affinities to NUF4 are in the order A4B4>A13c4>A17d4(1)>palivizumab.

TABLE 13
Average Binding Constants and Enthalpies of NUF4 to Antibodies

| Antibody | K_{b} | ΔH_{b} in kJ mol⁻¹ |
|---|---|---|
| A4B4 | 269 ± 74 × 10⁶ M⁻¹ or ~3.7 nM* | 92.8 ± 1.0 |
| A13c4 | 107 ± 28 × 10⁶ M⁻¹ or 9 nM | 67 ± 17 |
| A17d4(1) | 75 ± 14 × 10⁶ M⁻¹ or 13 nM | 68 ± 10 |
| palivizumab | 1.23 ± 0.17 × 10⁶ M⁻¹ or 810 nM | 71 ± 5 |

*Based only on the best titration run at 35° C.; 4.0 nM is ITC lower limit of 1/K_{b} range (ITC range is limited to [antibody]ₙ K_{b} = 500 where n is the stoichiometry and [antibody] is the concentration of the antibody in the cell).

### 6.8 Example

### Ultra-Potent Anti-RSV Antibodies

It is noted that the information in this Example further characterizes some of the antibodies presented in prior Examples, describes the production of some of those antibodies, and may include preliminary or additional results for certain assays for certain antibodies.

In this Example, increasing the affinity to RSV F protein by reducing antibody k_{off} translated very well into higher RSV neutralization ability for Fab fragments. Raising the affinity by increasing kₒₙ resulted in a great improvement in virus neutralization for both Fab and IgG forms. Additionally, bivalent binding to F protein, in either the IgG or F(ab′)₂ format, confers a substantial benefit in viral neutralization over monovalent binding by Fab.

### Materials and Methods

### F Protein

The extracellular domain of the F protein from RSV A2 was expressed by a baculovirus expression system (Wathen et al. (1989) J. Infect. Dis. 159, 255-264) and was purified by an antibody-based affinity column chromatography using a C-site specific, anti-RSV F protein, murine monoclonal antibody, 1331H (Beeler et al. (1989) J. Virol. 63, 2941-2950).

Cloning of Palivizumab V Region into Phage Vector

The palivizumab (palivizumab) V region was cloned into a phage expression vector, M13IX104CS, containing human CH1 and kappa constant regions, according to the method described (Wu et al. (1999) J. Mol. Biol. 294, 151-162; Kunkel et al. (1985) Proc. Natl. Acad. Sci. USA, 82, 488-492). Appropriate reverse primers and biotinylated forward primers were used to amplify palivizumab V_{H} and kappa light chain variable region (V_{K}) from a plasmid. PCR products were purified by agarose gel electrophoresis, electroeluted, and phosphorylated by T4 polynucleotide kinase (Roche). The minus single-stranded DNA encoding V_{H} or V_{K} was isolated by dissociation of the double-stranded PCR product with sodium hydroxide while the plus biotinylated strand was captured by streptavidin-coated magnetic beads. The isolated V_{H} and V_{K} single-stranded DNA were annealed to the uridinylated M13IX104CS template, and T4 DNA polymerase (Roche), T4 DNA ligase (Roche), and concentrated synthesis buffer were added to the annealed product. The synthesized DNA was electroporated into DH10B cells and titered on an XL-1 Blue lawn. Several independent plaques were isolated, and phage DNA was prepared and sequenced to confirm cloning. The resulting phage DNA encoding palivizumab Fab was termed IX-493.

### Modification of Framework 4 and Light Chain CDR1 Regions of Palivizumab

Several modifications were made to the palivizumab V region by site-directed mutagenesis (Kunkel et al. (1985) Proc. Natl. Acad. Sci. USA, 82, 488-492) prior to affinity maturation. Three oligonucleotides were synthesized, phosphorylated and annealed to the uridinylated IX-493 template to introduce mutations from K₂₄C₂₅Q₂₆L₂₇ to S₂₄A₂₅S₂₆S₂₇ in the LCDR1, L104 to V in the light chain FR4, and A105 to Q in the heavy chain FR4. For numbering used herein, please refer to Kabat et al. (1991) Sequences of proteins of immunological interest. (U.S. Department of Health and Human Services, Washington, D.C.) 5^{th} ed. The mutagenesis reaction was completed by adding DNA polymerase, DNA ligase, and synthesis buffer, and was electroporated into DH10B and titered on a lawn of XL-1 Blue. Many clones were screened by DNA sequencing, and the clone with all the desired mutations was termed 493L1FR. This clone was used as the template for the affinity maturation.

### Construction of Focused CDR Libraries and Combinatorial Libraries

Six CDR libraries encoding single modifications at each CDR position were constructed in M13IX104CS vector simultaneously according to the method described (Wu et al. (1998) Proc. Natl. Acad. Sci. USA, 95, 6037-6042; Glaser at al. (1992) J. Immunol. 149, 3903-3913; Wu and An (2003) Tailoring kinetics of antibodies using focused combinatorial libraries. In Methods in Molecular Biology, vol. 207: Recombinant Antibodies for Cancer Therapy: Methods and Protocols (Welschof, M. & Krauss, J., eds), pp. 213-233, Humana Press, Totowa, N.J.). The CDR regions were defined as indicated in Kabat et al. (1991) Sequences of proteins of immunological interest. (U.S. Department of Health and Human Services, Washington, D.C.) 5^{th} ed. Prior to library construction, each individual CDR was deleted by site-directed mutagenesis (Kunkel et al. (1985) Proc. Natl. Acad. Sci. USA, 82, 488-492) to avoid the contamination of the library by the parental clone. Mutagenized oligonucleotides were designed to replace individual CDR regions with a TAA stop codon and an extra nucleotide, A, to cause a frameshift. The resulting clone was used as a template for the construction of its corresponding CDR library. Oligonucleotides encoding single mutations were synthesized by introducing NNK at each CDR position as described (Glaser et al. (1992) J. Immunol. 149, 3903-3913) and were used in the mutagenesis reaction for the library constructions. The constructed libraries were electroporated into DH10B and plated onto XL-1Blue lawns for characterization and screening. The quality of the library was examined by plaque lift assay for Fab expression, and by DNA sequencing for the distribution of incorporated mutations. Appropriate distribution of mutations in each library was confirmed. Separate focused CDR libraries were constructed for the optimization of k_{off} and kₒₙ using 493L1FR and D95/G93 as a template respectively. D95/G93 clone was derived from mutagenesis of both HCDR3 and LCDR3 of one of the best k_{off}-optimized variants, AFFF(1). The mutation S95D on HCDR3 and F93G on LCDR3 moderately enhanced the kₒₙ of AFFF(1).

Combinatorial libraries were constructed to incorporate all beneficial mutations from each CDR. For optimization of k_{off}, degenerate oligonucleotides encoding both parental residue and beneficial mutations from HCDR1, HCDR3, LCDR2, and LCDR3 were synthesized and annealed to the uridinylated template of CDR-deleted 493L1FR, of which four related CDRs were deleted to prevent bias in the annealing. The annealed mixture was then processed as described (Wu et al. (1998) Proc. Natl. Acad. Sci. USA, 95, 6037-6042; Kunkel et al. (1985) Proc. Natl. Acad. Sci. USA, 82, 488-492). The quality of the combinatorial library was examined similarly as for focused CDR libraries. For optimization of kₒₙ, a similar mutagenesis strategy was used to incorporate beneficial mutations from HCDR1, HCDR2, HCDR3, LCDR1, and LCDR2 into clone D95/G93.

### Library Screening

Libraries containing palivizumab Fab variants were first screened by a capture lift approach (Watkins et al. (1998) Anal. Biochem. 256, 169-177). Nitrocellulose filters on which 10 μg/ml of mouse-adsorbed, goat anti-human kappa antibody (Southern Biotechnology Associates) was immobilized were applied to phage-infected bacterial lawns to capture expressed Fab variants. After overnight incubation in a 22° C. incubator, filters were removed and incubated in 4 ng/ml (˜0.07 nM) of F protein solution for 2 hours at room temperature. The filters were washed 4 times with 0.1% Tween 20/PBS buffer, then developed with an anti-F protein murine monoclonal antibody, 1331H (Beeler et al. (1989) J. Virol. 63, 2941-2950), conjugated with alkaline phosphatase for 1 hour at room temperature. The filters were washed, and developed with alkaline phosphatase substrate for 10-15 minutes.

Positive clones identified by capture lift assay were further screened by ELISA (Watkins et al. (1997) Anal. Biochem. 253, 37-45). This assay allowed the rapid assessment of the relative affinities of the Fab variants. For k_{off}-optimization, IMMULON-1 microtiter plates were coated with 2 μg/ml goat anti-human Fab, and blocked with 0.5% BSA in PBS. 50 μl of Escherichia coli culture supernatant containing Fab was added to each microtiter well for 1 hour at 37° C. The plates were washed 3 times with PBS containing 0.1% Tween 20, then incubated with F protein at 40 ng/ml for 1 hour at 37° C. The plates were washed, incubated with alkaline phosphatase-conjugated antibody, 1331H, for 1 hour at room temperature, washed again, and developed with alkaline phosphatase substrate.

For kₒₙ-optimization, a different ELISA screening approach using an antigen-enzyme precomplex was developed. In brief, IMMULON-1B plates were coated with 1 μg/ml goat anti-human kappa antibody, and blocked with 1% BSA in PBS. 200 μl of E. coli culture supernatant containing the Fab was added to each well for 2 hours at room temperature. The plates were washed three times with PBS containing 0.1% Tween 20. The antigen-enzyme precomplex was formed by mixing 0.5 nM biotinylated F protein with horseradish peroxidase-conjugated streptavidin, and biotinylated horseradish peroxidase at a 1:4:9 molar ratio for 30 minutes at 37° C. 50 μl of the antigen-enzyme precomplex was added to each well, and incubated for 10 minutes at room temperature. The plates were washed three times quickly in less than 30 seconds, and incubated with substrate for 15 minutes.

### Binding Analysis by ELISA

Palivizumab Fab variants were expressed by infecting 15 ml XL-1 Blue with M13 phage carrying the Fab gene (Watkins et al. (1997) Anal. Biochem. 253, 37-45). Periplasmic extracts containing variant Fabs were prepared as described (Wu and An (2003), supra) diluted serially fourfold, and applied to IMMUNOLN-1 microtiter plates coated with 500 ng/ml F protein in a carbonate coating buffer. Subsequently, the plates were washed and the binding of antibody was detected with a goat anti-human kappa-alkaline phosphatase conjugate diluted 1000-fold in PBS containing 0.05% Tween 20. Several purified palivizumab variants in Fab or IgG format were also titrated on immobilized F protein and on RSV-infected cells. For binding to purified F protein, the procedure was similar to what was just described except that 100 ng/ml F protein was coated on the plates, and the bound antibody was detected with a goat anti-human kappa-horseradish peroxidase conjugate. To prepare RSV-infected cells, 1×10³ HEp-2 cells (human laryngeal epithelial carcinoma) per well (100 μl) were infected with RSV Long strain at a multiplicity of infection of 0.25 for 3 days. Cells were then carefully washed once with PBS containing 0.1% Tween 20, and subsequently fixed with a cold solution containing 80% acetone and 20% PBS at 4° C. for 15 minutes. The fixing solution was removed and the cells were dried at room temperature for 20 minutes. Purified antibodies diluted serially 5-fold were applied to the fixed cells, the plates were incubated at 37° C. for 1 hour, washed three times, and the bound antibodies were detected with a goat anti-human kappa-horseradish peroxidase conjugate.

To test the binding specificity of the k_{off}-improved variants to F protein, bacterial periplasmic extracts containing 100 ng/ml of variant Fabs were mixed with equal volumes of four-fold serially diluted palivizumab IgGs starting at 224 μg/ml. The mixtures were added to 96-well plates coated with 500 ng/ml F protein. After incubation of the plates for 16 hours at room temperature the unbound antibodies were removed by washing, and bound Fabs were detected with an alkaline phosphatase-conjugated monoclonal antibody, which recognizes a decapeptide tag on the carboxyl terminus of the Fab heavy chain. Palivizumab IgG instead of Fab was used in the assay because recombinant palivizumab Fab has the same detecting peptide tag as its Fab variants and is not appropriate for the assay. To test the binding specificity of the kₒₙ-improved variants to F protein, 200 ng/ml of purified Fab variants were mixed with equal volumes of four-fold serially diluted palivizumab IgGs starting at 448 μg/ml. Similar procedure as for k_{off}-improved variants was then followed except that the incubation time for binding to F protein was shortened to 4 hours at 37° C., and bound Fabs were detected with an anti-his tag antibody conjugated with horseradish peroxidase.

### Fab Expression and Purification

Many Fab fragments were cloned into an over-expression vector under the control of the arabino se-regulated BAD promoter. In addition, a six-histidine tag was fused to the carboxyl terminus of the Fab heavy chain to facilitate purification. In general, a 1-liter bacterial culture was grown and the cells were harvested and resuspended in 10 ml buffer, pH 7.5, containing 20 mM NaH₂PO₄, 500 mM NaCl, and protease inhibitors of 0.1 mM AEBSF, 1 μM Pepstatin A, and 10 μM Leupeptin. The resuspended cells were sonicated, and then incubated with 1000 U DNase I (Sigma) for 30 minutes at 4° C. The Fab was purified from the cell extracts using nickel-chelating resins. The Fab was further purified by Mono S FPLC column. This usually resulted in >95% purity as determined by SDS-PAGE.

### IgG Expression and Purification

The VH regions of the palivizumab Fab variants were amplified by PCR from phage and then fused with another PCR product containing heavy chain signal sequence by overlapping PCR. The combined PCR product was then linked to the palivizumab heavy constant region (γ1). To do this, the PCR products were digested with HindIII and SacI, and combined with a 3,544 bp SacI-BglII fragment and a 2,142 bp BglII-HindIII fragment, both from a palivizumab heavy chain expression vector (pMI226), in a three-part ligation. This resulted in an expression vector for each palivizumab heavy chain variant under the transcriptional control of the human cytomegalovirus major immediate early enhancer/promoter and the SV40 early polyadenylation region.

Using a similar strategy, the light chain genes of the palivizumab variants were synthesized by combining VL genes amplified from phage with the signal sequence and kappa constant regions amplified from the palivizumab light chain expression vector (pMI223) using overlapping PCR. The combined PCR products were then cloned into the same palivizumab light chain expression vector using a three-part ligation approach. The resulting vectors contain each light chain gene under the transcriptional control of the human cytomegalovirus major immediate early enhancer/promoter and the SV40 early polyadenylation region. In addition, the vector also contains a glutamine synthetase gene in the backbone to be used as a selectable marker by permitting growth in a glutamine-free medium.

Transient transfection of both heavy and light chain expression vectors into HEK293 or COS cells was usually performed for small-scale production of IgG. For production on a larger scale, a stable NS0 cell line was generated. For this, a single expression vector was constructed by cloning a 4.2 kb BglII-SalI fragment, containing the entire heavy chain expression cassette from the heavy chain expression vector, into the BamHI-SalI sites of the light chain expression vector, downstream of the light chain expression cassette. The vector was linearized by SalI digestion prior to transfection into NS0 cells by electroporation. Transfected cells were grown in a glutamine-free medium for selection.

Antibodies from both transient transfections or from stable cell lines were purified by chromatography on protein A columns.

### Flow Cytometry

The binding of the IgGs of palivizumab, A4b4 and AFFF(1) to F protein on the surface of RSV-infected cells was examined by flow cytometry. HEp-2 cells were infected at a multiplicity of infection of 1.5 with RSV Long. At 24 hours post-infection, the cells were detached, washed, and resuspended in FACS buffer (DPBS containing 1% BSA). The resuspended cells (2×10⁶ cells per sample) were incubated with the antibodies at 3 μg/ml for 15-20 minutes at room temperature. The cells were then collected and washed with FACS buffer. Cell-surface bound antibody was detected with goat anti-human IgG (H+L) conjugated to Alexa 647, and analyzed by FACS.

### BIAcore Analysis

The kinetic interactions of palivizumab variants with RSV F protein were determined by surface plasmon resonance using a BIAcore 1000, 2000, or 3000 instrument (Biacore, Uppsala, Sweden). Purified recombinant, C-terminally truncated F protein was covalently coupled to a (1-ethyl-3-[3-dimethylaminopropyl]carbodiimide hydrochloride)/N-hydroxysuccinimide-activated CM5 sensor chip at a low protein density (Johnsson et al. (1991) Anal. Biochem. 198, 268-277). The unreacted active ester groups were blocked with 1 M ethanolamine. For use as a reference, when the BIAcore 2000 or 3000 instrument was used, a blank surface, containing no antigen, was prepared under identical immobilization conditions. To minimize binding variations caused by different lots of F proteins, most of the antibodies were measured against the same lot of F protein. In several cases when different lots of F proteins were used, their binding to palivizumab IgG was used as a reference to make sure that these lots had similar binding characteristics to the lot that we used mainly.

A serial 2-fold dilution series of purified antibodies, ranging from 0.2 to 100 nm in HBS/Tween 20 buffer (BIAcore), was injected over the F-protein and reference cell surfaces, which were connected in series. In each measurement, the residual antibody was removed from the sensor chip by a brief pulse of 100 mM HCl. The binding curves were globally fitted to a 1:1 Langmuir binding model using the BIAevaluation program. This algorithm calculates both kₒₙ and k_{off}. The apparent equilibrium dissociation constant, K_{d}, was deduced as the ratio of the two rate constants, k_{off}/kₒₙ.

### RSV Microneutralization

A RSV microneutralization assay was used to analyze the ability of purified palivizumab variants to inhibit RSV (Long strain) replication in vitro as described (Johnson et al. (1997) J. Infect. Dis. 176, 1215-1224). Antibody dilutions were made in duplicate to quadruplicate in a 96-well plate.

Approximately 100-1000 TCID₅₀ of RSV were added to the wells and incubated for 2 hours at 37° C. Low passage, RSV-susceptible HEp-2 cells (2.5×10⁴ cells) were then added to each well and cultured for four to five days at 37° C. in a humidified 5% CO₂ incubator. After incubation, the cells were washed with 0.1% Tween 20/PBS and fixed to the plate with 80% acetone in 20% PBS. RSV replication was determined by quantitation of expressed F protein using an F protein-specific ELISA. Fixed cells were incubated with anti-RSV F protein murine antibody, 1331H, then incubated with horseradish peroxidase-conjugated goat anti-mouse IgG. Substrate TMB (3,3′,5,5′-tetramethylbenzidine) was added to each well, and the plate was measured at 450 nm. The neutralizing titer (IC₅₀) is expressed as the antibody concentration resulting in a 50% reduction in the OD₄₅₀ value (background subtracted) of no neutralization. IC₅₀ values were deduced from 4-parameter curve fitting of the sigmoid dose-response curves using Sigma Plot program.

### Results

### Further Humanization of Palivizumab and Restoration of its Light Chain CDR1

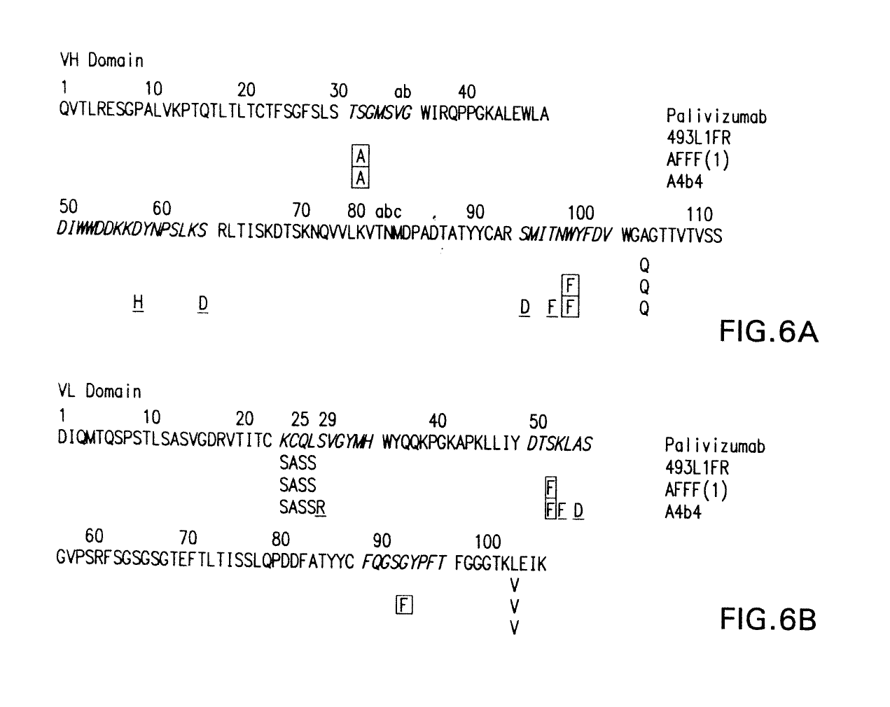

Prior to affinity maturation of palivizumab, a few modifications on the antibody were made. Amino acids KCQL, at positions 24 through 27 of the light chain CDR1 (LCDR1), were changed to the original murine monoclonal antibody 1129 sequence, SASS. The KCQL sequence represents four random, non-human, non-mouse residues that were introduced by a synthetic error during the previous humanization process (Johnson et al. (1997) J. Infect. Dis. 176, 1215-1224). In addition, we replaced the murine residues on the framework 4 (FR4) regions with human residues to reduce the possibility of immunogenicity. An amino acid substitution, A105Q, was made in the heavy chain FR4 to make a fully human JH6 germline sequence; an L104V substitution was made in the light chain FR4 to make a fully human JK4 germline sequence. The resulting clone, 493L1FR, contains fully human framework sequences (FIG. 6) and was expressed by a bacteriophage expression vector. Binding analysis of the 493L1FR Fab and palivizumab Fab by surface plasmon resonance using a BIAcore biosensor showed that both molecules bound RSV F protein with similar kinetics (Table 14). This result suggested that contrary to the earlier prediction based on structural modeling (Johnson et al. (1997) J. Infect. Dis. 176, 1215-1224) neither murine residue A105 on heavy chain FR4 nor L104 on light chain FR4 is involved significantly in F protein binding. Similarly, alteration of the first four LCDR1 residues to SASS does not substantially affect binding.

TABLE 14
Kinetics and viral neutralization of k_{off}-improved antibodies.

| | Sequence | | | | Fab | | | Fab | IgG |
|---|---|---|---|---|---|---|---|---|---|
| Clone | H1 | H3 | L2 | L3 | Kₒₙ (×10⁵₎ | K_{off} (×10⁴) | K_{d} | Microneutalization | |
| Kabat position | 32 | 100 | 52 | 93 | M⁻¹s⁻¹ | s⁻¹ | nM | (IC₅₀) μg/ml (nM)^{e} | |
| Palivizumab | S | W | S | G | 1.26 | 6.62 | 5.25 | 27.46 | 0.453 |
| | | | | | | | | (549.2) | (3.02) |
| Palivizumab^{a} | S | W | S | G | 1.19 | 7.22 | 6.07 | | |
| 493L1FR | — | — | — | — | 1.85 | 6.51 | 3.52 | 26.30 | n.d. |
| | | | | | | | | (526.0) | |
| Palivizumab^{a} | S | W | S | G | 1.19 | 7.22 | 6.07 | 26.30 | n.d. |
| | | | | | | | | (526.0) | |

Single Mutations

| | | | | | | | | | |
|---|---|---|---|---|---|---|---|---|---|
| S32A | A | — | — | — | 1.96 | 0.93 | 0.47 | 4.85 | 0.465 |
| | | | | | | | | (97.0) | (3.10) |
| S32P^{b} | P | — | — | — | n.d. | n.d. | n.d. | n.d. | n.d. |
| W100F | — | F | — | — | 1.65 | 0.84 | 0.51 | 2.60 | 0.876 |
| | | | | | | | | (52.0) | (5.84) |
| S52F | — | — | F | — | 2.06 | 1.75 | 0.85 | 7.27 | n.d. |
| | | | | | | | | (1454) | |
| S52Y | — | — | Y | — | 1.70 | 1.25 | 0.74 | 5.99 | n.d. |
| | | | | | | | | (119.8) | |
| G93F | — | — | — | F | 1.63 | 1.74 | 1.07 | 8.84 | n.d. |
| | | | | | | | | (176.8) | |
| G93Y | — | — | — | Y | 1.62 | 1.53 | 0.94 | 6.26 | n.d. |
| | | | | | | | | (125.2) | |
| G93W | — | — | — | W | 1.50 | 1.40 | 0.93 | 6.57 | n.d. |
| | | | | | | | | (131.4) | |

Combinatorial Mutations

| | | | | | | | | | |
|---|---|---|---|---|---|---|---|---|---|
| AHH(1)^{c} | A | F | F | F | 1.34 | ≦0.05^{d} | ≦0.037 | 0.0715 | 0.306 |
| | | | | | | | | (1.43) | (2.04) |
| AFFY^{c} | A | F | F | Y | 1.22 | ≦0.05^{d} | ≦0.041 | 0.0754 | 0.407 |
| | | | | | | | | (1.51) | (2.71) |
| PFFF | P | F | F | F | 1.10 | ≦0.05^{d} | ≦0.045 | n.d. | n.d. |
| AFSF^{c} | A | F | — | F | 1.13 | ≦0.05^{d} | ≦0.044 | 0.0908 | 0.521 |
| | | | | | | | | (1.82) | (3.47) |
| AFFG^{c} | A | F | F | — | 1.33 | ≦0.05^{d} | ≦0.038 | 0.249 | 0.453 |
| | | | | | | | | (4.98) | (3.02) |
| PFFY^{b} | P | F | F | Y | n.d. | n.d. | n.d. | n.d. | n.d. |
| PFFW^{b} | P | F | F | W | n.d. | n.d. | n.d. | n.d. | n.d. |
| PFYF^{b} | P | F | Y | F | n.d. | n.d. | n.d. | n.d. | n.d. |

n.d., not determined.;
H1, HCDR1;
H3, HCDR3;
L2, LCDR2;
L3, LCDR3.
^{a}This palivizumab Fab was prepared by papain cleavage of palivizumab IgG. Other palivizumab Fab used in this article was made by recombinant phage expression.
^{b}S32P was just a moderate beneficial mutation when compared with other single mutations by ELISA titration. It was therefore not further characterized by surface plasma resonance. Similarly for combinatorial variants, only the best five variants judged by ELISA titration were further characterized by surface plasma resonance, and PFFY, PFFW and PFYF were not among them.
^{c}The kinetics of these combinatorial variants in IgG format were also characterized by surface plasma resonance. Similarly to what were observed in their Fab formats, all of their k_{off} values are ≦5 × 10⁻⁶ because they have reached beyond the measurement limitation of BIAcore 3000 biosensor (5 × 10⁻⁶ s⁻¹), and could not be measured accurately. The kₒₙ values of these variants are: AFFF(1), 1.27 × 10⁵; AFFY, 1.44 × 10⁵; AFSF, 1.47 × 10⁵; AFFG, 1.47 × 10⁵;
^{d}The k_{off} value of these combinatorial clones reached beyond the measurement limitation of BIAcore 3000 biosensor (5 × 10⁻⁶ s⁻¹), and could not be measured accurately;
^{e}For comparison purpose, the IC₅₀ values were converted to nM unit and are shown in parenthesis.

k_{off}-Driven Affinity Maturation

An established directed evolution approach (Wu et al. (1998) Proc. Natl. Acad. Sci. USA, 95, 6037-6042) was used to improve the affinity of 493L1FR for the RSV F protein. The 493L1FR Fab was subjected to focused mutations at each residue in each of the six CDR regions. Separate libraries for each CDR were generated using a modified codon-based mutagenesis approach that consists of a codon doping strategy that allows the segregation of diversity into pools based on the degree of mutagenesis (Glaser et al. (1992) J. Immunol. 149, 3903-3913; Wu and An (2003), supra). Each CDR library was constructed to contain all possible single mutations at each CDR residue. These focused libraries, containing 140 to 320 variants, allowed us to explore easily the potential affinity improvements in all possible amino acids at every CDR position.

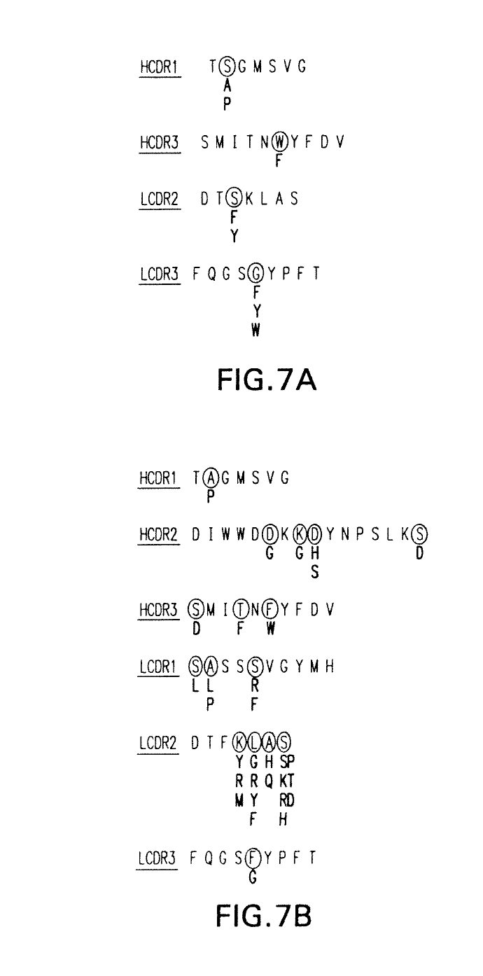

M13 plaques expressing 493L1FR Fab variants were screened for increased affinity to F protein, first by a filter-based capture lift method (Watkins et al. (1998) Anal. Biochem. 256, 169-177), and second by a semi-quantitative ELISA assay (Watkins et al. (1997) Anal. Biochem. 253, 37-45). The improved affinity of the identified clones was confirmed by an ELISA titration on immobilized F protein. DNA sequencing of the affinity-enhanced clones revealed eight distinct beneficial mutations at four CDR positions: S32A and S32P at heavy chain CDR1 (HCDR1), W100F at heavy chain CDR3 (HCDR3), S52F and S52Y at light chain CDR2 (LCDR2), and G93F, G93Y and G93W at light chain CDR3 (LCDR3) (FIG. 7A). To help visualize the three-dimensional positions of the CDR residues important for k_{off} or kₒₙ and to assist with comparison of their relative locations, these beneficial positions were shown in a molecular model (Guex et al. (1997) Electrophoresis, 18, 2714-2723) based on the crystal structure of the palivizumab Fab (data not shown). Analysis by BIAcore biosensor of seven of these mutants showed a 3 to 7-fold improvement in affinity compared with the 493L1FR Fab (Table 14). The affinity improvement was mainly driven by a lower k_{off}. The best single mutation, S32A had a K_{d} at 0.47 nM; while palivizumab Fab had at K_{d} at 5.25 nM.

During this particular experiment, we did not identify any significant mutations in heavy chain CDR2 (HCDR2) or LCDR1 that were beneficial. However, we cannot rule out the possibility that HCDR2 and LCDR1 may still play roles in binding since we set our screening threshold sufficiently high so that only clones with a substantial increase in affinity would be identified and selected for further characterization. Indeed, identified two additional beneficial mutations, A25L and S27V, in LCDR1 (data not shown). The A25L mutation was later identified again in a kₒₙ-biased screening approach (Table 15).

TABLE 15
Summary of beneficial kₒₙ mutations.

| | CDRs | | | | | | | | | | | | | | | |
|---|---|---|---|---|---|---|---|---|---|---|---|---|---|---|---|---|
| | H1 | H2 | | | | H3 | | | L1 | | | L2 | | | | L3 |
| | Kabat No. | | | | | | | | | | | | | | | |
| | 32 | 55 | 57 | 58 | 65 | 95 | 98 | 100 | 24 | 25 | 29 | 52 | 53 | 54 | 55 | 93 |
| Palvizumab | S | D | K | D | S | S | T | W | K | C | S | S | K | L | A | G |
| 493L1FR | S | D | K | D | S | S | T | W | S | A | S | S | K | L | A | G |
| AFFF(1)^{a} | A | D | K | D | S | S | T | F | S | A | S | F | K | L | A | F |
| D95/G93 | A | D | K | D | S | D | T | F | S | A | S | F | K | L | A | G |

Single Mutations

| | | | | | | | | | | | | | | | |
|---|---|---|---|---|---|---|---|---|---|---|---|---|---|---|---|
| P | G | G | H | D | D | F | W | L | L | R | Y | G | H | S | G |
| | | | S | | | | | | P | F | R | R | Q | K | |
| | | | | | | | | | | | M | Y | | R | |
| | | | | | | | | | | | | F | | H | |
| | | | | | | | | | | | | | | P | |
| | | | | | | | | | | | | | | T | |
| | | | | | | | | | | | | | | D | |

Combinatorial Mutations^{b}

| | | | | | | | | | | | | | | | | |
|---|---|---|---|---|---|---|---|---|---|---|---|---|---|---|---|---|
| Ale9 | A | G | K | H | D | D | F | W | S | L | R | F | K | L | S | G |
| Alh5 | A | G | K | H | D | D | F | W | S | L | S | F | F | H | R | G |
| A3e2 | A | G | G | H | D | D | F | W | S | A | S | F | Y | L | H | G |
| A4b4 | A | D | K | H | D | D | F | F | S | A | R | F | F | L | D | G |
| A8c7 | A | D | K | S | D | D | F | W | S | P | R | R | Y | Q | S | G |
| A12a6 | A | G | K | D | D | D | F | F | S | A | R | F | K | L | S | G |
| A13a11 | A | D | K | H | D | D | F | W | S | P | R | Y | R | H | S | G |
| A13c4 | A | G | K | S | D | D | F | F | S | L | R | M | Y | Q | S | G |
| A14a4 | A | D | K | S | D | D | T | W | L | L | R | Y | Y | Q | T | G |
| A16b4 | A | D | K | H | D | D | F | W | L | L | R | M | Y | Q | A | G |
| A17b5 | A | D | K | H | D | D | F | W | S | L | R | Y | Y | L | P | G |
| A17d4(1) | A | G | K | S | D | D | F | F | L | P | R | M | Y | Q | S | G |
| A17f5 | A | D | K | D | D | D | F | W | S | L | R | F | R | H | T | G |
| A17h4 | A | G | K | H | D | D | F | W | S | P | S | Y | Y | L | A | G |
| P11d4 | P | G | K | H | D | D | F | W | S | P | R | M | R | L | A | G |
| P12f2 | P | D | K | H | D | D | F | F | S | L | R | F | Y | L | S | G |
| P12f4 | P | G | K | H | D | D | F | F | S | L | R | R | G | L | P | G |

^{a}Clone AFFF(1) is the best combinatorial variant from k_{off}-driven affinity maturation of palivizumab in terms of affinity and the ability to neutralize virus. It was used as a starting template for kₒₙ mutagenesis.
^{b}Substantially more combinatorial mutants were identified. This table lists only the top seventeen variants based on kₒₙ improvement.

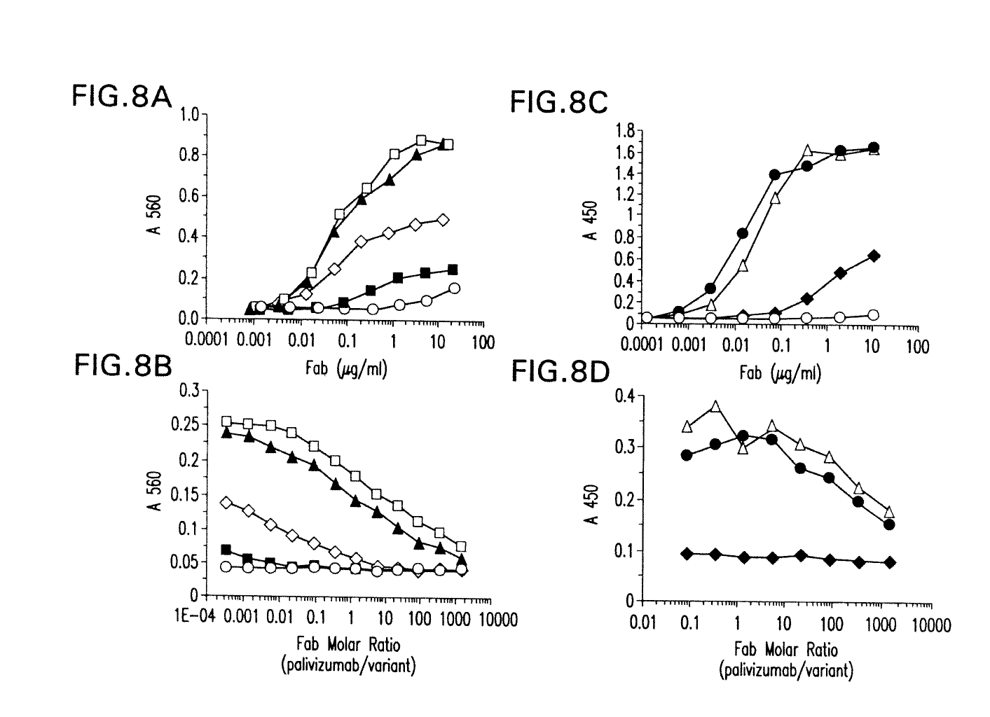

A combinatorial library combining the eight beneficial mutations was constructed by site-directed mutagenesis using degenerate oligonucleotides. Plaque lifts that detected the expression of the kappa light chain and a decapeptide tag fused at the end of the heavy chain CH1 indicated that ˜27% of the combinatorial library clones express Fab. Sequencing of the DNA of 25 random functional clones showed that the distribution of the majority of the mutations was as expected, except that S52Y in LCDR2, S32A in HCDR1, and W100F in HCDR3 were potentially under-represented. A capture lift screening of ≧2,400 clones followed by screening by ELISA led to the identification of 48 variants that had higher affinity than clone S32A, the best single-mutation variant. Further characterization by antigen titration and DNA sequencing revealed 20 unique combinatorial variants. Titrations of antigen showed that combinatorial variants have significantly enhanced affinity over S32A (FIG. 8A). The variants each contain two to four beneficial mutations, and there is a loose correlation between the affinity and the number of beneficial mutations (data not shown). The best clones contain a W100F mutation in HCDR3; no other obvious pattern was observed. Since the best single mutation, S32A, was under-represented in the combinatorial library, we decided to incorporate this mutation with the combinatorial information derived from the four best clones, PFFF, AFSF, AFFG, and PFFY (Table 14). This led to the construction of clones AFFF(1) (FIG. 6) and AFFY by site-directed mutagenesis. Both clones had a very high affinity, comparable to the four best clones; this suggests that the combinatorial library was not screened thoroughly enough to pick up the under-represented clones, even though many redundant clones were identified during the screening. The eight best variants are listed in Table 14. BIAcore analysis of the five best variants showed their affinity was more than 117-fold higher than that of the palivizumab Fab, and the affinity increase arises from the k_{off} improvement (Table 14). Clone AFFF(1) Fab has a K_{d} at ≦0.037 nM; while palivizumab Fab has a K_{d} at 5.25 nM. It should be recognized that these combinatorial palivizumab Fab variants bind so tightly to the immobilized F protein on the sensor chip that an accurate dissociation rate could not be determined (the k_{off} detection limit for BIAcore 3000 is 5×10⁻⁶ s⁻¹).

To verify the binding specificity of these variants with improved k_{off}, clones S32A, AFFF(1), AFFY, PFFF, AFSF, AFFG, and PFFY in periplasmic extracts were tested in ELISA for binding to the F protein in competition with palivizumab IgG. All variants tested competed with palivizumab and their ability to compete correlated with their affinity. Typical inhibition curves are shown in FIG. 8B.

Functional Characterization of k_{Off}-Improved Palivizumab Variants

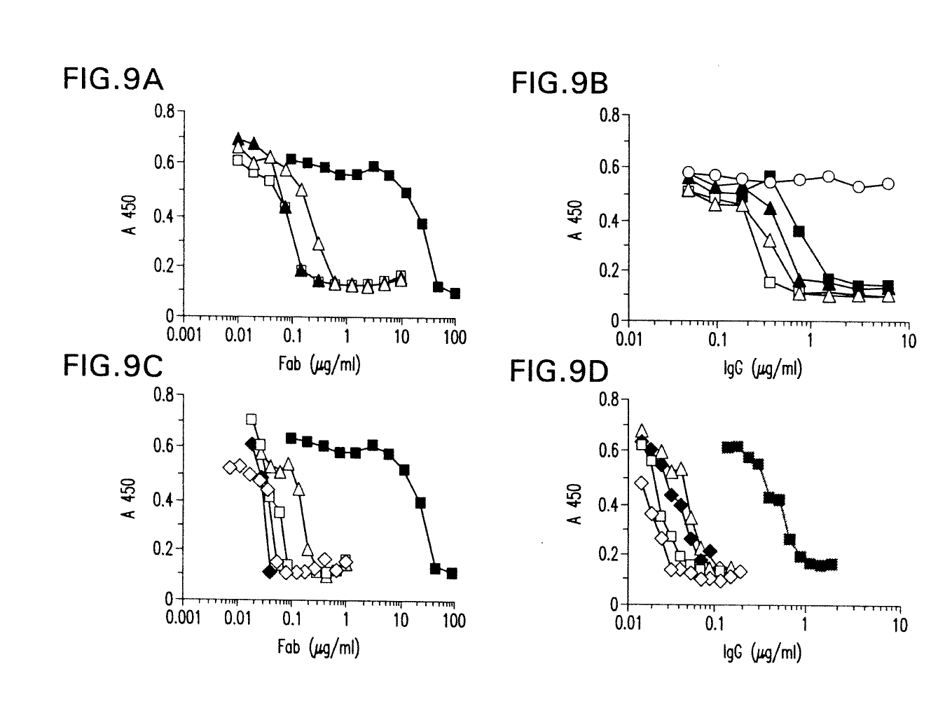

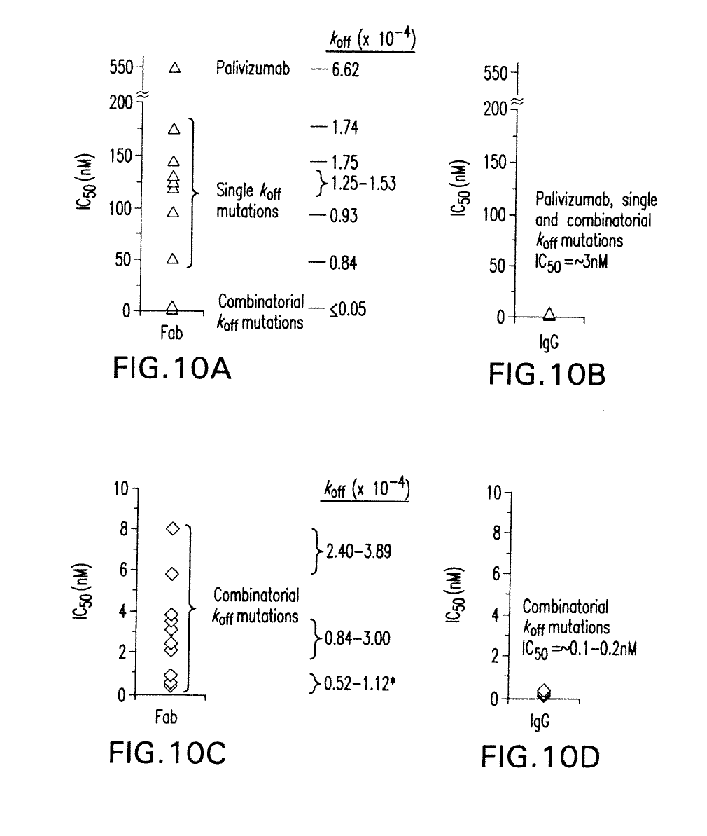

We used microneutralization of RSV as the primary assay to screen the palivizumab variants for improvement of biological function (Johnson et al. (1997) J. Infect. Dis. 176, 1215-1224; Anderson (1985) J. Clin. Microbiol. 22, 1050-1052). This assay has been used successfully to screen donors for RSV IVIg and yielded very few false positives (Sibe et al. (1992) J. Infect. Dis. 165, 456-463). Analysis by microneutralization of the purified palivizumab combinatorial Fab variants with improved k_{off} showed a 110- to 384-fold greater potency than recombinant palivizumab Fab (Table 14 and FIG. 9A). Among both the single-mutation and combinatorial Fab variants, we observed an excellent correlation between their affinities and their ability to neutralize RSV in vitro (Table 14 and FIG. 10A).

Based on the affinity to F protein and the ability to neutralize virus, the two best single-mutation Fab variants, S32A and W100F, and the four best combinatorial Fab variants, AFFF(1), AFFY, AFSF, and AFFG, were converted to intact IgG1 antibodies and expressed in NS0 cells. These purified, full-length antibodies were tested in the microneutralization assay and to our surprise there was little to no increase in the in vitro potency when compared to intact palivizumab (Table 14 and FIG. 9B). It should be noted that the microneutralization data of the combinatorial variants in Table 14 are averages from at least two independent experiments; the data shown in FIGS. 9A and B are from one typical neutralization curve.

kₒₙ Optimization with Novel ELISA Screen

Many of the k_{off} combinatorial mutants had high potency for neutralization of RSV in the Fab format but did not show any further increase in potency upon conversion of Fab to IgG. We thus next explored the potential of optimizing kₒₙ. We reasoned that theoretically an antibody with a faster kₒₙ should have a better chance to bind to and neutralize the virus before the virus has the opportunity to infect the cells.

An iterative mutagenesis approach that involved screening of about ten CDR mutation libraries was used to gradually improve the kₒₙ. Clone AFFF(1) (FIG. 6) was used as a template in the first round of kₒₙ mutagenesis. This combinatorial Fab showed one of the best improvements in k_{off} and was the most potent viral neutralizing clone derived from the k_{off}-driven affinity maturation. Libraries consisting of single mutations in HCDR3 or LCDR3, double mutations in HCDR3 or LCDR3, and double mutations with one in HCDR3 and one in LCDR3 were prepared and screened. To identify variants with increased kₒₙ, we developed a novel ELISA screening method. In principle, we wanted to reduce the interaction time between antibody and antigen as much as possible to favor the selection of variants with higher kₒₙ. In addition, after the antibody-antigen complex was formed, both the number of washes and the washing time were minimized to reduce the impact of antibody-antigen complex dissociation. Using a BIAcore kinetic simulation program, we modeled several kinetic and interaction parameters, such as kₒₙ, k_{off}, and association and dissociation times (Wu and An (2003), supra). Appropriate association and dissociation ELISA conditions were then determined, thus allowing the easy selection of high kₒₙ variants over low k_{off} variants in output signals. We screened using a 10-minute incubation time for antibody-antigen interaction followed by three quick washes in less than 30 seconds. We also eliminated the conventional second step of applying a secondary detection antibody. Instead, we precomplexed the biotinylated F protein with horseradish peroxidase-conjugated streptavidin and used it in the first step. To boost the ELISA signal, we also added biotinylated HRP to the precomplex.

Four heavy chain variants, S95D, S95F, S95L and S95N/M96S, were identified from the HCDR3 libraries, and three light chain variants, F93A, F93G, and F93W, were identified from a LCDR3 library. As estimated by BIAcore analysis, most of these mutations improved the association rate only marginally, by less than 80%. Interestingly, F93G mutation represents a reversion to a wild-type residue. It was mutated to an F in the clone AFFF(1) which was selected for its improved k_{off}. The mutation at light chain position 93 to W was also identified earlier, in the context of 493L1FR, for its improved k_{off}, with no kₒₙ benefit. A combinatorial library consisting of these beneficial kₒₙ mutations was subsequently constructed and screened. Two of the best combinatorial clones were the combinations of S95D with F93G or F93W.

The variant that contained S95D and F93G mutations, denoted as D95/G93 (or “DG”), was used as a template in the second round of kₒₙ mutagenesis. Six single-mutation CDR libraries based on D95/G93 were constructed and screened for F protein binding. Single mutations that resulted in enhanced affinity for the F protein arising from kₒₙ improvements of the Fabs were identified. These mutations and the earlier identified mutations, S95D and F93G, are listed in FIG. 7B and Table 15. Due to the relative small increase in kₒₙ, we did not characterize in detail the kinetic constants of these single mutations. The mutation to proline at position 32 on HCDR1, which was identified earlier in the first affinity maturation attempt in the context of 493L1FR, was identified here for its ability to improve kₒₙ (FIG. 7). Similarly, the mutation to tyrosine at position 52 on LCDR2 was also identified previously. In summary, all four positions that yielded improvements in k_{off}, including positions 32 (HCDR1), 100 (HCDR3), 52 (LCDR2) and 93 (LCDR3) could also be mutated to improve kₒₙ. Three-dimensional structural modeling (data not shown) shows that kₒₙ mutations are located in a broad area covering the entire CDR regions. In contrast, the k_{off} mutations are restricted to four positions.

Combinatorial libraries of these kₒₙ mutations were constructed and screened; this then lead to the identification of Fab variants (Table 15) with mostly 4- to 5-fold improvements in kₒₙ compared to the palivizumab Fab (Table 16). To verify the binding specificity of these kₒₙ variants, several purified combinatorial Fab variants were tested in ELISA for binding to the F protein with the presence of palivizumab IgG; in addition, titrations of the purified combinatorial Fab variants for binding to immobilized F protein were carried out. All the variants tested competed with palivizumab. Typical ELISA titration curves are shown in FIG. 8C, and inhibition curves shown in FIG. 8D.

TABLE 16
Kinetics and viral neutralization of kₒₙ-improved antibodies.

| | Fab | | | IgG | | | Fab | IgG |
|---|---|---|---|---|---|---|---|---|
| Clone^{a} | kₒₙ (×10⁵) | k_{off} (×10⁻⁴) | K_{d} | kₒₙ (×10⁵) | k_{off} (×10⁻⁴) | K_{d} | Microneutalization | |
| IC₅₀ | M⁻¹ₛ−1 | M⁻¹ₛ−1 | nM | M⁻¹ₛ−1 | M⁻¹ₛ−1 | nM | μg/ml (nM)^{c} | |
| Palivizumab | 1.26 | 6.62 | 5.25 | 1.27 | 4.300 | 3.386 | 27.46 | 0.453 |
| | | | | | | | (549.2) | (3.02) |
| AFFF(1)^{b} | 1.34 | ≦0.05 | ≦0.037 | 1.27 | ≦0.05 | ≦0.039 | 0.0715 | 0.306 |
| | | | | | | | (1.43) | (2.04) |
| D95/G93^{c} | n.d. | n.d. | n.d. | n.d. | n.d. | n.d. | 0.126 | n.d. |
| | | | | | | | (2.52) | |
| Ale9 | 5.23 | 1.13 | 0.216 | 4.33 | 1.430 | 0.330 | 0.157 | 0.0625 |
| | | | | | | | (3.14) | (0.42) |
| Alh5 | n.d. | n.d. | n.d. | n.d. | n.d. | n.d. | 0.194 | n.d. |
| | | | | | | | (3.88) | |
| A3e2 | 5.99 | 0.94 | 0.157 | n.d. | n.d. | n.d. | n.d. | n.d. |
| A4b4 | 6.04 | 0.52 | 0.086 | 5.53 | 0.151 | 0.027 | 0.414 | 0.104 |
| | | | | | | | (0.83) | (0.069) |
| A8c7 | 6.47 | 3.00 | 0.464 | 4.17 | 0.875 | 0.210 | 0.177 | 0.0337 |
| | | | | | | | (3.54) | (0.22) |
| A12a6 | 5.19 | 2.19 | 0.422 | 4.60 | 0.165 | 0.036 | 0.0287 | 0.0357 |
| | | | | | | | (0.57) | (0.24) |
| A13a11 | 6.80 | 2.29 | 0.337 | n.d. | n.d. | n.d. | 0.0120 | n.d. |
| | | | | | | | (2.40) | |
| A13c4 | 6.50 | 1.12 | 0.172 | 3.00 | 0.396 | 0.132 | 0.0264 | 0.0376 |
| | | | | | | | (0.53) | (0.25) |
| A14a4 | 3.32 | 2.40 | 0.723 | n.d. | n.d. | n.d. | >0.4^{d} | n.d. |
| | | | | | | | (>8.0) | |
| A16b4 | 4.90 | 1.05 | 0.214 | n.d. | n.d. | n.d. | n.d. | n.d. |
| A17b5 | 5.90 | 0.73 | 0.124 | n.d. | n.d. | n.d. | 0.406 | n.d. |
| | | | | | | | (0.92) | |
| A17d4(1) | 5.31 | 0.59 | 0.111 | 4.56 | 0.407 | 0.089 | 0.0179 | 0.0342 |
| | | | | | | | (0.36) | (0.23) |
| A17f5 | 5.44 | 0.84 | 0.154 | n.d. | n.d. | n.d. | 0.106 | n.d. |
| | | | | | | | (2.12) | |
| A17h4 | 5.19 | 1.05 | 0.202 | n.d. | n.d. | n.d. | n.d. | n.d. |
| P11d4 | 5.70 | 3.89 | 0.682 | 4.66 | 2.890 | 0.620 | 0.292 | 0.0217 |
| | | | | | | | (5.84) | (0.14) |
| P12f2 | 5.35 | 0.72 | 0.135 | 4.07 | 1.280 | 0.314 | 0.0407 | 0.0231 |
| | | | | | | | (0.81) | (0.15) |
| P12f4 | n.d. | n.d. | n.d. | 4.95 | 0.555 | 0.112 | 0.0464 | 0.242 |
| | | | | | | | (0.93) | (0.16) |

^{a}Several antibodies were analyzed by surface plasma resonance on several occasions. The average kₒₙ values with standard deviations and the number of independent measurements (n) of these antibodies are shown below: Palivizumab IgG, 1.27 ± 0.33 × 10⁵ (n = 6); AFFF(1) IgG, 1.27 ± 0.31 × 10⁵ (n = 4); A4b4 IgG, 5.53 ± 1.63 × 10⁵ (n = 3); A3e2 Fab, 5.99 ± 0.20 × 10⁵ (n = 2); A4b4 Fab, 6.04 ± 2.67 × 10⁵ (n = 2); A13c4 Fab, 6.50 ± 1.43 × 10⁵ (n = 5); A16b4 Fab, 4.90 ± 0.00 × 10⁵ (n = 2); A17b5 Fab, 5.90 ± 0.35 × 10⁵ (n = 2); A17d4(1), 5.31 ± 0.17 × 10⁵ (n = 2); A17f5 Fab, 5.44 ± 0.38 × 10⁵ (n = 2).
^{b}The k_{off}-combinatorial clone, AFFF(1), is included for comparison purpose.
^{c}The kinetics constants of D95/G93 were not characterized in detail due to the small relative increase in kₒₙ.
^{d}Fab A14a4 were tested in a RSV microneutralization assay at a concentration up to 0.4 μg/ml, and no inhibition of viral replication was observed. Higher concentrations were not tested since it was clear that this Fab variant was not the best among these combinatorial clones.
^{e}For comparison purpose, the IC₅₀ values were converted to nM unit and are shown in parenthesis.

The building of the improvement in kₒₙ in AFFF(1) significantly diminished the improvement in k_{off}. AFFF(1) Fab has a k_{off} two log better than that of the palivizumab Fab; while these kₒₙ combinatorial Fabs have a k_{off} only 2- to 13-fold better. This result was not surprising because some of the beneficial k_{off} mutations in AFFF(1) were replaced with kₒₙ mutations in these combinatorial clones. For example, clone P11d4 has the worst k_{off} among this group (Table 16), and all of its k_{off} mutations, such as A at position 32 on HCDR1, F at position 100 on HCDR3, F at position 52 on LCDR2 and F at position 93 on LCDR3, were replaced (Table 15).

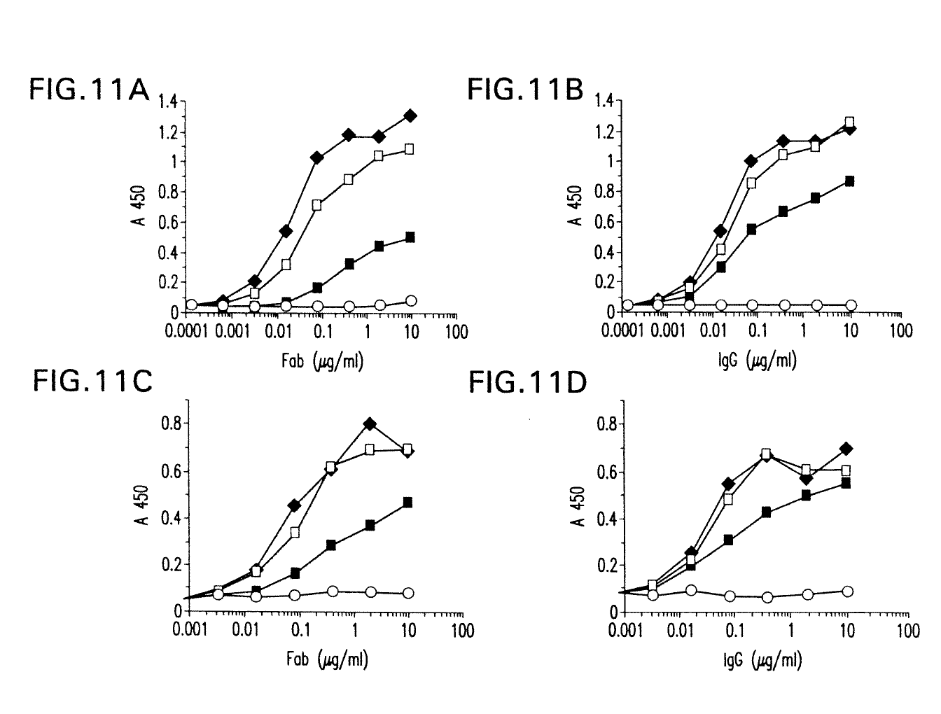

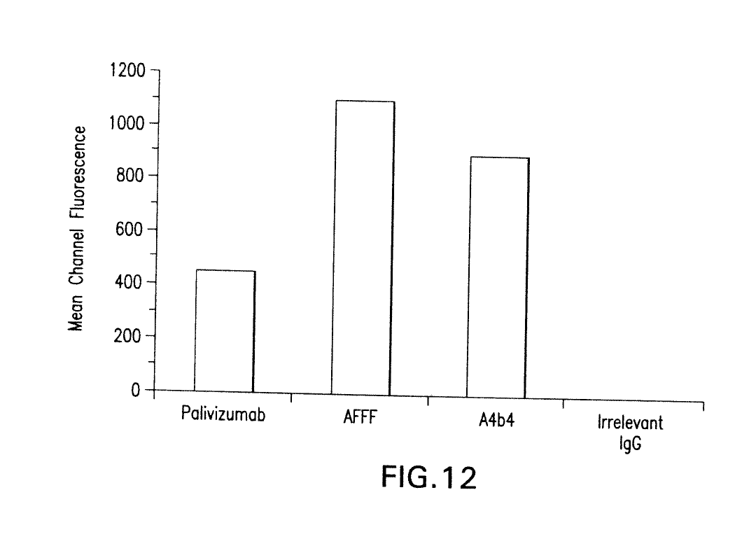

Several combinatorial clones were converted to full-length IgG1/kappa antibodies for further characterization. The converted full-length antibodies still retained the improved kₒₙ although these improvements were slightly reduced. This may be due to variations in BIAcore measurements, but is also possibly caused by the conversion to IgG. The IgG conversion does improve the k_{off} 3- to 13-times through increased avidity in some, but not all, of the converted antibodies. Marked improvements were seen with A4b4, A8c7, A12a6, and A13c4 but in contrast, with palivizumab and some variants, such as Ale9, A17d4(1), P11d4, and P12f2, there were only minor improvements in k_{off} upon conversion to IgG (Table 16). Palivizumab, A4b4 and AFFF(1) in the Fab and IgG format were further characterized for their binding to RSV-infected cells that expressed F protein on the cell surface (FIGS. 11C and D). ELISA titrations showed that both A4b4 and AFFF(1) bound substantially better than palivizumab to acetone-fixed HEp-2 cells infected with RSV Long. This increase in binding was similar to that observed in BIAcore analysis for their binding to immobilized F protein (Tables 14 and 16). The same antibodies were also titrated on affinity-purified recombinant F protein (FIGS. 11A and B). The binding profiles of these antibodies (whether in Fab or IgG format) were very similar whether assayed against purified F protein or cell-expressed F protein. In addition, a preliminary study by flow cytometry was conducted to measure the binding of palivizumab, A4b4 and AFFF(1) IgGs at 3 μg/ml to RSV-infected HEp-2 cells. The ability of the antibody to bind to the infected cells as measured by mean channel fluorescence correlated well with the ELISA titration results (FIG. 12).

Functional Characterization of kₒₙ-Improved Palivizumab Variants

Most of the combinatorial Fab variants selected by improvement of kₒₙ have a 4- to 5-fold better kₒₙ and a 2- to 13-fold better k_{off} than the parent clone, palivizumab Fab. Furthermore, the optimization of kₒₙ greatly improved the ability to neutralize virus relative to that of the parent clone. The improvement in neutralization activity for kₒₙ-improved Fab variants is, in general, substantially better than that of k_{off}-improved variants (Tables 14 and 16). Whilst the best k_{off}-improved Fab, AFFF(1), has a 384-fold improvement, the neutralization activity of seven out of fourteen characterized kₒₙ-improved Fab variants is improved beyond that of AFFF(1). The variant A17d4(1) Fab has a 1.534-fold better IC₅₀ than the palivizumab Fab. Variants A12a6 and A13c4 have about 1000-fold improvements, and variant A4b4, A17b5, P12f2 and P12f4 have about 600- to 700-fold improvements (Table 16 and FIG. 9C). Six out of these seven best Fab variants retain their beneficial k_{off} mutation, phenylalanine, at position 100 on HCDR3 (Table 15). For kₒₙ-improved variants in the Fab format, k_{off} appeared to contribute to the differences in their IC₅₀. For example, clone P11d4 with a similar kₒₙ to others has the fastest k_{off} value and one of the worst IC₅₀. Clone A17d4(1) has nearly the slowest k_{off} value among these variants, and its IC₅₀ is also the best (Table 16). When these kₒₙ-improved clones were converted to full-length antibodies, they continued to exhibit much higher ability to neutralize virus compared to palivizumab IgG (Table 16 and FIG. 9D). This result contrasts dramatically with the result obtained using k_{off}-combinatorial variants, where the functional improvement largely disappeared after IgG conversion (Table 14 and FIG. 9B). The best intact antibody overall in terms of the ability to neutralize virus in vitro is A4b4 (Table 16 and FIG. 6). The full-length A4b4 antibody has a 27 pM affinity for RSV F protein as estimated by BIAcore analysis, representing a 125-fold improvement over that of palivizumab. A4b4 IgG also exhibits a 44-fold greater potency in the microneutralization assay as compared to palivizumab. These K_{d} and IC₅₀ numbers are averages from at least two independent experiments. Detailed analysis of the data in Table 16 revealed that in one variant, P11d4, conversion of the Fab into IgG did not give rise to a significant improvement in k_{off}. Thus, the k_{off} and kₒₙ of P11d4 IgG are similar to those of its Fab fragment, yet the IC₅₀ of the IgG form is 42-fold better than that of the Fab fragment (5.84 nM for Fab vs. 0.14 nM for IgG). This large improvement in IC₅₀ upon conversion to IgG, without a concomitant increase in avidity, is similar to that observed upon conversion of the Fab form of palivizumab to IgG. In contrast, clone A12a6 exhibited a 13-fold improvement in k_{off} upon IgG conversion presumably due to avidity effect; however, this avidity improvement did not result in substantial improvement in the ability to neutralize virus (IC₅₀ 0.57 nM for Fab, and 0.24 nM for IgG). We also observed that, in general, variants that already had gained large improvements in IC₅₀ in the Fab format tended to gain less improvement in the values of IC₅₀ upon conversion to IgG; such examples are variants A12a6, A13c4 and A17d4(1) (Table 16). Their IC₅₀ values in Fab format are 0.36 to 0.57 nM which are about 1,000-1,500-fold better than that of palivizumab Fab. After conversion to IgG, their IC₅₀ are 0.23 to 0.25 nM, only a two-fold increase over the Fab forms. In contrast, variants such as A8c7 and P11d4 have IC₅₀ values of 3.54 nM and 5.84 nM respectively, which are about 94-155-fold better than that of palivizumab Fab (Table 16). Once converted to IgG, the values of the IC₅₀ are 0.22 and 0.14 nM, a 16-42-fold increase over the Fab form. A similar observation was made for the k_{off}-improved variants (Table 14).

### Discussion

Using a very efficient directed evolution approach based on phage expression (Wu et al. (1998) Proc. Natl. Acad. Sci. USA, 95, 6037-6042), we have fully humanized palivizumab, restored the unnatural residues on its LCDR1 to parental murine residues, and identified many variants with great improvements in k_{off} without the need for structural information. All k_{off}-beneficial mutations located on HCDR3 (W100F), LCDR2 (S52F, and S52Y), and LCDR3 (G93F, G93Y, and G93W) share one common feature: an aromatic side chain (FIG. 7A). Perhaps these mutations are engaged in aromatic stacking with the RSV F protein. However, examination of the palivizumab binding site on the F protein, which spans from position 260 to 275 as predicted by antibody-resistant RSV mutants (Crowe et al. (1998) Virology, 262, 373-375; Zhao et al. (2004) Virology, 318, 608-612) reveals no aromatic side chains that would favor such an interaction. Although these aromatic amino acids may interact with residue(s) adjacent to the binding site, in the absence of co-crystal structural information this interpretation remains speculative.

The combinatorial Fab variants containing three to four beneficial k_{off} mutations have an affinity at least 117-fold higher than that of the palivizumab Fab, and this results in a concomitant improvement in their ability to neutralize RSV virus to at least 110-fold higher (Table 14). However, once we converted these anti-RSV Fab variants into whole IgG molecules, the ideal drug format, the difference in potency largely disappeared (Table 14 and FIG. 10B), despite the fact that those IgG variants with a 5×10⁻⁶ s⁻¹ k_{off} still exhibit much higher affinities than palivizumab (Table 14: footnote c). The primary reason for the dramatic reduction in relative potencies is that palivizumab upon conversion from Fab to IgG was found to undergo a two-log increase in in vitro potency; in contrast, when some of the variants were converted to IgG no increase in potency was observed (Table 14, FIGS. 10A and B). For example, the IC₅₀ of palivizumab Fab is 549.2 nM, and that for palivizumab IgG is 3.02 nM, reflecting a 182-fold increase due to conversion to IgG. In contrast, the variant AFFF(1) Fab and IgG have IC₅₀ values of 1.43 nM and 2.04 nM respectively. Conversion of this k_{off}-improved variant from Fab to IgG confers no IC₅₀ improvement. The increase in avidity arising from the bivalency of the IgG does not explain the increase in neutralization shown by palivizumab IgG, since the K_{d} (and k_{off}) of palivizumab improved only minimally upon conversion to the IgG format; in contrast, the variant AFFF(1) as an IgG has an almost two-log higher binding avidity over palivizumab IgG but shows less than a 2-fold improvement in IC₅₀ (Table 14).

Due to these unexpected results from the k_{off}-improved clones, we decided to improve the kₒₙ of palivizumab. The best k_{off} variant, AFFF(1), was selected as the starting molecule for further engineering. Through iterative CDR mutations and screening, many beneficial kₒₙ single mutations were identified. As observed in this study and some earlier reports,^{12,20} selected k_{off} single mutations typically improve k_{off} 2- to 13-fold. In contrast, in this study kₒₙ single mutations were found to improve kₒₙ by only 20-80% (data not shown). After combining several kₒₙ mutations, the kₒₙ was improved overall up to 5-fold (Table 16). All of these combinatorial clones still showed improvement in their k_{off} though at a much reduced level. Fortunately, these kₒₙ-improved variants have shown great potency enhancement in both Fab and IgG formats (Table 16, FIGS. 10C and D).

To dissect the impact of antibody binding kinetics on the ability to neutralize virus, as indicated by IC₅₀, we analyzed the entire data set in Tables 14 and 16. We found a strong correlation between k_{off} and IC₅₀ for k_{off}-improved Fab variants (FIG. 10A). Moreover, for Fab fragments, we observed that both k_{off} and kₒₙ influence viral neutralization. However, kₒₙ appears to play a much more significant role in the enhancement of the ability to neutralize virus. Examples of this are the Fab variants W100F and A17f5. Both have an 8-fold improvement in k_{off}, and A17f5 has an additional 3-fold increase in kₒₙ over W100F. The 8-fold k_{off}-improvement resulted in an 11-fold improvement in IC₅₀ for W100F. However, the additional 3-fold increase in kₒₙ resulted in an additional 25-fold improvement in IC₅₀ for A17f5. A similar comparison can be made between variants S52Y and A1e9 with palivizumab Fab.

For full-length antibodies, kₒₙ maintains its influential role on IC₅₀ while the impact of k_{off} appears to be much less. When compared to palivizumab (Table 16), all the k_{off}-combinatorial IgG variants, such as AFFF(1), AFFY, and AFFG, exhibited much higher avidity, driven by ˜100-fold improvement in k_{off} (Table 14: footnote c), yet their IC₅₀ values show almost no improvement over that of palivizumab (Table 14). In addition, the kₒₙ-improved IgG clones, P11d4, P12f2, and P12f4, all have similar kₒₙ values but distinctive k_{off} values, ranging from 5.55×10⁻⁵ to 2.89×10⁻⁴ s⁻¹, but these differences in k_{off} did not result in differences in IC₅₀ (Table 16). In contrast, the kₒₙ-combinatorial IgG variant, P11d4, exhibits a 4-fold improvement in kₒₙ and only a slightly better k_{off} than palivizumab (2.89×10⁻⁴ vs. 4.3×10⁻⁴ s⁻¹), yet its IC₅₀ is dramatically increased 21-fold over palivizumab. This improvement in IC₅₀ is attributed largely to its kₒₙ improvement. It should be noted however, that when kₒₙ has already been improved, additional substantial improvement in k_{off} may confer an added beneficial effect on IC₅₀. An example of this is A4b4 IgG which has a 4-fold kₒₙ (similar to other kₒₙ clones) and 28-fold k_{off} improvement over palivizumab, and its IC₅₀ is increased 44-fold, indicating that k_{off} can influence the ability of kₒₙ-improved intact antibodies to neutralize virus. Our finding that kₒₙ plays a predominant role in RSV viral neutralization may be explained by the possibility that antibodies with higher kₒₙ values can bind to the virus more quickly and thus neutralize it before the virus has the chance to infect cells. However, we cannot rule out other possibilities since the mechanism by which palivizumab blocks RSV infection at the molecular level is still not well understood.

We also observed that the IC₅₀ values of palivizumab and all the k_{off}-improved variants appeared to converge to ˜3 nM upon conversion to IgG despite differences in k_{off} that ranged from ≦5×10⁻⁶ to 4.3×10⁻⁴ s⁻¹ (Table 14 and FIG. 10B). These clones, including palivizumab, have similar kₒₙ values at ˜1×10⁵ M⁻¹ s⁻¹ in both Fab and IgG formats. A similar behavior was also observed for the kₒₙ-improved variants where the IC₅₀ values for all the characterized kₒₙ variants converged to −0.1-0.2 nM upon conversion to IgG despite differences in k_{off} that ranged from 1.51×10⁻⁵ to 2.890×10⁻⁴ s⁻¹ (Table 16 and FIG. 10D). The kₒₙ values of these clones are similarly improved to ˜5×10⁵ M⁻¹s⁻¹ in Fab formats and ˜4×10⁵ M⁻¹s⁻¹ in IgG formats. Overall, for full-length antibodies, a 4-fold improvement in kₒₙ leads to a 15- to 30-fold improvement in the ability to neutralize virus regardless of the differences in k_{off}.

Based on our observations, two major factors appear to affect the IC₅₀ of intact antibodies in viral neutralization: kₒₙ and the bivalency of IgG. The influence of k_{off} differs substantially between the Fab and IgG formats, with a strong influence on the IC₅₀ in Fabs but with little effect on the IC₅₀ as IgG molecules. However, this conclusion should be limited to molecules with k_{off} below that of palivizumab. Palivizumab IgG has a k_{off} of 4.3×10⁻⁴ s⁻¹, which results in a theoretical dissociation half-life of the antigen-antibody complex of 27 minutes, as calculated by the formula T_{1/2}=ln2/k_{off}. It is possible that the contribution of k_{off} to viral neutralization is already at its maximum in palivizumab, and therefore, further improvements in the off-rate in variants simply cannot further increase the neutralization activity. For Fabs, which bind monovalently and are smaller in size, the k_{off} threshold required to effectively neutralize RSV may be elevated, and thus this may explain why we observed a significant role for k_{off} in the IC₅₀ of Fab variants.

As discussed earlier, upon the conversion of k_{off}-versus kₒₙ-improved variants from Fab to IgG we observed generally differences in their ability to neutralize virus (FIG. 9). The conversion from Fab to IgG increases both antibody size and the binding valence. To understand the contribution of these changes to viral neutralization, we prepared F(ab′)₂ fragments of palivizumab and one of its variants, and tested them in the microneutralization assay. In this study, the IC₅₀ values derived from averages of two independent experiments for palivizumab and its F(ab′)₂ are 3.6 and 1.4 nM respectively, and for the variant and its F(ab′)₂ are 0.23 and 0.15 nM respectively. We did not observe large differences in the IC₅₀ values for both of the F(ab′)₂ constructs compared to their respective parental IgG despite that their size was reduced from 150 kD to 100 kD. This indicates that antibody size in this range does not significantly affect viral neutralization. This suggests that the ability to bind bivalently as one of the causes for the change in IC₅₀ values upon conversion of Fab to IgG. AFFF(1) and other combinatorial k_{off} variants as IgG have much higher avidities than palivizumab, but all these molecules have similar IC₅₀ values as palivizumab. Bivalent binding appears to be able to influence viral neutralization through a mechanism unrelated to avidity. In another example, conversion of palivizumab or the variant, P11d4, from Fab to IgG, did not significantly alter the values of K_{d}, but did improve the IC₅₀ values 42- to 182-fold over their respective Fab fragments. This suggests that avidity is not playing a role, but still bivalent binding does alter viral neutralization. It is possible that we did not see a good correlation between antibody avidity and viral neutralization because the avidity values were measured by surface plasma resonance on immobilized F protein. In this artificial system, the viral epitopes displayed on the surface of sensor chip may not completely mimic natural presentation of such an epitope on the virions or the virus-infected cell surface expressing F protein. It is also possible that the Fab and IgG versions of the same antibody neutralize virus through different mechanisms, and this may account for the differences in RSV neutralization that were observed when palivizumab Fab variants were converted into IgGs.

### 6.9 Prophylaxis of Upper Respiratory Tract RSV Infection in Cotton Rats by A4B4L1Fr-S28R (MEDI-524)

Intramuscular dosing studies were conducted in cotton rats to compare the efficacy of A4B4L1FR-S28R (MEDI-524) and palivizumab in reducing upper respiratory tract RSV infection. For each experiment, juvenile cotton rats (Sigmodon hispidus, average weight 100 g) were separated into six groups of ten animals each, two groups each for MEDI-524, palivizumab, and bovine serum albumin (BSA) control. Animals were anesthetized with methoxyflurane and given 0.2 ml of purified mAb or BSA by intramuscular injection (i.m.), one group at 2.0 mg/kg body weight and one group at 20.0 mg/kg body weight for each test article. Twenty four hours later, animals were again anesthetized, bled for serum IgG quantitation, and challenged by intranasal instillation (i.n.) of 1×10⁵ pfu/animal RSV (Long strain). Four days later animals were sacrificed and their lungs and nasal turbinates were harvested. Lung and nasal turbinate homogenates were prepared in Hank's balanced salt solution (HBSS) and the resultant suspensions were used to determine viral titers by plaque assay utilizing confluent HEp-2 cells. Serum human IgG titers at the time of challenge, as well as lung homogenate and nasal turbinate homogenate human IgG titers at the time of sacrifice, were determined by an anti-human IgG-specific ELISA as described in Section 5.1.4.

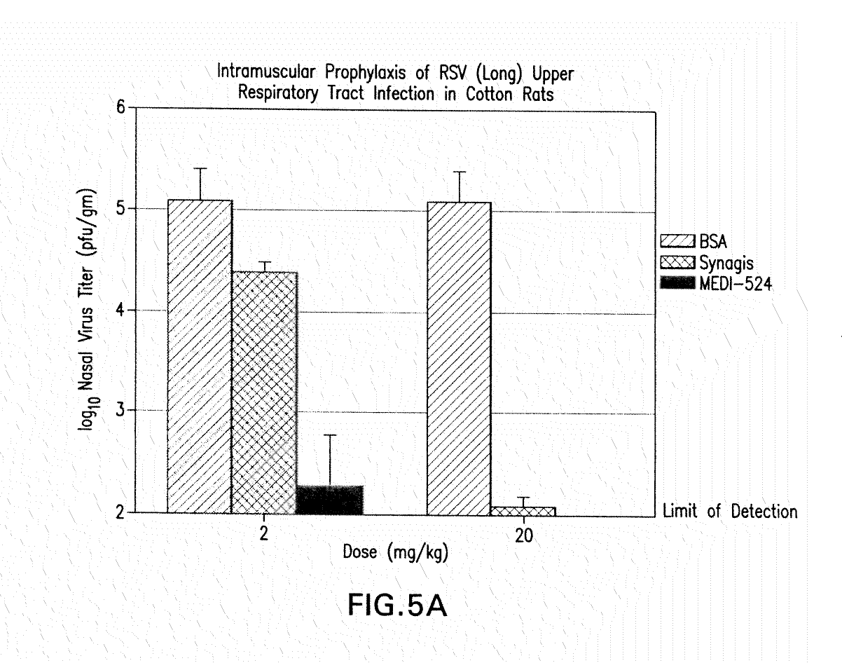

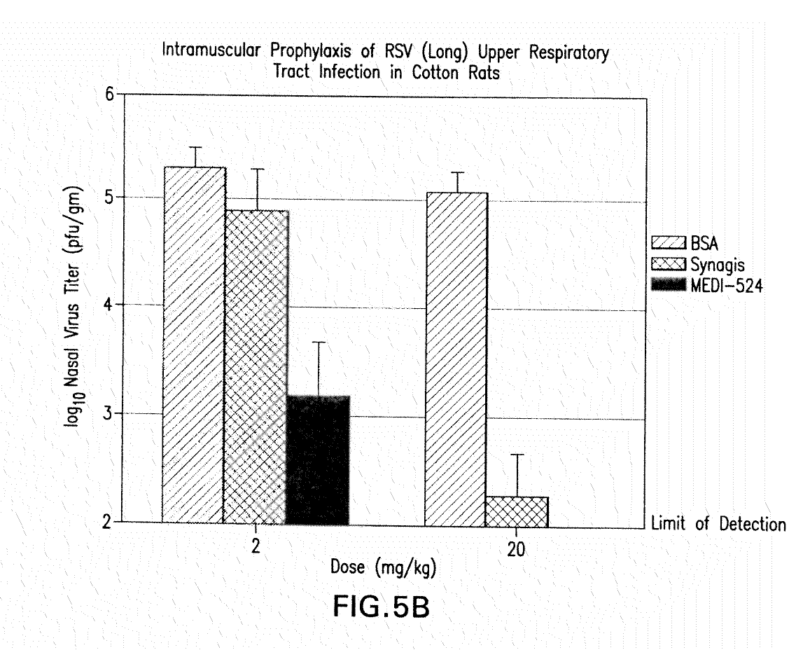

Results of two cotton rat prophylaxis experiments are presented in Tables 17 and 18, infra, and in FIGS. 5A and 5B, supra. The results of these studies show that when administered at equivalent doses, MEDI-524 and palivizumab attain equivalent levels in the serum, lungs, and nasal turbinates of cotton rats. At a dose of 2 mg/kg MEDI-524 effected a 50-100-fold greater reduction in upper respiratory RSV Long titers than did palivizumab. MEDI-524 reduced the nasal turbinate RSV titers by greater than 99% (>2 logs) as compared to the BSA control, while palivizumab effected only a 60%-80% (<1 log) reduction in nasal turbinate RSV.

TABLE 17
Intramuscular Prophylaxis of RSV (Long) Upper Respiratory Tract
Infection in Cotton Rats.

| | | | | | Lung Viral | Nasal Viral |
|---|---|---|---|---|---|---|
| | | Serum Human | Lung Human | Nasal Human | Titer | Titer |
| | | IgG at | IgG at | IgG at | Geometric | Geometric |
| | | Challenge, | Sacrifice | Sacrifice | Mean ± Std | Mean ± Std |
| | Dose | Mean ± Std | Mean ± Std | Mean ± Std | Error (log₁₀ | Error (log₁₀ |
| Treatment | (mg/kg) | Error (μg/ml) | Error (μg/ml) | Error (μg/ml) | pfu/g) | pfu/g) |
| MEDI-524 | 2 | 20.7 ± 2.4 | 0.258 ± 0.114 | 0.169 ± 0.036 | <2.0 ± 0.0* | 2.3 ± 0.5 |
| SYNAGIS | 2 | 16.1 ± 4.4 | 0.182 ± 0.080 | 0.126 ± 0.037 | 2.3 ± 0.3 | 4.4 ± 0.1 |
| BSA | 2 | 0.0 | 0.0 | 0.0 | 5.0 ± 0.3 | 5.1 ± 0.3 |
| MEDI-524 | 20 | 213.0 ± 71.7 | 2.0 ± 0.8 | 1.2 ± 0.6 | <2.0 ± 0.0* | <2.0 ± 0.0* |
| SYNAGIS | 20 | 166.0 ± 54.9 | 1.8 ± 0.9 | 1.1 ± 0.3 | <2.0 ± 0.0* | 2.1 ± 0.1 |
| BSA | 20 | 0.0 | 0.0 | 0.0 | 4.9 ± 0.4 | 5.1 ± 0.3 |

*Viral titers for all animals in this group were <100 pfu/gm, the lower limit of detection for the plaque assay.

TABLE 18
Prophylaxis of RSV (Long) Upper Respiratory Tract Infection in Cotton Rats

| | | | | | Lung Viral | Nasal Viral |
|---|---|---|---|---|---|---|
| | | Serum Human | Lung Human | Nasal Human | Titer | Titer |
| | | IgG at | IgG at | IgG at | Geometric | Geometric |
| | | Challenge, | Sacrifice | Sacrifice | Mean ± Std | Mean ± Std |
| | Dose | Mean ± Std | Mean ± Std | Mean ± Std | Error (log₁₀ | Error (log₁₀ |
| Treatment | (mg/kg) | Error (μg/ml) | Error (μg/ml) | Error (μg/ml) | pfu/g) | pfu/g) |
| MEDI-524 | 2 | 12.0 ± 1.9 | 0.246 ± 0.050 | 0.123 ± 0.016 | <2.0 ± 0.0* | 3.2 ± 0.5 |
| SYNAGIS | 2 | 15.8 ± 1.2 | 0.250 ± 0.057 | 0.118 ± 0.012 | <2.0 ± 0.0* | 4.9 ± 0.4 |
| BSA | 2 | 0.0 | 0.0 | 0.0 | 4.5 ± 0.1 | 5.3 ± 0.2 |
| MEDI-524 | 20 | 151.6 ± 41.8 | 2.7 ± 0.5 | 1.3 ± 0.1 | <2.0 ± 0.0* | <2.0 ± 0.0* |
| SYNAGIS | 20 | 149.2 ± 25.6 | 2.3 ± 0.4 | 1.1 ± 0.1 | <2.0 ± 0.0* | 2.3 ± 0.4 |
| BSA | 20 | 0.0 | 0.0 | 0.0 | 4.6 ± 0.4 | 5.1 ± 0.2 |

*Viral titers for all animals in this group were <100 pfu/gm, the lower limit of detection for the plaque assay.

### Results

The results of these experiments indicate that MEDI-524, compared to palivizumab, is more effective in preventing upper respiratory tract infections in vivo, as demonstrated by the experiments performed in the cotton rat experimental model and summarized in Tables 17 and 18, and FIGS. 5A and 5B. At a dose of 2 mg/kg, MEDI-524 effected a 50-100-fold greater reduction in upper respiratory RSV Long titers than did palivizumab. Further, MEDI-524 reduced the nasal turbinate RSV titers by greater than 99% (>2 logs) as compared to the BSA control, while palivizumab effected only a 60%-80% (<1 log) reduction in nasal turbinate RSV.

These results have important implications for the prevention of upper respiratory tract infections in humans, particularly in infants, and also for the prevention of the development of lower respiratory tract infections (generally affecting the lungs) from upper respiratory tract infections. It is estimated that 30-50% of infants are affected by lower respiratory infections caused by RSV. The use of MEDI-524 would be beneficial because it is significantly more potent at preventing upper respiratory tract infections at a lower dose than palivizumab. It is anticipated that such findings will result in a lower rate of upper and lower respiratory tract infections in infants, as well as a decrease in the number of physician visits.

### 6.10 Intramuscular Cotton Rat Studies

This experiment demonstrates that a greater reduction in RSV titer is achieved when A4b4, A4b4-F52S or A4b4/L1FR-S28R is administered intramuscularly to a cotton rat than when the same concentration of palivizumab is administered intramuscularly to a cotton rat.

### Materials & Methods

### Intramuscular Cotton Rat Prophylaxis

Cotton rats (S. hispidus, average weight 100 grams) were anesthetized with methoxyflurane and dosed with 0.1 ml of purified monoclonal antibody (mAb) or BSA control by intramuscular (i.m.) injection. Twenty-four hours later animals were again anesthetized, bled for serum mAb concentration determination, and challenged with 10⁵ PFU RSV long by intranasal (i.n.) instillation. Four days later animals were sacrificed, serum samples were obtained, and their lungs were harvested. Lungs were homogenized in 10 parts (wt/vol) of Hanks Balanced Salt solution and the resultant suspension was used to determine pulmonary viral titers by plaque assay.

### Intramuscular Cotton Rat Pharmacokinetics

Cotton rats (S. hispidus, average weight 100 grams) were anesthetized with methoxyflurane and dosed with 0.1 ml of purified mAb or BSA control by intramuscular (i.m) injection. Twenty-four hours later all of the animals were bled for serum mAb concentration determination, and half of the animals from each group were sacrificed to perform bronchoalveolar lavage (BAL). Four days later the remaining animals were sacrificed, serum samples were obtained and BAL performed.

### Results

As shown in Tables 19-21, a greater reduction in RSV titer is achieved with equivalent or lower lung levels of A4b4, A4b4-F52S, or A4b4/L1FR-S28R as with palivizumab.

TABLE 19
Intramuscular Cotton Rat Prophylaxis Data

| | 0.5 mg/kg | | | | 0.125 mg/kg | | | |
|---|---|---|---|---|---|---|---|---|
| | Serum | | Virus | log | | Lung | Virus | log |
| | IgG | Lung IgG | Titer | Virus | Serum IgG | IgG | Titer | Virus |
| | (μg/ml) | (μg/ml) | (pfu/gm) | Titer | (μg/ml) | (μg/ml) | (pfu/gm) | Titer |
| SYNAGTS | 3.4 | 0.099 | 7.3 × 10³ | 3.9 | 0.893 | 0.024 | 3.1 × 10⁴ | 4.5 |
| A4b4-F52S | 2.9 | 0.089 | 7.3 × 10² | 2.9 | 0.781 | 0.020 | 8.6 × 10³ | 3.9 |
| A4b4/L1FR- | 3.3 | 0.093 | 6.1 × 10² | 2.8 | 0.748 | 0.016 | 2.3 × 10⁴ | 4.4 |
| S28R | | | | | | | | |
| BSA | | | 5.9 × 10⁴ | 4.8 | | | | |

TABLE 20
Intramuscular Cotton Rat Prophylaxis Data

| | 0.5 mg/kg log(10) | | | 1 mg/kg log(10) | | |
|---|---|---|---|---|---|---|
| | Serum | | | Serum | | |
| | IgG | Lung IgG | Lung | IgG | Lung IgG | Lung |
| Molecule | (μg/ml) | (μg/ml) | Virus | (μg/ml) | (μg/ml) | Virus |
| A4b4 | 2.4 | 0.013 | 4.3 | 3.1 | 0.094 | 3.4 |
| SYNAGIS | 1.9 | 0.038 | 4.4 | 4.2 | 0.212 | 3.3 |
| BSA | | | | | | 4.4 |

TABLE 21
Intramuscular Cotton Rat Pharmacokinetics Data

| | 24 Hours | | 96 Hours | |
|---|---|---|---|---|
| | Serum IgG | BAL IgG | Serum IgG | BAL IgG |
| Molecule | (μg/ml) | (ng/ml) | (μg/ml) | (ng/ml) |
| A4b4 | 3.4 | 2.2 | 2.6 | 1.4 |
| SYNAGIS | 4.1 | 5.3 | 2.8 | 3.5 |

### 6.11 Clinical Trials

Antibodies of the invention tested in in vitro assays and animal models may be further evaluated for safety, tolerance and pharmacokinetics in groups of normal healthy adult volunteers. The volunteers are administered intramuscularly, intravenously or by a pulmonary delivery system a single dose of 0.5 mg/kg, 3 mg/kg, 5 mg/kg, 10 mg/kg, 15 mg/kg, 30 mg/kg, 45 mg/kg, or 60 mg/kg of an antibody of the invention which immunospecifically binds to a RSV antigen (e.g., RSV F antigen). Each volunteer is monitored at least 24 hours prior to receiving the single dose of the antibody and each volunteer will be monitored for at least 48 hours after receiving the dose at a clinical site. Then volunteers are monitored as outpatients on days 3, 7, 14, 21, 28, 35, 42, 49, and 56 postdose.

Blood samples are collected via an indwelling catheter or direct venipuncture using 10 ml red-top Vacutainer tubes at the following intervals: (1) prior to administering the dose of the antibody; (2) during the administration of the dose of the antibody; (3) 5 minutes, 10 minutes, 15 minutes, 20 minutes, 30 minutes, 1 hour, 2 hours, 4 hours, 8 hours, 12 hours, 24 hours, and 48 hours after administering the dose of the antibody; and (4) 3 days, 7 days 14 days, 21 days, 28 days, 35 days, 42 days, 49 days, and 56 days after administering the dose of the antibody. Samples are allowed to clot at room temperature and serum will be collected after centrifugation.

The antibody is partially purified from the serum samples and the amount of antibody in the samples will be quantitated by ELISA. Briefly, the ELISA consists of coating microtiter plates overnight at 4° C. with an antibody that recognizes the antibody administered to the volunteer. The plates are then blocked for approximately 30 minutes at room temperate with PBS-Tween-0.5% BSA. Standard curves are constructed using purified antibody, not administered to a volunteer. Samples are diluted in PBS-Tween-BSA. The samples and standards are incubated for approximately 1 hour at room temperature. Next, the bound antibody is treated with a labeled antibody (e.g., horseradish peroxidase conjugated goat-anti-human IgG) for approximately 1 hour at room temperature. Binding of the labeled antibody is detected, e.g., by a spectrophotometer.

The concentration of antibody levels in the serum of volunteers are corrected by subtracting the predose serum level (background level) from the serum levels at each collection interval after administration of the dose. For each volunteer the pharmacokinetic parameters are computed according to the model-independent approach (Gibaldi et al., eds., 1982, Pharmacokinetics, 2^{nd} edition, Marcel Dekker, New York) from the corrected serum antibody concentrations.

### 6.12 Phase 1 Clinical Trial of MEDI-524 in Healthy Adults

Background: RSV is a pathogen of infants and young children, causing annual epidemics of bronchiolitis and pneumonia worldwide and hospitalizations in approximately 2% of infected infants. Premature infants, infants with chronic lung disease (CLD) of prematurity, and infants with hemodynamically significant congenital heart disease are hospitalized 4-5 times more frequently and sustain increased morbidity and mortality compared to infants without these risk factors. Palivizumab (palivizumab), a humanized RSV monoclonal antibody directed against the F glycoprotein of RSV, is currently FDA-approved for the passive immunoprophylaxis of serious acute RSV disease in high-risk children. MEDI-524 has 20-100 fold increased activity against RSV in pre-clinical studies. The cotton rat model of RSV was used to select the palivizumab dose (15 mg/kg monthly) evaluated in efficacy trials. This dose was chosen in order to achieve a serum concentration that, in the cotton rat, was associated with a 2 log₁₀ reduction in pulmonary RSV. Prophylaxis of high-risk children with this dose resulted in ˜50% overall reduction in RSV hospitalization rates compared to placebo.

As described elsewhere herein, MEDI-524 is an enhanced potency RSV-specific monoclonal antibody derived by in vitro affinity maturation of the complementarity-determining regions of the heavy and light chains of palivizumab. Preclinical data demonstrate that MEDI-524's affinity to the F protein of RSV (BIAcore) is ˜70-fold higher compared to palivizumab, and MEDI-524 is ˜20-fold more potent in microneutralization studies. Studies in the cotton rat model, which are described in prior examples herein, demonstrate that, at comparable serum concentrations, MEDI-524 has 50-100 times greater anti-viral activity against RSV compared to palivizumab in the lower respiratory tract. In addition, MEDI-524 reduces RSV in the upper respiratory tract by 2-3 logs, whereas palivizumab has minimal effect.

Objective: This was an initial dosage study of MEDI-524 to evaluate its safety, immunogenicity, and pharmacokinetics (PK) in healthy adults.

Design/Methods: Healthy adults were separated into five treatment groups, with each treatment group containing 6 healthy adults. Groups 1-3 received MEDI-524 as a single IV dose of 3, 15, or 30 mg per kg of patient body weight, respectively. Group 4 received MEDI-524 as a single IM dose of 3 mg/kg IM. Group 5 received MEDI-524 as two doses of 3 mg/kg IM on days 0 and 30. Group 6 received a placebo.

A safety follow-up was conducted at 60 days following the final dose. PK and immunogenicity follow-up was conducted for 180 days following the final dose.

### Results

Safety: MEDI-524 was well-tolerated in all groups (4 SOIs), and there were no dose-limiting toxicities or serious adverse effects (SAEs) reported.

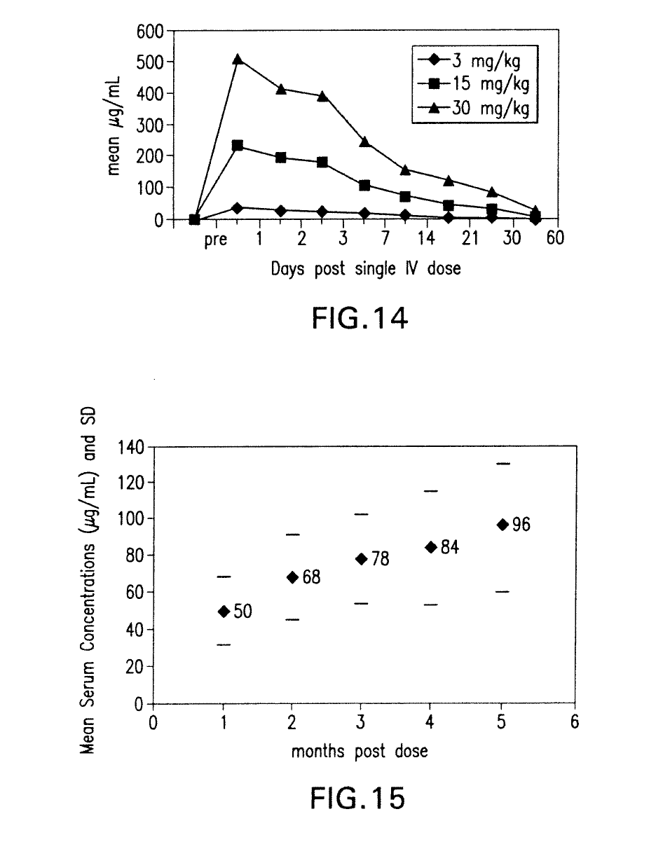

Pharmokinetics: The mean half-life of antibody was 15-18 days. Mean serum MEDI-524 trough concentrations of Groups 1-3 are presented in FIG. 14. The mean half-life was calculated to be 15-18 days.

Immunogenicity: Thirteen percent of patients had and anti-idiotype response. However, the anti-idiotypic response was not associated with and adverse events.

### Discussion

Conclusions: These results suggest that MEDI-524 is both safe and effective at these tested doses, and that follow-up repeat dosing studies are appropriate.

### 6.13 Phase 1/2 Repeat Dosing MEDI-524 Clinical Trial in High-Risk Children

Objective: This was a dose escalation, repeat dose study of MEDI-524 to evaluate its safety, immunogenicity, and pharmacokinetics (PK) in high risk children.

This study was the first trial of MEDI-524 conducted in a pediatric population. It was designed to describe the safety, tolerability, immunogenicity, and pharmacokinetics of escalating, repeated intramuscular (IM) injections of MEDI-524 during the RSV season in children with prematurity or CLD of prematurity.

Design/Methods: Preterm infants, GA 32-35 weeks (wks), age ≦6 months (m) received monthly IM doses of MEDI-524 at 3 mg/kg (N=6) or 15 mg/kg (N=24). Subsequently, infants ≦2 years with CLD of prematurity were included to receive 15 mg/kg. Clinical/lab adverse events (AEs), immunogenicity, and PK were evaluated through 150 days after final dose.

This was an open-label, Phase 1/2, dose-escalation study conducted during the respective RSV seasons in the northern and southern hemispheres. Children received at least 2 and up to 5 doses of study drug, given 30 days apart, depending on when in the RSV season a child was enrolled in the study.

TABLE 22
Subjects Enrolled and Treatments Administered

| Group | Treatment | IM Dosage | N | Subjects |
|---|---|---|---|---|
| 1 | MEDI-524 | 3 mg/kg | 6 | premature (≧32 to ≦35 weeks gestation) ≦6 |
| | | | | months of age |
| 2 | MEDI-524 | 15 mg/kg | 24^{a} | premature (≧32 to ≦35 weeks gestation) ≦6 |
| | | | | months of age |
| 3 | MEDI-524 | 15 mg/kg | 187^{b} | premature (≧32 to ≦35 weeks gestation) ≦6 |
| | | | | months of age OR ≦24 months of age with |
| | | | | CLD of prematurity, with stable or |
| | | | | decreasing doses of diuretics, steroids, or |
| | | | | bronchodilators within the previous 6 |
| | | | | months. |

^{a}Six children were enrolled; following acceptable safety review, the remaining 18 were enrolled
^{b}Following acceptable safety review of Groups 1 and 2, enrollment in Group 3 was begun

Evaluations are described in Table 23. Cumulative review of available safety data for all children was performed by the Medical Monitor, with a report submitted to the Safety Monitoring Committee every 30 days. Adverse events (AEs) included any adverse change from baseline condition, regardless of relationship to study drug. Serious adverse events (SAEs) included those that resulted in death; were life-threatening; resulted in inpatient hospitalization or prolongation of existing hospitalization; resulted in persistent or significant disability or incapacity; or were an important medical event. MEDI-524 serum concentrations (limit of detection 1.56 μg/mL) and immunogenicity were assayed using ELISA. For the detection of immune reactivity, wells were coated with MEDI-524 with the detection reagent consisting of horseradish peroxidase-conjugated MEDI-524.

TABLE 23
Schedule of Evaluations

| | DAYS | | | | | | | | |
|---|---|---|---|---|---|---|---|---|---|
| | | | | | | | | All patients | |
| | | | | | | | Additional | 30 days | 90 days |
| Procedures and | All patients | | | | | | doses | after final | after final |
| Evaluations | 0 | 2 | 7 | 30 | 32 | 37 | 60/90/120 | dose | dose |
| Dosing | X | | | X | | | X^{a} | | |
| CBC w/ differential | X | | X | X | | X | X^{b} | X | |
| ALT, AST, BUN | X | | X | X | | X | X^{b} | X | |
| Creatine | | | | | | | | | |
| Serum MEDI-524 | X | X | | X | | X | X^{b} | X | X |
| MEDI-524 | X | | | X | | X | X^{b} | X | X |
| immunogenicity | | | | | | | | | |
| Safety assessments | X | X | X | X | X | X | X | X | |

^{a}Additional doses depended on when the child was enrolled in the RSV season
^{b}Performed on Study Day 60

Patient Population: A total of 217 children entered the study (N=6 at 3 mg/kg; N=211 at 15 mg/kg): the first 40 children were enrolled in the US in late winter of 2004; the remaining 177 children were enrolled in S. America, during the 2004 RSV season in the southern hemisphere. A total of 205 (94%) children completed the study through 90 days after the final dose of study drug. 112 (52%) children received 5 doses of study drug.

The mean age of participating children was 3.0 months (range: 0.1-21.2) and mean weight was 4.1 kg (range: 1.8-12.1). The majority of children were Hispanic (167, 77%), followed by white/non-Hispanic (41, 19%); 129 (59%) were male; 32 (15%) children had CLD of prematurity.

### Results

Overview: 217 children (40 USA, 177 S America) received 2-5 doses of MEDI-524; follow-up is ongoing. Data from 194 children: mean age, 3m (range: 1-21m), mean GA, 33 wks (range: 25-35 wks), 62% male. AEs were typical of high risk children; 98% were mild/moderate severity. Potentially related AEs were transient injection site erythema (N=16), hypochromic anemia (N=2), SGOT increase (N=1). For all children, no related serious AEs or AE related dose discontinuations occurred. Mean trough serum drug levels 30 days after 1 and 2 doses of 15 mg/kg were 51 and 69 μg/mL, respectively, and only 1 child (of 185 tested) had evidence of immune reactivity (90 days after dose 3). This child remained clinically asymptomatic and target serum drug levels were maintained during dosing.

Safety—Adverse Events: Overall, 1006 AEs were reported in 200 children during this trial. Most were typical events characteristic of the underlying conditions of the participating children, and the incidence was generally similar to that previously reported in the Phase 3 placebo-controlled trial of palivizumab. No AEs resulted in discontinuation of study drug.

Nine AEs and 1 SAE (an inguinal hernia) were reported in the 6 children who received 3 mg/kg of study drug. None of the AEs that occurred in this low dose group were judged to be related to study drug. All AEs were Level 1 or 2 in severity.

Table 24 describes the AEs reported by Body System in this trial for children receiving 15 mg/kg. The AEs reported in the pivotal Phase 3 trial in premature infants and infants with CLD of prematurity who received palivizumab or placebo are included for comparison purposes (Pediatrics (1998) 102:531-537).

Ninety-three percent of children receiving repeated monthly doses of 15 mg/kg MEDI-524 reported at least one AE during the study. The majority of the AEs reported (945/997, 95%) were Level 1 or 2 in severity. The highest percentage of subjects had AEs referable to the following systems: Digestive (35%), Body as a Whole (46.6%), Hemic and Lymphatic (56%), and Respiratory (60%).

Digestive System: The commonly reported AEs were diarrhea (10.0%), AST increase (8.1%), infantile colic (7.1%), constipation (6.6%), gastroesophageal reflux disease (6.2%), ALT increase (6.2%), and vomiting (6.2%). All children with ALT (N=2), AST (N=7), ALT and AST elevations (N=11) were asymptomatic, with AEs detected during laboratory assessments. Two-thirds of these events were Level 1 or 2 severity. In most cases, the events were either transient and non-recurring with continued dosing or isolated elevations at the last study evaluation that resolved or improved within 1 month.

Body as a Whole: The commonly reported events were fever (16.1%), study drug injection site reactions (16.6%), and pain (11.8%). Only 2 cases of fever were associated temporally with study drug injection (occurring on the day of dose 4 and 1 day after dose 2, respectively, with no recurrences with subsequent dosing). The most common injection site reaction was erythema reported for 31 (14.7%) children. Injection site hemorrhage, pain, induration, and edema due to study drug were each reported for between 1 and 5 children. All injection site reactions were Level 1 in severity, transient, with most resolving within 1 day.

TABLE 24
Summary of Incidence of all Adverse Events by Body System

| | MI-CP104 | | MI-CP018 | | | |
|---|---|---|---|---|---|---|
| | MEDI-524 | | Palivizumab | | | |
| | 15 mg/kg | | 15 mg/kg | | Placebo | |
| Body System | (N = 211) | | (N = 1002) | | (N = 500) | |
| Total Number of AEs | 997 | | 5417 | | 2737 | |
| Total children | 196 | (92.9%) | 961 | (95.9%) | 482 | (96.4%) |
| reporting ≧1 AE | | | | | | |
| Body as a Whole | 98 | (46.4%) | 497 | (49.6%) | 247 | (49.4%) |
| Fever | 34 | (16.1%) | 272 | (27.1%) | 134 | (26.8%) |
| Site of Injection | 35 | (16.6%) | 27 | (2.7%) | 9 | (1.8%) |
| Reaction | | | | | | |
| Cardiovascular System | 11 | (5.2%) | 25 | (2.5%) | 19 | (3.8%) |
| Digestive System^{a} | 88 | (41.7%) | 456 | (45.5%) | 255 | (51.0%) |
| AST and/or ALT | 20 | (9.5%) | 75 | (6.9%) | 30 | (6.0%) |
| Increase | | | | | | |
| Endocrine System | 0 | | 1 | (0.1%) | 0 | |
| Hemic and Lymphatic | 117 | (55.5%) | 27 | (2.7%) | 15 | (3.0%) |
| System^{b} | | | | | | |
| Anemia^{c} | 107 | (50.7%) | NT | | NT | |
| Metabolic and | 10 | (4.7%) | 34 | (3.4%) | 16 | (3.2%) |
| Nutritional | | | | | | |
| Musculoskeletal | 1 | (0.5%) | 5 | (0.5%) | 3 | (0.6%) |
| Nervous System | 20 | (9.5%) | 134 | (13.4%) | 62 | (12.4%) |
| Respiratory System^{d} | 126 | (59.7%) | 835 | (83.3%) | 411 | (82.2%) |
| Skin and Appendages | 59 | (28.0%) | 326 | (32.5%) | 161 | (32.2%) |
| Special Senses | 35 | (16.6%) | 484 | (48.3%) | 233 | (46.6%) |
| Urogenital System | 3 | (1.4%) | 28 | (2.8%) | 17 | (3.4%) |

^{a}Both trials required routine liver function tests. More post dose time points were collected in the Phase 1/2 trial of MEDI-524 (5) compared to the Phase 3 palivizumab trial (1)
^{b}Study required CBC changes from baseline are included only in MEDI-524 group since CBCs were not collected in the Phase 3 palivizumab trial
^{c}Events coding to anemia, hemoglobin decreased, or neonatal anemia, NT = not tested per protocol
^{d}Includes respiratory infections in all groups

Respiratory System: The commonly reported AEs were nasopharyngitis (17.5%), upper respiratory tract infection (17.5%), bronchitis (16.1%), pharyngitis (7.6%), chronic bronchitis (7.1%), and wheezing (5.2%). Except for 2 cases of URI, no events were considered related to study drug; no wheezing events occurred within 2 days of study drug injection.

Hemic and Lymphatic System: The most commonly reported AE was anemia and other analogous events (50.7%). All but one event (final Hgb 8.6 g/dL) were Level 1 or 2 in severity; 101 (90%) of the children received iron supplementation. For most children (99, 88%), low hemoglobin levels resolved or improved by the last laboratory evaluation, and were consistent with anemia of prematurity.

AEs Judged to be Possibly Related: A total of 47 (22%) children experienced at least one AE considered potentially related to study drug. The majority (109/117, 93.2%) of related AEs were Level 1 or 2 in severity. The most common (>1%) were injection site reactions (30, 14.2%) and transaminase elevations (14, 6.6%).

Safety—Serious Adverse Events: Table 25 describes the SAEs reported by Body System in this trial for children receiving 15 mg/kg. The SAEs reported in the pivotal Phase 3 trial in premature infants and infants with CLD of prematurity who received palivizumab or placebo are included for comparison purposes. Twenty-two (10.4%) children in the 15 mg/kg dosage group experienced 26 SAES; most were respiratory hospitalizations (20, 77%). No SAEs resulted in permanent discontinuation of study drug. The rates of all SAEs by Body System seen in this trial appeared similar to or lower than those reported for palivizumab or placebo in the previous pivotal Phase 3 trial.

TABLE 25
Summary of Incidence of all Serious Adverse Events by Body System

| | MI-CP104 | | MI-CP018 | | | |
|---|---|---|---|---|---|---|
| | MEDI-524 | | Palivizumab | | | |
| | 15 mg/kg | | 15 mg/kg | | Placebo | |
| Body system | (N = 211) | | (N = 1002) | | (N = 500) | |
| Total Number of SAEs | 26 | | 475 | | 277 | |
| Total children reporting | 22 | (10.4%) | 298 | (29.7%) | 170 | (34.0%) |
| ≧1 SAE | | | | | | |
| Body as a Whole | 2 | (0.9%) | 101 | (10.1%) | 53 | (10.6%) |
| Cardiovascular System | 0 | | 3 | (0.3%) | 2 | (0.4%) |
| Digestive System | 1 | (0.5%) | 93 | (9.3%) | 42 | (8.4%) |
| Hemic and Lymphatic | 1 | (0.5%) | 2 | (0.2%) | 1 | (0.2%) |
| System | | | | | | |
| Metabolic and | 0 | | 5 | (0.5%) | 3 | (0.6%) |
| Nutritional | | | | | | |
| Musculoskeletal | 0 | | 1 | (0.1%) | 2 | (0.4%) |
| Nervous System | 0 | | 4 | (0.4%) | 2 | (0.4%) |
| Respiratory System | 18 | (8.5%) | 144 | (14.4%) | 82 | (16.4%) |
| Skin and Appendages | 0 | | 0 | | 2 | (0.4%) |
| Special Senses | 0 | | 25 | (2.5%) | 26 | (5.2%) |
| Urogenital System | 2 | (0.9%) | 4 | (0.4%) | 5 | (1.0%) |

One SAE was considered possibly related to study drug. This child, given a diagnosis of idiopathic thrombocytopenic purpura (ITP), had a transient significant decrease in platelets following dose 4 of study drug that resolved without treatment. No other child in this study had any platelet abnormalities noted during the trial.

Two children died during the study. Both deaths were judged to be unrelated to study drug. One was due to a RSV bronchopneumonia, in a child hospitalized 7 days after first dose. The other event was judged as SIDS by autopsy and occurred more than 2 months after the last dose of study drug (in the 3 mg/kg dose group).

Immunogenicity: No anti-MEDI-524 binding responses (defined as a titer ≧1:10) were detected in any child during the MEDI-524 dosing period. 7 (3.3%) children in the 15 mg/kg treatment group had anti-MEDI-524 reactivity detected after their last dose of MEDI-524: 3 (1.4%) at 30 days after dose 5, and 4 (1.9%) at 90 days after the final dose (1 each after 3 or 4 doses, and 2 after 5 doses. Immune reactivity at 30 days after dose 5 was associated with no detectable drug levels at this time point. These responses occurred in the absence of any significant adverse events during the study. The one child with ITP had anti-MEDI-524 binding activity detected ˜2 months after the event (90 days after the dose 4).

Pharmacokinetics: Mean serum MEDI-524 trough concentrations during monthly IM injections of 15 mg/kg are presented in FIG. 15. Concentrations ≧30 μg/mL were maintained throughout dosing in ≧90% of children and increased with continued dosing as expected. As shown in Table 26, a vast majority (>90%) of high risk children achieve target serum trough concentrations of ≧30 μg/ml throughout dosing.

TABLE 26
Serum Trough Concentrations of MEDI-524

| | % of Patients with Monthly Serum Trough | | | | |
|---|---|---|---|---|---|
| | Concentrations of ≧30 μg/ml | | | | |
| | Day 30 | Day 60 | Day 90 | Day 120 | Day 150 |
| MEDI-524 | 90% | 96% | 93% | 94% | 94% |

### Discussion

Conclusions: MEDI-524 given for up to 5 doses at 3 and 15 mg/kg to high-risk children appeared to be safe and well tolerated. Adverse events were typically Level 1 or 2 in severity, were consistent with the underlying conditions in this high-risk population, and were similar in incidence to that observed in previous trials of palivizumab. Transient Level 1 site of injection reactions were reported in 16.6%.

The incidence of immune reactivity was low (N=7, 3%) and was detected after completion of dosing (post dose 5 or 90 days after final dose). Immune reactivity detected after dose 5 (N=3) was associated with no detectable serum drug levels and no significant adverse events. The one child with ITP had anti-MEDI-524 binding activity detected ˜2 months after this event (90 days after the dose 4).

The pharmacokinetic profile was consistent with IgG₁. Ninety percent or more of children achieving target serum trough concentrations ≧30 μg/mL throughout dosing, with concentrations rising with each subsequent dose. In the previous successful pivotal Phase 3 trial of similarly dosed children given palivizumab, 79% and 87% achieved these levels after doses 2 and 4, respectively.

These data suggest that MEDI-524 given as repeat IM monthly 15 mg/kg doses in high risk children has a safety, immunogenicity, and PK profile similar to palivizumab. These data support continued evaluation of MEDI-524 for the prevention of RSV hospitalizations in high risk children., and support the evaluation of MEDI-524 for the prevention of RSV hospitalizations in these high-risk children.

### 6.14 Phase 1 Single Dosing MEDI-524 Clinical Trial Safety Study in Children with RSV Infection

Objective: This was single dose study of MEDI-524 to evaluate its safety, immunogenicity, and pharmacokinetics (PK) in children with RSV lower respiratory infection (LRI). This study was the second trial of MEDI-524 conducted in a pediatric population. It was designed to describe the safety, tolerability, immunogenicity, and pharmacokinetics of a single intravenous (IV) dose of MEDI-524 in patients that were hospitalized with RSV LRI. Further, as part of this Phase 1 safety study we assessed whether MEDI-524 would hasten the clearance of a naturally acquired RSV infection in children.

Design/Methods: Thirty children hospitalized with RSV LRI (bronchiolitis) were randomly divided into four treatment groups, and received intravenous administration of either placebo (n=15) or 3 mg/kg, 15 mg/kg, or 30 mg/kg of MEDI-524 (n=5/group). Clinical/lab adverse events (AEs), immunogenicity, and PK were evaluated. RSV was investigated by viral culture (PFU/mL), antigen detection (Binax) and quantitative RT-PCR, in nasal washes obtained before, and 1, 2, and 7 days after administration of placebo or MEDI-524.

### Results

Adverse effects of serious adverse effect were balanced between treatment groups and placebo group. Two patients reported a serious adverse effect, which was determined to be unrelated to the MEDI-524 administration, one of which was in the placebo group (EBV infection and respiratory failure) and one in the 30 mg/kg group (respiratory failure). There were no discernable differences in duration of hospitalization, use of supplemental oxygen, ICU needed or the need for mechanical ventilation

Serum and nasal titers of MEDI-524: MEDI-524 presence in serum and nasal secretions is presented in Table 28. As expected, the mean serum and nasal concentrations of MEDI-524 increase with increasing dosages.

TABLE 28
MEDI-524 Present in Serum and Nasal
Secretions in Children with RSV

Day 1 Nasal MEDI-524

| | Day 2 serum mean | Mean | |
|---|---|---|---|
| | MEDI-524 (μg/ml) | (μg/ml) | % Positive |
| Placebo | 0 | 0 | 0 |
| 3 mg/kg | 61.8 | 0.2 | 40 |
| 15 mg/kg | 170.8 | 0.9 | 60 |
| 30 mg/kg | 333.2 | 1.3 | 80 |

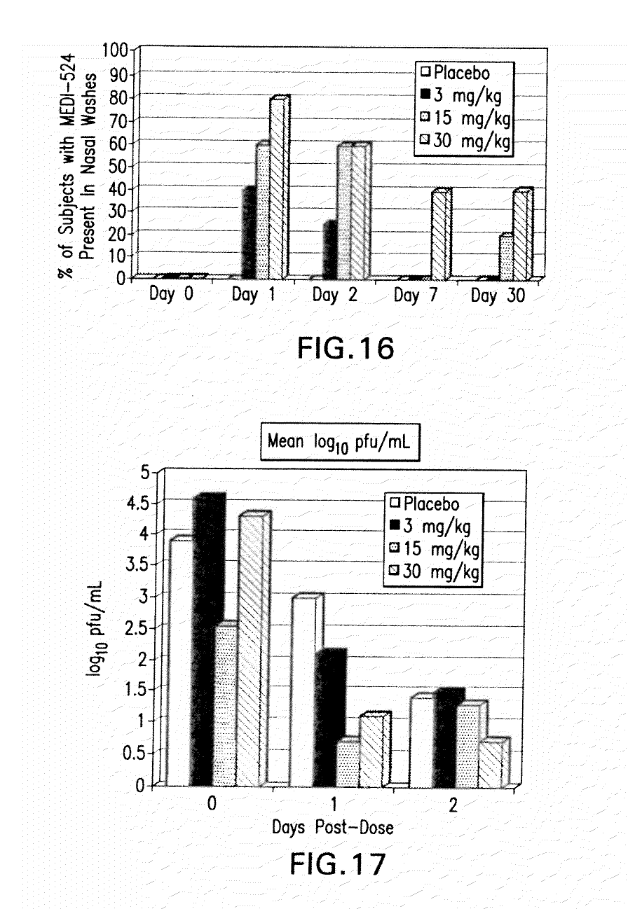

Pharmokinetics: The PK profile of MEDI-524 in nasal secretions following a single IV dose of MEDI-524 is shown in FIG. 16. The percent of subjects with MEDI-524 in nasal washes was directly proportional to the amount of MEDI-524 received. Patients receiving a 3 mg/kg dose had MEDI-524 present in nasal secretions at 2 days post-dose, whereas patients receiving 15 mg/kg or 30 mg/kg had MEDI-524 present in nasal washes for up to 30 days post-dose.

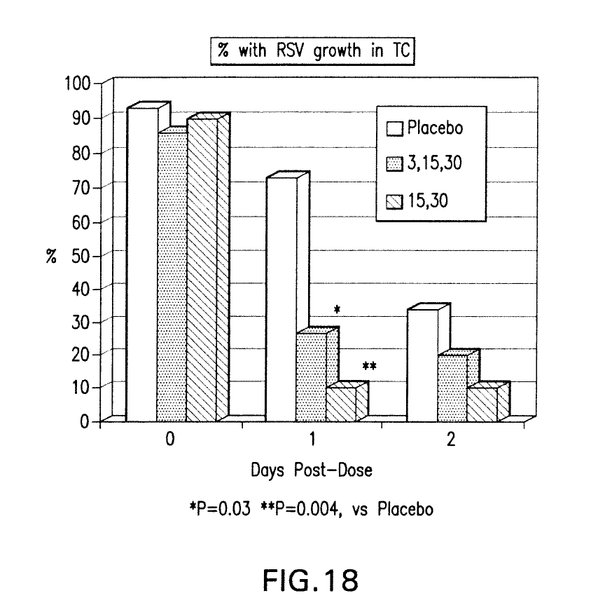

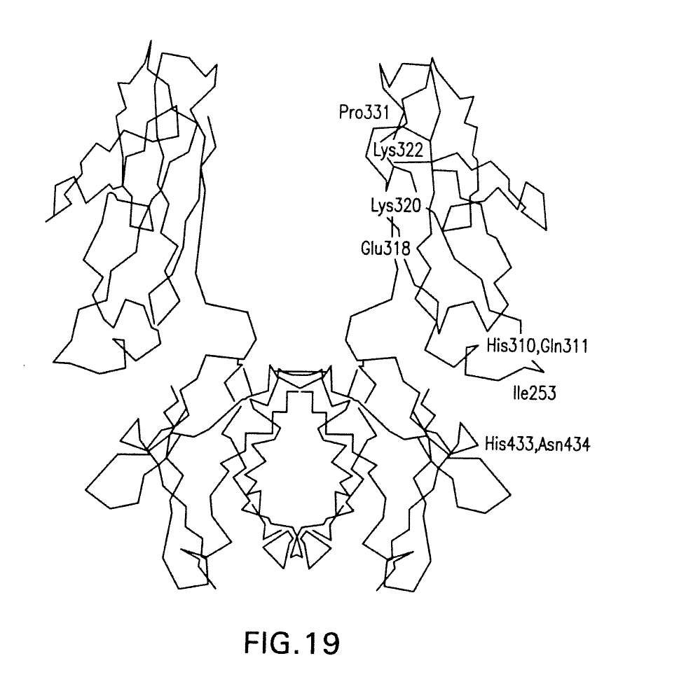

RSV viral titers: RSV viral titers were also assessed in the nasal secretions of children in the various groups at days 0, 1 and 2 post-dose (FIG. 17). Participants who received MEDI-524 (groups pooled) experienced a significant decrease in mean log₁₀ PFU/mL between Study Day 0 and 1 compared to placebo recipients (Mean=−2.6, SD=1.6, vs. −0.9, SD=1.7; p<0.05). In addition, more MEDI-524 than placebo-treated children were antigen negative by Study Day 1 (13/15 vs. 5/15, respectively) (data not shown). The recovery of viral RNA by RT-PCR on Study day 7 was also lower in MEDI-524 than in placebo-treated children (57% vs. 93%) (data not shown). When the nasal secretions were subjected to tissue culture, there was a statistically significant decrease in RSV in nasal secretions recovered from tissue culture in MEDI-524 as compared to placebo-treated patients (FIG. 18). These data indicate that administration of MEDI-524 has an impact on upper respiratory tract infections.

### Results

Conclusion: These data suggest that MEDI-524 given as a single IV 3 mg/kg, 15 mg/kg or 30 mg/kg dose in children hospitalized with RSV infection has a safety, immunogenicity, and PK profile similar to placebo. Additionally, these findings indicate that a single dose of MEDI-524 can reduce RSV levels in the upper airway. Improved clearance of RSV from the upper airway may have added benefits compared to current immunoprophylaxis, including increased efficacy in preventing lower respiratory tract disease, as well the prevention of other diseases or symptoms in which viruses play a role such as otitis media, asthma, wheezing, etc. These data support continued evaluation of MEDI-524 in children hospitalized with RSV LRI and/or URI, and support the evaluation of MEDI-524 for the decrease in the length of hospitalizations in these children.

### 6.15 Phase 3 Clinical Trial of MEDI-524 in High-Risk Children with Prematurity, Chronic Lung Disease of Prematurity, or Chronic Heart Disease

These studies will be conducted similar to those described above in Examples 6.13 and 6.14. Groups of children with prematurity or chronic lung disease of prematurity will be randomized into groups that receive palivizumab or MEDI-524 by single IM dose of 15 mg/kg on day 0 and then in 30 day intervals for months 1, 2, 3, and 4 (i.e., 5 doses total, each separated by 30 day intervals). Each group will be assessed for efficacy and safety throughout the dosage period and for 30 days following the last dose. Primary endpoint will be RSV hospitalization. Secondary endpoints include incidence of lower respiratory tract infection, RSV infection, RSV titers, and incidence and frequency of otitis media.

A follow-up, supportive study will also be conducted in children with complicated chronic heart disease (CHD), similar to those studies outlined above.

### 6.16 Prophylaxis of Otitis Media by A4B4L1Fr-S28R (MEDI-524)

Given the potency of A4B4L1FR-S28R (MEDI-524) at lower doses than palivizumab in preventing upper respiratory infections, similar dosing studies may be performed to determine the efficacy of MEDI-524 and palivizumab in preventing or treating otitis media in humans. Dosing studies may be performed with children at the age of 1 yr as well as with adults at risk for developing otitis media (e.g., adults that are immunocompromised or immunosuppressed). A range of doses (e.g., 2 mg/kg to 60 mg/kg as well as the frequency of doses to be administered may be tested to determine the efficacy of MEDI-524 and palivizumab in preventing or treating otitis media in the experimental groups as compared to a control group (e.g., human infants and adults who are determined to not have otitis media or who are determined to not be at risk for developing otitis media). The antibodies may be administered by any method known in the art, for example, by i.m. injection or intravenously (i.v.). It is anticipated that MEDI-524, at significantly lower doses than palivizumab, will be effective in preventing and/or treating otitis media in the experimental groups (e.g., human infants within the first year of life and in adults who are at risk for developing otitis media) as compared to control groups (e.g., human infants and adults who are determined to not have otitis media or who are determined to not be at risk for developing otitis media).

### 6.17 Production, Isolation, and Characterization of Modified Hinge-Fc Fragments

This example illustrate the production, isolation, and characterization of modified hinge-Fc fragments that have longer in vivo half-lives.

### 6.17.1 Library Construction

### 6.17.1.1 Reagents

All chemicals were of analytical grade. Restriction enzymes and DNA-modifying enzymes were purchased from New England Biolabs, Inc. (Beverly, Mass.). Oligonucleotides were synthesized by MWG Biotech, Inc. (High Point, N.C.). pCANTAB5E phagemid vector, anti-E-tag-horseradish peroxydase conjugate, TG1 E. Coli strain, IgG Sepharose 6 Fast Flow and HiTrap protein A columns were purchased from APBiotech, Inc. (Piscataway, N.J.). VCSM13 helper phage and the Quick change mutagenesis kit were obtained from Stratagene (La Jolla, Calif.). CJ236 E. coli strain was purchased from Bio-Rad (Richmond, Calif.). BCA Protein Assay Reagent Kit was obtained from Pierce (Rockford, Ill.). Lipofectamine 2000 was purchased from Invitrogen, Inc. (Carlsbad, Calif.).

### 6.17.1.2 Expression and Purification of Murine and Human FcRn

The amino acid sequences of human and mouse FcRn are SEQ ID NOS: 84 and 85, respectively (see also Firan et al., Intern. Immunol., 13:993-1002, 2001 and Popov et al., Mol. Immunol., 33:521-530, 1996, both of which are incorporated herein by reference in their entireties). Human FcRn was also obtained following isolation from human placenta cDNA (Clontech, Palo Alto, Calif.) of the genes for human β2-microglobulin (Kabat et al., 1991, Sequences of Proteins of Immunological Interest, U.S. Public Health Service, National Institutes of Health, Washington, D.C.) and codons-23 to 267 of the human a chain (Story et al., J. Exp. Med., 180:2377-2381, 1994) using standard PCR protocols. Light and heavy chains along with their native signal sequence (Kabat et al., 1991, supra; Story et al., supra) were cloned in pFastBac DUAL and pFastBacl bacmids, respectively, and viral stocks produced in Spodoptera frugiperda cells (Sf9) according to the manufacturer's instructions (Invitrogen, Carlsbad, Calif.). High-Five cells were infected at a multiplicity of infection of 3 with the baculoviruses encoding α and β2 chains using commercially available protocols (Invitrogen). Recombinant human FcRn was purified as follows: supernatant of infected insect cells was dialyzed into 50 mM MES (2-N-[Morpholino]ethansulfonic acid) pH 6.0 and applied to a 10 ml human IgG Sepharose 6 Fast Flow column (APBiotech, Piscataway, N.J.). Resin was washed with 200 ml 50 mM MES pH 6.0 and FcRn eluted with 0.1 M Tris-Cl pH 8.0. Purified FcRn was dialyzed against 50 mM MES pH 6.0, flash frozen and stored at −70° C. The purity of proteins was checked by SDS-PAGE and HPLC.

### 6.17.1.3 Preparation of TAA-Containing ssDNA Uracil Template

Construction of the libraries was based on a site directed mutagenesis strategy derived from the Kunkel method (Kunkel et al., Methods Enzymol. 154:367-382, 1987). A human hinge-Fc gene spanning amino acid residues 226-478 (Kabat et al. (1991) Sequences of proteins of immunological interest. (U.S. Department of Health and Human Services, Washington, D.C.) 5^{th} ed.) derived from MEDI-493 human IgG1 (Johnson et al., J. Infect. Disease, 176:1215-1224, 1997), was cloned into the pCANTAB5E phagemid vector as an SfiI/NotI fragment. Four libraries were generated by introducing random mutations at positions 251, 252, 254, 255, 256 (library 1), 308, 309, 311, 312, 314 (library 2), 385, 386, 387, 389 (library 3) and 428, 433, 434, 436 (library 4). Briefly, four distinct hinge-Fc templates were generated using PCR by overlap extension (Ho et al., Gene, 15:51-59, 1989), each containing one TAA stop codon at position 252 (library 1), 310 (library 2), 384 (library 3) or 429 (library 4), so that only mutagenized phagemids will give rise to Fc-displaying phage.

Each TAA-containing single-stranded DNA (TAAssDNA) was then prepared as follows: a single CJ236 E. coli colony harboring one of the four relevant TAA-containing phagemids was grown in 10 ml 2×YT medium supplemented with 10 μg/ml chloramphenicol and 100 μg/ml ampicillin. At OD₆₀₀=1, VCSM13 helper phage was added to a final concentration of 10¹⁰ pfu/ml. After 2 hours, the culture was transferred to 500 ml of 2×YT medium supplemented with 0.25 μg/ml uridine, 10 μg/ml chloramphenicol, 30 μg/ml kanamycin, 100 μg/ml ampicillin and grown overnight at 37° C. Phage were precipitated with PEG6000 using standard protocols (Sambrook et al., 1989, Molecular Cloning: A Laboratory Manual, Cold Spring Harbor Press, Cold Spring Harbor, N.Y., Vols. 1-3) and purified using the QIAPREP Spin M13 Kit (Qiagen, Valencia, Calif.) according to the manufacturer's instructions. 10 to 30 μg of each uracil-containing TAAssDNA template was then combined with 0.6 μg of the following phosphorylated oligonucleotides (randomized regions underlined) in 50 mM Tris-HCl, 10 mM MgCl₂, pH 7.5 in a final volume of 250 μl:

Library 1:

(SEQ ID NO: 378)

5′-CATGTGACCTCAGGSNNSNNSNNGATSNNSNNGGTGTCCTTGGGTT
TTGGGGGG-3′

Library 2:

(SEQ ID NO: 379)

5′-GCACTTGTACTCCTTGCCATTSNNCCASNNSNNGTGSNNSNNGGT
GAGGACGC-3′

Library 3:

(SEQ ID NO: 380)

5′-GGTCTTGTAGTTSNNCTCSNNSNNSNNATTGCTCTCCC-3′

Library 4:

(SEQ ID NO: 381)

5′-GGCTCTTCTGCGTSNNGTGSNNSNNCAGAGCCTCATGSNNCACGGA
GCATGAG-3′

where N=A, C, T or G and S=G or C.

### 6.17.1.4 Synthesis of Heteroduplex DNA

Appropriate, degenerate oligonucleotides were phosphorylated in the presence of T4 polynucleotide kinase using the standard protocol. Ten to 30 μg of ssDNA U template and 0.6 μg of phosphorylated oligonucleotide were combined in 50 mM Tris-HCl containing 10 mM MgCl₂, pH 7.5, to a final volume of 250 μl and incubated at 90° C. for 2 minutes, 50° C. for 3 minutes, and 20° C. for 5 minutes. Synthesis of the heteroduplex DNA was carried out by adding 30 units of both T4 DNA ligase and T7 DNA polymerase in the presence of 0.4 mM ATP, 1 mM dNTPs and 6 mM DTT and the mixture was incubated for 4 hours at 20° C. The heteroduplex DNA thus produced was then purified and desalted using Qiagen QIAQUICK® DNA purification Kit (Qiagen, CA).

### 6.17.1.5 Electroporation

Three hundred microliters of electrocompetent TG 1 E. coli cells were electroporated with 1 to 5 μg of the heteroduplex DNA in a 2.5 kV field using 200Ω and 25 μF capacitance until a library size of 1×10⁸ (library 1 and 2) or 1×10⁷ (library 3 and 4) was reached. The cells were resuspended in 2 ml SOC medium and the procedure was repeated 6 to 10 times. The diversity was assessed by titration of recombinant E. coli. The pulsed cells were incubated in 50 ml SOC medium for 30 minutes at 37° C. under agitation, centrifuged, and resuspended in 500 ml 2×YT containing 100 μg/ml ampicillin and 10¹⁰ pfu/ml of VCSM13 helper phage. The culture was incubated overnight at 37° C. and the cells were pelleted by centrifugation. The phage in the supernatant which express mutated hinge-Fc portion on its GIII-coat protein were precipitated with PEG6000 as previously described (Sambrook et al., 1989, supra) and resuspended in 5 ml of 20 mM MES, pH 6.0.

### 6.17.2 Panning of the Library

Phage were panned using an ELISA-based approach. A 96-well ELISA plate was coated with 100 μl/well of 0.01 mg/ml murine FcRn in sodium carbonate buffer, pH 9.0, at 4° C. overnight and then blocked with 4% skimmed milk at 37° C. for 2 hours. In each well of the coated plate, 100-150 μl of the phage suspension (about 10¹³ phage in total) in 20 mM MES, pH 6.0, containing 5% milk and 0.05% Tween 20, were placed and incubated at 37° C. for two to three hours with agitation.

After the incubation, the wells were washed with 20 mM MES, pH 6.0, containing 0.2% Tween 20 and 0.3 M NaCl about thirty times at room temperature. The bound phage were eluted with 100 μl/well of PBS, pH 7.4, at 37° C. for 30 minutes.

The eluted phage were then added to the culture of exponentially growing E. coli cells and propagation was carried out overnight at 37° C. in 250 ml 2×YT supplemented with 100 μg/ml ampicillin and 10¹⁰ pfu/ml of VCSM13 helper phage. Propagated phage were collected by centrifugation followed by precipitation with PEG and the panning process was repeated up to a total of six times.

For the phage library containing mutations in residues 308-314 (H310 and W313 fixed), the phage expressing hinge-Fc region with higher affinities for FcRn were enriched by each panning process as shown in Table 29. The panning results of the library for the mutations in the residues 251-256 (1253 fixed) and that of the library for the mutations in the residues 428-436 (H429, E430, A431, L432, and H435 fixed), are shown in Tables 30 and 31, respectively. Furthermore, the panning results of the library for the mutations in the residues 385-389 (E388 fixed) is shown in Table 32.

TABLE 29
PANNING OF LIBRARY (RESIDUES 308-314; H310 AND W313
FIXED) pCANTAB5E-KUNKEL-muFcRn (MURINE FcRn)

| | |
|---|---|
| OUTPUT | ENRICHMENT |

| PANNING | +FcRn | −FcRn | RATIO |
|---|---|---|---|
| 1st Round | 1.1 × 10⁵ | 0.5 × 10⁵ | 2 |
| 2nd Round | 1 × 10⁴ | 0.2 × 10⁴ | 5 |
| 3rd Round | 9 × 10⁴ | 0.3 × 10⁴ | 30 |
| 4th Round | 3 × 10⁵ | 2 × 10⁴ | 15 |

TABLE 30
PANNING OF LIBRARY (RESIDUES 251-256;
I253 FIXED) pCANTAB5E-KUNKEL-muFcRn

| | |
|---|---|
| OUTPUT | ENRICHMENT |

| PANNING | +FcRn | −FcRn | RATIO |
|---|---|---|---|
| 1st Round | 2.5 × 10⁵ | 1 × 10⁵ | 2.5 |
| 2nd Round | 6 × 10⁴ | 2 × 10⁴ | 3.0 |
| 3rd Round | 8 × 10⁵ | 4 × 10⁴ | 20 |
| 4th Round | 1.2 × 10⁶ | 5 × 10⁴ | 24 |
| 5th Round | 3.0 × 10⁶ | 6 × 10⁴ | 50 |

TABLE 31
PANNING OF LIBRARY (RESIDUES 428-436; H429, E430,
A431, L432, AND H435 FIXED) pCANTAB5E-KUNKEL-muFcRn

| | |
|---|---|
| OUTPUT | ENRICHMENT |

| PANNING | +FcRn | −FcRn | RATIO |
|---|---|---|---|
| 1st Round | 2.3 × 10⁵ | 0.9 × 10⁵ | 2.5 |
| 2nd Round | 3 × 10⁴ | 1 × 10⁴ | 3 |
| 3rd Round | 2 × 10⁵ | 2 × 10⁴ | 10 |
| 4th Round | 8 × 10⁵ | 5 × 10⁴ | 16 |

TABLE 32
PANNING OF LIBRARY (RESIDUES 385-389;
E388 FIXED) pCANTAB5E-KUNKEL-muFcRn

| | |
|---|---|
| OUTPUT | ENRICHMENT |

| PANNING | +FcRn | −FcRn | RATIO |
|---|---|---|---|
| 1st Round | 4.2 × 10⁵ | 3.8 × 10⁵ | 1.1 |
| 2nd Round | 5 × 10⁴ | 0.3 × 10⁴ | 17 |
| 3rd Round | 3.5 × 10⁵ | 1 × 10⁴ | 35 |
| 4th Round | 5.5 × 10⁵ | 4 × 10⁴ | 14 |
| 5th Round | 7.5 × 10⁵ | 5 × 10⁴ | 15 |
| 6th Round | 2 × 10⁶ | 1 × 10⁵ | 20 |

### 6.17.3 Identification of Isolated Clones from Panning

After each panning process, phage were isolated and the nucleic acids encoding the expressed peptides which bound to FcRn were sequenced by a standard sequencing method such as by dideoxynucleotide sequencing (Sanger et al., Proc. Natl. Acad. Sci. USA, 74:5463-5467, 1977) using a ABI3000 genomic analyzer (Applied Biosystems, Foster City, Calif.).

As a result of panning, two mutants were isolated from the phage library containing mutations in residues 308-314 (H310 and W313 fixed), thirteen mutants from the library for residues 251-256 (1253 fixed), six mutants from the library for residues 428-436 (H429, E430, A431, L432, and H435 fixed), and nine mutants from the library for residues 385-389 (E388 fixed). The mutants isolated from the libraries are listed in Table 33.

TABLE 33
MUTANTS ISOLATED BY PANNING

| | |
|---|---|
| LIBRARY | MUTANTS* |

| | |
|---|---|
| 251-256 | Leu Ile Thr Arg Glu (SEQ ID NO: 348) |
| | Leu [symbol] Ile Ser Arg Thr (SEQ ID NO: 349) |
| | Leu [symbol] Ile Ser Arg [symbol] (SEQ ID NO: 350) |
| | Leu [symbol] Ile Ser Arg [symbol] (SEQ ID NO: 351) |
| | Leu [symbol] Ile Ser Arg [symbol] (SEQ ID NO: 352) |
| | Leu [symbol] Ile Ser Arg Thr (SEQ ID NO: 353) |
| | Leu Tyr Ile Ser Leu Gln (SEQ ID NO: 354) |
| | Leu Phe Ile Ser Arg Asp (SEQ ID NO: 355) |
| | Leu Phe Ile Ser Arg Thr (SEQ ID NO: 356) |
| | Leu Phe Ile Ser Arg Arg (SEQ ID NO: 357) |
| | Leu Phe Ile Thr Gly Ala (SEQ ID NO: 358) |
| | Leu Ser Ile Ser Arg Glu (SEQ ID NO: 359) |
| | Arg Thr Ile Ser Ile Ser (SEQ ID NO: 360) |
| 308-314 | Thr Pro His Ser Asp Trp Leu (SEQ ID NO: 361) |
| | Ile Pro His Glu Asp Trp Leu (SEQ ID NO: 362) |
| 385-389 | Arg Thr Arg Glu Pro (SEQ ID NO: 363) |
| | [symbol] [symbol] Pro Glu [symbol] (SEQ ID NO: 364) |
| | Ser Asp Pro Glu Pro (SEQ ID NO: 365) |
| | Thr Ser His Glu Asn (SEQ ID NO: 366) |
| | Ser Lys Ser Glu Asn (SEQ ID NO: 367) |
| | His Arg Ser Glu Asn (SEQ ID NO: 368) |
| | Lys Ile Arg Glu Asn (SEQ ID NO: 369) |
| | Gly Ile Thr Glu Ser (SEQ ID NO: 370) |
| | Ser Met Ala Glu Pro (SEQ ID NO: 371) |
| 428-436 | Met His Glu Ala Leu [symbol] [symbol] His [symbol] (SEQ ID NO: 372) |
| | Met His Glu Ala Leu His Phe His His (SEQ ID NO: 373) |
| | Met His Glu Ala Leu Lys Phe His His (SEQ ID NO: 374) |
| | Met His Glu Ala Leu Ser Tyr His Arg (SEQ ID NO: 375) |
| | Thr His Glu Ala Leu His Tyr His Thr (SEQ ID NO: 376) |
| | Met His Glu Ala Leu His Tyr His Tyr (SEQ ID NO: 377) |

*Substituting residues are indicated in bold face

The underlined sequences in Table 33 correspond to sequences that occurred 10 to 20 times in the final round of panning and the sequences in italics correspond to sequences that occurred 2 to 5 times in the final round of panning. Those sequences that are neither underlined nor italicized occurred once in the final round of panning.

### 6.17.4 Expression and Purification of Soluble Mutant Hinge-Fc Region

The genes encoding mutated hinge-Fc fragments are excised with appropriate restriction enzymes and recloned into an expression vector, for example, VβpelBhis (Ward, J. Mol. Biol., 224:885-890, 1992). Vectors containing any other type of tag sequence, such as c-myc tag, decapeptide tag (Huse et al., Science, 246:1275-1281, 1989), FLAG™ (Immunex) tags, can be used. Recombinant clones, such as E. coli, are grown and induced to express soluble hinge-Fc fragments, which can be isolated from the culture media or cell lysate after osmotic shock, based on the tag used, or by any other purification methods well known to those skilled in the art and characterized by the methods as listed below.

### 6.17.5 Construction, Production and Purification of IgG1 Variants

Representative Fc mutations such as I253A, M252Y/S254T/T256E, M252W, M252Y, M252Y/T256Q, M252F/T256D, V308T/L309P/Q311S, G385D/Q386P/N389S, G385R/Q386T/P387R/N389P, H433K/N434F/Y436H, and N434F/Y436 were incorporated into the human IgG1 MEDI-493 (palivizumab) (Johnson et al., 1997, supra). The heavy chain was subjected to site-directed mutagenesis using a Quick Change Mutagenesis kit (Stratagene, La Jolla, Calif.) according to the manufacturer's instructions and sequences were verified by didoxynucleotide sequencing using a ABI3000 (Applied Biosystems, Foster City, Calif.) sequencer. The different constructions were expressed transiently in human embryonic kidney 293 cells using a CMV immediate-early promoter and dicistronic operon in which IgG1/V_{H} is cosecreted with IgG1/V_{L} (Johnson et al., 1997, supra). Transfection was carried out using Lipofectamine 2000 (Invitrogen, Carlsbad, Calif.) and standard protocols. IgGs were purified from the conditioned media directly on 1 ml HiTrap protein A columns according to the manufacturer's instructions (APBiotech).

### 6.17.6 Characterization of Mutated Hinge-Fc Region

### 6.17.6.1 In Vitro Characterization HPLC and SDS-Page

Following the purification, general characteristics such as molecular weight and bonding characteristics of the modified hinge-Fc fragments may be studied by various methods well known to those skilled in the art, including SDS-PAGE and HPLC.

### FcRn Binding Assay

Binding activity of modified hinge-Fc fragments can be measured by incubating radio-labeled wild-type hinge-Fc or modified hinge-Fc with the cells expressing either mouse or human FcRn. Typically, endothelial cell lines such as SV40 transformed endothelial cells (SVEC) (Kim et al., J. Immunol., 40:457-465, 1994) are used. After incubation with the hinge-Fc fragments at 37° C. for 16-18 hours, the cells are washed with medium and then detached by incubation with 5 mM Na₂EDTA in 50 mM phosphate buffer, pH 7.5, for 5 minutes. The radioactivity per 10⁷ cells is measured.

Then, the cells are resuspended in 2 ml of 2.5 mg/ml CHAPS, 0.1 M Tris-HCl pH 8.0 containing 0.3 mg/ml PMSF, 25 mg/ml pepstatin and 0.1 mg/ml aprotinin and incubated for 30 minutes at room temperature. The cell suspension is then centrifuged and the supernatant separated. The radioactivity of the supernatant is measured and used to calculate the amount of the hinge-Fc fragments extracted per 10⁷ cells.

The K_{d} for the interaction of wild type human IgG1 with murine and human FcRn (269 and 2527 nM, respectively) agree well with the values determined by others (265 and 2350 nM, respectively, Firan et al., 2001, supra). The 1253A mutation virtually abolishes binding to human and murine FcRn, as reported by others (Kim et al., Eur. J. Immunol., 29:2819-2825, 1991; Shields et al., J. Biol. Chem., 276:6591-6604, 2001). This is not the result of misfolding of the antibody as this mutant retains the same specific activity than the wild type molecule (palivizumab) in a microneutralization assay (Johnson et al., 1997, supra; data not shown).

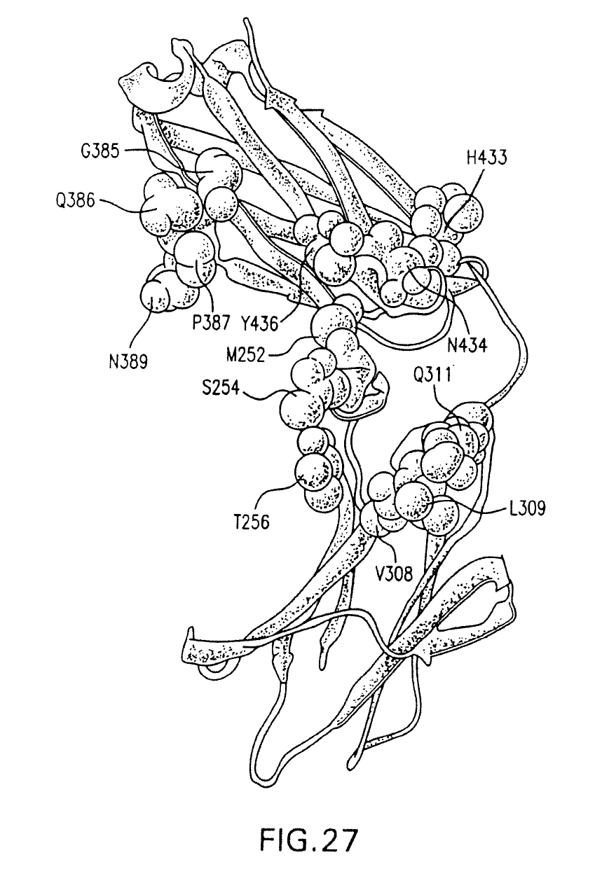

Human IgG1 mutants with increased binding affinity towards both murine and human FcRn were generated (Table 33). Improvements in complex stability were overall less marked for the human IgG1-human FcRn pair than for the human IgG1-murine FcRn compared to wild type IgG1 were 30-(ΔΔG=2.0 kcal/mol for N434F/Y436H) and 11-(ΔΔG=1.4 kcal/mol for M252Y/S254Y/S254T/T256E) fold, respectively. However, ranking of the most critical positions remain unchanged when comparing human and murine FcRn: the largest increases in IgG1-murine FcRn complex stability (ΔΔG>1.3 kcal/mol) occurred on mutations at positions 252, 254, 256 (M252Y/S254T/T256E and M252W) and 433, 434, 436 (H433K/N434F/Y436H and N434F/Y436H). Likewise, the same mutations were found to have the most profound impact on the IgG1-human FcRn interaction and also resulted in the largest increases in complex stability (ΔΔG>1.0 kcal/mol). Substitutions at positions 308, 309, 311, 385, 386, 387 and 389 had little or no effect on the stability of the complexes involving human or murine FcRn (ΔΔG<0.5 kcal/mol). Residues at the center of the Fc-FcRn combining site contribute significantly more to improvement in complex stability than residues at the periphery (FIG. 27).

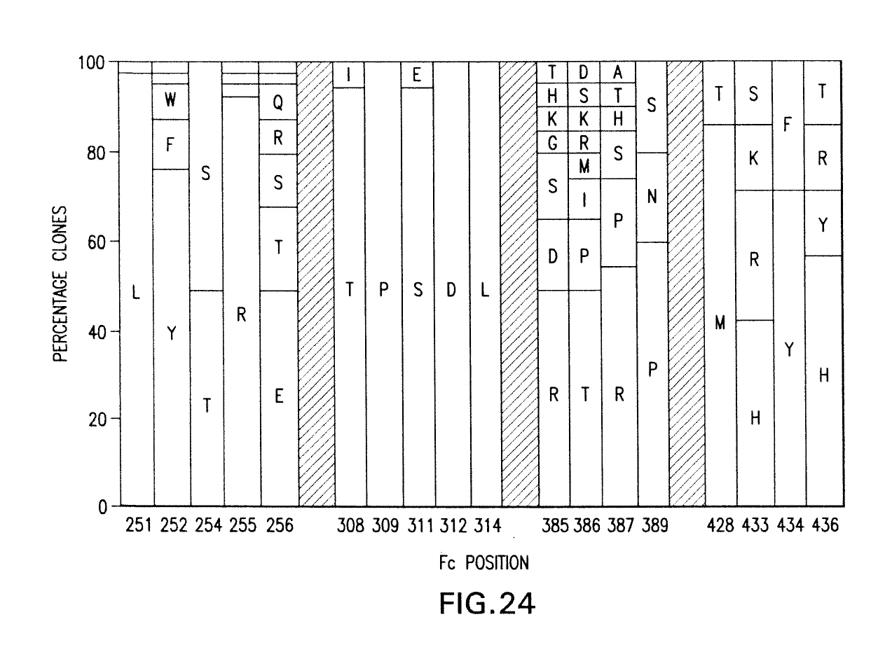

Efficient binding of human Fc to murine FcRn apparently requires the presence of several wild type Fc residues. For example, leucine is very conserved at 251, arginine at 255, aspartic acid at 310, leucine at 314 and methionine at 428 (FIG. 24). Another specificity trend is observed when one considers positions 308, 309, and 311 where threonine, proline, and serine, respectively, are very strongly favored over the corresponding wild type residues (FIG. 24). However, generation of this strong consensus sequences does not correlate with the magnitude of increase in affinity as V308T/L309P/Q311S binds less than 2-fold better than the wild type IgG1 to both human and murine FcRn (Table 34).

Increases in affinity can be strongly dependent upon residue substitution at one ‘hot spot’ position. For example, the single mutation M252Y causes an increase in binding to murine FcRn by 9-fold, whereas additional mutations bring little (M252Y/S254T/T256E) or no (M252Y/T256Q) added benefit. The same trend is observed for the human receptor, although to a lesser extent. Indeed, M252Y/S254T/T256E shows a marked improvement of 2.5-fold in affinity compared to M252Y. This probably reflects the differences between the binding site of human and murine FcRn (West and Bjorkman, Biochemistry, 39:9698-9708, 2000).

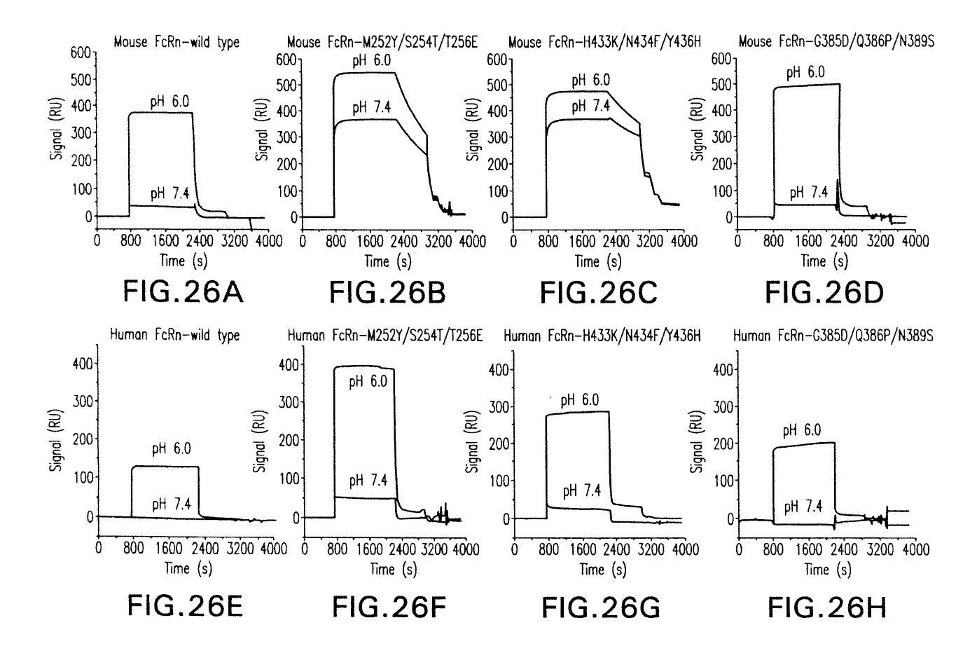

Phage-derived IgG1 mutants exhibiting a significant increase in affinity towards murine FcRn (ΔΔG>1.3 kcal/mol) also showed significant binding activity to the receptor at pH 7.2 when compared to wild type IgG1 (FIGS. 26A-26H). IgG1 mutants with moderate increase in affinity (ΔΔG<0.3 kcal/mol) bound very poorly at pH 7.2 (data not shown). In contrast, IgG1 mutants with large (ΔΔG>1.0 kcal/mol) increase in affinity towards human FcRn exhibited only minimal binding at pH 7.4 when compared to wild type IgG1 (FIGS. 26A-26H).

TABLE 34
DISSOCIATION CONSTANTS AND RELATIVE FREE
ENERGY CHANGES FOR THE BINDING OF IgG1/FC
MUTANTS TO MURINE AND HUMAN FcRn*

| | Dissociation | | Dissociation | |
|---|---|---|---|---|
| | Constant | ΔΔG | Constant | ΔΔG |
| | Fc/Murine | (kcal/ | Fc/Human | (kcal/ |
| MUTANT | FcRn (nM) | mol) | FcRn (mM) | mol) |
| wild type | 269 ± 1 | | 2527 ± 117 | |
| I253A | NB | NA | NB | NA |
| M252Y/S254T/T256E | 27 ± 6 | 1.4 | 225 ± 10 | 1.4 |
| M252W | 30 ± 1 | 1.3 | 408 ± 24 | 1.1 |
| M252Y | 41 ± 7 | 1.1 | 532 ± 37 | 0.9 |
| M252Y/T256Q | 39 ± 8 | 1.1 | 560 ± 102 | 0.9 |
| M252F/T256D | 52 ± 9 | 1.0 | 933 ± 170 | 0.6 |
| V308T/L309P/Q311S | 153 ± 23 | 0.3 | 1964 ± 84 | 0.1 |
| G385D/Q386P/N389S | 187 ± 10 | 0.2 | 2164 ± 331 | 0.1 |
| G385R/Q386T/P387R/ | 147 ± 24 | 0.4 | 1620 ± 61 | 0.3 |
| N389P | | | | |
| H433K/N434F/Y436H | 14 ± 2 | 1.8 | 399 ± 47 | 1.1 |
| N434F/Y436H | 9 ± 1 | 2.0 | 493 ± 7 | 1.0 |

*Affinity measurements were carried out by BIAcore as described above. Residue numbering is according to EU (Kabat et al., 1991, supra). Differences in free energy changes are calculated as the differences between the Δgs of wild type and mutant reactions (ΔΔG = ΔG_{wild type} − ΔGₘᵤₜₐₙₜ). NB, no binding. NA, not-applicable.

### FcRn-Mediated Transfer Assay

This assay follows the protocol disclosed in PCT publication WO 97/34631. Radiolabeled modified hinge-Fc fragments at various concentration (1 μg/ml-1 mg/ml) are added to the one side of the transwell and the transfer of the fragments mediated by FcRn-expressing monolayer of the cells can be quantitated by measuring the radioactivity on the other side of the transwell.

### 6.17.6.2 In Vivo Pharmacokinetic Study

In order to determine the half-life of the modified IgG hinge-Fc, modified hinge-Fc fragments are radiolabelled with ¹²⁵I (approximate specific activity of 10⁷ cpm/μg) and dissolved in saline (pH 7.2). The solution is injected intravenously into BALB/c mice (Harlan, Indianapolis, Ind.), which have been given NaI-containing water previously to block the thyroid, in a volume not more than 150 μl and with a radioactivity of 10×10⁶−50×10⁶ cpm. The mice are bled from the retro-orbital sinus at various time points, for example, at 3 minutes to 72 hours after the injection, into heparinized capillary tubes and the plasma collected from each sample is counted for radioactivity.

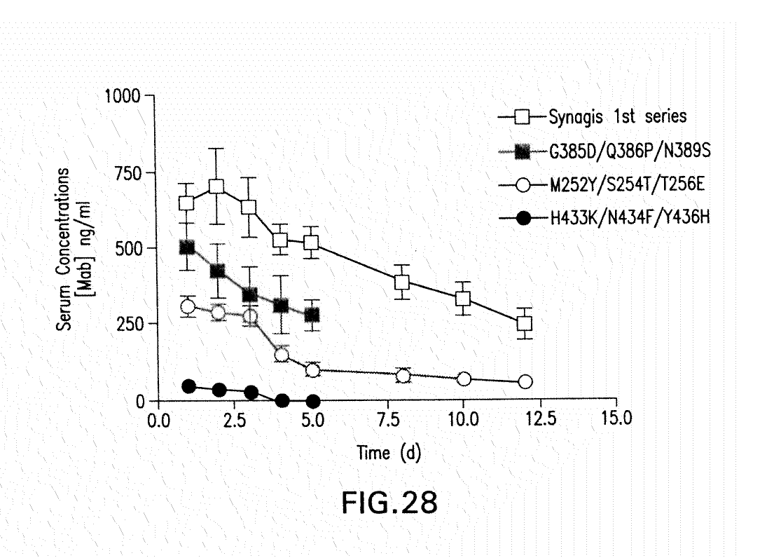

To generate the data provided in FIG. 28, 10 animals were used for each molecule assayed with 2.5 μg of antibody injected per animal. Antibody serum levels were determined using an anti-human IgG ELISA (FIG. 28). There seems to be an inverse correlation between affinity to mouse FcRn and persistence in serum. This might be due to the significant amount of binding of the mutants observed at pH 7.2, which leads to the sequestration (i.e., lack of release in the serum) of the molecules. Preliminary data (not shown) suggests increased transport of the mutants to the lung. Additionally, since the mutants exhibit lower levels of binding to human FcRn than murine FcRn (see FIGS. 26A-26H), antibody serum levels are expected to be higher in primates and humans.

### 6.17.6.3 Surface Plasmon Resonance Analyses

The interaction of soluble murine and human FcRn with immobilized human

IgG1 variants was monitored by surface plasmon resonance detection using a BIAcore 3000 instrument (Pharmacia Biosensor, Uppsala, Sweden). No aggregated material which could interfere with affinity measurements (van der Merwe et al., EMBO J., 12:4945-4954, 1993; van der Merwe et al., Biochemistry, 33:10149-10160, 1994) was detected by gel filtration. Protein concentrations were calculated by the bicinchoninic acid (BCA) method for both human and murine FcRn or using the 1% extinction coefficient at 280 nm of 1.5 for IgG1 wild type and variants. The latter were coupled to the dextran matrix of a CM5 sensor chip (Pharmacia Biosensor) using an Amine Coupling Kit as described (Johnsson et al. Anal. Biochem. 198 (1992) 268-277). The protein concentrations ranged from 3-5 μg/ml in 10 mM sodium acetate, pH 5.0. The activation period was set for 7 minutes at a flow rate of 10 μl/min and the immobilization period was set to between 10 and 20 minutes at a flow rate of 10 μl/min. Excess reactive esters were quenched by injection of 70 μl of 1.0 methanolamine hydrochloride, pH 8.5. This typically resulted in the immobilization of between 500 and 4000 resonance units (RU). Human and murine FcRn were buffer exchanged against 50 mM PBS buffer pH 6.0 containing 0.05% Tween 20. Dilutions were made in the same buffer. All binding experiments were performed at 25° C. with concentrations ranging from 120 to 1 μg/ml at a flow rate of 5 to 10 μl/min; data were collected for 25 to 50 minutes and three 1-minute pulses of PBS buffer pH 7.2 were used to regenerate the surfaces. FcRn was also flowed over an uncoated cell and the sensorgrams from these blank runs subtracted from those obtained with IgG1-coupled chips. Runs were analyzed using the software BIAevaluation 3.1 (Pharmacia). Association constants (K_{A}s) were determined from Scatchard analysis by measuring the concentration of free reactants and complex at equilibrium after correction for nonspecific binding. In equilibrium binding BIAcore experiments (Karlsson et al., 1991, supra; van der Merwe et al., 1993, supra; van der Merwe et al., 1994, supra; Raghavan et al., Immunity, 1:303-315, 1994; Malchiodi et al., J. Exp. Med., 182:1833-1845, 1995), the concentration of the complex can be assessed directly as the steady-state response. The concentration of free analyte (human or murine FcRn) is equal to the bulk analyte concentration since analyte is constantly replenished during sample injection. The concentration of free ligand on the surface of the sensor chip can be derived from the concentration of the complex and from the total binding capacity of the surface as K_{A}=R_{eq}/C(Rₘₐₓ−R_{eq}) where C is the free analyte concentration, R_{eq} is the steady-state response, and Rₘₐₓ is the total surface binding capacity. Rearranging, the equation reads: R_{eq}/C=K_{A} Rₘₐₓ−K_{A} R_{eq}.

A plot of R_{eq}/C versus R_{eq} at different analyte concentrations thus gives a straight line from which K_{A} can be calculated (see Table 34). Errors were estimated as the standard deviation for two or three independent determinations and were <20%.

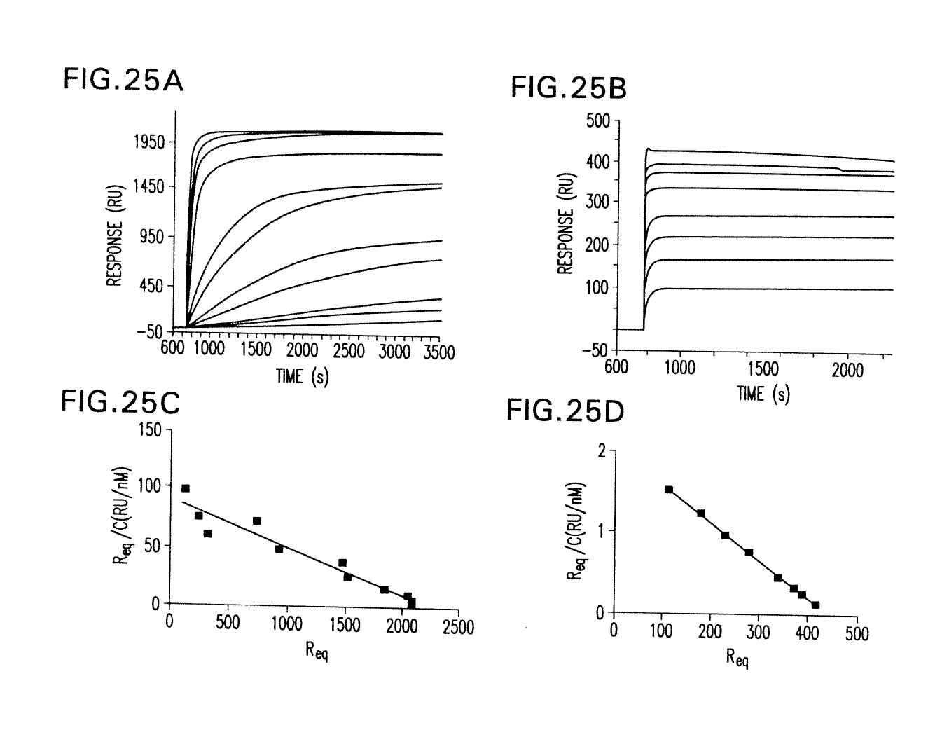

Representative mutations identified after panning libraries 1 through 4 (FIG. 24, Table 33) were introduced into the Fc portion of a human IgG1. Injection of different concentrations of human or murine FcRn over the immobilized IgG1 variants gave concentration-dependent binding. Typical resonance profiles for equilibrium binding of the mutant M252Y/S254T/T256E to murine and human FcRn are shown in FIGS. 25A and 25B. To estimate apparent K_{A}s, concentrations of FcRn ranging from 120 to 1 μg/ml were used. In all cases, equilibrium (or near-equilibrium) binding levels were reached within 50 minutes. To estimate the increase in RU resulting from the non specific effect of protein on the bulk refractive index, binding of FcRn to an uncoated cell was measured and the sensorgrams from these blank runs subtracted from those obtained with IgG1-coupled chips. The scatchard plots for the binding of the mutant M252Y/S254T/T256E to murine and human FcRn are shown in FIGS. 25C and 25D. The plots were all linear, and apparent K_{A}s were calculated from the relevant slopes. Measurements were carried out in duplicate or triplicate and confirmed that the immobilized IgGs retained their original binding activity.

Since there are two non-equivalent binding sites on mouse IgG1 for murine FcRn with affinities of <130 nM and 6 μM (Sanchez et al., Biochemistry, 38:9471-9476, 1999; Schuck et al., Mol. Immunol., 36:1117-1125, 1999; Ghetie and Ward, Ann. Rev. Immunol., 18:739-766, 2000), the receptor was used in solution to avoid avidity effects that arise when IgG1 binds to immobilized FcRn. Consistent with this, systematically higher affinities are observed when FcRn, rather than IgG, immobilized on the biosensor chip (Popov et al., 1996, supra; Vaughn and Bjorkman, Biochemistry, 36:9374-9380, 1997; Martin and Bjorkman, Biochemistry, 38:12639-12647; West and Bjorkman, Biochemistry, 39:9698-9708, 2000). Under our experimental BIAcore conditions, mainly interactions corresponding to the higher-affinity association (i.e., single liganded-receptor) are measured, according for the linearity of the scatchard plots (FIGS. 25C and 25D).

BIAcore analysis was also used to compare the affinity of wild type IgG1 and IgG1 mutants. Phage-derived IgG1 mutants exhibiting a significant increase in affinity towards murine FcRn at pH 6.0 (ΔΔG≧1.0 kcal/mol) also shoed significant binding to the mouse receptor at pH 7.2 with SPR signal_{pH7.4}/SPR signal_{pH6.0}>0.6 at saturation. IgG1 mutants with moderate increase in affinity towards murine FcRn at pH 6.0 (ΔG<0.4 kcal/mol) bound very poorly to the mouse receptor at pH 7.2. In contrast, IgG1 mutants exhibiting large affinity increase towards human FcRn at pH 6.0 (ΔΔG≧1.0 kcal/mol) only showed minimal binding to the human receptor at pH 7.4 with SPR signal_{pH7.4}/SPR signal_{pH6.0}<0.15 at saturation.

### 6.18 Generation OF A A4B4L1FR-S28R (MEDI-524) Modified Antibody

This example illustrate the generation of a A4B4L1FR-S28R (MEDI-524) M252Y/S254T/T256E (a YTE) variant.

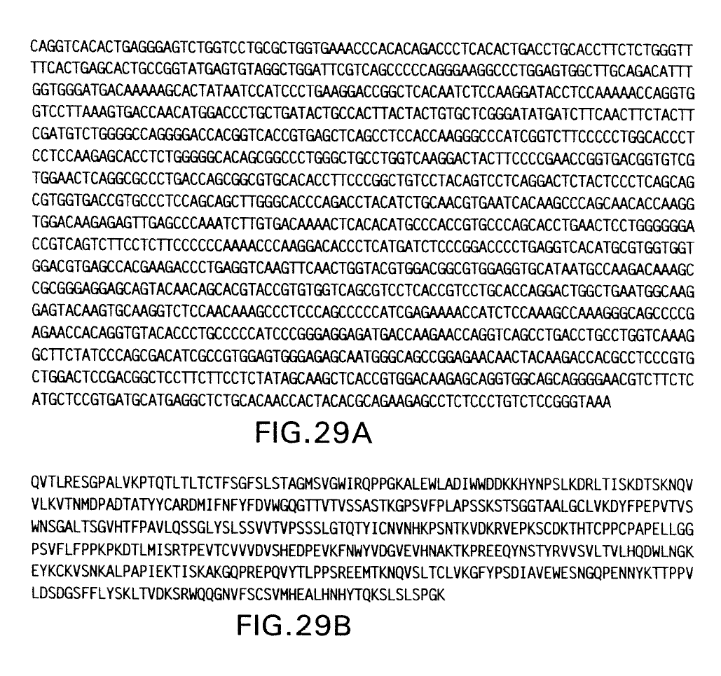

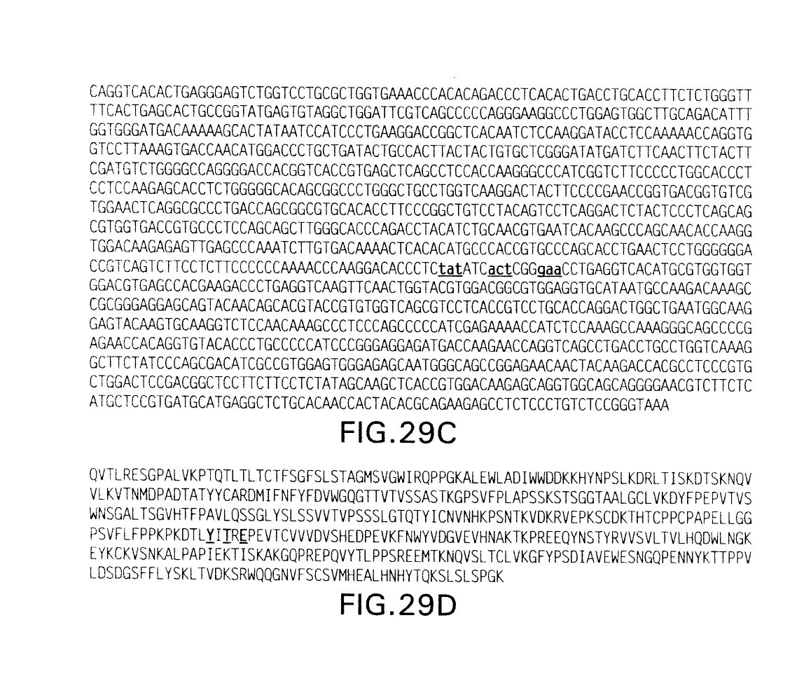

The heavy chain of a humanized MEDI-524 anti-RSV monoclonal antibody was cloned into a mammalian expression vector encoding a human cytomegalovirus major immediate early (hCMVie) enhancer, promoter and 5′-untranslated region (Boshart et al (1985) Cell 41:521-530.). In this system, a human γ1 chain is secreted along with a human κ chain (Johnson et al. (1997) Infect. Dis. 176:1215-1224). A combination of three mutations (M252Y/S254T/T256E; Example 6.17, and Dall'Acqua et al. (2002), J. Immunol. 169:5171-5180) was introduced into the heavy chain of MEDI-524. Generation of these three mutations (collectively referred to as “YTE”) at positions 252, 254 and 256 (EU Index, as in Kabat et al. (1991) Sequences of proteins of immunological interest. (U.S. Department of Health and Human Services, Washington, D.C.) 5^{th} ed., was carried out by site-directed mutagenesis using a Quick Change® XL Mutagenesis Kit (Stratagene, CA) and the primers: 5′-GCATGTGACCTCAGGTTCCCGAGTGATATAGAGGGTGTCCTTGGG-3′ (SEQ ID NO:382) and 5′-CCCAAGGACACCCTCTATATCACTCGGGAACCTGAGGTCACATGC-3′ (SEQ ID NO:383) according to the manufacturer's instructions. This generated “MEDI-524-YTE.” The sequences were verified using an ABI 3100 sequencer and are reported in FIG. 29. NS0 cells were then stably transfected with the corresponding antibody constructs, and the secreted immunoglobulins were expressed and purified using standard protocols.

### Surface Plasmon Resonance (BIAcore) Measurements

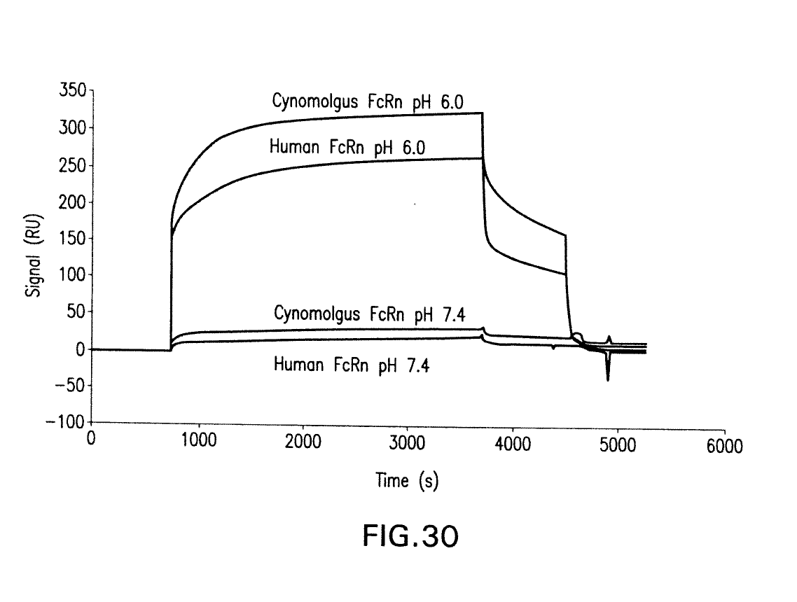

The interaction of soluble human and Cynomolgus Monkey FcRn with immobilized MEDI-524 and MEDI-524-YTE variant was monitored by surface plasmon resonance detection using a BIAcore 3000 instrument (Pharmacia Biosensor, Uppsala, Sweden). Protein concentrations were calculated by the bicinchoninic acid method for both human and Cynomolgus Monkey FcRn or using the 1% extinction coefficient at 280 nm of 1.47 for MEDI-524 and MEDI-524-YTE. Both IgGs were coupled to the dextran matrix of a CM5 sensor chip (Pharmacia Biosensor) using an Amine Coupling Kit as described (Johnsson et al. (1992) Anal. Biochem. 198:268-277) at a surface density of between 947 and 1244 RUs. Human and Cynomolgus Monkey FcRn were buffer-exchanged against 50 mM Phosphate Buffered Saline (PBS) pH 6.0 or 7.4 containing 0.05% Tween 20. Dilutions were made in the same buffers. All binding experiments were performed at 25° C. with FcRn concentrations typically ranging from 2.86 μM to 6 nM at a flow rate of 5 μL/min; data were collected for approximately 50 min and three 1-min pulses of PBS pH 7.4 containing 0.05% Tween 20 were used to regenerate the surfaces. FcRn was also flowed over an uncoated cell and the sensorgrams from these blank runs subtracted from those obtained with IgG-coupled chips. Runs were analyzed using the software BIAevaluation 3.1 (Pharmacia). Dissociation constants (K_{d}s) were determined by fitting the binding isotherms to a one-site binding model using GraphPad Prism (GraphPad Software, Inc., CA). Values are reported in Table 35 below. Errors were estimated as the mean standard deviation for at least 2 independent determinations. As shown in Table 35, MEDI-524-YTE exhibits an affinity increase of 11 and 9-fold towards human and Cynomolgus Monkey FcRn, respectively, when compared with MEDI-524. Furthermore, MEDI-524-YTE retains a significant pH dependency of binding to both human and Cynomolgus monkey FcRn, exhibiting only marginal binding at pH 7.4 (see FIG. 30).

TABLE 35
Dissociation constants for the binding of MEDI-524 and MEDI-
524-YTE variant to Human and Cynomolgus Monkey FcRn.

| | K_{d}-Cynomolgus FcRn | K_{d}-Human FcRn |
|---|---|---|
| Molecule | (nM) | (nM) |
| MEDI-524 | 1196 ± 240 | 2249 ± 84 |
| MEDI-524-YTE | 134 ± 7 | 210 ± 80 |

### Microneutralization Assay

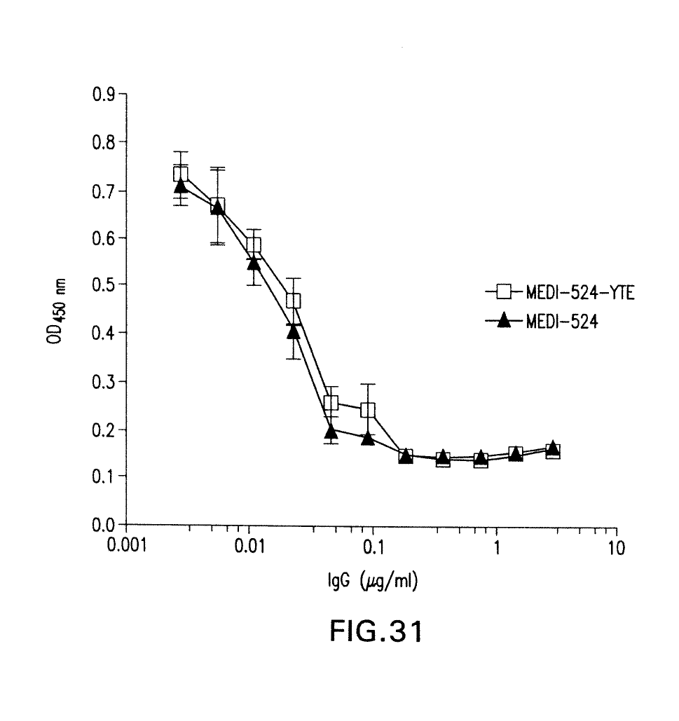

The microneutralization assay was carried out essentially as described (Johnson et al. (1997) Infect. Dis. 176:1215-1224). Briefly, dilutions of MEDI-524 or MEDI-524-YTE were made in quadruplicate in a 96-well plate. RSV (ATCC, Manassas, Va.) was added to each well and incubated for 2 h at 37° C. in 5% CO₂. 2×10⁴ Hep-2 cells (ATCC, Manassas, Va.) were then added to each well and incubated for 5 days at 37° C. in 5% CO₂. Cells were then washed three times with PBS containing 0.1% Tween 20 and fixed with acetone. Viral replication was quantified by successive incubations with a mouse anti-RSV monoclonal antibody (Chemicon, Temecula, Calif.) and a horse radish peroxidase conjugate of a goat anti-mouse IgG (TAGO, Burlingame, Calif.). Peroxidase activity was detected with 3,3′,5,5′-tetramethylbenzidine (TMB) and the reaction was quenched with 2 M H₂SO₄. The absorbance was read at 450 nm and plotted for each antibody concentration (See FIG. 31). As shown in FIG. 31, both MEDI-524 and MEDI-524-YTE exhibit undistinguishable RSV microneutralization properties.

### Cynomolgus Monkey Pharmacokinetics Study

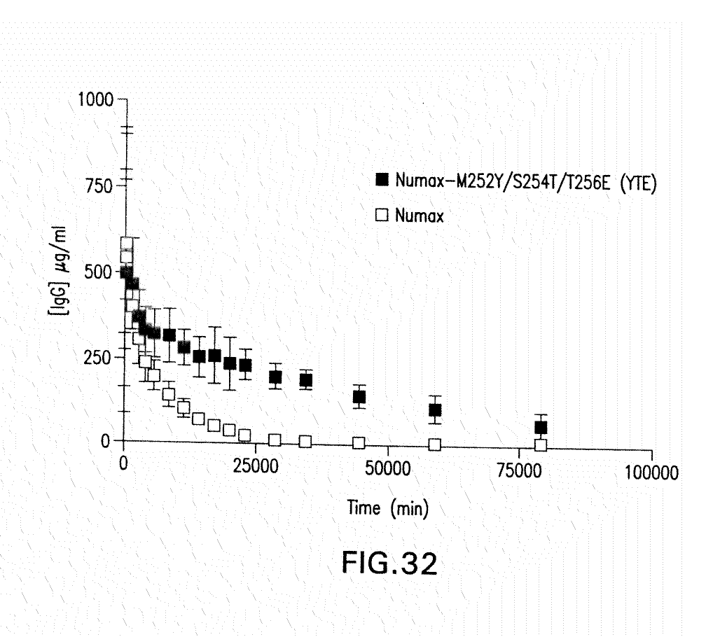

A pharmacokinetics (PK) study was conducted at Gene Logic (Gene Logic Laboratories, Gaithersburg, Md.). Twenty (20) male Cynomolgus Monkeys were randomized and assigned to one of two study groups. Each animal received a single intravenous dose of MEDI-524 (group 1) or MEDI-524-YTE (group 2) at 30 mg/kg. Blood samples were drawn prior to dosing on day 0, at 1 and 4 h after dosing, and at 1, 2, 3, 4, 6, 8, 10, 12, 14, 16, 20, 24, 31, 41 and 55 days after dosing. The concentrations of MEDI-524 or MEDI-524-YTE in serum samples were determined by an anti-human IgG enzyme-linked immunosorbent assay (ELISA). In this assay, MEDI-524 and MEDI-524-YTE are captured by a goat anti-MEDI-524 antibody (anti-idiotype, MedImmune, Inc.) coated to a microtiter plate. Any bound MEDI-524 or MEDI-524-YTE is detected using a goat anti-human IgG antibody linked to biotin. Streptavidin conjugated to horseradish peroxidase followed by tetramethylbenzidine (TMB) as substrate is used for the colorimetric reaction. The corresponding serum clearance curves are shown in FIG. 32. For each injection, a noncompartmental model was fitted for the serum concentration data of each animal. Descriptive statistics were calculated for each of the pharmacokinetics parameters and are reported in Table 36 below. The Wilcoxon test was used to compare the half-lives and AUCs (see footnote to Table 36) between the two treatment groups. As shown in Table 36, the half-lives in group 2 were nearly four times as much as those of group 1. Likewise, the AUCs in group 2 were nearly five times as much as those of group 1. The Wilcoxon test suggests the group differences in half-life and AUCs were statistically significant (p<0.001). The mean maximum serum antibody concentration is very similar between group 1 and 2, indicating that MEDI-524 and MEDI-524-YTE are distributed to the circulation in a similar fashion.

TABLE 36
Descriptive Summary of the Pharmacokinetics Parameters
for MEDI-524 and MEDI-524-YTE in Cynomolgus Monkeys.

| | β phase t_{1/2} ^{a} | C_{MAX} ^{b} | AUC ^{c} |
|---|---|---|---|
| Molecule | (days) | (μg/ml) | (μg · hr/mL) |
| MEDI-524 | 5.7 ± (1.4) | 644 ± (211) | 61172 ± (15529) |
| MEDI-524-YTE | 21.2 ± (9.1) | 639 ± (248) | 294836 ± (95212) |

^{a} Half-life of serum concentration.
^{b} Peak serum concentration.
^{c} Area under the serum concentration-time curve to infinity.
Numbers in parentheses are standard deviations.

### Conclusion

Thus, based upon the above results of introducing the Fc mutations

M252Y/S254T/T256E into MEDI-524, an ultra-potent anti-RSV mAb, the serum half-life will likely be similarly increased in human. It is likely that by combining the ultra-potency of MEDI-524 and other high potency (and/or high affinity and/or high avidity) antibodies with the half-life extension property of the Fc mutations, the modified antibodies of the invention, including MEDI-524-YTE, can be used as long-lasting drugs that require only one or two administrations for the entire treatment course, e.g., during a RSV season. The Cynomolgus Monkey study discussed above has already shown that such construct (MEDI-524 with Fc mutations (i.e., MEDI-524-YTE)) had an about fourfold increase in serum half-life when compared with the MEDI-524 wild-type antibody, and the concentration under the curve (PK) was also substantially increased (by a factor of about 5-fold) (see FIG. 32). Additionally the mutations on Fc did not alter the ability of MEDI-524 to neutralize RSV in a microneutralization assay nor did they affect the binding affinity to its cognate antigen.

### 6.19 Generation OF A A4B4L1FR-S28R (MEDI-524) Modified Antibody

### Introduction

The objective of this study was to evaluate potential cross-reactivity of A4b4 and L1FR-528R (MEDI-524) with cryosections of human lung and skin tissue. In the human tissue cross-reactivity studies, a fluoresceinated form of the A4b4 and L1FR-528R antibodies were used to evaluate binding: A4b4-FITC and L1FR-528R-FITC.

The preliminary studies which determined the reagent concentrations and staining conditions to be employed in the tissue cross reactivity study, and the tissue cross-reactivity study itself were conducted in accordance with PAI Standard Operating Procedures (SOPs) and in “the spirit” of the GLP regulations of the US FDA (21 CFR Part 58 and subsequent amendments). However, the study was considered to be a research study and was conducted in compliance with the GLP regulations. The reagent concentrations determined by the preliminary studies were validated by reproducibility of the positive controls in the tissue cross reactivity study.

### Materials and Methods

In order to detect binding, the unconjugated A4b4, and L1FR-S28R, or the FITC-conjugated A4b4-FITC and L1FR-S28R-FITC were applied to normal human tissues (one source per tissue) at two concentrations (10 μg/mL and 1 μg/mL). Tissues that had been obtained previously via necropsy or surgical biopsy were embedded in TISSUE-TEK® O.C.T. medium, frozen on dry ice, and stored in sealed plastic bags below −70° C. Tissues were sectioned at approximately 5 μm, fixed for 10 minutes in acetone, and placed in a desiccator to dry overnight. Slides were stored below −70° C. until staining. The slides were also fixed for 10 seconds in 10% NBF just prior to staining.

Purified RSV F UV-adhered to slides served as the positive control. Parathyroid hormone related protein (PTHrP) UV-adhered to slides was used as a negative control tissue. Other controls were produced by substitution of human antibody of the same immunoglobulin subclass (IgG1-kappa) but different antigenic specificity for the test article (negative control antibody), with or without conjugated FITC.

### Antibodies and Reagents

The following reagents were used in the study:
1. A4b4, a human IgG1 monoclonal antibody directed against RSV F protein, Lot No. 1411.153, PAI No. A3911, MedImmune, Inc, Gaithersburg, Md. The A4b4 antibody was FITC conjugated using routine methods known in the art, and was designated A4b4-FITC, Lot No. AD290502B, PAI No. A3843.
2. L1FR-S28R (MEDI-524), a human IgG1 monoclonal antibody directed against RSV F protein, Lot No. 1411.142, PAI No. A3912, MedImmune, Inc, Gaithersburg, Md. The L1FR-S28R antibody was FITC conjugated using routine methods known in the art, and was designated MEDI-493-FITC, Lot No. AD290502C, PAI No. A3842.
3. Negative control antibody human monoclonal IgG1 kappa antibody, Lot No. 071K9270, Sigma, St. Louis, Mo., PAI No. A3914. The unconjugated negative control antibody was FITC conjugated using routine methods known in the art, and was designated HuIgG-FITC, Lot No. AD280602, PAI No A3870.
4. Unconjugated mouse anti-fluorescein, Sigma, St. Louis, Mo., Lot No. 30K4884, PAI No. A3536.
5. ENVISION™ Kit, Dako, Carpinteria, Calif., Lot No. 06220, PAI No. K699.
6. Casein, Sigma, St. Louis, Mo., Lot No. 41K0165.
7. Sodium chloride, Sigma, St. Louis, Mo., Lot No. 121K16341.
8. Sodium phosphate, dibasic, Sigma, St. Louis, Mo., Lot No. 91K0117.
9. Potassium phosphate, monobasic, Sigma, St. Louis, Mo., Lot No. 101K0025.
10. Normal goat serum, Vector Laboratories, Burlingame, Calif., Lot No. N0805.
11. Acetone, VWR, West Chester, Pa., Lot No. 421622.
12.10% NBF, EM Science, Gibbstown, N.J., Lot No. 2145.
13. β-D(+) Glucose, Sigma, St. Louis, Mo., Lot No. 111K0024.
14. Human gamma-globulins, Sigma, St. Louis, Mo., Lot No. 12K7603.
15. Glucose Oxidase, Sigma, St. Louis, Mo., Lot No. 31K3800.
16. Bovine Serum Albumin, Sigma, St. Louis, Mo., Lot No. 22K1266.
17. Sodium Azide, Sigma, St. Louis, Mo., Lot No. 41K0236.
18. Trizma Base, Sigma, St. Louis, Mo., Lot No. 100K5433.
19. Avidin Biotin Blocking Kit, Vector Laboratories, Burlingame, Calif., Lot No. N0503.
20. Goat Anti-Human IgG, Fcγ fragment specific, Jackson Laboratories, West Grove, Pa., Lot No. 52408.
21. ABC “Elite” Kit, Vector Laboratories, Burlingame, Calif., Lot No. PK-6100, PAI No. K692.
22. DAB Tablets, Sigma, St. Louis, Mo., Lot No. 51K8211.
23. Norman Mouse Serum, Sigma, St. Louis, Mo., Lot No. 98F9407.
24. Sheared Salmon Sperm DNA, Eppendorf, Westbury, N.Y., Lot No. KL176A.
25. Hypercalcemia of Malignancy Factor (PTHrP) 1-34, Sigma, St. Louis, Mo., Lot No. 79H49582.
26. Frozen normal human tissues, National Research Disease Interchange, Philadelphia, Pa., Pathology Associates, Frederick, Md.
27. Frozen normal cotton rat tissues, supplied by Sponsor, MedImmune, Inc., Gaithersburg, Md.
28. Frozen normal cynomolgus monkey and chimpanzee lung, Pathology Associates—A Charles River Company, Frederick, Md.

### Tissue Staining Method

Table 37 depicts the immunoperoxidase staining method used.

TABLE 37
Immunoperoxidase Staining Method - Human Tissues

| | | Secondary | Tertiary | |
|---|---|---|---|---|
| Primary Antibody | | Antibody | Antibody | DAB |
| 1. | Test Article (MEDI-493-FITC, | X | X | X |
| | A4b4-FITC, or L1FR-S28R-FITC) | | | |
| | (2 concentrations) | | | |
| 2. | Negative Control Antibody | X | X | X |
| | (Human IgG1 kappa-FITC) | | | |
| | (2 concentrations) | | | |

An indirect immunoperoxidase procedure was performed. Acetone/formalin-fixed cryosections were rinsed in phosphate-buffered saline (PBS [0.3 M NaCl, pH 7.2]). Endogenous peroxidase was blocked by incubating the slides with the peroxidase solution provided in the Dako ENVISON™ Kit. Next, the slides were treated with a protein block designed to reduce nonspecific binding. The protein block was prepared as follows: PBS [0.3 M NaCl, pH 7.2]; 0.5% casein; 5% human gamma globulins; and 1 mg/mL heat-aggregated human IgG (prepared by heating a 5 mg/mL solution at 63° C. for 20 minutes and cooling to room temperature). Following the protein block, the test articles and the negative control antibody were applied to the slides for one hour at room temperature. Then, the slides were rinsed one time with TBS (0.15 M NaCl, pH 7.8) and two times with PBS (0.3 M NaCl, pH 7.2), and treated with the unconjugated secondary antibody (mouse anti-fluorescein) for 30 minutes at room temperature. Next, the slides were rinsed two times with PBS (0.3 M NaCl, pH 7.2), treated with the peroxidase-labeled goat anti-mouse IgG polymer supplied in the Dako ENVISION™ Kit for 30 minutes. Then, the slides were rinsed two times with PBS (0.3 M NaCl, pH 7.2), and treated with the substrate-chromogen (DAB) solution supplied in the Dako ENVISION™ Kit for 8 minutes. All slides were rinsed in water, counterstained with hematoxylin, dehydrated and coverslipped for interpretation.

TBS (0.15 M NaCl, pH 7.8)+5% human gamma globulins served as the diluent for all antibodies. In addition, 1 mg/mL heat-aggregated human IgG was added to the primary antibody diluent.

All slides were read by the a pathologist to identify the tissue or cell type stained and intensity of staining (graded±[equivocal], 1+[weak], 2+[moderate], 3+[strong], 4+[intense], Neg [negative]).

### Results and Discussion

### Human Tissue Positive and Negative Controls

The results are summarized in Table 38.

TABLE 38
Cross-Reactivity of A4b4-FITC, and L1FR-S28R-FITC with Normal
Human Tissues

| | | | | | | Negative | |
|---|---|---|---|---|---|---|---|
| | | | | | | Control | |
| | | | | | | α- | |
| | | | | L1FR- | | Human | |
| | | A4b4- | | S28R- | | IgG- | |
| | | FITC | | FITC | | FITC | |
| Tissue | Source | 10 μg/ml | 1 μg/ml | 10 μg/ml | 1 μg/ml | 10 μg/ml | 1 μg/ml |
| Purified RSV F | RSV F | 3-4+ | 2-3+ | 3-4+ | 2-3+ | Neg | Neg |
| Protein | Protein | | | | | | |
| (Positive Control) | | | | | | | |
| Purified PTHrP | PTHrP | Neg | Neg | Neg | Neg | Neg | Neg |
| Protein | Protein | | | | | | |
| (Negative Control) | | | | | | | |
| Lung | HT456 | | | | | | |
| Epithelium, alveolar | | 2-3+ | Neg | Neg | Neg | Neg | Neg |
| and bronchiolar | | (frequent)* | | | | | |
| (membrane and | | | | | | | |
| cytoplasm) | | | | | | | |
| Endothelium | | 2-3+ | Neg | Neg | Neg | Neg | Neg |
| (cytoplasm) | | (frequent)* | | | | | |
| Spindloid/dendritic | | 2-3+ | Neg | Neg | Neg | Neg | Neg |
| cells (cytoplasm) | | (occas)* | | | | | |
| Other elements | | Neg | Neg | Neg | Neg | Neg | Neg |
| Skin | HT545 | | | | | | |
| Epithelium, surface | | 1-2+ | Neg | Neg | Neg | Neg | Neg |
| and adnexal, basal | | (frequent)* | | | | | |
| layers (membrane and | | | | | | | |
| cytoplasm) | | | | | | | |
| Endothelium, dermal | | 1-2+ | Neg | Neg | Neg | Neg | Neg |
| vessels (cytoplasm) | | (frequent)* | | | | | |
| Other elements | | Neg | Neg | Neg | Neg | Neg | Neg |

1+ = weak,
2+ = moderate,
3+ = strong,
4+ = intense,
Neg = Negative,
*= Reactivity of uncertain specificity,

Using a direct immunoperoxidase method, the A4b4 and L1FR-S28R antibodies specifically stained the positive control purified RSV F, which had been UV-adhered to slides. Reactivity with positive control antigen and tissue elements was strong to intense at both concentrations examined (10 μg/mL and 1 μg/mL).

The he A4b4 and L1FR-S28R antibodies did not specifically react with negative control PTHrP which had been UV-adhered to slides. The negative control antibody HuIgG1-kappa, did not specifically react with either the positive control antigen (RSV F) or negative control antigen (PTHrP).

### Cross-Reactivity in Human Tissues

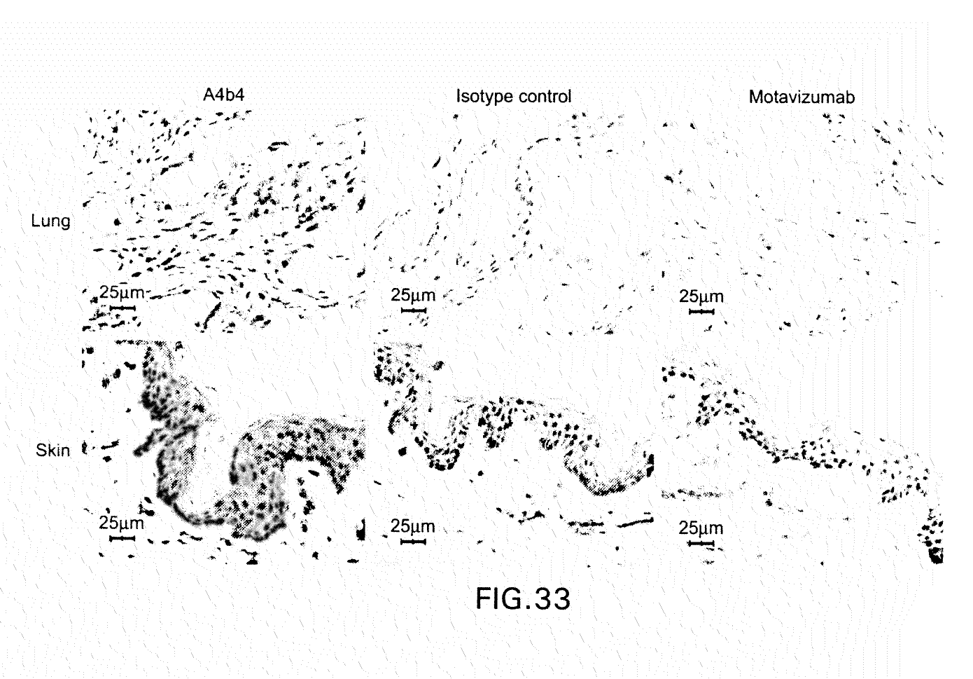

Results are shown in FIG. 33 and summarized in Table 38. The skin and lung tissues had staining that was interpreted to be cross-reactivity of uncertain specificity with the A4b4 antibody. The following cell types were observed to have this staining: alveolar and bronchiolar epithelium in the lung (moderate to strong staining at 10 μg/mL); surface and adnexal epithelium in the skin (weak to moderate staining at 10 μg/mL); and endothelium of dermal vessels (weak to moderate staining at 10 μg/mL). This staining was mostly observed at the highest concentration of test article.

However, in contrast to the results of A4b4, L1FR-S28R showed tissue cross-reactivity that was similar to the negative control isotype antibody.

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

### 8. SEQUENCE LISTING

The present specification is being filed with a computer readable form (CRF) copy of the Sequence Listing. The CRF entitled 10271-233-999_SeqList.txt, which was created on Oct. 31, 2005 and is 620,495 bytes in size, is identical to the paper copy of the Sequence Listing and is incorporated herein by reference in its entirety.

1. A method of preventing the progression of a respiratory syncytial virus (RSV) infection from the upper respiratory tract to the lower respiratory tract, the method comprising administering to a human patient an effective amount of an antibody that immunospecifically binds to a RSV F antigen, said antibody comprising:
(a) a heavy chain variable (VH) domain having the amino acid sequence SEQ ID NO:48;
(b) a VH chain having the amino acid sequence SEQ ID NO:254;
(c) VH domain comprising a VH CDR1 having the amino acid sequence SEQ ID NO:10, a VH CDR2 sequence having the amino acid sequence SEQ ID NO:19, and a VH CDR3 having the amino acid sequence SEQ ID NO:20;
(d) a light chain variable (VL) domain having the amino acid sequence SEQ ID NO:11;
(e) a VL chain having the amino acid sequence SEQ ID NO:255;
(f) VL domain comprising a VL CDR1 having the amino acid sequence SEQ ID NO:39, a VL CDR2 sequence having the amino acid sequence SEQ ID NO:5, and a VL CDR3 having the amino acid sequence SEQ ID NO:6;
(g) a VH domain having the amino acid sequence SEQ ID NO:48, and
a VL domain having the amino acid sequence SEQ ID NO:11;
(h) a VH domain having the amino acid sequence SEQ ID NO:48, and
a VL chain having the amino acid sequence of SEQ ID NO:255;
(i) a VH domain having the amino acid sequence SEQ ID NO:48, and
a VL domain comprising a VL CDR1 having the amino acid sequence SEQ ID NO:39, a VL CDR2 sequence having the amino acid sequence SEQ ID NO:5, and a VL CDR3 having the amino acid sequence SEQ ID NO:6;
(j) a VH chain having the amino acid sequence of SEQ ID NO:254, and
a VL domain having the amino acid sequence SEQ ID NO:11;
(k) a VH chain having the amino acid sequence of SEQ ID NO:254, and
a VL chain having the amino acid sequence of SEQ ID NO:255;
(l) a VH chain having the amino acid sequence of SEQ ID NO:254, and
a VL domain comprising a VL CDR1 having the amino acid sequence SEQ ID NO:39, a VL CDR2 sequence having the amino acid sequence SEQ ID NO:5, and a VL CDR3 having the amino acid sequence SEQ ID NO:6; or
(m) a VH CDR1 having the amino acid sequence SEQ ID NO:10, a VH CDR2 sequence having the amino acid sequence SEQ ID NO:19, a VH CDR3 having the amino acid sequence SEQ ID NO:20, a VL CDR1 having the amino acid sequence SEQ ID NO:39, a VL CDR2 sequence having the amino acid sequence SEQ ID NO:5, and a VL CDR3 having the amino acid sequence SEQ ID NO:6.

2. A method of preventing, managing, treating and/or ameliorating an RSV upper respiratory tract infection or otitis media, or a symptom thereof, the method comprising administering to a human patient an effective amount of an antibody that immunospecifically binds to a RSV F antigen, said antibody comprising:
(a) a heavy chain variable (VH) domain having the amino acid sequence SEQ ID NO:48;
(b) a VH chain having the amino acid sequence SEQ ID NO:254;
(c) VH domain comprising a VH CDR1 having the amino acid sequence SEQ ID NO:10, a VH CDR2 sequence having the amino acid sequence SEQ ID NO:19, and a VH CDR3 having the amino acid sequence SEQ ID NO:20;
(d) a light chain variable (VL) domain having the amino acid sequence SEQ ID NO:11;
(e) a VL chain having the amino acid sequence SEQ ID NO:255;
(f) VL domain comprising a VL CDR1 having the amino acid sequence SEQ ID NO:39, a VL CDR2 sequence having the amino acid sequence SEQ ID NO:5, and a VL CDR3 having the amino acid sequence SEQ ID NO:6:
(g) a VH domain having the amino acid sequence SEQ ID NO:48, and
a VL domain having the amino acid sequence SEQ ID NO:11;
(h) a VH domain having the amino acid sequence SEQ ID NO:48, and
a VL chain having the amino acid sequence of SEQ ID NO:255;
(i) a VH domain having the amino acid sequence SEQ ID NO:48, and
a VL domain comprising a VL CDR1 having the amino acid sequence SEQ ID NO:39, a VL CDR2 sequence having the amino acid sequence SEQ ID NO:5, and a VL CDR3 having the amino acid sequence SEQ ID NO:6;
(j) a VH chain having the amino acid sequence of SEQ ID NO:254, and
a VL domain having the amino acid sequence SEQ ID NO:11;
(k) a VH chain having the amino acid sequence of SEQ ID NO:254, and
a VL chain having the amino acid sequence of SEQ ID NO:255;
(l) a VH chain having the amino acid sequence of SEQ ID NO:254, and
a VL domain comprising a VL CDR1 having the amino acid sequence SEQ ID NO:39, a VL CDR2 sequence having the amino acid sequence SEQ ID NO:5, and a VL CDR3 having the amino acid sequence SEQ ID NO:6; or
(m) a VH CDR1 having the amino acid sequence SEQ ID NO:10, a VH CDR2 sequence having the amino acid sequence SEQ ID NO:19, a VH CDR3 having the amino acid sequence SEQ ID NO:20, a VL CDR1 having the amino acid sequence SEQ ID NO:39, a VL CDR2 sequence having the amino acid sequence SEQ ID NO:5, and a VL CDR3 having the amino acid sequence SEQ ID NO:6.

3. A method of preventing, managing, treating and/or ameliorating a respiratory condition associated with a RSV infection, the method comprising administering to a human patient an effective amount of an antibody that immuno specifically binds to a RSV F antigen, said antibody comprising:
(a) a heavy chain variable (VH) domain having the amino acid sequence SEQ ID NO:48;
(b) a VH chain having the amino acid sequence SEQ ID NO:254;
(c) VH domain comprising a VH CDR1 having the amino acid sequence SEQ ID NO:10, a VH CDR2 sequence having the amino acid sequence SEQ ID NO:19, and a VH CDR3 having the amino acid sequence SEQ ID NO:20;
(d) a light chain variable (VL) domain having the amino acid sequence SEQ ID NO:11;
(e) a VL chain having the amino acid sequence SEQ ID NO:255;
(f) VL domain comprising a VL CDR1 having the amino acid sequence SEQ ID NO:39, a VL CDR2 sequence having the amino acid sequence SEQ ID NO:5, and a VL CDR3 having the amino acid sequence SEQ ID NO:6;
(g) a VH domain having the amino acid sequence SEQ ID NO:48, and
a VL domain having the amino acid sequence SEQ ID NO:11;
(h) a VH domain having the amino acid sequence SEQ ID NO:48, and
a VL chain having the amino acid sequence of SEQ ID NO:255;
(i) a VH domain having the amino acid sequence SEQ ID NO:48, and
a VL domain comprising a VL CDR1 having the amino acid sequence SEQ ID NO:39, a VL CDR2 sequence having the amino acid sequence SEQ ID NO:5, and a VL CDR3 having the amino acid sequence SEQ ID NO:6;
(j) a VH chain having the amino acid sequence of SEQ ID NO:254, and
a VL domain having the amino acid sequence SEQ ID NO:11;
(k) a VH chain having the amino acid sequence of SEQ ID NO:254, and
a VL chain having the amino acid sequence of SEQ ID NO:255;
(l) a VH chain having the amino acid sequence of SEQ ID NO:254, and
a VL domain comprising a VL CDR1 having the amino acid sequence SEQ ID NO:39, a VL CDR2 sequence having the amino acid sequence SEQ ID NO:5, and a VL CDR3 having the amino acid sequence SEQ ID NO:6; or
(m) a VH CDR1 having the amino acid sequence SEQ ID NO:10, a VH CDR2 sequence having the amino acid sequence SEQ ID NO:19, a VH CDR3 having the amino acid sequence SEQ ID NO:20, a VL CDR1 having the amino acid sequence SEQ ID NO:39, a VL CDR2 sequence having the amino acid sequence SEQ ID NO:5, and a VL CDR3 having the amino acid sequence SEQ ID NO:6.

4. The method of claim 1, wherein antibody comprises an IgG constant domain.

5. The method of claim 4, wherein the IgG constant domain is a human IgG1 constant domain.

6. The method of claim 5, wherein the IgG1 constant domain comprises a tyrosine at position 252, a threonine at position 254 is a threonine, and a glutamic acid at position 256, numbered according to the EU Index as in Kabat et al. (1991). Sequences of proteins of immunological interest. (U.S. Department of Health and Human Services, Washington, D.C.) 5^{th} ed.

7-26. (canceled)

27. The method of claim 1, wherein the human has had a bone marrow transplant, or has cystic fibrosis, bronchopulmonary dysplasia, congenital heart disease, chronic heart disease, chronic lung disease, congenital immunodeficiency or acquired immunodeficiency, or is an elderly human.

28. The method of claim 6, wherein the human has had a bone marrow transplant, or has cystic fibrosis, bronchopulmonary dysplasia, congenital heart disease, chronic heart disease, chronic lung disease, congenital immunodeficiency or acquired immunodeficiency, or is an elderly human.

29. The method of claim 1, wherein the human is a human infant or a human infant born prematurely.

30. The method of claim 6, wherein the human is a human infant or a human infant born prematurely.

31-42. (canceled)

43. The method of claim 2, wherein antibody comprises an IgG constant domain.

44. The method of claim 43, wherein the IgG constant domain is a human IgG1 constant domain.

45. The method of claim 44, wherein the IgG1 constant domain comprises a tyrosine at position 252, a threonine at position 254 is a threonine, and a glutamic acid at position 256, numbered according to the EU Index as in Kabat et al. (1991). Sequences of proteins of immunological interest. (U.S. Department of Health and Human Services, Washington, D.C.) 5^{th} ed.

46. The method of claim 2, wherein the human has had a bone marrow transplant, or has cystic fibrosis, bronchopulmonary dysplasia, congenital heart disease, chronic heart disease, chronic lung disease, congenital immunodeficiency or acquired immunodeficiency, or is an elderly human.

47. The method of claim 45, wherein the human has had a bone marrow transplant, or has cystic fibrosis, bronchopulmonary dysplasia, congenital heart disease, chronic heart disease, chronic lung disease, congenital immunodeficiency or acquired immunodeficiency, or is an elderly human.

48. The method of claim 2, wherein the human is a human infant or a human infant born prematurely.

49. The method of claim 45, wherein the human is a human infant or a human infant born prematurely.

50. The method of claim 3, wherein antibody comprises an IgG constant domain.

51. The method of claim 50, wherein the IgG constant domain is a human IgG1 constant domain.

52. The method of claim 51, wherein the IgG1 constant domain comprises a tyrosine at position 252, a threonine at position 254 is a threonine, and a glutamic acid at position 256, numbered according to the EU Index as in Kabat et al. (1991). Sequences of proteins of immunological interest. (U.S. Department of Health and Human Services, Washington, D.C.) 5^{th} ed.

53. The method of claim 3, wherein the human has had a bone marrow transplant, or has cystic fibrosis, bronchopulmonary dysplasia, congenital heart disease, chronic heart disease, chronic lung disease, congenital immunodeficiency or acquired immunodeficiency, or is an elderly human.

54. The method of claim 52, wherein the human has had a bone marrow transplant, or has cystic fibrosis, bronchopulmonary dysplasia, congenital heart disease, chronic heart disease, chronic lung disease, congenital immunodeficiency or acquired immunodeficiency, or is an elderly human.

55. The method of claim 3, wherein the human is a human infant or a human infant born prematurely.

56. The method of claim 52, wherein the human is a human infant or a human infant born prematurely.
